SEQUENCE LISTING (194 sequences)

SEQ ID NO: 1 - protein, 671 aa, synthetic construct
MGWSCIILFLVATATGVHSEVQLVESGGGLVQPGGSLRLSCAASGSTFYTAVMGWVRQAPGKGLEWVAAIRWTALTTSYADSVKGRFTISRDGAKTTLYLQMNSLRPEDTAVYYCAARGTLGLFTTADSYDYWGQGTLVTVSSGGGGSGGGSGGVYCGPEFDESVGCMGGGGSGGGLSGRSDAGSPLGLAGSGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSEVQLVESGGGLVQPGGSLRLSCAASGFSFSNYYMAWVRQAPGKGLEWVASISTGGGNIYYRDSVKGRFTISRDNAKNSLYLQMNSLRAEDTAVYYCARQTAYYVMDAWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 2 - protein, 275 aa, synthetic construct
MGWSCIILFLVATATGVHSGGWSCAPDEDTWLCPAGGGGSGGLSGRSDAGSPLGLAGSGGSDIQMTQSPSSLSASVGDTVTITCLATEDIFSYLAWYQQKPGKAPKLLIYGANRLKDTVPSRFSGGGSGTEYSLTISGLQPEDFGTYYCLQGAKFPLTFGQGTKLDMKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 19 aa, synthetic construct
MGWSCIILFLVATATGVHS

SEQ ID NO: 4 - protein, 5 aa, synthetic construct
TAVMG

SEQ ID NO: 5 - protein, 18 aa, synthetic construct
AAIRWTALTTSYADSVKG

SEQ ID NO: 6 - protein, 15 aa, synthetic construct
RGTLGLFTTADSYDY

SEQ ID NO: 7 - protein, 18 aa, synthetic construct
GGVYCGPEFDESVGCMGG

SEQ ID NO: 8 - protein, 15 aa, synthetic construct
LSGRSDAGSPLGLAG

SEQ ID NO: 9 - protein, 4 aa, synthetic construct
KYAM

SEQ ID NO: 10 - protein, 19 aa, synthetic construct
RIRSKYNNYATYYADSVKD

SEQ ID NO: 11 - protein, 14 aa, synthetic construct
HGNFGNSYISYWAY

SEQ ID NO: 12 - protein, 8 aa, synthetic construct
GSSTGAVT

SEQ ID NO: 13 - protein, 7 aa, synthetic construct
GTKFLAP

SEQ ID NO: 14 - protein, 9 aa, synthetic construct
VLWYSNRWV

SEQ ID NO: 15 - protein, 5 aa, synthetic construct
NYYMA

SEQ ID NO: 16 - protein, 17 aa, synthetic construct
SISTGGGNIYYRDSVKG

SEQ ID NO: 17 - protein, 9 aa, synthetic construct
QTAYYVMDA

SEQ ID NO: 18 - protein, 18 aa, synthetic construct
GGWSCAPDEDTWLCPAGG

SEQ ID NO: 19 - protein, 15 aa, synthetic construct
LSGRSDAGSPLGLAG

SEQ ID NO: 20 - protein, 11 aa, synthetic construct
LATEDIFSYLA

SEQ ID NO: 21 - protein, 7 aa, synthetic construct
GANRLKD

SEQ ID NO: 22 - protein, 9 aa, synthetic construct
LQGAKFPLT

SEQ ID NO: 23 - protein, 19 aa, synthetic construct
GGWFCAPDENTWLCPNAGG

SEQ ID NO: 24 - protein, 107 aa, synthetic construct
EIQMTQSHKTFSVSTGQRVTITCKASQDVSIAVAWYQQKPGKAPKLLIYWASTRQTGVPSRFSGSGSGTDFTLTISCVQSEDFAVYYCQQYSSYPFTFGQGTKLEIK

SEQ ID NO: 25 - protein, 107 aa, synthetic construct
DIQMTQSPSFLSASVGDRVTITCKASQDVSIAVAWYQQKPGKAPKLLIYWASTRQTGVPSRFSGSGSGTDFTLTISSLQPEDFATYFCQQYSSYPFTFGQGTKLEIK

SEQ ID NO: 26 - protein, 107 aa, synthetic construct
DIQMTQSPSFLSASVGERVTITCKASQDVSIAVAWYQQKPGKAPKLLIYWASTRQTGVPSRFSGSGSGTDFTLTISSLQPEDIATYFCQQYSSYPFTFGQGTKLEIK

SEQ ID NO: 27 - protein, 107 aa, synthetic construct
DIQMTQSPSFLSASVGDRVTITCKASQDVSIAVAWYQQKPGKAPKLLIYWASTRQTGVPSRFSGSGSGTDFTLTISSLQPEDSATYFCQQYSSYPFTFGQGTKVDIK

SEQ ID NO: 28 - protein, 11 aa, synthetic construct
KASQDVSIAVA

SEQ ID NO: 29 - protein, 7 aa, synthetic construct
WASTRQT

SEQ ID NO: 30 - protein, 9 aa, synthetic construct
QQYSSYPFT

SEQ ID NO: 31 - protein, 107 aa, synthetic construct
EIQMTQSQSTLSVSLGDRVTITCKASQNVVTAVAWYQQKPGQAPKLLIYSASHRFTGVPSRFSGSGSGTDFTLIISCMQSEDFAVYYCQQYSSYPYTFGQGTKLEIK

SEQ ID NO: 32 - protein, 107 aa, synthetic construct
DIQMTQSPSFLSASVGDRVTITCKASQNVVTAVAWYQQKPGKAPKLLIYSASHRFTGVPSRFSGSGSGTDFTLTISNLQSEDLATYFCQQYSSYPYTFGQGTKLEIK

SEQ ID NO: 33 - protein, 107 aa, synthetic construct
DIQMTQSPSFLSASVGDRVTITCKASQNVVTAVAWYQQKPGKAPKLLIYSASHRFTGVPSRFSGSGSGTDFTLTISNLQPEDSATYFCQQYSSYPYTFGQGTKLEIK

SEQ ID NO: 34 - protein, 107 aa, synthetic construct
DIQMTQSPSFLSASVGDRVTITCKASQNVVTAVAWYQQKPGKAPKLLIYSASHRFTGVPSRFSGSGSGTDFTLTISNLQPDDAATYFCQQYSSYPYTFGQGTKLEIK

SEQ ID NO: 35 - protein, 11 aa, synthetic construct
KASQNVVTAVA

SEQ ID NO: 36 - protein, 7 aa, synthetic construct
SASHRFT

SEQ ID NO: 37 - protein, 9 aa, synthetic construct
QQYSSYPYT

SEQ ID NO: 38 - protein, 107 aa, synthetic construct
AIQMTQSHKSFSVSTGQRVTITCKASQDVSTTVAWYQQKPGQAPKLLIYWASTRQTGVPSRFTGSGSGTDFTLTISCVQSEDFAVYYCQQYSSYPFTFGQGTKLEIK

SEQ ID NO: 39 - protein, 107 aa, synthetic construct
DIQMTQSPSFLSASVGDRVTITCKASQDVSTTVAWYQQKPGKAPKLLIYWASTRQTGVPSRFSGSGSGTDFTLTISNLQPEDFATYFCQQYSSYPFTFGQGTKLEIK

SEQ ID NO: 40 - protein, 107 aa, synthetic construct
DIQMTQSPSFLSASVGERVTITCKASQDVSTTVAWYQQKPGKAPKLLIYWASTRQTGVPSRFSGSGSGTDFTLTINSLQPEDVATYFCQQYSSYPFTFGQGTKLEIK

SEQ ID NO: 41 - protein, 107 aa, synthetic construct
DIQMTQSPSFLSASVGERVTITCKASQDVSTTVAWYQQKPGKAPKLLIYWASTRQTGVPSRFSGSGSGTDFTLTISNLQPEDVATYFCQQYSSYPFTFGQGTKLEIK

SEQ ID NO: 42 - protein, 11 aa, synthetic construct
KASQDVSTTVA

SEQ ID NO: 43 - protein, 107 aa, synthetic construct
DIQMTQSPSSVSLTVGQRVTITCLATEDIFSYLAWYQQKPGKAPRLLIYGANRLKDGVPSRFSGSGSGTQYTLRISSMQPEDFGVYYCLQGAKFPLTFGPGTKLEIK

SEQ ID NO: 44 - protein, 107 aa, synthetic construct
DIQMTQSPSSLSASIGDTVTITCLATEDIFSYLAWYQQRPGKAPKLLIYGANRLKDGVPSRFSGSGSGTDYTLTISGLQPEDFVTYYCLQGAKFPLTFGQGTKLDMK

SEQ ID NO: 45 - protein, 107 aa, synthetic construct
DIQMTQSPSSLSASVGDTVTITCLATEDIFSYLAWYQQKPGKAPKLLIYGANRLKDGVPSRFSGSGSGTDYTLTISGLQPEDFGTYYCLQGAKFPLTFGQGTKLEIK

SEQ ID NO: 46 - protein, 107 aa, synthetic construct
DIQMTQSPSSLSASVGDTVTITCLATEDIFSYLAWYQQKPGKAPKLLIYGANRLKDGVPSRFSGGGSGTEYSLTISGLQPEDFGTYYCLQGAKFPLTFGQGTKLDMK

SEQ ID NO: 47 - protein, 108 aa, synthetic construct
DIQMTQSPSSLSASVGDTVTITCLATEDIFSYLAWYQQKPGKAPKLLIYGANRLKDTVPSRFSGGGSGTEYSLTISGLQPEDFGTYYCLQGAKFPLTFGQGTKLDMKR

SEQ ID NO: 48 - protein, 122 aa, synthetic construct
QVQLLESGGELVQPGGSLRLSCAASGFTFSDYYMFWIRQTPQKRLEWVAYISNGGGNTYYPDTVKGRFTISRDNAKNQLYLQMRSLTPEDTAIYYCASPEARYYGNYPFPYWGQGTLVTVSS

SEQ ID NO: 49 - protein, 122 aa, synthetic construct
QVQLVESGGGLVKPGGSLRLSCAASGFTFSDYYMFWIRQAPGKGLEWVAYISNGGGNTYYPDTVKGRFTISRDNAKNTLYLQMNSLRAEDTAVYYCASPEARYYGNYPFPYWGQGTLVTVSS

SEQ ID NO: 50 - protein, 122 aa, synthetic construct
QVQLVESGGGVVKPGGSLRLSCAASGFTFSDYYMFWIRQAPGKGLEWVAYISNGGGNTYYPDTVKGRFTISRDNAKNTLYLQMNSLRAEDTAVYYCASPEARYYGNYPFPYWGQGTLVTVSS

SEQ ID NO: 51 - protein, 122 aa, synthetic construct
QVQLVESGGGVVQPGGSLRLSCAASGFTFSDYYMFWIRQAPGKGLEWVAYISNGGGNTYYPDTVKGRFTISRDNAKNTLYLQMNSLRAEDTAVYYCASPEARYYGNYPFPYWGQGTLVTVSS

SEQ ID NO: 52 - protein, 5 aa, synthetic construct
DYYMF

SEQ ID NO: 53 - protein, 17 aa, synthetic construct
YISNGGGNTYYPDTVKG

SEQ ID NO: 54 - protein, 13 aa, synthetic construct
PEARYYGNYPFPY

SEQ ID NO: 55 - protein, 116 aa, synthetic construct
QVQLLQSGPGLVKPSATLSLTCTVSGFSLTTYGVHWIRQPPGKGLEWMGVIWSGGSTDYNAAFISRLTISKDNSKNQVFLQLYSLRAEDTAIYYCARTSHWYFDVWGRGTLVTVSS

SEQ ID NO: 56 - protein, 116 aa, synthetic construct
QVQLQESGPGLVKPSQTLSLTCTVSGFSLTTYGVHWVRQSPGKGLEWLGVIWSGGSTDYNAAFISRLSISKDNSKSQVFLEMTSLTAADTAIYYCARTSHWYFDVWGRGTLVTVSS

SEQ ID NO: 57 - protein, 116 aa, synthetic construct
QVQLQESGPGLVKPSQTLSLTCTVSGFSLTTYGVHWVRQAPEKGLEWLGVIWSGGSTDYNAAFISRLSISKDNSKSQVFLEMTSLTADDTAIYYCARTSHWYFDVWGRGTLVTVSS

SEQ ID NO: 58 - protein, 116 aa, synthetic construct
QVQLQESGPGLVKPSQTLSLTCTVSGFSLTTYGVHWVRQTPEKGLEWLGVIWSGGSTDYNAAFISRLSISKDNSKSQVFLEMTSLTADDTAIYYCARTSHWYFDVWGRGTLVTVSS

SEQ ID NO: 59 - protein, 5 aa, synthetic construct
TYGVH

SEQ ID NO: 60 - protein, 16 aa, synthetic construct
VIWSGGSTDYNAAFIS

SEQ ID NO: 61 - protein, 8 aa, synthetic construct
TSHWYFDV

SEQ ID NO: 62 - protein, 122 aa, synthetic construct
QVQLLESGGGLVQPGGSLRLSCAASGFTFSDYYMYWIRQTPQKRLEWVAYISNGGGNTYYSDTVKGRFTISRDNAKNQLYLQMRSLRPEDTAIYYCASPEARYYGNFPFPYWGQGTLVTVSS

SEQ ID NO: 63 - protein, 122 aa, synthetic construct
QVQLVESGGGLVKPGGSLRLSCAASGFTFSDYYMYWIRQAPGKGLEWVAYISNGGGNTYYSDTVKGRFTISRDNAKNTLYLQMNSLRAEDTAVYYCASPEARYYGNFPFPYWGQGTLVTVSS

SEQ ID NO: 64 - protein, 122 aa, synthetic construct; identical to SEQ ID NO: 63

SEQ ID NO: 65 - protein, 122 aa, synthetic construct; identical to SEQ ID NO: 63

SEQ ID NO: 66 - protein, 5 aa, synthetic construct
DYYMY

SEQ ID NO: 67 - protein, 17 aa, synthetic construct
YISNGGGNTYYSDTVKG

SEQ ID NO: 68 - protein, 13 aa, synthetic construct
PEARYYGNFPFPY

SEQ ID NO: 69 - protein, 118 aa, synthetic construct
QVQLVESGGGVVQPGRSLRLSCAASGFSFSNYYMAWVRQAPGKGLEWVASISTGGGNIYYRDSVKGRFTISRDNSKSTLYLQMRSLRSEDTAIYYCARQTAYYVMDAWGQGTMVTVSS

SEQ ID NO: 70 - protein, 118 aa, synthetic construct
EVQLVESGGGLVQPGGSLRLSCAASGFSFSNYYMAWVRQAPGKGLEWVASISTGGGNIYYRDSVKGRFTISRDNAKNSLYLQMNSLRAEDTAVYYCARQTAYYVMDAWGQGTTVTVSS

SEQ ID NO: 71 - protein, 118 aa, synthetic construct; identical to SEQ ID NO: 70

SEQ ID NO: 72 - protein, 118 aa, synthetic construct; identical to SEQ ID NO: 70

SEQ ID NO: 73 - protein, 107 aa, synthetic construct
DIQMTQSPASLSVYLGETVSIECLATEDIFSYLAWYQQKPGKSPQLLIYGANRLKDGVPSRFSGSGTGTQYSLRISGMQPEDEGDYYCLQGAKFPLTFGSGTKLEIK

SEQ ID NO: 74 - protein, 107 aa, synthetic construct
DIQMTQSPASLSASLGETVSIECLASEDIYSYLAWFQQKSGKSPQLLIYAANRLQDGVPSRFSGSGSGTQYSLRISGMQPEDEGDYFCLQGSQFPWTFGGGTKLELK

SEQ ID NO: 75 - protein, 107 aa, synthetic construct
DIQMTQSPASLSASLGETVSIECLASEDIHNKLAWYQQKPGKSPQLLIYYGSNLQDGVPSRFSGSGSGTQYYLKINSLESEDVATYFCLQDSKNPWTFGGGTKLEMK

SEQ ID NO: 76 - protein, 110 aa, synthetic construct
DIVLTQSPALDVSLGQRATISCSASQSVSISRYNLIHWYQQKPGQQPKLLIWRTSILTSGIPARFSGRGSGTDFTLTINPVQADDIATYYCQQSRESPFTFGAGTRLELK

SEQ ID NO: 77 - protein, 107 aa, synthetic construct
NIVLTQSPATLSVTPGESVSLSCRASQSISTGIHWYQQKSNESPRLLIKFASQSISGIPSRFSGSGSGTDFTLSINRVESEDFSIYYCQQRDSSLFTFGAGTKLELK

SEQ ID NO: 78 - protein, 108 aa, synthetic construct
TYELIQPPSTSVTLGNTVSLTCVGDDLPRRYAYWYQQKPDQSIVRVIYEDSKRPSGISDRFSGSSSGTTATLTIRDAQAEDEADYYCHSTYSDDKVRVFGGGTKLTVL

SEQ ID NO: 79 - protein, 118 aa, synthetic construct
EVQLVESGGGLVQPGRSMRLSCAASGFSFSNYYMAWVRQAPRKGLDWVASISTGGGNIYYRDSVKGRFTISRDNAKSTLYLQMDSLRSEDTATYYCARQTAYYVMDAWGQGASVTVSS

SEQ ID NO: 80 - protein, 119 aa, synthetic construct
EVQLVESGGGLVQPGRSLKLSCAASGFTFSNYDMAWVRQVPTKGLEWVASISPRGGRIYYRDSVKGRFTVSRDNAKSCLYLQMESLRSDDTATYYCARIHHGYWYFDFWGPGTMVTVSS

SEQ ID NO: 81 - protein, 121 aa, synthetic construct
EVKLLESGGGLVQPGGSMRLSCAASGFTFTDFYMIWIRQPAGKAPEWLGFIRNKANGYTTDYNPSVKGRFTISRDNTQNMLYLQMITLRAEDTATYYCARPLYYGYTPRYWGQGVMVTVSS

SEQ ID NO: 82 - protein, 120 aa, synthetic construct
EVQLVESGGGLVQPGRPLKLSCAASGFSFSHYDMAWVRQAPTKGLEWVAAISPSGGSTYYRDSVKGRFTVSRDKAKNSLYLQMDSLRSEDTATYYCARQGPSYGYYFDYWGQGVMVTVSS

SEQ ID NO: 83 - protein, 116 aa, synthetic construct
EVQLVESGGGLVQPGRSLKVSCAASGFTFSNYDMAWVRQAPTKGLEWVASINPGGISTYYRDSVKGRFTVSRDNEKSTLYLQMDSLRSEDTATYYCARRQPYFDYWGQGVMVTVSS

SEQ ID NO: 84 - protein, 119 aa, synthetic construct
QVTLKESGTGILQPSQALSLTCSISGFSLNTTGICVSWIRQPLGQGLEWLADICWDDGKGYNPSLKNRLSISKDTSNNQAFLKITRVDTTDTATYYCARNYGGNPFDYWGQGVMVTVSS

SEQ ID NO: 85 - protein, 11 aa, synthetic construct
LASEDIYSYLA

SEQ ID NO: 86 - protein, 7 aa, synthetic construct
AANRLQD

SEQ ID NO: 87 - protein, 9 aa, synthetic construct
LQGSQFPWT

SEQ ID NO: 88 - protein, 11 aa, synthetic construct
LASEDIHNKLA

SEQ ID NO: 89 - protein, 7 aa, synthetic construct
YGSNLQD

SEQ ID NO: 90 - protein, 9 aa, synthetic construct
LQDSKNPWT

SEQ ID NO: 91 - protein, 15 aa, synthetic construct
SASQSVSISRYNLIH

SEQ ID NO: 92 - protein, 7 aa, synthetic construct
RTSILTS

SEQ ID NO: 93 - protein, 9 aa, synthetic construct
QQSRESPFT

SEQ ID NO: 94 - protein, 11 aa, synthetic construct
RASQSISTGIH

SEQ ID NO: 95 - protein, 7 aa, synthetic construct
FASQSIS

SEQ ID NO: 96 - protein, 9 aa, synthetic construct
QQRDSSLFT

SEQ ID NO: 97 - protein, 11 aa, synthetic construct
VGDDLPRRYAY

SEQ ID NO: 98 - protein, 7 aa, synthetic construct
EDSKRPS

SEQ ID NO: 99 - protein, 11 aa, synthetic construct
HSTYSDDKVRV

SEQ ID NO: 100 - protein, 5 aa, synthetic construct
NYDMA

SEQ ID NO: 101 - protein, 17 aa, synthetic construct
SISPRGGRIYYRDSVKG

SEQ ID NO: 102 - protein, 10 aa, synthetic construct
IHHGYWYFDF

SEQ ID NO: 103 - protein, 5 aa, synthetic construct
DFYMI

SEQ ID NO: 104 - protein, 19 aa, synthetic construct
FIRNKANGYTTDYNPSVKG

SEQ ID NO: 105 - protein, 10 aa, synthetic construct
PLYYGYTPRY

SEQ ID NO: 106 - protein, 5 aa, synthetic construct
HYDMA

SEQ ID NO: 107 - protein, 17 aa, synthetic construct
AISPSGGSTYYRDSVKG

SEQ ID NO: 108 - protein, 11 aa, synthetic construct
QGPSYGYYFDY

SEQ ID NO: 109 - protein, 17 aa, synthetic construct
SINPGGISTYYRDSVKG

SEQ ID NO: 110 - protein, 7 aa, synthetic construct
RQPYFDY

SEQ ID NO: 111 - protein, 11 aa, synthetic construct
VGDDLPRRYAY

SEQ ID NO: 112 - protein, 7 aa, synthetic construct
EDSKRPS

SEQ ID NO: 113 - protein, 11 aa, synthetic construct
HSTYSDDKVRV

SEQ ID NO: 114 - protein, 122 aa, synthetic construct
EVQLQESGGDLVQPGGSLKLSCAASGFTFSDYYMFWIRQTPQKRLEWVAYISNGGGNTYYPDTVKGRFTISRDNAKNTLYLQMSRLKSEDTAMYYCASPEARYYGNYPFPYWGQGTLVTVSA

SEQ ID NO: 115 - protein, 107 aa, synthetic construct
DIVMTQSHKFMSTSVGGRVTITCKASQDVSIAVAWYQQKPGQSPKLLIYWASTRQTGVPDRFAGSGSGTDFTLTISNVQSEDLADYFCQQYSSYPFTFGSGTKLEIK

SEQ ID NO: 116 - protein, 116 aa, synthetic construct
QVQLKQSGPGLVQPSQSLSITCTVSGFSLTTYGVHWVRQSPGKGLEWLGVIWSGGSTDYNAAFISRLSISKDNSKSQVFFEMYSLQADDTAIYYCARTSHWYFDVWGTGTTVTVSS

SEQ ID NO: 117 - protein, 107 aa, synthetic construct
DIVMTQSQKFMSTTVGDRVSITCKASQNVVTAVAWYQQKPGQSPKLLIYSASHRFTGVPDRFTGSGSGTDFTLIISNMQSEDLADYFCQQYSSYPYTFGGGTKLEIK

SEQ ID NO: 118 - protein, 122 aa, synthetic construct
EVQLQESGGGLVQPGGSLKLSCAASGFTFSDYYMYWIRQTPQKRLEWVAYISNGGGNTYYSDTVKGRFTISRDNAKNTLYLQMSRLKSEDTAMYYCASPEARYYGNFPFPYWGQGTLVTVSA

SEQ ID NO: 119 - protein, 107 aa, synthetic construct
DIVMTQSHKFMSTSVGGRVTITCKASQDVSTTVAWYQQKPGQSPKLLIYWASTRQTGVPDRFTGSGSGTDFTLTISNVQSEDLADYFCQQYSSYPFTFGSGTKLEIK

SEQ ID NO: 120 - protein, 8 aa, synthetic construct
GFTFSDYY

SEQ ID NO: 121 - protein, 15 aa, synthetic construct
ASPEARYYGNYPFPY

SEQ ID NO: 122 - protein, 8 aa, synthetic construct
GFSLTTYG

SEQ ID NO: 123 - protein, 10 aa, synthetic construct
ARTSHWYFDV

SEQ ID NO: 124 - protein, 15 aa, synthetic construct
ASPEARYYGNFPFPY

SEQ ID NO: 125 - protein, 18 aa, synthetic construct
GGWTCADDEDTWLCPIGG

SEQ ID NO: 126 - protein, 18 aa, synthetic construct
GGWACAPDEDTWLCPIGG

SEQ ID NO: 127 - protein, 214 aa, synthetic construct
EIQMTQSHKTFSVSTGQRVTITCKASQDVSIAVAWYQQKPGKAPKLLIYWASTRQTGVPSRFSGSGSGTDFTLTISCVQSEDFAVYYCQQYSSYPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 128 - protein, 214 aa, synthetic construct
DIQMTQSPSFLSASVGDRVTITCKASQDVSIAVAWYQQKPGKAPKLLIYWASTRQTGVPSRFSGSGSGTDFTLTISSLQPEDFATYFCQQYSSYPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 129 - protein, 214 aa, synthetic construct
DIQMTQSPSFLSASVGERVTITCKASQDVSIAVAWYQQKPGKAPKLLIYWASTRQTGVPSRFSGSGSGTDFTLTISSLQPEDIATYFCQQYSSYPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 130 - protein, 214 aa, synthetic construct
DIQMTQSPSFLSASVGDRVTITCKASQDVSIAVAWYQQKPGKAPKLLIYWASTRQTGVPSRFSGSGSGTDFTLTISSLQPEDSATYFCQQYSSYPFTFGQGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 131 - protein, 214 aa, synthetic construct
EIQMTQSQSTLSVSLGDRVTITCKASQNVVTAVAWYQQKPGQAPKLLIYSASHRFTGVPSRFSGSGSGTDFTLIISCMQSEDFAVYYCQQYSSYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 132 - protein, 214 aa, synthetic construct
DIQMTQSPSFLSASVGDRVTITCKASQNVVTAVAWYQQKPGKAPKLLIYSASHRFTGVPSRFSGSGSGTDFTLTISNLQSEDLATYFCQQYSSYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 133 - protein, 214 aa, synthetic construct
DIQMTQSPSFLSASVGDRVTITCKASQNVVTAVAWYQQKPGKAPKLLIYSASHRFTGVPSRFSGSGSGTDFTLTISNLQPEDSATYFCQQYSSYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 134 - protein, 214 aa, synthetic construct
DIQMTQSPSFLSASVGDRVTITCKASQNVVTAVAWYQQKPGKAPKLLIYSASHRFTGVPSRFSGSGSGTDFTLTISNLQPDDAATYFCQQYSSYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 135 - protein, 214 aa, synthetic construct
AIQMTQSHKSFSVSTGQRVTITCKASQDVSTTVAWYQQKPGQAPKLLIYWASTRQTGVPSRFTGSGSGTDFTLTISCVQSEDFAVYYCQQYSSYPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 136 - protein, 214 aa, synthetic construct
DIQMTQSPSFLSASVGDRVTITCKASQDVSTTVAWYQQKPGKAPKLLIYWASTRQTGVPSRFSGSGSGTDFTLTISNLQPEDFATYFCQQYSSYPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 137 - protein, 214 aa, synthetic construct
DIQMTQSPSFLSASVGERVTITCKASQDVSTTVAWYQQKPGKAPKLLIYWASTRQTGVPSRFSGSGSGTDFTLTINSLQPEDVATYFCQQYSSYPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 138 - protein, 214 aa, synthetic construct
DIQMTQSPSFLSASVGERVTITCKASQDVSTTVAWYQQKPGKAPKLLIYWASTRQTGVPSRFSGSGSGTDFTLTISNLQPEDVATYFCQQYSSYPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 139 - protein, 214 aa, synthetic construct
DIQMTQSPSSVSLTVGQRVTITCLATEDIFSYLAWYQQKPGKAPRLLIYGANRLKDGVPSRFSGSGSGTQYTLRISSMQPEDFGVYYCLQGAKFPLTFGPGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 140 - protein, 214 aa, synthetic construct
DIQMTQSPSSLSASIGDTVTITCLATEDIFSYLAWYQQRPGKAPKLLIYGANRLKDGVPSRFSGSGSGTDYTLTISGLQPEDFVTYYCLQGAKFPLTFGQGTKLDMKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 141 - protein, 214 aa, synthetic construct
DIQMTQSPSSLSASVGDTVTITCLATEDIFSYLAWYQQKPGKAPKLLIYGANRLKDGVPSRFSGSGSGTDYTLTISGLQPEDFGTYYCLQGAKFPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 142 - protein, 214 aa, synthetic construct
DIQMTQSPSSLSASVGDTVTITCLATEDIFSYLAWYQQKPGKAPKLLIYGANRLKDGVPSRFSGGGSGTEYSLTISGLQPEDFGTYYCLQGAKFPLTFGQGTKLDMKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 143 - protein, 214 aa, synthetic construct
DIQMTQSPSSLSASVGDTVTITCLATEDIFSYLAWYQQKPGKAPKLLIYGANRLKDTVPSRFSGGGSGTEYSLTISGLQPEDFGTYYCLQGAKFPLTFGQGTKLDMKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 144 - protein, 452 aa, synthetic construct
QVQLLESGGELVQPGGSLRLSCAASGFTFSDYYMFWIRQTPQKRLEWVAYISNGGGNTYYPDTVKGRFTISRDNAKNQLYLQMRSLTPEDTAIYYCASPEARYYGNYPFPYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 145 - protein, 452 aa, synthetic construct
QVQLVESGGGLVKPGGSLRLSCAASGFTFSDYYMFWIRQAPGKGLEWVAYISNGGGNTYYPDTVKGRFTISRDNAKNTLYLQMNSLRAEDTAVYYCASPEARYYGNYPFPYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 146 - protein, 452 aa, synthetic construct
QVQLVESGGGVVKPGGSLRLSCAASGFTFSDYYMFWIRQAPGKGLEWVAYISNGGGNTYYPDTVKGRFTISRDNAKNTLYLQMNSLRAEDTAVYYCASPEARYYGNYPFPYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 147 - protein, 452 aa, synthetic construct
QVQLVESGGGVVQPGGSLRLSCAASGFTFSDYYMFWIRQAPGKGLEWVAYISNGGGNTYYPDTVKGRFTISRDNAKNTLYLQMNSLRAEDTAVYYCASPEARYYGNYPFPYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 148 - protein, 446 aa, synthetic construct
QVQLLQSGPGLVKPSATLSLTCTVSGFSLTTYGVHWIRQPPGKGLEWMGVIWSGGSTDYNAAFISRLTISKDNSKNQVFLQLYSLRAEDTAIYYCARTSHWYFDVWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 149 - protein, 446 aa, synthetic construct
QVQLQESGPGLVKPSQTLSLTCTVSGFSLTTYGVHWVRQSPGKGLEWLGVIWSGGSTDYNAAFISRLSISKDNSKSQVFLEMTSLTAADTAIYYCARTSHWYFDVWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 150 - protein, 446 aa, synthetic construct
QVQLQESGPGLVKPSQTLSLTCTVSGFSLTTYGVHWVRQAPEKGLEWLGVIWSGGSTDYNAAFISRLSISKDNSKSQVFLEMTSLTADDTAIYYCARTSHWYFDVWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 151 - protein, 446 aa, synthetic construct
QVQLQESGPGLVKPSQTLSLTCTVSGFSLTTYGVHWVRQTPEKGLEWLGVIWSGGSTDYNAAFISRLSISKDNSKSQVFLEMTSLTADDTAIYYCARTSHWYFDVWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 152 - protein, 452 aa, synthetic construct
QVQLLESGGGLVQPGGSLRLSCAASGFTFSDYYMYWIRQTPQKRLEWVAYISNGGGNTYYSDTVKGRFTISRDNAKNQLYLQMRSLRPEDTAIYYCASPEARYYGNFPFPYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 153 - protein, 452 aa, synthetic construct
QVQLVESGGGLVKPGGSLRLSCAASGFTFSDYYMYWIRQAPGKGLEWVAYISNGGGNTYYSDTVKGRFTISRDNAKNTLYLQMNSLRAEDTAVYYCASPEARYYGNFPFPYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 154 - protein, 452 aa, synthetic construct; identical to SEQ ID NO: 153

SEQ ID NO: 155 - protein, 452 aa, synthetic construct; identical to SEQ ID NO: 153

SEQ ID NO: 156 - protein, 448 aa, synthetic construct
QVQLVESGGGVVQPGRSLRLSCAASGFSFSNYYMAWVRQAPGKGLEWVASISTGGGNIYYRDSVKGRFTISRDNSKSTLYLQMRSLRSEDTAIYYCARQTAYYVMDAWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 157 - protein, 448 aa, synthetic construct
EVQLVESGGGLVQPGGSLRLSCAASGFSFSNYYMAWVRQAPGKGLEWVASISTGGGNIYYRDSVKGRFTISRDNAKNSLYLQMNSLRAEDTAVYYCARQTAYYVMDAWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 158 - protein, 448 aa, synthetic construct; identical to SEQ ID NO: 157

SEQ ID NO: 159 - protein, 448 aa, synthetic construct; identical to SEQ ID NO: 157

SEQ ID NO: 160 - DNA, 642 bp, synthetic construct
  source 1..642: mol_type = other DNA
gagatccagatgacccagagccacaagaccttcagcgtgagcaccggccagagggtgaccatcacctgcaaggccagccaggacgtgagcatcgccgtggcctggtaccagcagaagcccggcaaggcccccaagctgctgatctactgggccagcaccaggcagaccggcgtgcccagcaggttcagcggcagcggcagcggcaccgacttcaccctgaccatcagctgcgtgcagagcgaggacttcgccgtgtactactgccagcagtacagcagctaccccttcaccttcggccagggcaccaagctggagatcaagcgaacggtggctgccccctccgtgttcatcttcccccccagcgatgagcagctgaagagcggcacagccagcgtggtgtgcctgctgaacaacttctaccccagggaggccaaggtgcagtggaaggtggacaacgccctgcagagcggcaacagccaggagtccgtgaccgagcaggacagcaaggacagcacctacagcctgagcagcacactgaccctgagcaaggccgactacgagaagcacaaggtgtacgcctgcgaggtgacccatcagggcctgagcagccccgtgaccaagagcttcaacaggggcgagtgc

SEQ ID NO: 161 - DNA, 642 bp, synthetic construct
  source 1..642: mol_type = other DNA
gacatccagatgacccagagccccagcttcctgagcgccagcgtgggcgacagggtgaccatcacctgcaaggccagccaggacgtgagcatcgccgtggcctggtaccagcagaagcccggcaaggcccccaagctgctgatctactgggccagcaccaggcagaccggcgtgcccagcaggttcagcggcagcggcagcggcaccgacttcaccctgaccatcagcagcctgcagcccgaggacttcgccacctacttctgccagcagtacagcagctaccccttcaccttcggccagggcaccaagctggagatcaagcgaacggtggctgccccctccgtgttcatcttcccccccagcgatgagcagctgaagagcggcacagccagcgtggtgtgcctgctgaacaacttctaccccagggaggccaaggtgcagtggaaggtggacaacgccctgcagagcggcaacagccaggagtccgtgaccgagcaggacagcaaggacagcacctacagcctgagcagcacactgaccctgagcaaggccgactacgagaagcacaaggtgtacgcctgcgaggtgacccatcagggcctgagcagccccgtgaccaagagcttcaacaggggcgagtgc

SEQ ID NO: 162 - DNA, 642 bp, synthetic construct
  source 1..642: mol_type = other DNA
gacatccagatgacccagagccccagcttcctgagcgccagcgtgggcgagagggtgaccatcacctgcaaggccagccaggacgtgagcatcgccgtggcctggtaccagcagaagcccggcaaggcccccaagctgctgatctactgggccagcaccaggcagaccggcgtgcccagcaggttcagcggcagcggcagcggcaccgacttcaccctgaccatcagcagcctgcagcccgaggacatcgccacctacttctgccagcagtacagcagctaccccttcaccttcggccagggcaccaagctggagatcaagcgaacggtggctgccccctccgtgttcatcttcccccccagcgatgagcagctgaagagcggcacagccagcgtggtgtgcctgctgaacaacttctaccccagggaggccaaggtgcagtggaaggtggacaacgccctgcagagcggcaacagccaggagtccgtgaccgagcaggacagcaaggacagcacctacagcctgagcagcacactgaccctgagcaaggccgactacgagaagcacaaggtgtacgcctgcgaggtgacccatcagggcctgagcagccccgtgaccaagagcttcaacaggggcgagtgc

SEQ ID NO: 163 - DNA, 642 bp, synthetic construct
  source 1..642: mol_type = other DNA
gacatccagatgacccagagccccagcttcctgagcgccagcgtgggcgacagggtgaccatcacctgcaaggccagccaggacgtgagcatcgccgtggcctggtaccagcagaagcccggcaaggcccccaagctgctgatctactgggccagcaccaggcagaccggcgtgcccagcaggttcagcggcagcggcagcggcaccgacttcaccctgaccatcagcagcctgcagcccgaggacagcgccacctacttctgccagcagtacagcagctaccccttcaccttcggccagggcaccaaggtggacatcaagcgaacggtggctgccccctccgtgttcatcttcccccccagcgatgagcagctgaagagcggcacagccagcgtggtgtgcctgctgaacaacttctaccccagggaggccaaggtgcagtggaaggtggacaacgccctgcagagcggcaacagccaggagtccgtgaccgagcaggacagcaaggacagcacctacagcctgagcagcacactgaccctgagcaaggccgactacgagaagcacaaggtgtacgcctgcgaggtgacccatcagggcctgagcagccccgtgaccaagagcttcaacaggggcgagtgc

SEQ ID NO: 164 - DNA, 642 bp, synthetic construct
  source 1..642: mol_type = other DNA
gagatccagatgacccagagccagagcaccctgagcgtgagcctgggcgacagggtgaccatcacctgcaaggccagccagaacgtggtgaccgccgtggcctggtaccagcagaagcccggccaggcccccaagctgctgatctacagcgccagccacaggttcaccggcgtgcccagcaggttcagcggcagcggcagcggcaccgacttcaccctgatcatcagctgcatgcagagcgaggacttcgccgtgtactactgccagcagtacagcagctacccctacaccttcggccagggcaccaagctggagatcaagcgaacggtggctgccccctccgtgttcatcttcccccccagcgatgagcagctgaagagcggcacagccagcgtggtgtgcctgctgaacaacttctaccccagggaggccaaggtgcagtggaaggtggacaacgccctgcagagcggcaacagccaggagtccgtgaccgagcaggacagcaaggacagcacctacagcctgagcagcacactgaccctgagcaaggccgactacgagaagcacaaggtgtacgcctgcgaggtgacccatcagggcctgagcagccccgtgaccaagagcttcaacaggggcgagtgc

SEQ ID NO: 165 - DNA, 642 bp, synthetic construct
  source 1..642: mol_type = other DNA
gacatccagatgacccagagccccagcttcctgagcgccagcgtgggcgacagggtgaccatcacctgcaaggccagccagaacgtggtgaccgccgtggcctggtaccagcagaagcccggcaaggcccccaagctgctgatctacagcgccagccacaggttcaccggcgtgcccagcaggttcagcggcagcggcagcggcaccgacttcaccctgaccatcagcaacctgcagagcgaggacctggccacctacttctgccagcagtacagcagctacccctacaccttcggccagggcaccaagctggagatcaagcgaacggtggctgccccctccgtgttcatcttcccccccagcgatgagcagctgaagagcggcacagccagcgtggtgtgcctgctgaacaacttctaccccagggaggccaaggtgcagtggaaggtggacaacgccctgcagagcggcaacagccaggagtccgtgaccgagcaggacagcaaggacagcacctacagcctgagcagcacactgaccctgagcaaggccgactacgagaagcacaaggtgtacgcctgcgaggtgacccatcagggcctgagcagccccgtgaccaagagcttcaacaggggcgagtgc

SEQ ID NO: 166 - DNA, 642 bp, synthetic construct
  source 1..642: mol_type = other DNA
gacatccagatgacccagagccccagcttcctgagcgccagcgtgggcgacagggtgaccatcacctgcaaggccagccagaacgtggtgaccgccgtggcctggtaccagcagaagcccggcaaggcccccaagctgctgatctacagcgccagccacaggttcaccggcgtgcccagcaggttcagcggcagcggcagcggcaccgacttcaccctgaccatcagcaacctgcagcccgaggacagcgccacctacttctgccagcagtacagcagctacccctacaccttcggccagggcaccaagctggagatcaagcgaacggtggctgccccctccgtgttcatcttcccccccagcgatgagcagctgaagagcggcacagccagcgtggtgtgcctgctgaacaacttctaccccagggaggccaaggtgcagtggaaggtggacaacgccctgcagagcggcaacagccaggagtccgtgaccgagcaggacagcaaggacagcacctacagcctgagcagcacactgaccctgagcaaggccgactacgagaagcacaaggtgtacgcctgcgaggtgacccatcagggcctgagcagccccgtgaccaagagcttcaacaggggcgagtgc

SEQ ID NO: 167 - DNA, 642 bp, synthetic construct
  source 1..642: mol_type = other DNA
gacatccagatgacccagagccccagcttcctgagcgccagcgtgggcgacagggtgaccatcacctgcaaggccagccagaacgtggtgaccgccgtggcctggtaccagcagaagcccggcaaggcccccaagctgctgatctacagcgccagccacaggttcaccggcgtgcccagcaggttcagcggcagcggcagcggcaccgacttcaccctgaccatcagcaacctgcagcccgacgacgccgccacctacttctgccagcagtacagcagctacccctacaccttcggccagggcaccaagctggagatcaagcgaacggtggctgccccctccgtgttcatcttcccccccagcgatgagcagctgaagagcggcacagccagcgtggtgtgcctgctgaacaacttctaccccagggaggccaaggtgcagtggaaggtggacaacgccctgcagagcggcaacagccaggagtccgtgaccgagcaggacagcaaggacagcacctacagcctgagcagcacactgaccctgagcaaggccgactacgagaagcacaaggtgtacgcctgcgaggtgacccatcagggcctgagcagccccgtgaccaagagcttcaacaggggcgagtgc

SEQ ID NO: 168 - DNA, 642 bp, synthetic construct
  source 1..642: mol_type = other DNA
gccatccagatgacccagagccacaagagcttcagcgtgagcaccggccagagggtgaccatcacctgcaaggccagccaggacgtgagcaccaccgtggcctggtaccagcagaagcccggccaggcccccaagctgctgatctactgggccagcaccaggcagaccggcgtgcccagcaggttcaccggcagcggcagcggcaccgacttcaccctgaccatcagctgcgtgcagagcgaggacttcgccgtgtactactgccagcagtacagcagctaccccttcaccttcggccagggcaccaagctggagatcaagcgaacggtggctgccccctccgtgttcatcttcccccccagcgatgagcagctgaagagcggcacagccagcgtggtgtgcctgctgaacaacttctaccccagggaggccaaggtgcagtggaaggtggacaacgccctgcagagcggcaacagccaggagtccgtgaccgagcaggacagcaaggacagcacctacagcctgagcagcacactgaccctgagcaaggccgactacgagaagcacaaggtgtacgcctgcgaggtgacccatcagggcctgagcagccccgtgaccaagagcttcaacaggggcgagtgc

SEQ ID NO: 169 - DNA, 642 bp, synthetic construct
  source 1..642: mol_type = other DNA
gacatccagatgacccagagccccagcttcctgagcgccagcgtgggcgacagggtgaccatcacctgcaaggccagccaggacgtgagcaccaccgtggcctggtaccagcagaagcccggcaaggcccccaagctgctgatctactgggccagcaccaggcagaccggcgtgcccagcaggttcagcggcagcggcagcggcaccgacttcaccctgaccatcagcaacctgcagcccgaggacttcgccacctacttctgccagcagtacagcagctaccccttcaccttcggccagggcaccaagctggagatcaagcgaacggtggctgccccctccgtgttcatcttcccccccagcgatgagcagctgaagagcggcacagccagcgtggtgtgcctgctgaacaacttctaccccagggaggccaaggtgcagtggaaggtggacaacgccctgcagagcggcaacagccaggagtccgtgaccgagcaggacagcaaggacagcacctacagcctgagcagcacactgaccctgagcaaggccgactacgagaagcacaaggtgtacgcctgcgaggtgacccatcagggcctgagcagccccgtgaccaagagcttcaacaggggcgagtgc

SEQ ID NO: 170 - DNA, 642 bp, synthetic construct
  source 1..642: mol_type = other DNA
gacatccagatgacccagagccccagcttcctgagcgccagcgtgggcgagagggtgaccatcacctgcaaggccagccaggacgtgagcaccaccgtggcctggtaccagcagaagcccggcaaggcccccaagctgctgatctactgggccagcaccaggcagaccggcgtgcccagcaggttcagcggcagcggcagcggcaccgacttcaccctgaccatcaacagcctgcagcccgaggacgtggccacctacttctgccagcagtacagcagctaccccttcaccttcggccagggcaccaagctggagatcaagcgaacggtggctgccccctccgtgttcatcttcccccccagcgatgagcagctgaagagcggcacagccagcgtggtgtgcctgctgaacaacttctaccccagggaggccaaggtgcagtggaaggtggacaacgccctgcagagcggcaacagccaggagtccgtgaccgagcaggacagcaaggacagcacctacagcctgagcagcacactgaccctgagcaaggccgactacgagaagcacaaggtgtacgcctgcgaggtgacccatcagggcctgagcagccccgtgaccaagagcttcaacaggggcgagtgc

SEQ ID NO: 171 - DNA, 642 bp, synthetic construct
  source 1..642: mol_type = other DNA
gacatccagatgacccagagccccagcttcctgagcgccagcgtgggcgagagggtgaccatcacctgcaaggccagccaggacgtgagcaccaccgtggcctggtaccagcagaagcccggcaaggcccccaagctgctgatctactgggccagcaccaggcagaccggcgtgcccagcaggttcagcggcagcggcagcggcaccgacttcaccctgaccatcagcaacctgcagcccgaggacgtggccacctacttctgccagcagtacagcagctaccccttcaccttcggccagggcaccaagctggagatcaagcgaacggtggctgccccctccgtgttcatcttcccccccagcgatgagcagctgaagagcggcacagccagcgtggtgtgcctgctgaacaacttctaccccagggaggccaaggtgcagtggaaggtggacaacgccctgcagagcggcaacagccaggagtccgtgaccgagcaggacagcaaggacagcacctacagcctgagcagcacactgaccctgagcaaggccgactacgagaagcacaaggtgtacgcctgcgaggtgacccatcagggcctgagcagccccgtgaccaagagcttcaacaggggcgagtgc

SEQ ID NO: 172 - DNA, 642 bp, synthetic construct
  source 1..642: mol_type = other DNA
gacatccagatgacccagagccccagcagcgtgagcctgaccgtgggccagagggtgaccatcacctgcctggccaccgaggacatcttcagctacctggcctggtaccagcagaagcccggcaaggcccccaggctgctgatctacggcgccaacaggctgaaggacggcgtgcccagcaggttcagcggcagcggcagcggcacccagtacaccctgaggatcagcagcatgcagcccgaggacttcggcgtgtactactgcctgcagggcgccaagttccccctgaccttcggccccggcaccaagctggagatcaagcgaacggtggctgccccctccgtgttcatcttcccccccagcgatgagcagctgaagagcggcacagccagcgtggtgtgcctgctgaacaacttctaccccagggaggccaaggtgcagtggaaggtggacaacgccctgcagagcggcaacagccaggagtccgtgaccgagcaggacagcaaggacagcacctacagcctgagcagcacactgaccctgagcaaggccgactacgagaagcacaaggtgtacgcctgcgaggtgacccatcagggcctgagcagccccgtgaccaagagcttcaacaggggcgagtgc

SEQ ID NO: 173 - DNA, 642 bp, synthetic construct
  source 1..642: mol_type = other DNA
gacatccagatgacccagagccccagcagcctgagcgccagcatcggcgacaccgtgaccatcacctgcctggccaccgaggacatcttcagctacctggcctggtaccagcagaggcccggcaaggcccccaagctgctgatctacggcgccaacaggctgaaggacggcgtgcccagcaggttcagcggcagcggcagcggcaccgactacaccctgaccatcagcggcctgcagcccgaggacttcgtgacctactactgcctgcagggcgccaagttccccctgaccttcggccagggcaccaagctggacatgaagcgaacggtggctgccccctccgtgttcatcttcccccccagcgatgagcagctgaagagcggcacagccagcgtggtgtgcctgctgaacaacttctaccccagggaggccaaggtgcagtggaaggtggacaacgccctgcagagcggcaacagccaggagtccgtgaccgagcaggacagcaaggacagcacctacagcctgagcagcacactgaccctgagcaaggccgactacgagaagcacaaggtgtacgcctgcgaggtgacccatcagggcctgagcagccccgtgaccaagagcttcaacaggggcgagtgc

SEQ ID NO: 174 - DNA, 642 bp, synthetic construct
  source 1..642: mol_type = other DNA
gacatccagatgacccagagccccagcagcctgagcgccagcgtgggcgacaccgtgaccatcacctgcctggccaccgaggacatcttcagctacctggcctggtaccagcagaagcccggcaaggcccccaagctgctgatctacggcgccaacaggctgaaggacggcgtgcccagcaggttcagcggcagcggcagcggcaccgactacaccctgaccatcagcggcctgcagcccgaggacttcggcacctactactgcctgcagggcgccaagttccccctgaccttcggccagggcaccaagctggagatcaagcgaacggtggctgccccctccgtgttcatcttcccccccagcgatgagcagctgaagagcggcacagccagcgtggtgtgcctgctgaacaacttctaccccagggaggccaaggtgcagtggaaggtggacaacgccctgcagagcggcaacagccaggagtccgtgaccgagcaggacagcaaggacagcacctacagcctgagcagcacactgaccctgagcaaggccgactacgagaagcacaaggtgtacgcctgcgaggtgacccatcagggcctgagcagccccgtgaccaagagcttcaacaggggcgagtgc

SEQ ID NO: 175 - DNA, 642 bp, synthetic construct
  source 1..642: mol_type = other DNA
gacatccagatgacccagagccccagcagcctgagcgccagcgtgggcgacaccgtgaccatcacctgcctggccaccgaggacatcttcagctacctggcctggtaccagcagaagcccggcaaggcccccaagctgctgatctacggcgccaacaggctgaaggacggcgtgcccagcaggttcagcggcggcggcagcggcaccgagtacagcctgaccatcagcggcctgcagcccgaggacttcggcacctactactgcctgcagggcgccaagttccccctgaccttcggccagggcaccaagctggacatgaagcgaacggtggctgccccctccgtgttcatcttcccccccagcgatgagcagctgaagagcggcacagccagcgtggtgtgcctgctgaacaacttctaccccagggaggccaaggtgcagtggaaggtggacaacgccctgcagagcggcaacagccaggagtccgtgaccgagcaggacagcaaggacagcacctacagcctgagcagcacactgaccctgagcaaggccgactacgagaagcacaaggtgtacgcctgcgaggtgacccatcagggcctgagcagccccgtgaccaagagcttcaacaggggcgagtgc

SEQ ID NO: 176 - DNA, 642 bp, synthetic construct
  source 1..642: mol_type = other DNA
gacatccagatgacccagagccccagcagcctgagcgccagcgtgggcgacaccgtgaccatcacctgcctggccaccgaggacatcttcagctacctggcctggtaccagcagaagcccggcaaggcccccaagctgctgatctacggcgccaacaggctgaaggacaccgtgcccagcaggttcagcggcggcggcagcggcaccgagtacagcctgaccatcagcggcctgcagcccgaggacttcggcacctactactgcctgcagggcgccaagttccccctgaccttcggccagggcaccaagctggacatgaagcgaacggtggctgccccctccgtgttcatcttcccccccagcgatgagcagctgaagagcggcacagccagcgtggtgtgcctgctgaacaacttctaccccagggaggccaaggtgcagtggaaggtggacaacgccctgcagagcggcaacagccaggagtccgtgaccgagcaggacagcaaggacagcacctacagcctgagcagcacactgaccctgagcaaggccgactacgagaagcacaaggtgtacgcctgcgaggtgacccatcagggcctgagcagccccgtgaccaagagcttcaacaggggcgagtgc

SEQ ID NO: 177 - DNA, 1356 bp, synthetic construct
  source 1..1356: mol_type = other DNA
caggtgcagctgctggagagcggcggcgagctggtgcagcccggcggcagcctgaggctgagctgcgccgccagcggcttcaccttcagcgactactacatgttctggatcaggcagaccccccagaagaggctggagtgggtggcctacatcagcaacggcggcggcaacacctactaccccgacaccgtgaagggcaggttcaccatcagcagggacaacgccaagaaccagctgtacctgcagatgaggagcctgacccccgaggacaccgccatctactactgcgccagccccgaggccaggtactacggcaactaccccttcccctactggggccagggcaccctggtgaccgtgagcagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 178 - DNA, 1356 bp, synthetic construct
  source 1..1356: mol_type = other DNA
caggtgcagctggtggagagcggcggcggcctggtgaagcccggcggcagcctgaggctgagctgcgccgccagcggcttcaccttcagcgactactacatgttctggatcaggcaggcccccggcaagggcctggagtgggtggcctacatcagcaacggcggcggcaacacctactaccccgacaccgtgaagggcaggttcaccatcagcagggacaacgccaagaacaccctgtacctgcagatgaacagcctgagggccgaggacaccgccgtgtactactgcgccagccccgaggccaggtactacggcaactaccccttcccctactggggccagggcaccctggtgaccgtgagcagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 179 - DNA, 1356 bp, synthetic construct
  source 1..1356: mol_type = other DNA
caggtgcagctggtggagagcggcggcggcgtggtgaagcccggcggcagcctgaggctgagctgcgccgccagcggcttcaccttcagcgactactacatgttctggatcaggcaggcccccggcaagggcctggagtgggtggcctacatcagcaacggcggcggcaacacctactaccccgacaccgtgaagggcaggttcaccatcagcagggacaacgccaagaacaccctgtacctgcagatgaacagcctgagggccgaggacaccgccgtgtactactgcgccagccccgaggccaggtactacggcaactaccccttcccctactggggccagggcaccctggtgaccgtgagcagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 180 - DNA, 1356 bp, synthetic construct
  source 1..1356: mol_type = other DNA
caggtgcagctggtggagagcggcggcggcgtggtgcagcccggcggcagcctgaggctgagctgcgccgccagcggcttcaccttcagcgactactacatgttctggatcaggcaggcccccggcaagggcctggagtgggtggcctacatcagcaacggcggcggcaacacctactaccccgacaccgtgaagggcaggttcaccatcagcagggacaacgccaagaacaccctgtacctgcagatgaacagcctgagggccgaggacaccgccgtgtactactgcgccagccccgaggccaggtactacggcaactaccccttcccctactggggccagggcaccctggtgaccgtgagcagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 181 - DNA, 1338 bp, synthetic construct
  source 1..1338: mol_type = other DNA
caggtgcagctgctgcagagcggccccggcctggtgaagcccagcgccaccctgagcctgacctgcaccgtgagcggcttcagcctgaccacctacggcgtgcactggatcaggcagccccccggcaagggcctggagtggatgggcgtgatctggagcggcggcagcaccgactacaacgccgccttcatcagcaggctgaccatcagcaaggacaacagcaagaaccaggtgttcctgcagctgtacagcctgagggccgaggacaccgccatctactactgcgccaggaccagccactggtacttcgacgtgtggggcaggggcaccctggtgaccgtgagcagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 182 - DNA, 1338 bp, synthetic construct
  source 1..1338: mol_type = other DNA
caggtgcagctgcaggagagcggccccggcctggtgaagcccagccagaccctgagcctgacctgcaccgtgagcggcttcagcctgaccacctacggcgtgcactgggtgaggcagagccccggcaagggcctggagtggctgggcgtgatctggagcggcggcagcaccgactacaacgccgccttcatcagcaggctgagcatcagcaaggacaacagcaagagccaggtgttcctggagatgaccagcctgaccgccgccgacaccgccatctactactgcgccaggaccagccactggtacttcgacgtgtggggcaggggcaccctggtgaccgtgagcagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 183 - DNA, 1338 bp, synthetic construct
  source 1..1338: mol_type = other DNA
caggtgcagctgcaggagagcggccccggcctggtgaagcccagccagaccctgagcctgacctgcaccgtgagcggcttcagcctgaccacctacggcgtgcactgggtgaggcaggcccccgagaagggcctggagtggctgggcgtgatctggagcggcggcagcaccgactacaacgccgccttcatcagcaggctgagcatcagcaaggacaacagcaagagccaggtgttcctggagatgaccagcctgaccgccgacgacaccgccatctactactgcgccaggaccagccactggtacttcgacgtgtggggcaggggcaccctggtgaccgtgagcagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 184 - DNA, 1338 bp, synthetic construct
  source 1..1338: mol_type = other DNA
caggtgcagctgcaggagagcggccccggcctggtgaagcccagccagaccctgagcctgacctgcaccgtgagcggcttcagcctgaccacctacggcgtgcactgggtgaggcagacccccgagaagggcctggagtggctgggcgtgatctggagcggcggcagcaccgactacaacgccgccttcatcagcaggctgagcatcagcaaggacaacagcaagagccaggtgttcctggagatgaccagcctgaccgccgacgacaccgccatctactactgcgccaggaccagccactggtacttcgacgtgtggggcaggggcaccctggtgaccgtgagcagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 185 - DNA, 1356 bp, synthetic construct
  source 1..1356: mol_type = other DNA
caggtgcagctgctggagagcggcggcggcctggtgcagcccggcggcagcctgaggctgagctgcgccgccagcggcttcaccttcagcgactactacatgtactggatcaggcagaccccccagaagaggctggagtgggtggcctacatcagcaacggcggcggcaacacctactacagcgacaccgtgaagggcaggttcaccatcagcagggacaacgccaagaaccagctgtacctgcagatgaggagcctgaggcccgaggacaccgccatctactactgcgccagccccgaggccaggtactacggcaacttccccttcccctactggggccagggcaccctggtgaccgtgagcagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 186 - DNA, 1356 bp, synthetic construct
  source 1..1356: mol_type = other DNA
caggtgcagctggtggagagcggcggcggcctggtgaagcccggcggcagcctgaggctgagctgcgccgccagcggcttcaccttcagcgactactacatgtactggatcaggcaggcccccggcaagggcctggagtgggtggcctacatcagcaacggcggcggcaacacctactacagcgacaccgtgaagggcaggttcaccatcagcagggacaacgccaagaacaccctgtacctgcagatgaacagcctgagggccgaggacaccgccgtgtactactgcgccagccccgaggccaggtactacggcaacttccccttcccctactggggccagggcaccctggtgaccgtgagcagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 187 - DNA, 1356 bp, synthetic construct; identical to SEQ ID NO: 186
  source 1..1356: mol_type = other DNA

SEQ ID NO: 188 - DNA, 1356 bp, synthetic construct; identical to SEQ ID NO: 186
  source 1..1356: mol_type = other DNA

SEQ ID NO: 189 - DNA, 1344 bp, synthetic construct
  source 1..1344: mol_type = other DNA
caggtgcagctggtggagagcggcggcggcgtggtgcagcccggcaggagcctgaggctgagctgcgccgccagcggcttcagcttcagcaactactacatggcctgggtgaggcaggcccccggcaagggcctggagtgggtggccagcatcagcaccggcggcggcaacatctactacagggacagcgtgaagggcaggttcaccatcagcagggacaacagcaagagcaccctgtacctgcagatgaggagcctgaggagcgaggacaccgccatctactactgcgccaggcagaccgcctactacgtgatggacgcctggggccagggcaccatggtgaccgtgagcagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 190 - DNA, 1344 bp, synthetic construct
  source 1..1344: mol_type = other DNA
gaggtgcagctggtggagagcggcggcggcctggtgcagcccggcggcagcctgaggctgagctgcgccgccagcggcttcagcttcagcaactactacatggcctgggtgaggcaggcccccggcaagggcctggagtgggtggccagcatcagcaccggcggcggcaacatctactacagggacagcgtgaagggcaggttcaccatcagcagggacaacgccaagaacagcctgtacctgcagatgaacagcctgagggccgaggacaccgccgtgtactactgcgccaggcagaccgcctactacgtgatggacgcctggggccagggcaccaccgtgaccgtgagcagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 191 - DNA, 1344 bp, synthetic construct; identical to SEQ ID NO: 190
  source 1..1344: mol_type = other DNA

SEQ ID NO: 192 - DNA, 1344 bp, synthetic construct; identical to SEQ ID NO: 190
  source 1..1344: mol_type = other DNA

SEQ ID NO: 193 - DNA, 768 bp, synthetic construct
  source 1..768: mol_type = other DNA
ggaggatggtcctgcgcccccgacgaggacacctggctgtgccccgccggcggaggaggctctggaggactgtctggcaggtccgacgccggctcccctctgggcctggctggcagcggcggctctgacatccagatgacacagtccccatccagcctgtccgcctccgtgggcgacaccgtgacaatcacctgtctggccaccgaggacatcttctcctacctggcttggtatcagcagaagccaggcaaggcccccaagctgctgatctacggcgctaataggctgaaggacacagtgccatcccggttcagcggaggaggctccggcacagagtattctctgaccatctccggcctgcagcctgaggattttggcacctactattgcctgcagggcgccaagttcccactgacatttggccagggcaccaagctggacatgaagagaacagtggccgctccctccgtgttcatctttccccctagcgatgagcagctgaagagcggcaccgcttctgtggtgtgcctgctgaacaatttctaccctcgcgaggccaaggtgcagtggaaggtggacaacgctctgcagtctggcaattcccaggagagcgtgacagagcaggactctaaggattccacctatagcctgtcttccacactgaccctgtctaaggccgattacgagaagcacaaggtgtatgcttgcgaggtgacacatcagggcctgtcctcccccgtgaccaagtcctttaacagaggcgagtgt

SEQ ID NO: 194 - DNA, 1956 bp, synthetic construct
  source 1..1956: mol_type = other DNA
gaggtgcagctggtggagtccggaggaggactggtgcagcctggcggctccctgagactgagctgtgccgcttctggctccacattctacaccgccgtgatgggatgggtgaggcaggctccaggcaagggcctggagtgggtggctgctatcaggtggacagccctgaccacatcttatgctgactccgtgaagggcagattcaccatctcccgcgatggcgccaagaccacactgtacctgcagatgaacagcctgagacctgaggacacagccgtgtactattgcgctgctcgcggcaccctgggactgtttaccacagctgactcctacgattattggggccagggcacactggtgaccgtgtccagcggcggaggaggcagcggaggaggctctggcggcgtgtactgcggcccagagttcgacgagtccgtgggctgtatgggcggcggaggcagcggaggaggactgtccggcagaagcgatgctggctccccactgggcctggctggctccggaggaggcagcgaagtccagctggtggagagcggcggcggcctggtccagcctggcggctctctgaagctgtcctgtgccgcctccggcttcacctttaacaagtatgccatgaattgggtgcgccaggctcccggcaagggcctggagtgggtagccaggatcaggtccaagtacaacaattatgccacctactacgccgactccgtgaaggataggttcacaatctctcgggacgattccaagaacaccgcctacctgcagatgaacaatctgaagacagaggacaccgccgtgtactattgcgtgaggcacggcaactttggcaattcttacatctcctattgggcttactggggtcagggcacactggtcaccgtgtcttccggaggaggaggctccggcggcggaggcagcggcggcggcggctctcagacagtggtgacccaggagccaagcctgaccgtgtctcccggcggcaccgtgacactgacctgtggcagctctacaggagctgtgaccagcggaaactatccaaattgggtgcagcagaagcctggccaggctcctagaggcctgatcggaggcacaaagttcctggccccaggcaccccagctcgctttagcggctctctgctgggaggcaaggccgctctgaccctgagcggagtgcagccagaggatgaggccgagtactattgcgtgctgtggtactctaacagatgggtgtttggcggcggcacaaagctgaccgtgctgggaggaggaggcagcgaagtgcagctggtcgagtctggcggcggcttagtccaacctggcggctccctgaggctgtcttgcgccgcttctggcttctcctttagcaactactatatggcttgggtgcggcaggctcctggcaagggcctggagtgggtcgcctctatctccacaggcggcggcaatatctactatcgggactctgtgaagggcaggttcaccatctccagggacaacgctaagaatagcctgtatctgcagatgaactccctgagggccgaagatactgccgtgtactactgcgcccggcagaccgcttactatgtgatggatgcctggggccagggcaccacagtgacagtgtccagcgcctccaccaagggccctagcgtgttccctctggctccatcttccaagagcacatctggaggcaccgccgctctgggatgtctggtgaaggactacttccccgagcctgtgaccgtgagctggaactctggcgccctgacatctggcgtgcacacctttcccgctgtgctgcagtcctccggcctgtattccctgtccagcgtggtgacagtgccttcttccagcctgggcacacagacctacatctgcaacgtgaatcataagcctagcaataccaaggtggataagaaggtggagccaaagtcttgt

(19) United States
(12) Patent Application Publication
(10) Pub. No.: US 2026/0226165 A1
(43) Pub. Date: Aug. 6, 2026

(54) COMPOSITIONS AND METHODS RELATED TO TUMOR ACTIVATED MOLECULES TARGETING NECTIN-4 AND EFFECTOR CELL ANTIGENS

(71) Applicants: Merck Sharp & Dohme LLC, Rahway, NJ (US); Janux Therapeutics, Inc., San Diego, CA (US)

(72) Inventors: Andrew B. Waight, Belmont, CA (US); Rebecca Goldstein, Albany, CA (US); Mehabaw Getahun Derebe, Burlingame, CA (US); Daniel Malashock, Santa Clara, CA (US); Karin Vroom, San Mateo, CA (US); Sandra Isabel Vanegas, Fremont, CA (US); David Alan Campbell, San Diego, CA (US); Thomas Robert DiRaimondo, San Diego, CA (US); Iliana Tenvooren, San Francisco, CA (US)

(73) Assignees: Merck Sharp & Dohme LLC, Rahway, NJ (US); Janux Therapeutics, Inc., San Diego, CA (US)

(21) Appl. No.: 19/419,529

(22) Filed: Dec. 15, 2025

Related U.S. Application Data
(60) Provisional application No. 63/735,003, filed on Dec. 17, 2024.

(51) Int. Cl. C07K 16/28; A61K 39/00; A61P 35/00; C07K 16/18

(52) U.S. Cl. CPC C07K 16/2803; A61P 35/00; C07K 16/18; C07K 16/2809; A61K 2039/505; C07K 2317/24; C07K 2317/31; C07K 2317/34; C07K 2317/55; C07K 2317/565; C07K 2317/73; C07K 2317/92

(57) ABSTRACT

Disclosed herein are polypeptides and polypeptide complexes that are capable of binding tumor cell antigens and effector cell antigens. In some embodiments, the polypeptides and polypeptide complexes bind the tumor cell antigens and the effector cell antigens in a tumor microenvironment.

### CROSS-REFERENCE TO RELATED APPLICATIONS

This application claims the benefit of U.S. Provisional Patent Application No. 63/735,003, filed Dec. 17, 2024, the entire contents of which are incorporated by reference herein.

### REFERENCE TO SEQUENCE LISTING SUBMITTED ELECTRONICALLY

The contents of the electronic sequence listing (26126-WO-PCT_SL.xml; Size: 221,037 bytes, created on Jan. 24, 2025) are herein incorporated by reference in their entirety.

### FIELD

This disclosure relates generally to molecules that contain an antibody or antibody fragments and that are capable of binding to Nectin-4 and an effector cell antigen.

### BACKGROUND

T cell engager (TCE) therapeutics have several benefits including they are not cell therapies and thus can be offered as off-the-shelf therapies as opposed to chimeric antigen receptor T cell (CAR T cell) therapies. While TCE therapeutics have displayed potent anti-tumor activity in hematological cancers, developing TCEs to treat solid tumors has faced challenges due to the limitations of prior TCE technologies, namely (i) overactivation of the immune system leading to cytokine release syndrome (CRS), (ii) on-target, healthy tissue toxicities and (iii) poor pharmacokinetics (PK) leading to short half-life. CRS arises from the systemic activation of T cells and can result in life-threatening elevations in inflammatory cytokines such as interleukin-6 (IL-6). Severe and acute CRS leading to dose limited toxicities and deaths have been observed upon the dosing of T cell engagers developed using other platforms to treat cancer patients in clinical studies. This toxicity restricts the maximum blood levels of T cell engagers that can be safely dosed. T cell engager effectiveness has also been limited because of on-target, healthy tissue toxicity. T cell engagers developed using a platform not designed for tumor-specification activation have resulted in clinicals holds and dose-limiting toxicities resulting from target expression in healthy tissues. T cell engagers have also been limited by short half-lives. T cell engagers quickly reach sub-therapeutic levels after being administered as they are quickly eliminated from the body due to their short exposure half-lives. For this reason, T cell engagers such as blinatumomab are typically administered by a low-dose, continuous infusion pump over a period of weeks to overcome the challenge of a short half-life and to maintain therapeutic levels of drug in the body. A continuous dosing regimen represents a significant burden for patients.

### SUMMARY

The present disclosure relates to polypeptide chains and polypeptide complexes that comprise at least one polypeptide chain. In some embodiments, the polypeptide complexes comprise two polypeptide chains. In some embodiments, the polypeptide complexes are capable of binding to a tumor cell antigen and an effector cell antigen in a tumor microenvironment (TME).

In an aspect, a polypeptide complex is provided, comprising:
(i) a first polypeptide chain comprising: an anti-albumin (ALB) heavy chain variable region comprising a CDRH1 comprising the amino acid sequence of SEQ ID NO: 4, a CDRH2 comprising the amino acid sequence of SEQ ID NO: 5, and a CDRH3 comprising the amino acid sequence of SEQ ID NO: 6; a first mask sequence comprising the amino acid sequence of SEQ ID NO: 7; a first protease cleavage site comprising the amino acid sequence of SEQ ID NO: 8; an anti-CD3 heavy chain variable region comprising a CDRH1 comprising the amino acid sequence of SEQ ID NO: 9, a CDRH2 comprising the amino acid sequence of SEQ ID NO: 10, and a CDRH3 comprising the amino acid sequence of SEQ ID NO: 11; an anti-CD3 light chain variable region comprising a CDRL1 comprising the amino acid sequence of SEQ ID NO: 12, a CDRL2 comprising the amino acid sequence of SEQ ID NO: 13, and a CDRL3 comprising the amino acid sequence of SEQ ID NO: 14; and an anti-Nectin-4 heavy chain variable region comprising a CDRH1 comprising the amino acid sequence of SEQ ID NO: 15, a CDRH2 comprising the amino acid sequence of SEQ ID NO: 16, a CDRH3 comprising the amino acid sequence of SEQ ID NO: 17; and
(ii) a second polypeptide chain comprising: a second mask sequence comprising the amino acid sequence of SEQ ID NO: 18; a second protease cleavage site comprising the amino acid sequence of SEQ ID NO: 19; and an anti-Nectin-4 light chain variable region comprising a CDRL1 comprising the amino acid sequence of SEQ ID NO: 20, a CDRL2 comprising the amino acid sequence of SEQ ID NO: 21, and a CDRL3 comprising the amino acid sequence of SEQ ID NO: 22.

In an aspect, a polypeptide complex is provided, comprising: a first polypeptide chain comprising an amino acid sequence that is at least 95% identical to SEQ ID NO: 1; and a second polypeptide chain comprising an amino acid sequence that is at least 95% identical to SEQ ID NO: 2; wherein the polypeptide complex binds to Nectin-4 and CD3.

In an aspect, a polypeptide complex is provided, comprising:
(i) a first polypeptide chain comprising: an anti-albumin (ALB) heavy chain variable region comprising a CDRH1 comprising the amino acid sequence of SEQ ID NO: 4, a CDRH2 comprising the amino acid sequence of SEQ ID NO: 5, and a CDRH3 comprising the amino acid sequence of SEQ ID NO: 6; a first mask sequence comprising the amino acid sequence of SEQ ID NO: 7; a first protease cleavage site comprising the amino acid sequence of SEQ ID NO: 8; an anti-CD3 heavy chain variable region comprising a CDRH1 comprising the amino acid sequence of SEQ ID NO: 9, a CDRH2 comprising the amino acid sequence of SEQ ID NO: 10, and a CDRH3 comprising the amino acid sequence of SEQ ID NO: 11; an anti-CD3 light chain variable region comprising a CDRL1 comprising the amino acid sequence of SEQ ID NO: 12, a CDRL2 comprising the amino acid sequence of SEQ ID NO: 13, and a CDRL3 comprising the amino acid sequence of SEQ ID NO: 14; and
a Nectin-4 heavy chain variable region comprising:
   (1) a CDRH1 comprising the amino acid sequence of SEQ ID NO: 52, a CDRH2 comprising the amino acid sequence of SEQ ID NO: 53, and a CDRH3 comprising the amino acid sequence of SEQ ID NO: 54;
   (2) a CDRH1 comprising the amino acid sequence of SEQ ID NO: 59, a CDRH2 comprising the amino acid sequence of SEQ ID NO: 60, and a CDRH3 comprising the amino acid sequence of SEQ ID NO: 61; or
   (3) a CDRH1 comprising the amino acid sequence of SEQ ID NO: 66, a CDRH2 comprising the amino acid sequence of SEQ ID NO: 67, and a CDRH3 comprising the amino acid sequence of SEQ ID NO: 68; and
(ii) a second polypeptide chain comprising: a second mask sequence comprising the amino acid sequence of any one of SEQ ID NOs: 18, 23, 125, and 126; a second protease cleavage site comprising the amino acid sequence of SEQ ID NO: 19; and
an anti-Nectin-4 light chain variable region comprising:
   (1) a CDRL1 comprising the amino acid sequence of SEQ ID NO: 28, a CDRL2 comprising the amino acid sequence of SEQ ID NO: 29, and a CDRL3 comprising the amino acid sequence of SEQ ID NO: 30;
   (2) a CDRL1 comprising the amino acid sequence of SEQ ID NO: 35, a CDRL2 comprising the amino acid sequence of SEQ ID NO: 36, and a CDRL3 comprising the amino acid sequence of SEQ ID NO: 37; or
   (3) a CDRL1 comprising the amino acid sequence of SEQ ID NO: 42, a CDRL2 comprising the amino acid sequence of SEQ ID NO: 29, and a CDRL3 comprising the amino acid sequence of SEQ ID NO: 30.

In an aspect, a polypeptide complex is provided, comprising:
(i) a first polypeptide chain comprising the formula: HA-M₁-P₁-EA-TH, wherein: HA comprises an antibody or antibody fragment that binds to a half-life extending molecule, M₁ comprises a first mask sequence, P₁ comprises a first protease cleavage site, EA comprises an antibody or antibody fragment that binds to an effector cell antigen, and TH comprises a heavy chain variable region that binds to a tumor cell antigen, wherein TH comprises:
   (1) an anti-Nectin-4 heavy chain variable region comprising: a CDRH1 comprising the amino acid sequence of SEQ ID NO: 15; a CDRH2 comprising the amino acid sequence of SEQ ID NO: 16; and a CDRH3 comprising the amino acid sequence of SEQ ID NO: 17;
   (2) an anti-Nectin-4 heavy chain variable region comprising a CDRH1 comprising the amino acid sequence of SEQ ID NO: 52; a CDRH2 comprising the amino acid sequence of SEQ ID NO: 53; and a CDRH3 comprising the amino acid sequence of SEQ ID NO: 54;
   (3) an anti-Nectin-4 heavy chain variable region comprising a CDRH1 comprising the amino acid sequence of SEQ ID NO: 59; a CDRH2 comprising the amino acid sequence of SEQ ID NO: 60; and a CDRH3 comprising the amino acid sequence of SEQ ID NO: 61; or
   (4) an anti-Nectin-4 heavy chain variable region comprising a CDRH1 comprising the amino acid sequence of SEQ ID NO: 66; a CDRH2 comprising the amino acid sequence of SEQ ID NO: 67; and a CDRH3 comprising the amino acid sequence of SEQ ID NO: 68, and
(ii) a second polypeptide chain comprising the formula M₂-P₂-TL, wherein: M₂ comprises a second mask sequence, P₂ comprises a second protease cleavage site and TL comprises a light chain variable region that binds a tumor cell antigen, wherein TL comprises:
   (1) an anti-Nectin-4 light chain variable region comprising a CDRL1 comprising the amino acid sequence of SEQ ID NO: 20; a CDRL2 comprising the amino acid sequence of SEQ ID NO: 21; and a CDRL3 comprising the amino acid sequence of SEQ ID NO: 22;
   (2) an anti-Nectin-4 light chain variable region comprising a CDRL1 comprising the amino acid sequence of SEQ ID NO: 28; a CDRL2 comprising the amino acid sequence of SEQ ID NO: 29; and a CDRL3 comprising the amino acid sequence of SEQ ID NO: 30;
   (3) an anti-Nectin-4 light chain variable region comprising a CDRL1 comprising the amino acid sequence of SEQ ID NO: 35; a CDRL2 comprising the amino acid sequence of SEQ ID NO: 36; and a CDRL3 comprising the amino acid sequence of SEQ ID NO: 37; or
   (4) an anti-Nectin-4 light chain variable region comprising a CDRL1 comprising the amino acid sequence of SEQ ID NO: 42; a CDRL2 comprising the amino acid sequence of SEQ ID NO: 29; and a CDRL3 comprising the amino acid of SEQ ID NO: 30.

In an aspect, a composition is provided comprising any of the polypeptide complexes described herein and a pharmaceutically acceptable carrier.

In an aspect, a method of treating cancer in a subject in need thereof is provided comprising administering to the subject an effective amount of any of the polypeptide complexes described herein or any of the compositions described herein.

In an aspect, a polynucleotide is provided wherein the polynucleotide encodes any of the first polypeptide chains disclosed herein.

In an aspect, a polynucleotide is provided wherein the polynucleotide encodes any of the second polypeptide chains disclosed herein.

In an aspect, a polynucleotide is provided wherein the polynucleotide encodes any of the polypeptide complexes disclosed herein.

In an aspect, an expression vector is provided wherein the expression vector comprises any of the polynucleotides disclosed herein.

In an aspect, a host cell is provided comprising any of the polynucleotides disclosed herein or any of the expression vectors disclosed herein.

In an aspect, a method of producing a polypeptide complex that binds to Nectin-4 and CD3 is provided comprising:
(a) culturing a host cell in culture medium, wherein the host cell comprises one or more vectors comprising a polynucleotide encoding any of the first polypeptide chains disclosed herein and a polynucleotide encoding any of the second polypeptide chains disclosed herein, under conditions that allow expression of the polypeptide chains, and
(b) optionally recovering the polypeptide complex from the host cell or culture medium.

In an aspect, any of the polypeptide complexes or compositions described herein are used in treating cancer.

In an aspect, any of the polypeptide complexes or compositions described herein are used for the manufacture of a medicament to treat cancer.

The summary of the technology described above is non-limiting and other features and advantages of the technology will be apparent from the following detailed description, and from the claims.

### BRIEF DESCRIPTION OF THE DRAWINGS

FIG. 1 provides fluorescence-activated cell sorting (FACS) data showing purified rat antibodies that target Nectin-4 (See Example 2).

FIGS. 2A and 2B provide binding data of purified rat antibodies to Nectin-4 (See Example 2).

FIGS. 3A-3D, 4A-4D, and 5A-5D provide data showing in vitro cytotoxicity on T cells, when the cells are treated with the Nectin-4 TRACTr molecule (See Example 9).

FIGS. 6A-6B provide data comparing in vitro cytotoxicity between (i) T cells and (ii) Nectin-4 knockout T cells, after treatment with the Nectin-4 TRACTr molecule (See Example 9).

FIGS. 7A-7D provide data showing in vitro cytotoxicity on peripheral blood mononuclear cells (PBMCs), when the cells are treated with the Nectin-4 TRACTr molecule (See Example 9).

FIGS. 8A-8D and 9A-9D provide data showing cytokine concentration from TDCC supernatants after treatment with the TRACTr molecule (See Example 9).

FIGS. 10A-10D, 11A-11C, and 12A-12C provide data showing in vitro cytotoxicity on PBMCs, when the cells are treated with the Nectin-4 TRACTr molecule (See Example 9).

FIG. 13 provides an exemplary embodiment of a polypeptide complex outside of the tumor microenvironment.

FIG. 14 provides an exemplary embodiment of a polypeptide complex inside of the tumor microenvironment.

FIG. 15 provides an exemplary embodiment of a polypeptide complex.

FIG. 16 provides data evaluating in vivo tumor growth inhibition in a human PBMC-transferred Panc08.13 xenograft mouse tumor model, after treatment with (i) the Nectin-4 TRACTr molecule at different concentrations and (ii) various control treatments (See Example 11).

FIG. 17 provides data evaluating in vivo tumor growth inhibition in a human T-cell transferred NCI-H322 xenograft tumor model, after treatment with (i) the Nectin-4 TRACTr molecule at different concentrations and (ii) various control treatments (See Example 11).

### DETAILED DESCRIPTION

Biological molecules known in the art capable of bringing effector cells and tumor cells in proximity to each other are often toxic to non-cancerous cells. For example, as known in the art and as shown herein, T-cell engagers can be highly toxic to non-cancerous cells such as effector cells. Disclosed herein are biological molecules that are effective in bringing effector cells and tumor cells in proximity to each other that exhibit significantly reduced toxicity relative to T cell engagers.

In an aspect, the biological molecules are polypeptides and polypeptide complexes that comprise one or more polypeptide chains. In some embodiments, the polypeptide complexes comprise two polypeptide chains. In some embodiments, the two polypeptide chains comprise antibodies or antibody fragments capable of binding to effector cell antigens and tumor antigens. In some embodiments, the polypeptide complexes comprise one or more antibodies or antibody fragments capable of binding an effector cell antigen and a tumor cell antigen. In some embodiments, outside of a tumor microenvironment (TME), the one or more antibodies or antibody fragments are obstructed such that they are unable to bind to an effector cell antigen and a tumor cell antigen.

In some embodiments, binding of the one or more antibodies or antibody fragments are obstructed through one or more masks. In some embodiments, outside of a TME, the one or more masks bind the one or more antibodies or antibody fragments. In some embodiments, binding of the one or more masks to the one or more antibodies or antibody fragments results in a reduction in toxicity on non-cancerous cells (e.g., effector cells), relative to similar a polypeptide complex that does not contain the one or more masks.

In an aspect, a polypeptide complex is disclosed that comprises one or more binding moieties capable of binding to (i) a tumor cell antigen and (ii) an effector cell antigen. In some embodiments, the tumor cell antigen comprises Nectin-4. In some embodiments, the effector cell antigen comprises CD3. In some embodiments, the binding moieties of (i) and (ii) are obstructed such that the polypeptide complex is incapable of binding to a tumor cell antigen and an effector cell antigen. In some embodiments, the binding moieties of (i) and (ii) are obstructed outside of a TME. In some embodiments, inside a TME, the obstructions to the binding moieties of (i) and (ii) are removed to allow binding to a tumor cell antigen and an effector cell antigen. In some embodiments, the obstructions are removed from the binding moieties inside of a TME. In some embodiments, the polypeptide complex comprises an additional binding moiety that binds to a half-life extending molecule. In some embodiments, the binding moieties of the polypeptide complex are antibodies or antibody fragments.

In an aspect, disclosed herein are polypeptide chains and polypeptide complexes that contain one or more antibodies or antibody fragments. In some embodiments, the one or more antibodies or antibody fragments bind a tumor cell antigen (e.g., Nectin-4). In some embodiments, the one or more antibodies or antibody fragments are capable of binding to an effector cell antigen (e.g., CD3). In some embodiments, the one or more antibodies or antibody fragments bind a half-life extending molecule (e.g., albumin).

In some embodiments, the polypeptide complexes described herein comprise one or more mask sequences. In some embodiments, the one or more mask sequences obstruct binding of antibodies or antibody fragments in the polypeptide complex to tumor cell antigen (e.g., Nectin-4). In some embodiments, the one or more mask sequences obstruct binding of antibodies or antibody fragments in the polypeptide complex to tumor cell antigen, outside of a TME. In some embodiments, the one or more mask sequences obstruct binding of the antibodies or antibody fragments to effector cell antigens (e.g., CD3). In some embodiments, the one or more mask sequences obstruct binding of the antibodies or antibody fragments to effector cell antigens, outside of a TME.

In some embodiments, the one or more mask sequences are removed from the polypeptide complexes, when the polypeptide complexes are in a TME. In some embodiments, removal of these masks sequences results in activation of the polypeptide complexes. In some embodiments, activation of the polypeptide complexes results in binding of the one or more antibodies or antibody fragments to a tumor cell antigen. In some embodiments, the tumor cell antigen is Nectin-4. In some embodiments, activation of the polypeptide complexes results in binding of the one or more antibodies or antibody fragments to an effector cell antigen. In some embodiments, the effector cell antigen is CD3.

### Definitions

Listed below are definitions of various terms used herein. These definitions apply to the terms as they are used throughout this specification and claims, unless otherwise limited in specific instances, either individually or as part of a larger group.

Unless defined otherwise, all technical and scientific terms used herein have the same meaning as commonly understood by one of ordinary skill in the art. Generally, the nomenclature used herein and the laboratory procedures in cell culture, molecular genetics, organic chemistry, and peptide chemistry are those well-known and commonly employed in the art.

As used herein, the articles “a” and “an” refer to one or to more than one (i.e., to at least one) of the grammatical object of the article. By way of example, “an element” means one element or more than one element. Furthermore, use of the term “including” as well as other forms, such as “include,” “includes,” and “included,” is not limiting.

As used herein, the term “about” or “approximately” in quantitative terms refers to plus or minus 10% of the value it modifies (rounded up to the nearest whole number if the value is not sub-dividable, such as a number of molecules or nucleotides).

All ranges disclosed herein are inclusive of the recited endpoint and independently combinable (for example, the range of “from 50 mg to 500 mg” is inclusive of the endpoints, 50 mg and 500 mg, and all the intermediate values). The endpoints of the ranges and any values disclosed herein are not limited to the precise range or value; they are sufficiently imprecise to include values approximating these ranges and/or values.

As used herein, the term “comprising” may include the embodiments “consisting of” and “consisting essentially of.” The terms “comprise(s),” “include(s),” “having,” “has,” “may,” “contain(s),” and variants thereof, as used herein, are intended to be open-ended transitional phrases, terms, or words that require the presence of the named ingredients/steps and permit the presence of other ingredients/steps. However, such description should be construed as also describing compositions or processes as “consisting of” and “consisting essentially of” the enumerated components, which allows the presence of only the named components or compounds, along with any acceptable carriers or fluids, and excludes other components or compounds.

Ranges disclosed herein are understood to include all of the values within the range. For example, a range of 1-10 is understood to include all of the integers 1, 2, 3, 4, 5, 6, 7, 8, 9, and 10, as well as all intervening decimal values between the integers, for example, 1.1, 1.2, 1.3, 1.4, 1.5, 1.6, 1.7, 1.8, and 1.9.

As used herein, the term “antigen” refers to any molecule capable of inducing an immune response.

The term “immunoglobulin” refers to a class of proteins that contains two pairs polypeptide chains: (1) one pair of light chains and (2) one pair of heavy chains.

The term “antibody” refers to a type “immunoglobulin” that has specificity for a specific antigen. Structurally antibodies contain two identical light chains and two identical heavy chains that are interconnected through disulfide bonds. Each heavy chain includes a heavy chain variable region. Each light chain includes a light chain variable region. Each heavy chain variable region includes three CDRs (CDRH1, CDRH2, and CDRH3). Each light chain variable region includes three CDRs (CDRL1, CDRL2, and CDRL3).

As use herein, the term “CDR” refers to complementarity-determining region. CDRs are defined as hypervariable regions within heavy chain variable regions and the light chain variable regions, of an antibody. CDRs are responsible for the binding affinity of the antibody to an antigen.

The term “polynucleotide” refers to a chain of nucleotides that are linked through sugar phosphate backbones.

The terms “% identical”, “% identity” or similar terms are intended to refer, in particular to the percentage of nucleotides or amino acids which are identical in an optimal alignment between the sequences to be compared and introducing gaps, if necessary, to achieve maximum percent sequence identity. Said percentage is purely statistical, and the differences between the two sequences may be but are not necessarily randomly distributed over the entire length of the sequences to be compared. Comparisons of two sequences are usually carried out by comparing said sequences, after optimal alignment, with respect to a segment or “window of comparison”, in order to identify local regions of corresponding sequences. Two sequences can be optimally aligned for comparison even when the sequences are of different lengths, which includes optimally aligning sequences in which one sequence is truncated on either or both of its 5′ and 3′ ends relative to the sequence to which it is being aligned. The optimal alignment for a comparison may be carried out manually or with the aid of the local homology algorithm by Smith and Waterman, 1981, Ads App. Math. 2, 482, with the aid of the local homology algorithm by Needleman and Wunsch, 1970, J. Mol. Biol. 48, 443, with the aid of the similarity search algorithm by Pearson and Lipman, 1988, Proc. Natl Acad. Sci. USA 88, 2444, or with the aid of computer programs using said algorithms (GAP, BESTFIT, FASTA, and TFASTA in Wisconsin Genetics Software Package, Genetics Computer Group, 575 Science Drive, Madison, Wis.).

As used herein, the term “fragment” refers to a peptide or a polypeptide that contains less than the full-length amino acid sequence of the peptide or the polypeptide.

As used herein, “TME” refers to a tumor microenvironment.

As used herein, the term “antibody fragment” refers to an antibody that contains less than the full-length sequence of either the heavy chain sequence of the antibody or the light chain sequence of the antibody. An “antibody fragment” can include regions of the antibody that carry out the antibody's binding functions and/or non-binding regions of an antibody. In some embodiments, an “antibody fragment” comprises a light chain variable region or a fragment thereof. In some embodiments, an “antibody fragment” comprises a heavy chain variable region or a fragment thereof. In some embodiments, an “antibody fragment” comprises one or more complementarity-determining regions (CDR) of the antibody or portions of one or more CDRs. An “antibody fragment” can result from truncations of the amino terminus, truncations of the carboxy terminus, or truncations of both the amino and carboxy termini. Examples of antibody fragments include, but are not limited to Fab, Fab′, F(ab′) 2, and Fv fragments. In some embodiments, an “antibody fragment” retains the ability to bind to an antigen that is bound by the full-length antibody, e.g., fragments that contain one or more CDR regions.

As used herein, the term “antigen-binding site” refers to a peptide or a polypeptide, or a fragment thereof that interacts with an antigen.

As used herein, the term “effector cell” refers to any cell that responds to a stimulus and carries out a specific activity in response to the stimulus. In some embodiments, an “effector” cell is a T-cell. In some embodiments, an effector cell is a peripheral blood mononuclear cell (PBMC).

As used herein, the term “CD3” refers to cluster of differentiation 3.

As used herein, the term “mask,” “mask domain,” or “mask sequence” refers to an amino acid sequence that is capable of obstructing an antibody or antibody fragment from binding to an antigen. In some embodiments, a “mask” binds to an antibody or antibody fragment, thereby obstructing an antibody or antibody fragment from binding to an antigen. In some embodiments, the antigen is a tumor cell antigen. In some embodiments, the antigen is an effector cell antigen.

As used herein, the term “tumor cell antigen” means any antigen that is produced in a tumor cell and that is capable of triggering an immune response in the host.

As used herein, the term “effector cell antigen” means an antigen on an “effector cell” capable of activating the “effector cell.”

As used herein, the term “effector cell” means any cell that is capable carrying out a function in response to a stimulus.

As used herein, the term “ALB” refers to albumin.

As used herein, the term “administering” means physically delivering an exogenous substance (e.g., any of the polypeptides or polypeptide complexes described herein) into a subject or a patient. Physically delivering the exogenous substance into the subject or the patient can occur through any method described herein such as, for example, topical administration, intramuscular administration, intravenous administration, oral administration, or subcutaneous administration.

As used herein, the term “prophylactic treatment” means preventing, either in whole or in part, the onset, recurrence, or spread of a disease and/or symptoms that are associated with the disease. In some embodiments, the disease comprises a cancer. In some embodiments, the cancer includes cells that express Nectin-4. In some embodiments, the cancer is any cancer described herein.

As used herein, the term “therapeutic treatment” means treating, managing, or ameliorating a disease or any of the symptoms associated with the disease. In some embodiments, the disease comprises a cancer. In some embodiments, the cancer includes cells that express Nectin-4. In some embodiments, the cancer is any cancer described herein. Unless otherwise specified, use of the term “treatment” is intended to refer to therapeutic treatment.

As used herein, the term “half-life extending molecule” refers to a molecule that increases the therapeutic half-life of a protein to which the “half-life extending molecule” is attached.

As used herein, a “patient” (alternatively referred to herein as a “subject”) refers to a mammal capable of suffering from a disease or disorder and/or one or more symptoms that are associated with the disease or disorder. In embodiments, the patient is a human. A patient can be treated prophylactically or therapeutically. Those “in need of treatment” include those diagnosed with or suspected of having a disease or disorder and/or one or more symptoms that are associated with a disease or disorder, and those who were previously suffering from a disease or disorder, or one or more symptoms thereof, and any person in which prevention of recurrence, lessening in the number or severity of symptoms, or a delay of the progression, onset or reduction in the likelihood of progression or onset of a disease, disorder or the symptoms thereof is desired.

As used herein, a “conservative amino acid substitution” refers to substitutions of amino acids in a protein with other amino acids having similar characteristics (e.g., charge, side-chain size, hydrophobicity/hydrophilicity, backbone conformation and rigidity, etc.), such that the changes can frequently be made without altering the biological activity of the protein. Those of skill in this art recognize that, in general, single amino acid substitutions in non-essential regions of a polypeptide do not substantially alter biological activity (see, e.g., Watson et al. (1987) Molecular Biology of the Gene, The Benjamin/Cummings Pub. Co., p. 224 (4th Ed.)). In addition, substitutions of structurally or functionally similar amino acids are less likely to disrupt biological activity. Exemplary conservative substitutions are set forth in Table 1.

TABLE 1
Exemplary Conservative Amino Acid Substitutions

| Original | Conservative |
|---|---|
| residue | substitution |
| Ala (A) | Gly; Ser |
| Arg (R) | Lys; His |
| Asn (N) | Gln; His |
| Asp (D) | Glu; Asn |
| Cys (C) | Ser; Ala |
| Gln (Q) | Asn |
| Glu (E) | Asp; Gln |
| Gly (G) | Ala |
| His (H) | Asn; Gln |
| Ile (I) | Leu; Val |
| Leu (L) | Ile; Val |
| Lys (K) | Arg; His |
| Met (M) | Leu; Ile; Tyr |
| Phe (F) | Tyr; Met; Leu |
| Pro (P) | Ala |
| Ser (S) | Thr |
| Thr (T) | Ser |
| Trp (W) | Tyr; Phe |
| Tyr (Y) | Trp; Phe |
| Val (V) | Ile; Leu |

### Anti-Nectin-4 Antibodies

Disclosed herein are polypeptide chains and polypeptide complexes that include anti-Nectin-4 antibodies or anti-Nectin-4 antibody fragments. In some embodiments, the anti-Nectin-4 antibodies or anti-Nectin-4 antibody fragments are derived from any species. In some embodiments, the anti-Nectin-4 antibodies and anti-Nectin-4 antibody fragments are derived from a rat. In some embodiments, the anti-Nectin-4 antibodies and anti-Nectin-4 antibody fragments are derived from a mouse. In some embodiments, anti-Nectin-4 antibodies and anti-Nectin-4 antibody fragments are humanized using any technique known in the art that is used to humanize antibodies.

In some embodiments, the anti-Nectin-4 antibodies comprise a heavy chain that is at least 90% identical to any one of SEQ ID NOs: 144, 145, 146, 147, 148, 149, 150, 151, 152, 153, 154, 155, 156, 157, 158, or 159, for example, about 90% identical, about 91% identical, about 92% identical, about 93% identical, about 94% identical, about 95% identical, about 96% identical, about 97% identical, about 98% identical, or about 99% identical to any one of SEQ ID NOs: 144, 145, 146, 147, 148, 149, 150, 151, 152, 153, 154, 155, 156, 157, 158, or 159. In some embodiments, the anti-Nectin-4 antibodies comprise a heavy chain that comprises any one of SEQ ID NOs: 144, 145, 146, 147, 148, 149, 150, 151, 152, 153, 154, 155, 156, 157, 158, or 159.

In some embodiments, the anti-Nectin-4 antibodies comprise a light chain at least 90% identical to any one of SEQ ID NOs: 160, 161, 162, 163, 164, 165, 166, 167, 168, 169, 170, 171, 172, 173, 174, 175, and 176, for example, about 90% identical, about 91% identical, about 92% identical, about 93% identical, about 94% identical, about 95% identical, about 96% identical, about 97% identical, about 98% identical, or about 99% identical to any one of SEQ ID NOs: 160, 161, 162, 163, 164, 165, 166, 167, 168, 169, 170, 171, 172, 173, 174, 175, and 176. In some embodiments, the Nectin-4 antibodies comprise a light chain that comprises any one of SEQ ID NOs: 160, 161, 162, 163, 164, 165, 166, 167, 168, 169, 170, 171, 172, 173, 174, 175, and 176.

### Polypeptide Complexes

In an aspect, disclosed herein are polypeptide chains that include an anti-Nectin-4 antibody fragment. In some embodiments, the polypeptide chain comprises a heavy chain of an anti-Nectin-4 antibody. In some embodiments, the polypeptide chain comprises a light chain of an anti-Nectin-4 antibody. In some embodiments, the polypeptide chain comprises a heavy chain of an anti-Nectin-4 antibody.

In an aspect, disclosed herein are polypeptide complexes that comprise a first polypeptide chain and a second polypeptide chain. In some embodiments, the first polypeptide chain comprises a heavy chain of an anti-Nectin-4 antibody. In some embodiments, the second polypeptide chain comprises a light chain of an anti-Nectin-4 antibody.

In an aspect, disclosed herein are polypeptide chains and polypeptide complexes that contain one or more antibodies or antibody fragments capable of binding to a tumor cell antigen and an effector cell antigen.

In some embodiments, the effector cell antigen comprises a Nectin-4 heavy chain variable region. In some embodiments, the Nectin-4 heavy chain variable region comprises at least 90% sequence identity to any one or more of SEQ ID NOs: 48, 49, 50, 51, 55, 56, 57, 58, 62, 63, 64, 65, 69, 70, 71, and 72, for example, about 90% sequence identity, about 91% sequence identity, about 92% sequence identity, about 93% sequence identity, about 94% sequence identity, about 95% sequence identity, about 96% sequence identity, about 97% sequence identity, about 98% sequence identity, or about 99% sequence identity to any one or more of SEQ ID NOs: 48, 49, 50, 51, 55, 56, 57, 58, 62, 63, 64, 65, 69, 70, 71, and 72, wherein the polypeptide complex comprising the Nectin-4 heavy chain variable region binds Nectin-4. In some embodiments, the Nectin-4 heavy chain variable region comprises any one or more of SEQ ID NOs: 48, 49, 50, 51, 55, 56, 57, 58, 62, 63, 64, 65, 69, 70, 71, and 72.

In some embodiments, the Nectin-4 heavy chain variable region comprises a complementarity determining region 1 (CDRH1), wherein CDRH1 comprises any one or more of SEQ ID NOs: 52, 59, 66, and 15. In some embodiments, CDRH1 comprises a sequence that has one or two amino acid substitutions relative to any one or more of SEQ ID NOs: 52, 59, 66, and 15, wherein the polypeptide complex comprising the Nectin-4 heavy chain variable region binds to Nectin-4. In some embodiments, the one or two amino acid substitutions are conservative amino acid substitutions.

In some embodiments, the Nectin-4 heavy chain comprises CDRH2, wherein CDRH2 comprises any one or more of SEQ ID NOs: 53, 60, 67, and 16. In some embodiments, CDRH2 comprises a sequence that has one or two amino acid substitutions relative to any one or more of SEQ ID NOs: 53, 60, 67, and 16, wherein the polypeptide complex comprising the Nectin-4 heavy chain binds to Nectin-4. In some embodiments, the one or two amino acid substitutions are conservative amino acid substitutions.

In some embodiments, the Nectin-4 heavy chain comprises CDRH3, wherein CDRH3 comprises any one or more of SEQ ID NOs: 54, 61, 68, and 17. In some embodiments, CDRH3 comprises a sequence that has one or two amino acid substitutions relative to any one or more of SEQ ID NOs: 54, 61, 68, and 17, wherein the polypeptide complex comprising the Nectin-4 heavy chain binds to Nectin-4. In some embodiments, the one or two amino acid substitutions are conservative amino acid substitutions.

In some embodiments, the effector cell antigen comprises a Nectin-4 light chain variable region. In some embodiments, the Nectin-4 light chain variable region comprises at least 90% sequence identity to any one or more of SEQ ID NOs: 24, 25, 26, 27, 31, 32, 33, 34, 38, 39, 40, 41, 43, 44, 45, 46 and 47, for example, about 90% sequence identity, about 91% sequence identity, about 92% sequence identity, about 93% sequence identity, about 94% sequence identity, about 95% sequence identity, about 96% sequence identity, about 97% sequence identity, about 98% sequence identity, or about 99% sequence identity to any one or more of SEQ ID NOs: 24, 25, 26, 27, 31, 32, 33, 34, 38, 39, 40, 41, 43, 44, 45, 46, and 47, wherein the polypeptide complex comprising the Nectin-4 light chain variable region binds to Nectin-4. In some embodiments, the Nectin-4 light chain variable region comprises any one or more of SEQ ID NOs: 24, 25, 26, 27, 31, 32, 33, 34, 38, 39, 40, 41, 43, 44, 45, 46, and 47.

In some embodiments, the Nectin-4 light chain variable region comprises CDRL1, wherein CDRL1 comprises any one or more of SEQ ID NOs: 28, 35, 42, and 20. In some embodiments, CDRL1 comprises a sequence that has one or two amino acid substitutions relative to any one or more of SEQ ID NOs: 28, 35, 42, and 20, wherein the polypeptide complex comprising the Nectin-4 light chain variable region binds to Nectin-4. In some embodiments, the one or two amino acid substitutions are conservative amino acid substitutions.

In some embodiments, the Nectin-4 light chain comprises CDRL2, wherein CDRL2 comprises any one or more of SEQ ID NOs: 29, 36, and 21. In some embodiments, CDRL2 comprises a sequence that has one or two amino acid substitutions relative to any one or more of SEQ ID NOs: 29, 36, and 21, wherein the polypeptide complex comprising the Nectin-4 light chain binds Nectin-4. In some embodiments, the one or two amino acid substitutions are conservative amino acid substitutions.

In some embodiments, the Nectin-4 heavy chain comprises CDRL3, wherein CDRL3 comprises any one or more of SEQ ID NOs: 30, 37, and 22. In some embodiments, CDRL3 comprises a sequence that has one or two amino acid substitutions relative to any one or more of SEQ ID NOs: 30, 37, and 22, wherein the polypeptide complex comprising the Nectin-4 heavy chain binds Nectin-4. In some embodiments, the one or two amino acid substitutions are conservative amino acid substitutions.

In some embodiments, the polypeptide chains and polypeptide complexes comprise one or more mask domains.

In some embodiments, the one or more mask domains obstruct one or more of the antibodies or antibody fragments from binding to a tumor cell antigen. In some embodiments, the one or more mask domains bind to the one or more antibodies or antibody fragments thereby obstructing the one or more of the antibodies or antibody fragments from binding to a tumor cell antigen. In some embodiments, the tumor cell antigen comprises Nectin-4. In some embodiments, the mask domain comprises any one of SEQ ID NOs: 18, 23, 125, and 126. In some embodiments, the mask domain comprises a sequence that has at least one or two amino acid substitutions relative to any one or more of SEQ ID NOs: 18, 23, 125, and 126, wherein the mask domains bind to one or more antibodies or antibody fragments that bind Nectin-4. In some embodiments, the at least one or two amino acid substitutions are conservative amino acid substitutions.

In some embodiments, the one or more mask domains obstruct one or more antibodies or antibody fragments from binding to an effector cell antigen. In some embodiments, the one or more mask domains binds to one or more antibodies or antibody fragments, thereby preventing binding or reducing binding affinity of the one or more antibodies or antibody fragments. In some embodiments, the effector cell antigen comprises CD3. In some embodiments, the mask domain comprises SEQ ID NO: 7. In some embodiments, the mask domain comprises a sequence that has at least one or two amino acid substitutions relative to SEQ ID NO: 7, wherein the mask domain binds to one or more antibodies or antibody fragments that target CD3. In some embodiments, the at least one or two amino acid substitutions are conservative amino acid substitutions.

In some embodiments, the one or more mask domains are cleaved from the polypeptide chain or polypeptide complex in a tumor cell microenvironment (TME). In some embodiments, cleaving of the one or more mask domains results in binding of the one or more antibodies or antibody fragments to a tumor cell antigen. In some embodiments, cleaving of the one or more mask domains results in binding of antibodies or antibody fragments to Nectin-4. In some embodiments, cleaving of the one or more masks results in binding of one or more antibodies or antibody fragments to an effector cell antigen. In some embodiments, cleaving of the one or more mask domains results in binding of the one or more antibodies or antibody fragments to CD3.

In some embodiments, the polypeptide chains and polypeptide complexes comprise one or more protease cleavage sites. In some embodiments, cleavage of the one or more protease cleavage sites results in removal of the one or more mask domains. In some embodiments, cleavage of the one or more cleavage sites occurs in a tumor microenvironment.

### Polypeptide Chains Comprising Formula I

In an aspect, a polypeptide chain is provided comprising the following formula: HA-M₁-P₁-EA-TH (Formula I). In some embodiments, a polypeptide chain comprising Formula I is part of a polypeptide complex comprising Formula II.

In some embodiments, HA comprises an antibody or antibody fragment capable of binding a half-life extending molecule. In some embodiments, the half-life extending molecule comprises albumin (ALB). In some embodiments, HA comprises an antibody or antibody fragment comprising an anti-ALB heavy chain variable region comprising CDRH1, CDRH2, and CDRH3, wherein CDRH1 comprises SEQ ID NO: 4, CDRH2 comprises SEQ ID NO: 5, and CDRH3 comprises SEQ ID NO: 6. In some embodiments, CDRH1 comprises a sequence with one or two amino acid substitutions relative to SEQ ID NO: 4, wherein the polypeptide complex comprising the anti-ALB heavy chain variable region binds ALB. In some embodiments, CDRH2 comprises a sequence with one or two amino acid substitutions relative to SEQ ID NO: 5, wherein the polypeptide complex comprising the anti-ALB heavy chain variable region binds binds ALB. In some embodiments, CDRH3 comprises a sequence with one or two amino acid substitutions relative to SEQ ID NO: 6, wherein the polypeptide complex comprising the anti-ALB heavy chain variable region binds ALB. In some embodiments, the one or two amino acid substitutions in CDRH1, CDRH2, and/or CDRH3 are conservative amino acid substitutions.

In some embodiments, M₁ comprises a first mask domain. In some embodiments, M₁ comprises SEQ ID NO: 7. In some embodiments, M₁ comprises a sequence with at least one or two amino acid substitutions relative to SEQ ID NO: 7, wherein the sequence binds one or more antibodies or antibody fragments that targets CD3.

In some embodiments, P comprises a protease cleavage site. In some embodiments, P comprises SEQ ID NO: 8. In some embodiments, P comprises a sequence with one or two amino acid substitutions relative to SEQ ID NO: 8, wherein the sequence is capable of being targeted by a protease.

In some embodiments, EA comprises an antibody or antibody fragment capable of binding an effector cell antigen. In some embodiments, EA comprises a light chain variable region capable of binding an effector cell antigen. In some embodiments, EA comprises a heavy chain variable region capable of binding an effector cell antigen. In some embodiments, EA comprises a light chain variable region and a heavy chain variable region that bind an effector cell antigen.

In some embodiments, the effector cell antigen comprises CD3. In some embodiments, EA comprises an antibody or antibody fragment comprising an anti-CD3 heavy chain variable region comprising CDRH1, CDRH2, and CDRH3, wherein CDRH1 comprises SEQ ID NO: 9, CDRH2 comprises SEQ ID NO: 10, and CDRH3 comprises SEQ ID NO: 11. In some embodiments, CDRH1 comprises a sequence with one or two amino acid substitutions relative to SEQ ID NO: 9, wherein the polypeptide complex comprising the anti-CD3 heavy chain variable region binds CD3. In some embodiments, CDRH2 comprises a sequence with one or two amino acid substitutions relative to SEQ ID NO: 10, wherein the polypeptide complex comprising the anti-CD3 heavy chain variable region binds CD3. In some embodiments, CDRH3 comprises a sequence with one or two amino acid substitutions relative to SEQ ID NO: 11, wherein the anti-CD3 polypeptide complex comprising the heavy chain variable region binds CD3. In some embodiments, the one or two amino acid substitutions in CDRH1, CDRH2, and/or CDRH3 are conservative amino acid substitutions.

In some embodiments, EA comprises an antibody or antibody fragment comprising an anti-CD3 light chain variable region comprising CDRL1, CDRL2, and CDRL3, wherein CDRL1 comprises SEQ ID NO: 12, CDRL2 comprises SEQ ID NO: 13, and CDRL3 comprises SEQ ID NO: 14. In some embodiments, CDRL1 comprises a sequence with one or two amino acid substitutions relative to SEQ ID NO: 12, wherein the polypeptide complex comprising the anti-CD3 light chain variable region binds to CD3. In some embodiments, CDRL2 comprises a sequence with one or two amino acid substitutions relative to SEQ ID NO: 13, wherein the polypeptide complex comprising the anti-CD3 light chain variable region binds CD3. In some embodiments, CDRL3 comprises a sequence with one or two amino acid substitutions relative to SEQ ID NO: 14, wherein the polypeptide complex comprising the anti-CD3 light chain variable region binds CD3. In some embodiments, the one or two amino acid substitutions in CDRL1, CDRL2, and/or CDRL3 are conservative amino acid substitutions.

In some embodiments, TH comprises an antibody or an antibody fragment capable of binding to a tumor cell antigen. In some embodiments, TH comprises a heavy chain variable region capable of binding to a tumor cell antigen.

In some embodiments, the tumor cell antigen is Nectin-4.

In some embodiments, TH comprises an anti-Nectin-4 heavy chain variable region comprising CDRH1, CDRH2, and CDRH3, wherein CDRH1 comprises SEQ ID NO: 15, CDRH2 comprises SEQ ID NO: 16, and CDRH3 comprises SEQ ID NO: 17. In some embodiments, CDRH1 comprises a sequence with one or two amino acid substitutions relative to SEQ ID NO: 15, wherein the polypeptide complex comprising the anti-Nectin-4 heavy chain variable region binds Nectin-4. In some embodiments, CDRH2 comprises a sequence with one or two amino acid substitutions relative to SEQ ID NO: 16, wherein the polypeptide complex comprising the anti-Nectin-4 heavy chain variable region binds Nectin-4. In some embodiments, CDRH3 comprises a sequence with one or two amino acid substitutions relative to SEQ ID NO: 17, wherein the anti-Nectin-4 polypeptide complex comprising the heavy chain variable region binds Nectin-4. In some embodiments, the one or two amino acid substitutions in CDRH1, CDRH2, and/or CDRH3 are conservative amino acid substitutions.

In some embodiments, TH comprises an anti-Nectin-4 heavy chain variable region comprising CDRH1, CDRH2, and CDRH3, wherein CDRH1 comprises SEQ ID NO: 52, CDRH2 comprises SEQ ID NO: 53, and CDRH3 comprises SEQ ID NO: 54. In some embodiments, CDRH1 comprises a sequence with one or two amino acid substitutions relative to SEQ ID NO: 52, wherein the polypeptide complex comprising the anti-Nectin-4 heavy chain variable region binds Nectin-4. In some embodiments, CDRH2 comprises a sequence with one or two amino acid substitutions relative to SEQ ID NO: 53, wherein the polypeptide complex comprising the anti-Nectin-4 heavy chain variable region binds to Nectin-4. In some embodiments, CDRH3 comprises a sequence with one or two amino acid substitutions relative to SEQ ID NO: 54, wherein the polypeptide complex comprising the anti-Nectin-4 heavy chain variable region binds to Nectin-4. In some embodiments, the one or two amino acid substitutions in CDRH1, CDRH2, and/or CDRH3 are conservative amino acid substitutions.

In some embodiments, TH comprises an anti-Nectin-4 heavy chain variable region comprising CDRH1, CDRH2, and CDRH3, wherein CDRH1 comprises SEQ ID NO: 59, CDRH2 comprises SEQ ID NO: 60, and CDRH3 comprises SEQ ID NO: 61. In some embodiments, CDRH1 comprises a sequence with one or two amino acid substitutions relative to SEQ ID NO: 59, wherein the polypeptide complex comprising the anti-Nectin-4 heavy chain variable region binds Nectin-4. In some embodiments, CDRH2 comprises a sequence with one or two amino acid substitutions relative to SEQ ID NO: 60, wherein the polypeptide complex comprising the anti-Nectin-4 heavy chain variable region binds Nectin-4. In some embodiments, CDRH3 comprises a sequence with one or two amino acid substitutions relative to SEQ ID NO: 61, wherein the polypeptide complex comprising the anti-Nectin-4 heavy chain variable region binds to Nectin-4. In some embodiments, the one or two amino acid substitutions in CDRH1, CDRH2, and/or CDRH3 are conservative amino acid substitutions.

In some embodiments, TH comprises an anti-Nectin-4 heavy chain variable region comprising CDRH1, CDRH2, and CDRH3, wherein CDRH1 comprises SEQ ID NO: 66, CDRH2 comprises SEQ ID NO: 67, and CDRH3 comprises SEQ ID NO: 68. In some embodiments, CDRH1 comprises a sequence with one or two amino acid substitutions relative to SEQ ID NO: 66, wherein the polypeptide complex comprising the anti-Nectin-4 heavy chain variable region binds Nectin-4. In some embodiments, CDRH2 comprises a sequence with one or two amino acid substitutions relative to SEQ ID NO: 67, wherein the polypeptide complex comprising the anti-Nectin-4 heavy chain variable region binds Nectin-4. In some embodiments, CDRH3 comprises a sequence with one or two amino acid substitutions relative to SEQ ID NO: 68, wherein the polypeptide complex comprising the anti-Nectin-4 heavy chain variable region binds Nectin-4. In some embodiments, the one or two amino acid substitutions in CDRH1, CDRH2, and/or CDRH3 are conservative amino acid substitutions.

In some embodiments, TH comprises an anti-Nectin-4 heavy chain variable region comprising a sequence that has at least 90% sequence identity to any one of SEQ ID NOs: 48, 49, 50, 51, 55, 56, 57, 58, 62, 63, 64, 65, 69, 70, 71, and 72, for example, at least 90% sequence identity, at least 91% sequence identity, at least 92% sequence identity, at least 93% sequence identity, at least 94% sequence identity, at least 95% sequence identity, at least 96% sequence identity at least 97% sequence identity, at least 98% sequence identity, or at least 99% sequence identity to any one of SEQ ID NOs: 48, 49, 50, 51, 55, 56, 57, 58, 62, 63, 64, 65, 69, 70, 71, and 72, wherein the polypeptide complex comprising the anti-Nectin-4 heavy chain variable region binds to Nectin-4. In some embodiments, the anti-Nectin-4 heavy chain variable region comprises any one of SEQ ID NOs: 48, 49, 50, 51, 55, 56, 57, 58, 62, 63, 64, 65, 69, 70, 71, and 72.

In some embodiments, TH comprises an anti-Nectin-4 heavy chain comprising a sequence that has at least 90% sequence identity to any one or more of SEQ ID NOs: 144, 145, 146, 147, 148, 149, 150, 151, 152, 153, 154, 155, 156, 157, 158, and 159, for example, at least 90% sequence identity, at least 91% sequence identity, at least 92% sequence identity, at least 93% sequence identity, at least 94% sequence identity, at least 95% sequence identity, at least 96% sequence identity at least 97% sequence identity, at least 98% sequence identity, or at least 99% sequence identity to any one of SEQ ID NOs: 144, 145, 146, 147, 148, 149, 150, 151, 152, 153, 154, 155, 156, 157, 158, and 159, wherein the polypeptide complex comprising the anti-Nectin-4 heavy chain binds Nectin-4. In some embodiments, the anti-Nectin-4 heavy chain comprises any one of SEQ ID NOs: 144, 145, 146, 147, 148, 149, 150, 151, 152, 153, 154, 155, 156, 157, 158, and 159.

### Polypeptide Chains Comprising Formula II

In an aspect, a polypeptide chain is provided comprising the following formula: M₂-P₂-TL (Formula II). In some embodiments, a polypeptide chain comprising Formula II is part of a polypeptide complex comprising Formula I.

In some embodiments, M₂ comprises a mask domain. In some embodiments, M₂ comprises SEQ ID NO: 18. In some embodiments, M₂ comprises a sequence with at least one or two amino acid substitutions relative to SEQ ID NO: 18, wherein the sequence binds one or more antibodies or antibody fragments that target Nectin-4. In some embodiments M₂ comprises any one of SEQ ID NOs: 23, 125, and 126. In some embodiments, M₂ comprises a sequence with at least one or two amino acid substitutions relative to any one of SEQ ID NOs: 23, 125, and 126, wherein the sequence is binds one or more antibodies or antibody fragments that target Nectin-4.

In some embodiments, P₂ comprises a second protease cleavage site. In some embodiments, P₂ comprises SEQ ID NO: 19. In some embodiments, P₂ comprises a sequence with one or two amino acid substitutions relative to SEQ ID NO: 19, wherein the sequence is capable of being targeted by a protease.

In some embodiments, TL comprises an antibody or an antibody fragment. In some embodiments, TL comprises a light chain variable region that binds a tumor cell antigen. In some embodiments, the tumor cell antigen comprises Nectin-4.

In some embodiments, TL comprises an anti-Nectin-4 light chain variable region comprising CDRL1, CDRL2, and CDRL3, wherein CDRL1 comprises SEQ ID NO: 20, CDRL2 comprises SEQ ID NO: 21, and CDRL3 comprises SEQ ID NO: 22. In some embodiments, CDRL1 comprises a sequence with one or two amino acid substitutions relative to SEQ ID NO: 20, wherein the polypeptide complex comprising the anti-Nectin-4 light chain variable region is binds Nectin-4. In some embodiments, CDRL2 comprises a sequence with one or two amino acid substitutions relative to SEQ ID NO: 21, wherein the polypeptide complex comprising the anti-Nectin-4 light chain variable region binds Nectin-4. In some embodiments, CDRL3 comprises a sequence with one or two amino acid substitutions relative to SEQ ID NO: 22, wherein the polypeptide complex comprising the anti-Nectin-4 light chain variable region binds Nectin-4. In some embodiments, the one or two amino acid substitutions in CDRL1, CDRL2, and/or CDRL3 are conservative amino acid substitutions.

In some embodiments, TL comprises an anti-Nectin-4 light chain variable region comprising CDRL1, CDRL2, and CDRL3, wherein CDRL1 comprises SEQ ID NO: 28, CDRL2 comprises SEQ ID NO: 29, and CDRL3 comprises SEQ ID NO: 30. In some embodiments, CDRL1 comprises a sequence with one or two amino acid substitutions relative to SEQ ID NO: 28, wherein the polypeptide complex comprising the anti-Nectin-4 light chain variable region binds Nectin-4. In some embodiments, CDRL2 comprises a sequence with one or two amino acid substitutions relative to SEQ ID NO: 29, wherein the polypeptide complex comprising the anti-Nectin-4 light chain variable region binds to Nectin-4. In some embodiments, CDRL3 comprises a sequence with one or two amino acid substitutions relative to SEQ ID NO: 30, wherein the polypeptide complex comprising the anti-Nectin-4 light chain variable region binds to Nectin-4. In some embodiments, the one or two amino acid substitutions in CDRL1, CDRL2, and/or CDRL3 are conservative amino acid substitutions.

In some embodiments, TL comprises an anti-Nectin-4 light chain variable region comprising CDRL1, CDRL2, and CDRL3, wherein CDRL1 comprises SEQ ID NO: 35, CDRL2 comprises SEQ ID NO: 36, and CDRL3 comprises SEQ ID NO: 37. In some embodiments, CDRL1 comprises a sequence with one or two amino acid substitutions relative to SEQ ID NO: 35, wherein the polypeptide complex comprising the anti-Nectin-4 light chain variable region binds Nectin-4. In some embodiments, CDRL2 comprises a sequence with one or two amino acid substitutions relative to SEQ ID NO: 36, wherein the polypeptide complex comprising the anti-Nectin-4 light chain variable region binds Nectin-4. In some embodiments, CDRL3 comprises a sequence with one or two amino acid substitutions relative to SEQ ID NO: 37, wherein the polypeptide complex comprising the anti-Nectin-4 light chain variable region binds Nectin-4. In some embodiments, the one or two amino acid substitutions in CDRL1, CDRL2, and/or CDRL3 are conservative amino acid substitutions.

In some embodiments, TL comprises an anti-Nectin-4 light chain variable region comprising CDRL1, CDRL2, and CDRL3, wherein CDRL1 comprises SEQ ID NO: 42, CDRL2 comprises SEQ ID NO: 29, and CDRL3 comprises SEQ ID NO: 30. In some embodiments, CDRL1 comprises a sequence with one or two amino acid substitutions relative to SEQ ID NO: 42, wherein the polypeptide complex comprising the anti-Nectin-4 light chain variable region binds Nectin-4. In some embodiments, CDRL2 comprises a sequence with one or two amino acid substitutions relative to SEQ ID NO: 29, wherein the polypeptide complex comprising the anti-Nectin-4 light chain variable region binds Nectin-4. In some embodiments, CDRL3 comprises a sequence with one or two amino acid substitutions relative to SEQ ID NO: 30, wherein the polypeptide complex comprising the anti-Nectin-4 light chain variable region binds Nectin-4. In some embodiments, the one or two amino acid substitutions in CDRL1, CDRL2, and/or CDRL3 are conservative amino acid substitutions.

In some embodiments, the anti-Nectin-4 light chain variable region comprises a sequence that has at least 90% sequence identity to any one of SEQ ID NOs: 24, 25, 26, 27, 31, 32, 33, 34, 38, 39, 40, 41, 43, 44, 45, 46, and 47, for example, at least 90% sequence identity, at least 91% sequence identity, at least 92% sequence identity, at least 93% sequence identity, at least 94% sequence identity, at least 95% sequence identity, at least 96% sequence identity at least 97% sequence identity, at least 98% sequence identity, or at least 99% sequence identity to any one of SEQ ID NOs: 24, 25, 26, 27, 31, 32, 33, 34, 38, 39, 40, 41, 43, 44, 45, 46, and 47, wherein the polypeptide complex comprising the anti-Nectin-4 light chain variable binds Nectin-4. In some embodiments, the anti-Nectin-4 light chain variable region comprises any one of SEQ ID NOs: 24, 25, 26, 27, 31, 32, 33, 34, 38, 39, 40, 41, 43, 44, 45, 46, and 47.

In some embodiments, TH comprises an anti-Nectin-4 light chain comprising a sequence that has at least 90% sequence identity to any one or more of SEQ ID NOs: 127, 128, 129, 130, 131, 132, 133, 134, 135, 136, 137, 138, 139, 140, 141, 142, and 143, for example, at least 90% sequence identity, at least 91% sequence identity, at least 92% sequence identity, at least 93% sequence identity, at least 94% sequence identity, at least 95% sequence identity, at least 96% sequence identity at least 97% sequence identity, at least 98% sequence identity, or at least 99% sequence identity to any one of SEQ ID NOs: 127, 128, 129, 130, 131, 132, 133, 134, 135, 136, 137, 138, 139, 140, 141, 142, and 143, wherein the polypeptide complex comprising the anti-Nectin-4 light chain binds to Nectin-4. In some embodiments, the anti-Nectin-4 light chain comprises any one of SEQ ID NOs: 127, 128, 129, 130, 131, 132, 133, 134, 135, 136, 137, 138, 139, 140, 141, 142, and 143.

### Exemplary Embodiments of Polypeptide Complexes that Include Formula I and Formula II

In an aspect, a polypeptide complex is provided comprising a first polypeptide chain and a second polypeptide chain, wherein the first polypeptide chain comprises formula I (HA-M₁-P₁-EA-TH) and the second polypeptide chain comprises formula II (M₂-P₂-TL).

In some embodiments, the polypeptide complex that includes formula I and formula II is expressed in a host cell. In some embodiments, the host cell is a non-cancerous cell. In some embodiments, the host cell is an effector cell. In some embodiments, the effector cell is a T cell. In some embodiments, the effector cell is a PBMC.

In some embodiments M₁ and M₂ are mask sequences. In some embodiments, a polypeptide complex comprising M₁ and M₂ result in reduced toxicity on the host cell relative to a similar polypeptide complex that lacks M₁ and M₂. In some embodiments, the reduction in toxicity of polypeptide complexes described herein relative to equivalent polypeptide complexes that lack M₁ and M₂ is between about a 10× reduction in toxicity and a 100× reduction in toxicity, for example, between about a 10× reduction in toxicity and about a 20× reduction in toxicity, between about a 20× reduction in toxicity and about a 30× reduction in toxicity, between about a 30× reduction in toxicity and about a 40× reduction in toxicity, between about a 40× reduction in toxicity and about a 50× reduction in toxicity, between about a 50× reduction in toxicity and about a 60× reduction in toxicity, between about a 70× reduction in toxicity and about a 80× reduction in toxicity, between about a 80× reduction in toxicity and about a 90× reduction in toxicity, or between about a 90× reduction in toxicity and about a 100× reduction in toxicity.

In some embodiments M₁ comprises a mask domain and EA comprises an antibody or antibody fragment that binds an effector cell antigen. In some embodiments, M₁ binds to EA. In some embodiments, M₁ binds to EA outside of a tumor microenvironment (TME). In some embodiments, binding of M₁ to EA prevents EA from binding an effector cell antigen. In some embodiments, EA is prevented from binding an effector cell antigen outside of a TME.

In some embodiments, M₂ comprises a mask domain and TL comprises an antibody or antibody fragment that binds a tumor cell antigen. In some embodiments, M₂ binds to TL. In some embodiments, M₂ binds to TL outside of a TME. In some embodiments, binding of M₂ to TL prevents TL from binding a tumor cell antigen. In some embodiments, TL is prevented from binding a tumor cell antigen outside of a TME.

In some embodiments, HA comprises an antibody or antibody fragment that binds a half-life extending molecule. In some embodiments, the half-life extending molecule comprises albumin (ALB). In some embodiments, HA comprises an antibody or antibody fragment comprising an anti-ALB heavy chain variable region comprising CDRH1, CDRH2, and CDRH3, wherein CDRH1 comprises SEQ ID NO: 4, CDRH2 comprises SEQ ID NO: 5, and CDRH3 comprises SEQ ID NO: 6. In some embodiments, CDRH1 comprises a sequence with one or two amino acid substitutions relative to SEQ ID NO: 4, wherein the polypeptide complex comprising the anti-ALB heavy chain variable region binds to ALB. In some embodiments, CDRH2 comprises a sequence with one or two amino acid substitutions relative to SEQ ID NO: 5, wherein the polypeptide complex comprising the anti-ALB heavy chain variable region binds ALB. In some embodiments, CDRH3 comprises a sequence with one or two amino acid substitutions relative to SEQ ID NO: 6, wherein the polypeptide complex comprising the anti-ALB heavy chain variable region binds to ALB. In some embodiments, the one or two amino acid substitutions in CDRH1, CDRH2, and/or CDRH3 are conservative amino acid substitutions.

In some embodiments, M₁ comprises SEQ ID NO: 7. In some embodiments, M₁ comprises a sequence with at least one or two amino acid substitutions relative to SEQ ID NO: 7, wherein the sequence binds to an antibody or antibody fragment that targets CD3.

In some embodiments, P comprises a protease cleavage site. In some embodiments, P comprises SEQ ID NO: 8. In some embodiments, P comprises a sequence with at least one or two amino acid substitutions relative to SEQ ID NO: 8, wherein the sequence is capable of being targeted by a protease.

In some embodiments, EA comprises a light chain variable region that binds an effector cell antigen. In some embodiments, EA comprises a heavy chain variable region that binds an effector cell antigen. In some embodiments, EA comprises a light chain variable region and a heavy chain variable region that binds an effector cell antigen.

In some embodiments, the effector cell antigen comprises CD3. In some embodiments, EA comprises an antibody or antibody fragment comprising an anti-CD3 heavy chain variable region comprising CDRH1, CDRH2, and CDRH3, wherein CDRH1 comprises SEQ ID NO: 9, CDRH2 comprises SEQ ID NO: 10, and CDRH3 comprises SEQ ID NO: 11. In some embodiments, CDRH1 comprises a sequence with one or two amino acid substitutions relative to SEQ ID NO: 9, wherein the polypeptide complex comprising the anti-CD3 heavy chain variable region binds CD3. In some embodiments, CDRH2 comprises a sequence with one or two amino acid substitutions relative to SEQ ID NO: 10, wherein the polypeptide complex comprising the anti-CD3 heavy chain variable region binds CD3. In some embodiments, CDRH3 comprises a sequence with one or two amino acid substitutions relative to SEQ ID NO: 11, wherein the polypeptide complex comprising the anti-CD3 heavy chain variable region binds CD3. In some embodiments, the one or two amino acid substitutions in CDRH1, CDRH2, and/or CDRH3 are conservative amino acid substitutions.

In some embodiments, EA comprises an antibody or antibody fragment comprising an anti-CD3 light chain variable region comprising CDRL1, CDRL2, and CDRL3, wherein CDRL1 comprises SEQ ID NO: 12, CDRL2 comprises SEQ ID NO: 13, and CDRL3 comprises SEQ ID NO: 14. In some embodiments, CDRL1 comprises a sequence with one or two amino acid substitutions relative to SEQ ID NO: 12, wherein the polypeptide complex comprising the anti-CD3 light chain variable region is capable of binding to CD3. In some embodiments, CDRL2 comprises a sequence with one or two amino acid substitutions relative to SEQ ID NO: 13, wherein the polypeptide complex comprising the anti-CD3 light chain variable region binds CD3. In some embodiments, CDRL3 comprises a sequence with one or two amino acid substitutions relative to SEQ ID NO: 14, wherein the polypeptide complex comprising the anti-CD3 light chain variable region binds CD3. In some embodiments, the one or two amino acid substitutions in CDRL1, CDRL2, and/or CDRL3 are conservative amino acid substitutions.

In some embodiments, EA comprises an antibody or antibody fragment comprising an anti-CD3 heavy chain variable region and an anti-CD3 light chain variable region. In some embodiments, the anti-CD3 heavy chain variable region comprises CDRH1, CDRH2, and CDRH3, wherein CDRH1 comprises SEQ ID NO: 9, CDRH2 comprises SEQ ID NO: 10, and CDRH3 comprises SEQ ID NO: 11. In some embodiments, CDRH1 comprises a sequence with one or two amino acid substitutions relative to SEQ ID NO: 9, wherein the polypeptide complex comprising the anti-CD3 heavy chain variable region binds CD3. In some embodiments, CDRH2 comprises a sequence with one or two amino acid substitutions relative to SEQ ID NO: 10, wherein the polypeptide complex comprising the anti-CD3 heavy chain variable region binds CD3. In some embodiments, CDRH3 comprises a sequence with one or two amino acid substitutions relative to SEQ ID NO: 11, wherein the polypeptide complex comprising the anti-CD3 heavy chain variable region binds CD3. In some embodiments, the anti-CD3 light chain variable region comprises CDRL1, CDRL2, and CDRL3, wherein CDRL1 comprises SEQ ID NO: 12, CDRL2 comprises SEQ ID NO: 13, and CDRL3 comprises SEQ ID NO: 14. In some embodiments, CDRL1 comprises a sequence with one or two amino acid substitutions relative to SEQ ID NO: 12, wherein the polypeptide complex comprising the anti-CD3 light chain variable region binds to CD3. In some embodiments, CDRL2 comprises a sequence with one or two amino acid substitutions relative to SEQ ID NO: 13, wherein the polypeptide complex comprising the anti-CD3 light chain variable region binds to CD3. In some embodiments, CDRL3 comprises a sequence with one or two amino acid substitutions relative to SEQ ID NO: 14, wherein the polypeptide complex comprising the anti-CD3 light chain variable region binds CD3. In some embodiments, the one or two amino acid substitutions in CDRH1, CDRH2, CDRH3, CDRL1, CDRL2, and/or CDRL3 are conservative amino acid substitutions.

In some embodiments, TH comprises an antibody or an antibody fragment that binds to a tumor cell antigen. In some embodiments, TH comprises a heavy chain variable region that binds to a tumor cell antigen. In some embodiments, the tumor cell antigen is Nectin-4.

In some embodiments, TH comprises an anti-Nectin-4 heavy chain variable region comprising CDRH1, CDRH2, and CDRH3, wherein CDRH1 comprises SEQ ID NO: 15, CDRH2 comprises SEQ ID NO: 16, and CDRH3 comprises SEQ ID NO: 17. In some embodiments, CDRH1 comprises a sequence with one or two amino acid substitutions relative to SEQ ID NO: 15, wherein the polypeptide complex comprising the anti-Nectin-4 heavy chain variable region binds Nectin-4. In some embodiments, CDRH2 comprises a sequence with one or two amino acid substitutions relative to SEQ ID NO: 16, wherein the polypeptide complex comprising the anti-Nectin-4 heavy chain variable region binds Nectin-4. In some embodiments, CDRH3 comprises a sequence with one or two amino acid substitutions relative to SEQ ID NO: 17, wherein the polypeptide complex comprising the anti-Nectin-4 heavy chain variable region binds to Nectin-4. In some embodiments, the one or two amino acid substitutions in CDRH1, CDRH2, and/or CDRH3, are conservative amino acid substitutions.

In some embodiments, TH comprises an anti-Nectin-4 heavy chain variable region comprising CDRH1, CDRH2, and CDRH3, wherein CDRH1 comprises SEQ ID NO: 52, CDRH2 comprises SEQ ID NO: 53, and CDRH3 comprises SEQ ID NO: 54. In some embodiments, CDRH1 comprises a sequence with one or two amino acid substitutions relative to SEQ ID NO: 52, wherein the polypeptide complex comprising the anti-Nectin-4 heavy chain variable region binds Nectin-4. In some embodiments, CDRH2 comprises a sequence with one or two amino acid substitutions relative to SEQ ID NO: 53, wherein the polypeptide complex comprising the anti-Nectin-4 heavy chain variable region binds Nectin-4. In some embodiments, CDRH3 comprises a sequence with one or two amino acid substitutions relative to SEQ ID NO: 54, wherein the polypeptide complex comprising the anti-Nectin-4 heavy chain variable region binds Nectin-4. In some embodiments, the one or two amino acid substitutions in CDRH1, CDRH2, and/or CDRH3, are conservative amino acid substitutions.

In some embodiments, TH comprises an anti-Nectin-4 heavy chain variable region comprising CDRH1, CDRH2, and CDRH3, wherein CDRH1 comprises SEQ ID NO: 59, CDRH2 comprises SEQ ID NO: 60, and CDRH3 comprises SEQ ID NO: 61. In some embodiments, CDRH1 comprises a sequence with one or two amino acid substitutions relative to SEQ ID NO: 59, wherein the polypeptide complex comprising the anti-Nectin-4 heavy chain variable region binds Nectin-4. In some embodiments, CDRH2 comprises a sequence with one or two amino acid substitutions relative to SEQ ID NO: 60, wherein the polypeptide complex comprising the anti-Nectin-4 heavy chain variable region binds Nectin-4. In some embodiments, CDRH3 comprises a sequence with one or two amino acid substitutions relative to SEQ ID NO: 61, wherein the polypeptide complex comprising the anti-Nectin-4 heavy chain variable region binds Nectin-4. In some embodiments, the one or two amino acid substitutions in CDRH1, CDRH2, and/or CDRH3, are conservative amino acid substitutions.

In some embodiments, TH comprises an anti-Nectin-4 heavy chain variable region comprising CDRH1, CDRH2, and CDRH3, wherein CDRH1 comprises SEQ ID NO: 66, CDRH2 comprises SEQ ID NO: 67, and CDRH3 comprises SEQ ID NO: 68. In some embodiments, CDRH1 comprises a sequence with one or two amino acid substitutions relative to SEQ ID NO: 66, wherein the polypeptide complex comprising the anti-Nectin-4 heavy chain variable region binds Nectin-4. In some embodiments, CDRH2 comprises a sequence with one or two amino acid substitutions relative to SEQ ID NO: 67, wherein the polypeptide complex comprising the anti-Nectin-4 heavy chain variable region binds Nectin-4. In some embodiments, CDRH3 comprises a sequence with one or two amino acid substitutions relative to SEQ ID NO: 68, wherein the polypeptide complex comprising the anti-Nectin-4 heavy chain variable region binds Nectin-4. In some embodiments, the one or two amino acid substitutions in CDRH1, CDRH2, and/or CDRH3, are conservative amino acid substitutions.

In some embodiments, the anti-Nectin-4 heavy chain variable region comprises a sequence that has at least 90% sequence identity to any one of SEQ ID NOs: 48, 49, 50, 51, 55, 56, 57, 58, 62, 63, 64, 65, 69, 70, 71, and 72, for example, at least 90% sequence identity, at least 91% sequence identity, at least 92% sequence identity, at least 93% sequence identity, at least 94% sequence identity, at least 95% sequence identity, at least 96% sequence identity at least 97% sequence identity, at least 98% sequence identity, or at least 99% sequence identity to any one of SEQ ID NOs: 48, 49, 50, 51, 55, 56, 57, 58, 62, 63, 64, 65, 69, 70, 71, and 72, wherein the polypeptide complex comprising the anti-Nectin-4 heavy chain variable region binds Nectin-4. In some embodiments, the anti-Nectin-4 heavy chain variable region comprises any one of SEQ ID NOs: 48, 49, 50, 51, 55, 56, 57, 58, 62, 63, 64, 65, 69, 70, 71, and 72.

In some embodiments, TH comprise an anti-Nectin-4 heavy chain comprising a sequence that has at least 90% sequence identity to any one or more of SEQ ID NOs: 144, 145, 146, 147, 148, 149, 150, 151, 152, 153, 154, 155, 156, 157, 158, and 159, for example, at least 90% sequence identity, at least 91% sequence identity, at least 92% sequence identity, at least 93% sequence identity, at least 94% sequence identity, at least 95% sequence identity, at least 96% sequence identity at least 97% sequence identity, at least 98% sequence identity, or at least 99% sequence identity to any one of SEQ ID NOs: 144, 145, 146, 147, 148, 149, 150, 151, 152, 153, 154, 155, 156, 157, 158, and 159, wherein the polypeptide complex comprising the anti-Nectin-4 heavy chain binds Nectin-4. In some embodiments, the anti-Nectin-4 heavy chain comprises any one of SEQ ID NOs: 144, 145, 146, 147, 148, 149, 150, 151, 152, 153, 154, 155, 156, 157, 158, and 159.

In some embodiments, M₂ comprises SEQ ID NO: 18. In some embodiments, M₂ comprises a sequence with at least one or two amino acid substitutions relative to SEQ ID NO: 18, wherein the sequence binds to an antibody or antibody fragment that targets Nectin-4. In some embodiments M₂ comprises any one of SEQ ID NOs: 23, 125, and 126. In some embodiments, M₂ comprises a sequence with at least one or two amino acid substitutions relative to any one of SEQ ID NOs: 23, 125, and 126, wherein the sequence binds to an antibody or antibody fragment that targets Nectin-4.

In some embodiments, P₂ comprises a second protease cleavage site. In some embodiments, P₂ comprises SEQ ID NO: 19. In some embodiments, P₂ comprises a sequence with one or two amino acid substitutions relative to SEQ ID NO: 19, wherein a protease is capable of targeting the sequence.

In some embodiments, TL comprises an antibody or an antibody fragment. In some embodiments, TL comprises a light chain that binds a tumor cell antigen. In some embodiments, the tumor cell antigen comprises Nectin-4.

In some embodiments, TL comprises an anti-Nectin-4 light chain variable region comprising CDRL1, CDRL2, and CDRL3, wherein CDRL1 comprises SEQ ID NO: 20, CDRL2 comprises SEQ ID NO: 21, and CDRL3 comprises SEQ ID NO: 22. In some embodiments, CDRL1 comprises a sequence with one or two amino acid substitutions relative to SEQ ID NO: 20, wherein the polypeptide complex comprising the anti-Nectin-4 light chain variable region binds Nectin-4. In some embodiments, CDRL2 comprises a sequence with one or two amino acid substitutions relative to SEQ ID NO: 21, wherein the polypeptide complex comprising the anti-Nectin-4 light chain variable region binds Nectin-4. In some embodiments, CDRL3 comprises a sequence with one or two amino acid substitutions relative to SEQ ID NO: 22, wherein the polypeptide complex comprising the anti-Nectin-4 light chain variable region binds Nectin-4. In some embodiments, the one or two amino acid substitutions in CDRL1, CDRL2, and/or CDRL3, are conservative amino acid substitutions.

In some embodiments, TL comprises an anti-Nectin-4 light chain variable region comprising CDRL1, CDRL2, and CDRL3, wherein CDRL1 comprises SEQ ID NO: 28, CDRL2 comprises SEQ ID NO: 29, and CDRL3 comprises SEQ ID NO: 30. In some embodiments, CDRL1 comprises a sequence with one or two amino acid substitutions relative to SEQ ID NO: 28, wherein the polypeptide complex comprising the anti-Nectin-4 light chain variable region binds Nectin-4. In some embodiments, CDRL2 comprises a sequence with one or two amino acid substitutions relative to SEQ ID NO: 29, wherein the polypeptide complex comprising the anti-Nectin-4 light chain variable region binds Nectin-4. In some embodiments, CDRL3 comprises a sequence with one or two amino acid substitutions relative to SEQ ID NO: 30, wherein the polypeptide complex comprising the anti-Nectin-4 light chain variable region binds Nectin-4.

In some embodiments, TL comprises an anti-Nectin-4 light chain variable region comprising CDRL1, CDRL2, and CDRL3, wherein CDRL1 comprises SEQ ID NO: 35, CDRL2 comprises SEQ ID NO: 36, and CDRL3 comprises SEQ ID NO: 37. In some embodiments, CDRL1 comprises a sequence with one or two amino acid substitutions relative to SEQ ID NO: 35, wherein the polypeptide complex comprising the anti-Nectin-4 light chain variable region binds Nectin-4. In some embodiments, CDRL2 comprises a sequence with one or two amino acid substitutions relative to SEQ ID NO: 36, wherein the polypeptide complex comprising the anti-Nectin-4 light chain variable region binds Nectin-4. In some embodiments, CDRL3 comprises a sequence one or two amino acid substitutions relative to SEQ ID NO: 37, wherein the polypeptide complex comprising the anti-Nectin-4 light chain variable region binds Nectin-4. In some embodiments, the one or two amino acid substitutions in CDRL1, CDRL2, and/or CDRL3, are conservative amino acid substitutions.

In some embodiments, TL comprises an anti-Nectin-4 light chain variable region comprising CDRL1, CDRL2, and CDRL3, wherein CDRL1 comprises SEQ ID NO: 42, CDRL2 comprises SEQ ID NO: 29, and CDRL3 comprises SEQ ID NO: 30. In some embodiments, CDRL1 comprises a sequence with one or two amino acid substitutions relative to SEQ ID NO: 42, wherein the polypeptide complex comprising the anti-Nectin-4 light chain variable region binds Nectin-4. In some embodiments, CDRL2 comprises a sequence with one or two amino acid substitutions relative to SEQ ID NO: 29, wherein the polypeptide complex comprising the anti-Nectin-4 light chain variable region binds Nectin-4. In some embodiments, CDRL3 comprises a sequence with one or two amino acid substitutions relative to SEQ ID NO: 30, wherein the polypeptide complex comprising the anti-Nectin-4 light chain variable region binds Nectin-4. In some embodiments, the one or two amino acid substitutions in CDRL1, CDRL2, and/or CDRL3, are conservative amino acid substitutions.

In some embodiments, the anti-Nectin-4 light chain variable region comprises a sequence that has at least 90% sequence identity to any one of SEQ ID NOs: 24, 25, 26, 27, 31, 32, 33, 34, 38, 39, 40, 41, 43, 44, 45, 46, and 47, for example, at least 90% sequence identity, at least 91% sequence identity, at least 92% sequence identity, at least 93% sequence identity, at least 94% sequence identity, at least 95% sequence identity, at least 96% sequence identity at least 97% sequence identity, at least 98% sequence identity, or at least 99% sequence identity to any one of SEQ ID NOs: 24, 25, 26, 27, 31, 32, 33, 34, 38, 39, 40, 41, 43, 44, 45, 46, and 47, wherein the polypeptide complex comprising the anti-Nectin-4 light chain variable region binds Nectin-4. In some embodiments, the anti-Nectin-4 light chain variable region comprises any one of SEQ ID NOs: 24, 25, 26, 27, 31, 32, 33, 34, 38, 39, 40, 41, 43, 44, 45, 46, and 47.

In some embodiments, TH comprises an anti-Nectin-4 light chain comprising a sequence that has at least 90% sequence identity to any one or more of SEQ ID NOs: 127, 128, 129, 130, 131, 132, 133, 134, 135, 136, 137, 138, 139, 140, 141, 142, and 143, for example, at least 90% sequence identity, at least 91% sequence identity, at least 92% sequence identity, at least 93% sequence identity, at least 94% sequence identity, at least 95% sequence identity, at least 96% sequence identity at least 97% sequence identity, at least 98% sequence identity, or at least 99% sequence identity to any one of SEQ ID NOs: 127, 128, 129, 130, 131, 132, 133, 134, 135, 136, 137, 138, 139, 140, 141, 142, and 143, wherein the polypeptide complex comprising the anti-Nectin-4 light chain binds Nectin-4. In some embodiments, the anti-Nectin-4 light chain comprises any one of SEQ ID NOs: 127, 128, 129, 130, 131, 132, 133, 134, 135, 136, 137, 138, 139, 140, 141, 142, and 143.

In some embodiments the first polypeptide chain further comprises a leader sequence. In some embodiments, the leader sequence comprises SEQ ID NO: 3. In some embodiments, the leader sequence comprises a sequence that has one or two amino acid substitutions relative to SEQ ID NO: 3. In some embodiments, the leader sequence on the first polypeptide chain is upstream of HA.

In some embodiments the second polypeptide chain further comprises a leader sequence. In some embodiments, the leader sequence comprises SEQ ID NO: 3. In some embodiments, the leader sequence comprises a sequence that has one or two amino acid substitutions relative to SEQ ID NO: 3. In some embodiments, the leader sequence on the second polypeptide chain is upstream of M₂.

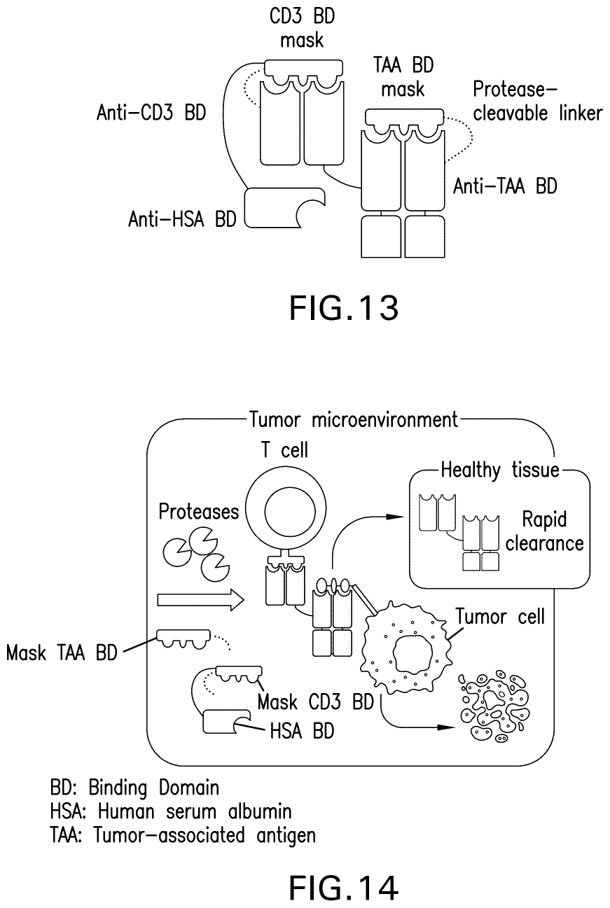

### Embodiments of Polypeptide Complexes as Exemplified in FIGS. 13, 14, and 15

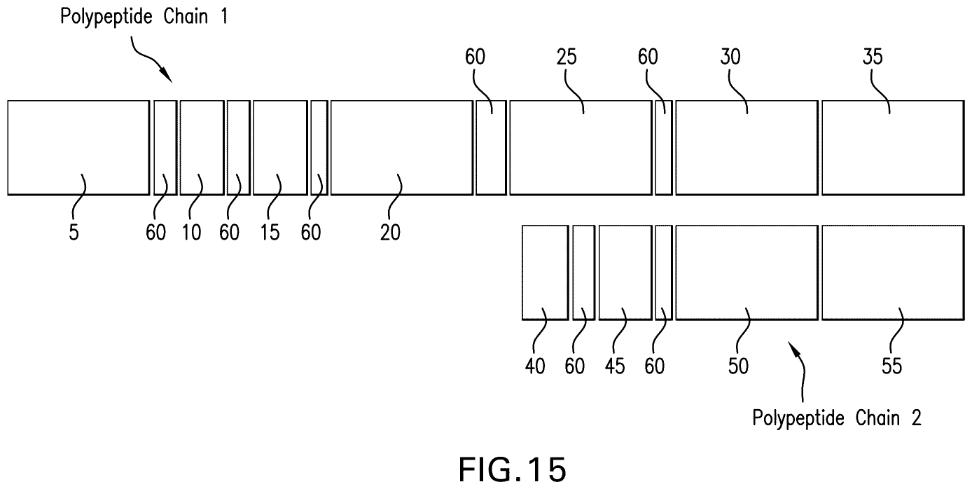

In an aspect, a polypeptide complex comprising polypeptide chain 1 and polypeptide chain 2 is provided, as exemplified in FIG. 15.

In some embodiments, polypeptide chain 1 comprises the elements of:
(i) an antibody or antibody fragment that binds a half-life extending molecule (5);
(ii) a first mask domain (10);
(iii) a first protease cleavage site (15);
(iv) an antibody fragment that binds an effector cell antigen when part of the polypeptide complex (20);
(v) an antibody fragment that binds an effector cell antigen when part of the polypeptide complex (25);
(vi) an antibody fragment that binds a tumor cell antigen when part of the polypeptide complex (30); and
(vii) a human IgG1 heavy chain 1^{st} chain constant region (C_{H}1) (35);

In some embodiments, polypeptide chain 2 comprises the elements of:
(i) a second mask domain (40);
(ii) second protease cleavage site (45);
(iii) an antibody fragment that binds a tumor cell antigen when part of the polypeptide complex (50); and
(iv) a human IgG1 light chain kappa region (55).

In some embodiments, linker sequences (60) connect certain elements of the polypeptide chains.

In some embodiments, the first mask domain binds to the antibody fragments (20), (25). In some embodiments, the first mask domain is bound to the antibody fragments (20), (25), in a tumor microenvironment (TME). In some embodiments, the first mask domain (10) comprises SEQ ID NO: 7. In some embodiments, the first mask domain (10) comprises a sequence with at least one or two amino acid substitutions relative to SEQ ID NO: 7, wherein the sequence binds the antibody fragments (20), (25).

In some embodiments, the first protease cleavage site (15) comprises SEQ ID NO: 8. In some embodiments, the first protease cleavage site (15) comprises a sequence with one or two amino acid substitutions relative to SEQ ID NO: 8, wherein the sequence is capable of being targeted by a protease.

In some embodiments, the antibody fragment capable of binding an effector cell antigen (20) comprises an anti-CD3 heavy chain variable region comprising CDRH1, CDRH2, and CDRH3, wherein CDRH1 comprises SEQ ID NO: 9, CDRH2 comprises SEQ ID NO: 10, and CDRH3 comprises SEQ ID NO: 11. In some embodiments, CDRH1 comprises a sequence with one or two amino acid substitutions relative to SEQ ID NO: 9, wherein the polypeptide complex comprising the anti-CD3 heavy chain variable region binds CD3. In some embodiments, CDRH2 comprises a sequence with one or two amino acid substitutions relative to SEQ ID NO: 10, wherein the polypeptide complex comprising the anti-CD3 heavy chain variable region binds CD3. In some embodiments, CDRH3 comprises a sequence with one or two amino acid substitutions relative to SEQ ID NO: 11, wherein the polypeptide complex comprising the anti-CD3 heavy chain variable region binds CD3. In some embodiments, the one or two amino acid substitutions in CDRH1, CDRH2, and/or CDRH3, are conservative amino acid substitutions.

In some embodiments, the antibody fragment that binds an effector cell antigen (25) comprises an anti-CD3 light chain variable region comprising CDRL1, CDRL2, and CDRL3, wherein CDRL1 comprises SEQ ID NO: 12, CDRL2 comprises SEQ ID NO: 13, and CDRL3 comprises SEQ ID NO: 14. In some embodiments, CDRL1 comprises a sequence with one or two amino acid substitutions relative to SEQ ID NO: 12, wherein the polypeptide complex comprising the anti-CD3 light chain variable region binds CD3. In some embodiments, CDRL2 comprises a sequence with one or two amino acid substitutions relative to SEQ ID NO: 13, wherein the polypeptide complex comprising the anti-CD3 light chain variable region binds CD3. In some embodiments, CDRL3 comprises a sequence with one or two amino acid substitutions relative to SEQ ID NO: 14, wherein the polypeptide complex comprising the anti-CD3 light chain variable region binds CD3. In some embodiments, the one or two amino acid substitutions in CDRH1, CDRH2, and/or CDRH3, are conservative amino acid substitutions.

In some embodiments, the antibody fragment capable of binding a tumor cell antigen (30) comprises an anti-Nectin-4 heavy chain variable region comprising CDRH1, CDRH2, and CDRH3, wherein CDRH1 comprises SEQ ID NO: 15, CDRH2 comprises SEQ ID NO: 16, and CDRH3 comprises SEQ ID NO: 17. In some embodiments, CDRH1 comprises a sequence with one or two amino acid substitutions relative to SEQ ID NO: 15, wherein the polypeptide complex comprising the anti-Nectin-4 heavy chain variable region binds Nectin-4. In some embodiments, CDRH2 comprises a sequence with one or two amino acid substitutions relative to SEQ ID NO: 16, wherein the polypeptide complex comprising the anti-Nectin-4 heavy chain variable region binds Nectin-4. In some embodiments, CDRH3 comprises a sequence with one or two amino acid substitutions relative to SEQ ID NO: 17, wherein the polypeptide complex comprising the anti-Nectin-4 heavy chain variable region binds Nectin-4. In some embodiments, the one or two amino acid substitutions in CDRH1, CDRH2, and/or CDRH3, are conservative amino acid substitutions.

In some embodiments, the second mask domain (40) comprises SEQ ID NO: 18. In some embodiments, the second mask domain (40) comprises a sequence with at least one or two amino acid substitutions relative to SEQ ID NO: 18, wherein the sequence binds an antibody or antibody fragment that targets Nectin-4.

In some embodiments, the second protease cleavage site (45) comprises SEQ ID NO: 18. In some embodiments, the second protease cleavage site (45) comprises a sequence with one or two amino acid substitutions relative to SEQ ID NO: 18, wherein the sequence is capable of being targeted by a protease.

In some embodiments, the antibody fragment capable of binding a tumor cell antigen (50) comprises an anti-Nectin-4 light chain variable region comprising CDRL1, CDRL2, and CDRL3, wherein CDRL1 comprises SEQ ID NO: 20, CDRL2 comprises SEQ ID NO: 21, and CDRL3 comprises SEQ ID NO: 22. In some embodiments, CDRL1 comprises a sequence with one or two amino acid substitutions relative to SEQ ID NO: 20, wherein the polypeptide complex comprising the anti-Nectin-4 light chain variable region binds Nectin-4. In some embodiments, CDRL2 comprises a sequence with one or two amino acid substitutions relative to SEQ ID NO: 21, wherein the polypeptide complex comprising the anti-Nectin-4 light chain variable region binds Nectin-4. In some embodiments, CDRL3 comprises a sequence with one or two amino acid substitutions relative to SEQ ID NO: 22, wherein the polypeptide complex comprising the anti-Nectin-4 light chain variable region binds Nectin-4. In some embodiments, the one or two amino acid substitutions in CDRL1, CDRL2, and/or CDRL3, are conservative amino acid substitutions.

FIG. 13 provides an exemplary embodiment of a polypeptide complex outside of a tumor microenvironment. As shown in FIG. 13, the polypeptide includes two masks. One of the masks binds to an anti-CD3 binding domain (BD) (CD3 BD mask). One of the masks binds to an anti-tumor-associated antigen (TAA) binding domain (TAA BD mask). The two masks are connected to the anti-CD3 binding domain and the anti-TAA binding domain through protease cleavable linkers.

FIG. 14 provides an exemplary embodiment of a polypeptide complex inside of a tumor microenvironment. FIG. 14 shows proteases removing the two masks through cleaving them at the protease cleavable linkers. Removal of the masks allows binding of the binding domain of the anti-TAA binding domain to the tumor-associated antigen and allows binding of the binding domain of the anti-CD3 binding domain to CD3.

### Exemplary Embodiments of Polypeptide Complexes Including Various Polypeptide Chains

In an aspect, a polypeptide complex is provided, comprising:
(a) a first polypeptide chain comprising:
   (i) an anti-albumin (ALB) heavy chain variable region;
   (ii) a first mask sequence;
   (iii) a first protease cleavage site;
   (iv) an anti-CD3 heavy chain variable region;
   (iv) an anti-CD3 light chain variable region; and
   (v) an anti-Nectin-4 heavy chain variable region,

and
(b) a second polypeptide chain comprising:
   (i) a second mask sequence;
   (ii) a second protease cleavage site; and
   (iii) an anti-Nectin-4 light chain variable region.

In some embodiments, the polypeptide complex is in a host cell. In some embodiments, the host cell is a non-cancerous cell. In some embodiments, the host cell is an effector cell. In some embodiments, the effector cell is a T cell. In some embodiments, the effector cells is a PBMC.

In some embodiments, the polypeptide complex exhibits reduced cytotoxicity on the host cells relative to equivalent polypeptide complex that lacks the first mask sequence and the second mask sequence. In some embodiments, the reduction in toxicity of polypeptide complex relative to equivalent polypeptide complex that lacks the first mask sequence and the second mask sequence is between about a 10× reduction in toxicity and a 10,000× reduction in toxicity, for example, between about a 10× reduction in toxicity and about a 100× reduction in toxicity, between about a 100× reduction in toxicity and about a 1,000× reduction in toxicity, between about a 1,000× reduction in toxicity and about a 2,000× reduction in toxicity, between about a 2,000× reduction in toxicity and about a 3,000× reduction in toxicity, between about a 3,000× reduction in toxicity and about a 4,000× reduction in toxicity, between about a 4,000× reduction in toxicity and about a 5,000× reduction in toxicity, between about a 5,000× reduction in toxicity and about a 6,000× reduction in toxicity, between about a 6,000× reduction in toxicity and about a 7,000× reduction in toxicity, between about a 7,000× reduction in cytotoxicity and about a 8,000× reduction in cytotoxicity, between about a 8,000× reduction in cytotoxicity and about a 9,000× reduction in cytotoxicity, and between about a 9,000× reduction in toxicity and about a 10,000× reduction in cytotoxicity

In some embodiments, the anti-ALB heavy chain comprises a CDRH1 comprising SEQ ID NO: 4, a CDRH2 comprising SEQ ID NO: 5, and a CDRH3 comprising SEQ ID NO: 6.

In some embodiments, the first mask sequence binds to the anti-CD3 heavy chain variable region and the anti-CD3 light chain variable region. In some embodiments, binding of the first mask sequence to the anti-CD3 heavy chain variable region and the anti-CD3 light chain variable region results in prevention of binding of the anti-CD3 heavy chain variable region and the anti-CD3 light chain variable region to CD3. In some embodiments, binding of the first mask sequence to the anti-CD3 heavy chain variable region and the anti-CD3 light chain variable region results in reduction in binding affinity of the anti-CD3 heavy chain variable region and the anti-CD3 light chain variable region for CD3.

In some embodiments, the first mask sequence comprises SEQ ID NO: 7.

In some embodiments, the first protease cleavage site comprises SEQ ID NO: 8.

In some embodiments, the anti-CD3 heavy chain variable region comprises a CDRH1 comprising SEQ ID NO: 9, a CDRH2 comprising SEQ ID NO: 10, and a CDRH3 comprising SEQ ID NO: 11.

In some embodiments, the anti-CD3 light chain variable region comprises a CDRL1 comprising SEQ ID NO: 12, a CDRL2 comprising SEQ ID NO: 13, and a CDRL3 comprising SEQ ID NO: 14.

In some embodiments, the anti-Nectin-4 heavy chain variable region comprises a CDRH1 comprising SEQ ID NO: 15, a CDRH2 comprising SEQ ID NO: 16, and a CDRH3 comprising SEQ ID NO: 17.

In some embodiments, the anti-Nectin-4 heavy chain variable region comprises a CDRH1 comprising SEQ ID NO: 52, a CDRH2 comprising SEQ ID NO: 53, and a CDRH3 comprising SEQ ID NO: 54.

In some embodiments, the anti-Nectin-4 heavy chain variable region comprises a CDRH1 comprising SEQ ID NO: 59, a CDRH2 comprising SEQ ID NO: 60, and a CDRH3 comprising SEQ ID NO: 61.

In some embodiments, the anti-Nectin-4 heavy chain variable region comprises a CDRH1 comprising SEQ ID NO: 66, a CDRH2 comprising SEQ ID NO: 67, and a CDRH3 comprising SEQ ID NO: 68.

In some embodiments, the second mask sequence binds to the anti-Nectin-4 light chain variable region outside of a TME. In some embodiments, binding of the second mask sequence to the anti-Nectin-4 light chain variable region results in prevention of binding of the Nectin-4 light chain variable region to Nectin-4. In some embodiments, binding of the second mask sequence to the anti-Nectin-4 light chain variable region results in a reduction in binding affinity of the Nectin-4 light chain variable region to Nectin-4.

In some embodiments, the second mask sequence comprises SEQ ID NO: 18. In some embodiments, the second mask sequence comprises SEQ ID NO: 23. In some embodiments, the second mask sequence comprises SEQ ID NO: 125. In some embodiments, the second mask sequence comprises SEQ ID NO: 126.

In some embodiments, the second protease cleavage site comprises SEQ ID NO: 19.

In some embodiments, the anti-Nectin-4 light chain variable region comprises a CDRL1 comprising SEQ ID NO: 20, a CDRL2 comprising SEQ ID NO: 21, and a CDRL3 comprising SEQ ID NO: 22.

In some embodiments, the anti-Nectin-5 light chain variable region comprises a CDRL1 comprising SEQ ID NO: 28, a CDRL2 comprising SEQ ID NO: 29, and a CDRL3 comprising a SEQ ID NO: 30.

In some embodiments, the anti-Nectin-5 light chain variable region comprises a CDRL1 comprising SEQ ID NO: 35, a CDRL2 comprising SEQ ID NO: 36, and a CDRL3 comprising SEQ ID NO: 37.

In some embodiments, the anti-Nectin-5 light chain variable region comprises a CDRL1 comprising SEQ ID NO: 42, a CDRL2 comprising SEQ ID NO: 29, and a CDRL3 comprising SEQ ID NO: 30.

In some embodiments, the first polypeptide sequence further comprises a leader sequence that is upstream of the anti-ALB heavy chain variable region. In some embodiments, the leader sequence comprises SEQ ID NO: 3.

In some embodiments, the second polypeptide sequence further comprises a leader sequence that is upstream of the second mask sequence. In some embodiments, the leader sequence comprises SEQ ID NO: 3.

In some embodiments, the first mask sequence binds to the anti-CD3 heavy chain variable region and the anti-CD3 light chain variable region. In some embodiments, the first mask sequence binds to the anti-CD3 heavy chain variable region and the anti-CD3 light chain variable region, outside of a tumor microenvironment.

In some embodiments, the second mask sequence binds to the anti-Nectin-4 light chain variable region. In some embodiments, the second mask sequence binds to the anti-Nectin-4 light chain variable region, outside of a tumor microenvironment.

In an aspect, a polypeptide complex is provided comprising:
(a) a first polypeptide chain comprising an amino acid sequence that is at least 80% identical to SEQ ID NO: 1, for example, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, or at least 99% identical to SEQ ID NO: 1; and
(b) a second polypeptide comprising an amino acid sequence that is at least 80% identical to SEQ ID NO: 2, for example, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, or at least 99% identical to SEQ ID NO: 2, wherein the polypeptide complex binds to Nectin-4 and CD3.

In some embodiments, the first polypeptide chain comprises SEQ ID NO: 1.

In some embodiments, the second polypeptide chain comprises SEQ ID NO: 2.

In some embodiments, the first polypeptide chain comprises SEQ ID NO: 1 and the second polypeptide chain comprises SEQ ID NO: 2.

### Polypeptide Complexes in Host Cells

In some embodiments, any of the polypeptide complexes described herein are expressed in host cells. In some embodiments, the host cell is an effector cell. In some embodiments, the effector cell is a T-cell. In some embodiments, the effector cell is peripheral blood mononuclear cell.

In some embodiments, the polypeptide complexes exhibit reduced cytotoxicity in host cell cells relative to equivalent polypeptide complexes that lack mask sequences. In some embodiments, the reduction in toxicity of polypeptide complexes described herein relative to equivalent polypeptide complexes that lack mask sequences is between about a 10× reduction in toxicity and a 100× reduction in toxicity, for example, between about a 10× reduction in toxicity and about a 20× reduction in toxicity, between about a 20× reduction in toxicity and about a 30× reduction in toxicity, between about a 30× reduction in toxicity and about a 40× reduction in toxicity, between about a 40× reduction in toxicity and about a 50× reduction in toxicity, between about a 50× reduction in toxicity and about a 60× reduction in toxicity, between about a 70× reduction in toxicity and about a 80× reduction in toxicity, between about a 80× reduction in toxicity and about a 90× reduction in toxicity, or between about a 90× reduction in toxicity and about a 100× reduction in toxicity.

### Polynucleotides

In some embodiments, disclosed herein are polynucleotides that encode any of the polypeptides or portions of the polypeptides disclosed herein.

In some embodiments, disclosed herein are polynucleotides that encode any of the polypeptide complexes disclosed herein.

In some embodiments, polynucleotides are disclosed that encode SEQ ID NO: 1. In some embodiments, the polynucleotide that encodes SEQ ID NO: 1 comprises a sequence that is at least 90% identical to SEQ ID NO: 194, for example, at about 90% identical, about 91% identical, about 92% identical, about 93% identical, about 94% identical, about 95% identical, about 96% identical, about 97% identical, about 98% identical, or about 99% identical to SEQ ID NO: 194. In some embodiments, the polynucleotide that encodes SEQ ID NO: 1 comprises SEQ ID NO: 194.

In some embodiments, polynucleotides are disclosed that encode SEQ ID NO: 12. In some embodiments, the polynucleotide that encodes SEQ ID NO: 2 comprises a sequence that is at least 90% identical to SEQ ID NO: 193, for example, at about 90% identical, about 91% identical, about 92% identical, about 93% identical, about 94% identical, about 95% identical, about 96% identical, about 97% identical, about 98% identical, or about 99% identical to SEQ ID NO: 193. In some embodiments, the polynucleotide that encodes SEQ ID NO: 2 comprises SEQ ID NO: 193.

In some embodiments, polynucleotides are disclosed that encode any of the light chains of the Nectin-4 antibodies described herein. In some embodiments, the polynucleotides that encode any of the light chains of the Nectin-4 antibodies comprises a sequence that is at least 90% identical to any one of SEQ ID NOs: 160, 161, 162, 163, 164, 165, 166, 167, 168, 169, 170, 171, 172, 173, 174, 175, and 176, for example, about 90% identical, about 91% identical, about 92% identical, about 93% identical, about 94% identical, about 95% identical, about 96% identical, about 97% identical, about 98% identical, or about 99% identical to any one of SEQ ID NOs: 160, 161, 162, 163, 164, 165, 166, 167, 168, 169, 170, 171, 172, 173, 174, 175, and 176.

In some embodiments, polynucleotides are disclosed that encode any of the heavy chains of the Nectin-4 antibodies described herein. In some embodiments, the polynucleotides that encode any of the heavy chains of the Nectin-4 antibodies comprises a sequence that is at least 90% identical to any one of SEQ ID NOs: 177, 178, 179, 180, 181, 182, 183, 184, 185, 186, 187, 188, 189, 190, 191, and 192 for example, about 90% identical, about 91% identical, about 92% identical, about 93% identical, about 94% identical, about 95% identical, about 96% identical, about 97% identical, about 98% identical, or about 99% identical to any one of SEQ ID NOs: 177, 178, 179, 180, 181, 182, 183, 184, 185, 186, 187, 188, 189, 190, 191, and 192.

### Pharmaceutical Compositions

Disclosed herein are pharmaceutical compositions that include any of the polypeptide complexes disclosed herein and a pharmaceutically acceptable carrier or excipient. Formulations of pharmaceutical compositions may be prepared by mixing any of the polypeptide complexes described herein with an acceptable carrier, excipient, or stabilizer in the form, e.g., lyophilized powders, slurries, aqueous solutions, or suspensions (see, e.g., Hardman, et al. (2001) Goodman and Gilman's The Pharmacological Basis of Therapeutics, McGraw-Hill, New York, NY; Gennaro (2000) Remington: The Science and Practice of Pharmacy, Lippincott, Williams, and Wilkins, New York, NY; Avis, et al. (eds.) (1993) Pharmaceutical Dosage Forms: Parenteral Medications, Marcel Dekker, NY; Lieberman, et al. (eds.) (1990) Pharmaceutical Dosage Forms: Tablets, Marcel Dekker, NY; Lieberman, et al. (eds.) (1990) Pharmaceutical Dosage Forms: Disperse Systems, Marcel Dekker, NY; Weiner and Kotkoskie (2000) Excipient Toxicity and Safety, Marcel Dekker, Inc., New York, NY).

### Methods of Treatment

In some embodiments, the polypeptides and polypeptide complexes disclosed herein are used in methods of treatment. In some embodiments, the polypeptides and polypeptide complexes disclosed herein are used in methods of treating cancers. In some embodiments, the cancers treated by the polypeptides or polypeptide complexes described herein include cells that express Nectin-4.

In some embodiments, the invention comprises a method of treating cancer in a patient in need thereof comprising administering to the patient an effective amount of any polypeptide complex or pharmaceutical composition disclosed herein.

In some embodiments, the cancer is head and neck cancer. In some embodiments, the cancer is skin cancer (e.g., melanoma). In some embodiments, the cancer is breast cancer (e.g., triple negative breast cancer) In some embodiments, the cancer is lung cancer (e.g., non-small cell lung cancer). In some embodiments, the cancer is urothelial cancer. In some embodiments, the cancer is colorectal cancer. In some embodiments, the cancer is cervical cancer. In some embodiments, the cancer is pancreatic cancer. In some embodiments, the cancer is bladder cancer. In some embodiments, the cancer is ovarian cancer. In some embodiments, the cancer is head and neck squamous cell carcinoma (HNSCC).

In some embodiments, the cancers treated by the polypeptides or polypeptide complexes described herein include any one or more of head and neck cancer, skin cancer (e.g., melanoma), breast cancer (e.g., triple negative breast cancer), lung cancer (e.g., non-small cell lung cancer), urothelial, colorectal cancer, cervical cancer, pancreatic cancer, bladder cancer, and ovarian cancer. In some embodiments, the head and neck cancer is head and neck squamous cell carcinoma (HNSCC).

In some embodiments, the cancers treated by the polypeptides or polypeptide complex described herein are carcinoma or sarcoma.

In some embodiments, the polypeptides and polypeptide complexes described herein are used in prophylactic treatments. In some embodiments, the polypeptides and polypeptide complexes described herein are used in therapeutic treatments.

In some embodiments, the polypeptides or polypeptide complexes disclosed herein are administered to patients via any of topical administration, intramuscular administration, intravenous administration, oral administration, subcutaneous administration, or bolus injection. In some embodiments, the polypeptides or polypeptide complexes disclosed herein are administered through any method known in the art. In some embodiments, the polypeptides or polypeptide complexes disclosed herein are administered between one time and five times weekly as a topical administration, intramuscular administration, intravenous administration, oral administration, subcutaneous administration, or bolus injection. In some embodiments, the polypeptides or polypeptide complexes described herein are administered as a continuous infusion, for example, a continuous intravenous infusion.

In some embodiments, the polypeptides or polypeptide complexes disclosed herein have a half-life of between about 12 hours and about 300 hours. In some embodiments, the polypeptides or polypeptide complexes disclosed herein have a half-life of at least 12 hours, at least 24 hours, at least 36 hours, at least 48 hours, at least 60 hours, at least 72 hours, at least 84 hours, at least 96 hours, at least 100 hours, at least 108 hours, at least 120 hours, at least 140 hours, at least 160 hours, at least 180 hours, at least 200 hours, or more than 200 hours. In some embodiments, the half-life of the polypeptides or polypeptide complexes is in a range of about 12 hours to about 300 hours, about 20 hours to about 280 hours, about 40 hours to about 240 hours, about 60 hours to about 200 hours, or about 80 hours to about 140 hours.

### Methods of Producing the Polypeptides and Polypeptide Complexes

The polypeptides and polypeptide complexes described herein may be produced by any method known in the art. In some embodiments, nucleic acids encoding polypeptides disclosed herein (e.g., the polypeptide of formula I and the polypeptide of formula II) can be inserted into a vector and expressed in a recombinant host cell. In some embodiments, a nucleic acid comprising SEQ ID NO: 193 and a nucleic acid comprising SEQ ID NO: 194 can be inserted into one or more vectors and expressed in a recombinant host cell to produce any of the polypeptide complexes described herein.

Mammalian cell lines available as hosts for expression of the antibodies or fragments disclosed herein are well known in the art and include many immortalized cell lines available from the American Type Culture Collection (ATCC). These include, inter alia, Chinese hamster ovary (CHO) cells, NSO, SP2 cells, HeLa cells, baby hamster kidney (BHK) cells, monkey kidney cells (COS), human hepatocellular carcinoma cells (e.g., Hep G2), A549 cells, 3T3 cells, HEK-293 cells and a number of other cell lines. Cell lines of particular preference are selected through determining which cell lines have high expression levels. Other cell lines that may be used are insect cell lines, such as Sf9 cells, amphibian cells, bacterial cells, plant cells and fungal cells.

When recombinant expression vectors encoding polypeptides disclosed herein (e.g., the polypeptide of formula I and the polypeptide of formula II) are introduced into host cells any of the polypeptide complexes disclosed herein are produced by culturing the host cells by culturing the host cells for a sufficient time period to allow for expression of a polypeptide complex disclosed herein. In some embodiments, the polypeptide complex is secreted into the culture media in which the host cells are grown. Polypeptide complex can be recovered from the culture media using standard protein purification methods.

In an aspect, a method of producing a polypeptide complex is provided comprising: (a) culturing a host cell in culture medium, wherein the host cell comprises one or more vectors comprising a polynucleotide encoding the first polypeptide chain and a polynucleotide encoding the second polypeptide chain, under conditions that allow expression of the polypeptide chains, and (b) optionally recovering a polypeptide complex from the host cell or culture medium. In alternative embodiments, the first polypeptide chain and the second polypeptide chain are encoded by a single polynucleotide sequence.

In some embodiments, the polynucleotide encoding the first polypeptide chain comprises SEQ ID NO: 193. In some embodiments, the polynucleotide comprises a sequence that encodes the first polypeptide chain that is at least 90% identical to SEQ ID NO 193, for example, about 90% identical, about 91% identical, about 92% identical, about 93% identical, about 94% identical, about 95% identical, about 96% identical, about 97% identical, about 98% identical, or about 99% identical to SEQ ID NO: 193. In some embodiments, the polynucleotide encoding the first polypeptide chain comprises SEQ ID NO: 193.

In some embodiments, the polynucleotide encoding the second polypeptide chain comprises SEQ ID NO: 194. In some embodiments, the polynucleotide comprises a sequence that encodes the second polypeptide chain that is at least 90% identical to SEQ ID NO: 194, for example, about 90% identical, about 91% identical, about 92% identical, about 93% identical, about 94% identical, about 95% identical, about 96% identical, about 97% identical, about 98% identical, or about 99% identical to SEQ ID NO: 194. In some embodiments, the polynucleotide encoding the second polypeptide chain comprises SEQ ID NO: 194.

In some embodiments, the first polypeptide chain comprises any embodiment of formula I as described herein. In some embodiments, the first polypeptide chain comprises a sequence that is at least 90% identical to SEQ ID NO: 1, for example, about 90% identical, about 91% identical, about 92% identical, about 93% identical, about 94% identical, about 95% identical, about 96% identical, about 97% identical, about 98% identical, or about 99% identical to SEQ ID NO: 1. In some embodiments, the first polypeptide chain comprises SEQ ID NO: 1.

In some embodiments, the second polypeptide chain comprises any embodiment of formula II as described here. In some embodiments, the second polypeptide chain comprises a sequence that is at least 90% identical to SEQ ID NO: 2, for example, about 90% identical, about 91% identical, about 92% identical, about 93% identical, about 94% identical, about 95% identical, about 96% identical, about 97% identical, about 98% identical, or about 99% identical to SEQ ID NO: 2. In some embodiments, the second polypeptide chain comprises SEQ ID NO: 2.

### EXAMPLES

The following examples are meant to be illustrative and should not be construed as further limiting of the embodiments described herein.

### Example 1: Synthesis and Characterization of Mouse Antibodies that Target Nectin-4

### Mouse Antibody Discovery Against Nectin-4

BALB/cJ and SJL/J mice were immunized with Nectin4-Fc immunogen using a standard 28-Day RIMMS protocol and 50 μg doses of the immunogen administered subcutaneously. The mice with the highest titer from each strain after RIMMs protocol were boosted with 25 μg immunogen. 3 to 5 days later mice splenocytes and lymphocytes were harvested and fused with NS1 myeloma cells using standard PEG fusion procedures. Fusion cell products were plated into 96 well plates and expanded in hybridoma medium. 10 to 11 days after plating supernatants were screened for their ability to bind plate coated human and cynomolgus monkey Nectin-4 antigen in a standard ELISA format using an anti-mouse secondary detection antibody. Positive wells were selected and hybridoma pools expanded. 3 to 5 days later a confirmatory ELISA was performed prior to subcloning the hybridoma pools. Each parental hybridoma pool of interest was subcloned into 96 well plates by the standard limiting dilution method. Subcloning plates were allowed to grow for 10 to 11 days prior to screening supernatants for Nectin-4 binding by ELISA. Clones were selected and further expanded. 10 mL supernatants of selected clones were purified using disposable proteinG drip columns using a standard bind, wash, elute method. Eluted antibodies were brought to neutral pH and measured for protein concentration prior to binding affinity measurements against human and cyno Nectin-4. Purified hybridoma antibodies were also characterized for their ability to bind particular domains within human Nectin-4. Clonal hybridomas cell lines of interest were then sequenced and cryopreserved. Hybridoma antibody sequences were determined using the standard 5′-RACE method. The light chain variable sequences and the light chain CDR sequences of the hybridoma antibody sequences are shown in Table 2. The heavy chain variable sequences and the heavy chain CDR sequences are shown in Table 3.

TABLE 2
Light Chain Variable and Light Chain CDR Sequences (Mouse Hybridoma
Clones)

| Mouse | Light chain variable | | | |
|---|---|---|---|---|
| Clone | sequences | CDRL1 | CDRL2 | CDRL3 |
| 14E4.H3 | DIVMTQSHKFMSTSVGGRVT | KASQDVSIAVA | WASTRQT | QQYSSYPFT |
| | ITCKASQDVSIAVAWYQQKP | (SEQ ID NO: 28) | (SEQ ID NO: 29) | (SEQ ID NO: 30) |
| | GQSPKLLIYWASTRQTGVPD | | | |
| | RFAGSGSGTDFTLTISNVQSE | | | |
| | DLADYFCQQYSSYPFTFGSG | | | |
| | TKLEIK (SEQ ID NO: 115) | | | |
| 14F6.B1 | DIVMTQSQKFMSTTVGDRVS | KASQNVVTAVA | SASHRFT | QQYSSYPYT |
| | ITCKASQNVVTAVAWYQQK | (SEQ ID NO: 35) | (SEQ ID NO: 36) | (SEQ ID NO: 37) |
| | PGQSPKLLIYSASHRFTGVPD | | | |
| | RFTGSGSGTDFTLIISNMQSE | | | |
| | DLADYFCQQYSSYPYTFGG | | | |
| | GTKLEIK (SEQ ID NO: 117) | | | |
| 16H8.B1 | DIVMTQSHKFMSTSVGGRV | KASQDVSTTVA | WASTRQT | QQYSSYPFT |
| | TITCKASQDVSTTVAWYQQ | (SEQ ID NO: 42) | (SEQ ID NO: 29) | (SEQ ID NO: 30) |
| | KPGQSPKLLIYWASTRQTG | | | |
| | VPDRFTGSGSGTDFTLTISN | | | |
| | VQSEDLADYFCQQYSSYPF | | | |
| | TFGSGTKLEIK (SEQ ID NO: | | | |
| | 119) | | | |

TABLE 3
Heavy Chain Variable and Heavy Chain CDR Sequences (Mouse Hybridoma
Clones)

| Mouse | Heavy chain variable | | | |
|---|---|---|---|---|
| Clone | sequence | CDRH1 | CDRH2 | CDRH3 |
| 14E4.H3 | EVQLQESGGDLVQPGGSLK | GFTFSDYY | YISNGGGNTYY | ASPEARYYGN |
| | LSCAASGFTFSDYYMFWIR | (SEQ ID NO: 120) | PDTVKG | YPFPY |
| | QTPQKRLEWVAYISNGGGN | | (SEQ ID NO: 53) | (SEQ ID NO: |
| | TYYPDTVKGRFTISRDNAK | | | 121) |
| | NTLYLQMSRLKSEDTAMY | | | |
| | YCASPEARYYGNYPFPYW | | | |
| | GQGTLVTVSA (SEQ ID NO: | | | |
| | 114) | | | |
| 14F6.B1 | QVQLKQSGPGLVQPSQSLSI | GFSLTTYG | VIWSGGSTDYN | ARTSHWYFDV |
| | TCTVSGFSLTTYGVHWVRQ | (SEQ ID NO: 122) | AAFIS | (SEQ ID NO: |
| | SPGKGLEWLGVIWSGGSTD | | (SEQ ID NO: 60) | 123) |
| | YNAAFISRLSISKDNSKSQV | | | |
| | FFEMYSLQADDTAIYYCAR | | | |
| | TSHWYFDVWGTGTTVTVS | | | |
| | S (SEQ ID NO: 116) | | | |
| 16H8.B1 | EVQLQESGGGLVQPGGSLK | GFTFSDYY | YISNGGGNTYY | ASPEARYYGN |
| | LSCAASGFTFSDYYMYWIR | (SEQ ID NO: 120) | SDTVKG | FPFPY |
| | QTPQKRLEWVAYISNGGGN | | (SEQ ID NO: 67) | (SEQ ID NO: |
| | TYYSDTVKGRFTISRDNAK | | | 124) |
| | NTLYLQMSRLKSEDTAMY | | | |
| | YCASPEARYYGNFPFPYWG | | | |
| | QGTLVTVSA (SEQ ID NO: | | | |
| | 118) | | | |

### Hybridoma Antibody Epitope Mapping

Purified hybridoma antibodies were screened for their ability to bind full length Nectin-4 antigen and truncated versions thereof fused to human Fc, including Nectin-4 V-C1 domains, and Nectin-4 C1-C2 domains in a standard ELISA format. Full length and truncated Nectin-4 antigens were plate coated, washed, blocked, and washed prior to addition of titrated purified clonal hybridoma antibodies. After a short incubation with purified hybridoma antibodies plates were washed again and bound antibody was detected using a horse radish peroxidase conjugated anti-mouse secondary antibody. Results are shown in Table 4.

TABLE 4
Antibody Epitope Mapping (Mouse Hybridoma Clones)
Domain mapping (ELISA EC50 mM)

| Hybridoma | Full-length | Nectin-4 VC1 | Nectin-4 C1-C2 |
|---|---|---|---|
| Clone | Nectin-4 | domain | domain |
| 14.E4H3 | 0.02 | 0.03 | 0.03 |
| 14.F6B1 | 0.03 | 129.10 | 0.03 |
| 16H8.B1 | 0.03 | 0.03 | 0.03 |

### Hybridoma Antibody Binding Specificity

Purified hybridoma antibodies were screened for their binding specificity by comparing binding potency to closely related family members of Nectin-4 (Nectin-1, Nectin-2, and Nectin-3) by ELISA. Full length Nectin-4 antigens fused to a human Fc were plate coated, washed, blocked, and washed prior to addition of titrated purified clonal hybridoma antibodies (Full length EGFR fused to a human Fc was used as a control). After a short incubation with purified hybridoma antibodies plates were washed again and bound antibody was detected using a horse radish peroxidase conjugated anti-mouse secondary antibody. Results of these ELISA experiments are shown in Table 5.

TABLE 5
Antibody Binding Specificity (Mouse Hybridoma Clones)

| | Selectivity of Binding to Nectin-1, Nectin-2, | | | |
|---|---|---|---|---|
| | and Nectin-3 (ELISA EC50 mM | | | |
| Mouse Clone | EGFR Fc | Nectin-1 Fc | Nectin-2 Fc | Nectin-3 Fc |
| 14E4.E12 | 2067 | 1629 | 1004 | 2294 |
| 14F6.B1 | 556.4 | 584.8 | 319.5 | 39.34 |
| 16H8.B1 | 8459 | 3561 | 3103 | 4597 |

Kinetic binding of polypeptide molecules to human and cynomolgus monkey Nectin-4 was evaluated by bio-layer interferometry using an Octet instrument. Briefly, biosensors were loaded with antigen and baselined in buffer. Polypeptide molecules were titrated in solution at 50 nM, 25 nM, 12.5 nM, and 6.25 nM then associated onto the antigen loaded sensors. After a short association period, sensors were transferred into buffer and the dissociation of bound polypeptide molecules was measured. Association and dissociation signals were recorded in real time and analyzed using a 1:1 binding model within the instrument software. Analysis using a 1:1 binding model enabled the calculation of the on and off rate constants as well as affinity, KD. Results of the Octet binding assays are shown in Tables 6 and 7.

TABLE 6
Octet Binding Assays - Human Nectin-4
(Mouse Hybridoma Clones)

| | Human Nectin-4 (Octet) | | | |
|---|---|---|---|---|
| Mouse Clone | KD (M) | kon (1/Ms) | koff (½) | t½ min |
| 14E4.E12 | 1.22E−09 | 3.97E+05 | 4.82E−04 | 23.97 |
| 14F6.B1 | 3.34E−09 | 1.15E+05 | 3.85E−04 | 30.01 |
| 16H8.B1 | 3.07E−09 | 2.67E+05 | 8.19E−04 | 14.11 |

TABLE 7
Octet Binding Assays - Cyano Nectin-4
(Mouse Hybridoma Clones)

| | Cyano Nectin-4 (Octet) | | | |
|---|---|---|---|---|
| Mouse Clone | KD (M) | kon (1/Ms) | koff (½) | t½ min |
| 14E4.E12 | 8.56E−10 | 4.14E+05 | 3.54E−04 | 32.63 |
| 14F6.B1 | 2.13E−09 | 1.55E+05 | 3.30E−04 | 35.01 |
| 16H8.B1 | 2.68E−09 | 2.96E+05 | 7.95E−04 | 14.53 |

### Example 2: Synthesis and Characterization of Rat Antibodies that Target Nectin-4

### Immunization and Hybridoma Screening

Six-week-old Lewis rats were co-immunized with a 1:1 mixed of human and mouse His tagged Nectin-4 recombinant protein (R&D systems human Nectin-4 catalog #2659-N4 and mouse Nectin-4 catalog #3116-N4.) The rats were injected weekly, for a total of seven injections, using a total of 25 μg of protein across three injection sites: subcutaneous, intraperitoneal, and footpad. Positive immunization titers were confirmed using protein ELISA with the human and mouse Nectin-4 recombinant protein. Splenocytes and lymph nodes were harvested from the rats and cells were isolated using gentle homogenization with a glass mortal/pestle. Red blood cells were then lysed using ACK lysing buffer for 5 minutes. Harvested cells were counted and fused with the myeloma partner cell line, SP2/0-Ag14, using a Nepa Gene Bulldog electroporator, 2-pulse setting. The fused hybridoma cells were then plated in 96-well tissue culture plates using limited dilution. After two weeks of growth, the hybridoma supernatants were screened for binding to human/mouse Nectin-4 by protein ELISA and by Biacore, to human Nectin-1 recombinant protein by protein ELISA, and to human Nectin-4 BaF3 cells by flow cytometry.

### Candidate Selection

Candidates that showed strong binding to human and mouse Nectin-4 protein and human Nectin-4 BAF3 cells and no binding to human Nectin-1 protein at the hybridoma supernatant stage were selected for hybridoma subcloning. Antibodies from the selected hybridomas were either produced recombinantly from small scale hybridoma cultures or as rat human chimeric antibodies using sequences derived from barcoded Next Generation Sequencing. The purified antibodies were then titrated and tested for binding to human/mouse Nectin-4 by protein ELISA and to T47D endogenous cells using flow cytometry. Additionally, the purified antibodies were characterized for affinity using Biacore and epitope binning using the Octet. The hybridoma candidates were narrowed down to six clones (SV011.3H9.1G1; SV011.55D10.1C1; SVO11.15H3.1A1; SV011.54H11.1A4; SV011.52E1.1B2; and SV011.54E9.1F1) with strong protein and cell binding properties to human Nectin-4 protein and cells that bound to diverse epitopes on the Nectin-4 IgV, IgC1, and IgC2 domains. The light variable sequences and light chain CDR sequences of the clones are shown in Table 8. The heavy chain variable sequences and the heavy chain CDR sequences of the clones are shown in Table 9.

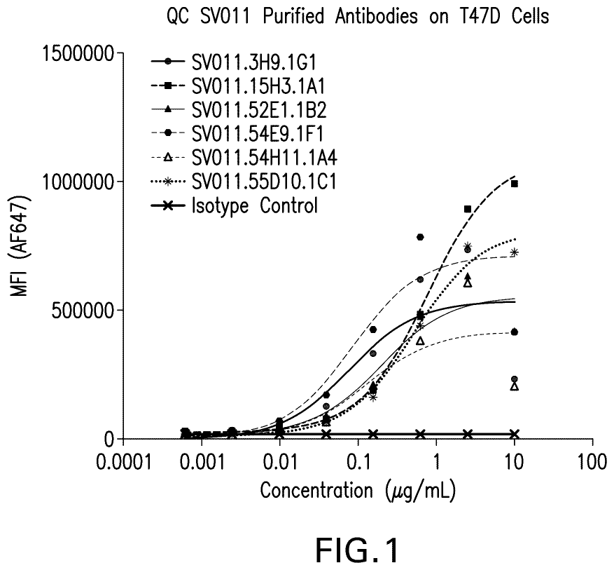

FIG. 1 shows FACS binding data of the purified antibodies from each of the six clones (SV011.3H9.1G1; SV011.55D10.1C1; SVO11.15H3.1A1; SV011.54H11.1A4; SV011.52E1.1B2; and SV011.54E9.1F1) to T47D cells.

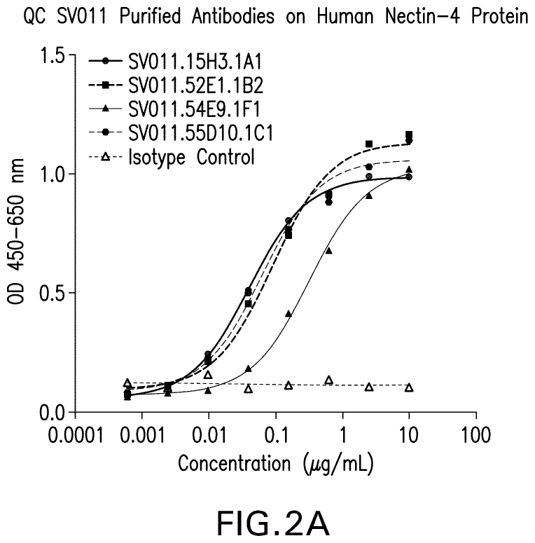

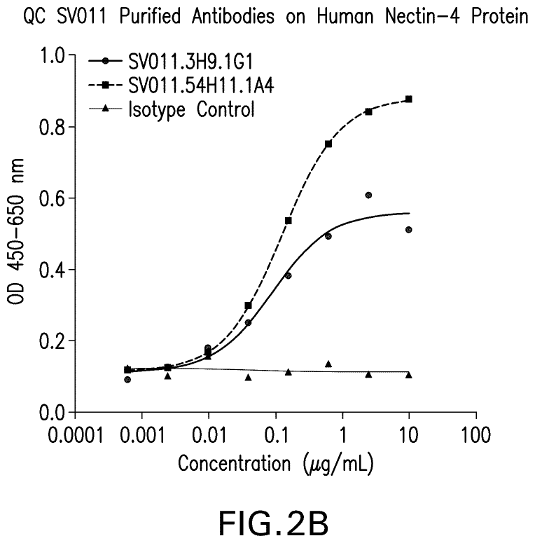

FIGS. 2A and 2B show ELISA binding to of the purified antibodies of the six clones (SV011.3H9.1G1; SV011.55D10.1C1; SVO11.15H3.1A1; SV011.54H11.1A4; SV011.52E1.1B2; and SV011.54E9.1F1) to the human Nectin-4 protein. A summary of the EC50 values is shown in Table 10.

TABLE 8
Light Chain Variable and Light Chain CDR Sequences (Rat Hybridoma Clones)

| Rat Clone | Light chain variable sequence | CDRL1 | CDRL2 | CDRL3 |
|---|---|---|---|---|
| 3H9.1G1 | DIQMTQSPASLSVYLGETVSIECLATEDIFSY | LATEDIFSY | GANRLK | LQGAKFPL |
| | LAWYQQKPGKSPQLLIYGANRLKDGVPSRF | LA (SEQ ID | D (SEQ ID | T (SEQ ID |
| | SGSGTGTQYSLRISGMQPEDEGDYYCLQGA | NO: 20) | NO: 21) | NO: 22) |
| | KFPLTFGSGTKLEIK (SEQ ID NO: 73) | | | |
| 55D10.1C1 | DIQMTQSPASLSASLGETVSIECLASEDIYSY | LASEDIYSY | AANRLQ | LQGSQFP |
| | LAWFQQKSGKSPQLLIYAANRLQDGVPSRF | LA (SEQ ID | D (SEQ ID | WT (SEQ |
| | SGSGSGTQYSLRISGMQPEDEGDYFCLQGS | NO: 85) | NO: 86) | ID NO: 87) |
| | QFPWTFGGGTKLELK (SEQ ID NO: 74) | | | |
| 15H3.1A1 | DIQMTQSPASLSASLGETVSIECLASEDIHNK | LASEDIHN | YGSNLQ | LQDSKNP |
| | LAWYQQKPGKSPQLLIYYGSNLQDGVPSRF | KLA (SEQ | D (SEQ ID | WT (SEQ |
| | SGSGSGTQYYLKINSLESEDVATYFCLQDSK | ID NO: 88) | NO: 89) | ID NO: 90) |
| | NPWTFGGGTKLEMK (SEQ ID NO: 75) | | | |
| 54H11.1A4 | DIVLTQSPALDVSLGQRATISCSASQSVSISR | SASQSVSIS | RTSILTS | QQSRESPF |
| | YNLIHWYQQKPGQQPKLLIWRTSILTSGIPA | RYNLIH | (SEQ ID | T (SEQ ID |
| | RFSGRGSGTDFTLTINPVQADDIATYYCQQS | (SEQ ID NO: | NO: 92) | NO: 93) |
| | RESPFTFGAGTRLELK (SEQ ID NO: 76) | 91) | | |
| 52E1.1B2 | NIVLTQSPATLSVTPGESVSLSCRASQSISTGI | RASQSISTG | FASQSIS | QQRDSSLF |
| | HWYQQKSNESPRLLIKFASQSISGIPSRFSGS | IH (SEQ ID | (SEQ ID | T (SEQ ID |
| | GSGTDFTLSINR VESEDFSIYYCQQRDSSLFT | NO: 94) | NO: 95) | NO: 96) |
| | FGAGTKLELK (SEQ ID NO: 77) | | | |
| 54E9.1F1 | TYELIQPPSTSVTLGNTVSLTCVGDDLPRRY | VGDDLPRR | EDSKRPS | HSTYSDD |
| | AYWYQQKPDQSIVR VIYEDSKRPSGISDRFS | YAY (SEQ | (SEQ ID | KVRV (SEQ |
| | GSSSGTTATLTIRDAQAEDEADYYCHSTYS | ID NO: 97) | NO: 98) | ID NO: 99) |
| | DDKVRVFGGGTKLTVL (SEQ ID NO: 78) | | | |

TABLE 9
Heavy Chain Variable and Heavy Chain CDR Sequences (Rat Hybridoma Clones)

| Rat Clone | Heavy chain variable sequence | CDRH1 | CDRH2 | CDRH3 |
|---|---|---|---|---|
| 3H9.1G1 | EVQLVESGGGLVQPGRSMRLSCAASGFSFS | NYYMA | SISTGGG | QTAYYVM |
| | NYYMAWVRQAPRKGLDWVASISTGGGNIY | (SEQ ID NO: | NIYYRD | DA (SEQ ID |
| | YRDSVKGRFTISRDNAKSTLYLQMDSLRSE | 15) | SVKG | NO: 17) |
| | DTATYYCARQTAYYVMDAWGQGASVTVS | | (SEQ ID | |
| | S (SEQ ID NO: 79) | | NO: 16) | |
| 55D10.1C1 | EVQLVESGGGLVQPGRSLKLSCAASGFTFS | NYDMA | SISPRGG | IHHGYWYF |
| | NYDMAWVRQVPTKGLEWVASISPRGGRIY | (SEQ ID NO: | RIYYRD | DF (SEQ ID |
| | YRDSVKGRFTVSRDNAKSCLYLQMESLRSD | 100) | SVKG | NO: 102) |
| | DTATYYCARIHHGYWYFDFWGPGTMVTVS | | (SEQ ID | |
| | S (SEQ ID NO: 80) | | NO: 101) | |
| 15H3.1A1 | EVKLLESGGGLVQPGGSMRLSCAASGFTFT | DFYMI | FIRNKA | PLYYGYTP |
| | DFYMIWIRQPAGKAPEWLGFIRNKANGYTT | (SEQ ID NO: | NGYTTD | RY (SEQ ID |
| | DYNPSVKGRFTISRDNTQNMLYLQMITLRA | 103) | YNPSVK | NO: 105) |
| | EDTATYYCARPLYYGYTPRYWGQGVMVT | | G (SEQ | |
| | VSS (SEQ ID NO: 81) | | ID NO: | |
| | | | 104) | |
| 54H11.1A4 | EVQLVESGGGLVQPGRPLKLSCAASGFSFS | HYDMA | AISPSGG | QGPSYGYY |
| | HYDMAWVRQAPTKGLEWVAAISPSGGSTY | (SEQ ID NO: | STYYRD | FDY (SEQ |
| | YRDSVKGRFTVSRDKAKNSLYLQMDSLRSE | 106) | SVKG | ID NO: 108) |
| | DTATYYCARQGPSYGYYFDYWGQGVMVT | | (SEQ ID | |
| | VSS (SEQ ID NO: 82) | | NO: 107) | |
| 52E1.1B2 | EVQLVESGGGLVQPGRSLKVSCAASGFTFS | NYDMA | SINPGGI | RQPYFDY |
| | NYDMAWVRQAPTKGLEWVASINPGGISTY | (SEQ ID NO: | STYYRD | (SEQ ID NO: |
| | YRDSVKGRFTVSRDNEKSTLYLQMDSLRSE | 100) | SVKG | 110) |
| | DTATYYCARRQPYFDYWGQGVMVTVSS | | (SEQ ID | |
| | (SEQ ID NO: 83) | | NO: 109) | |
| 54E9.1F1 | QVTLKESGTGILQPSQALSLTCSISGFSLNTT | VGDDLPRR | EDSKRP | HSTYSDDK |
| | GICVSWIRQPLGQGLEWLADICWDDGKGY | YAY (SEQ | S (SEQ | VRV (SEQ |
| | NPSLKNRLSISKDTSNNQAFLKITRVDTTDT | ID NO: 111) | ID NO: | ID NO: 113) |
| | ATYYCARNYGGNPFDYWGQGVMVTVSS | | 112) | |
| | (SEQ ID NO: 84) | | | |

TABLE 10
EC50 values for purified Nectin-4 antibodies binding to T47D
cells and human Nectin-4 protein (Rat Hybridoma Clones)

| | T47D cells | Human-Nectin4 |
|---|---|---|
| | EC50 | EC50 |
| Clone | (ug/mL) | (ug/mL) |
| SV011.3H9.1G1 | 0.077 | 0.090 |
| SV011.15H3.1A1 | 0.772 | 0.040 |
| SV011.52E1.1B2 | 0.195 | 0.090 |
| SV011.54E9.1F1 | 0.085 | 0.319 |
| SV011.54H11.1A4 | 0.131 | 0.127 |
| SV011.55D10.1C1 | 0.524 | 0.063 |
| Isotype Control | NB | NB |

### Hybridoma Supernatant or Purified Antibody Protein ELISA Assay

96-well half area plates were coated with either 25 μL/well with recombinant protein (1 μg/mL in PBS buffer) and incubated overnight at 4° C. The next day, plates were washed 3 times with PBST (PBS+0.05% Tween 20) and blocked with 25 μL/well of blocking buffer (PBS with 5% FBS) for 30 minutes at room temperature. Either hybridoma supernatant or titrated purified antibody was then transferred at 25 μl/well to the 96-well plates and incubated for 60 minutes at room temperature. The plates were then washed 3 times with PBST. Then 25 μl/well of anti-rat or anti-human IgG HRP conjugate (diluted in blocking buffer) was added to the plates and incubated for 60 minutes at room temperature. Finally, the plates were washed 5 times with PBST and developed by adding TMB reagent (Thermo cat #34029) to the plates for 2-3 minutes. The reactions were stopped with 0.16M sulfuric acid and the absorbance read at 450 nm and 650 nm using a spectrophometer.

### Hybridoma Supernatant or Purified Antibody Cell Binding Assay

Either recombinant BaF3 cells expressing human Nectin-4 or T47D endogenous cells were cultured for flow staining. The harvested cells were stained with either 50 μL of titrated purified antibodies or hybridoma supernatant for 30 minutes and then spun down and washed 1× with flow buffer (5% fetal bovine serum in PBS.) Next, the cells were stained with a fluorescently labeled secondary antibody (specific to the Fc domain of the hybridoma supernatant or purified recombinant antibody) for 30 minutes and spun down and washed 2× with flow buffer. Cells resuspended in 50 μL flow buffer were then analyzed using an Intellicyt.

### Next Generation Sequencing of Lysed Hybridomas

Hybridoma candidates of interest were lysed in 85 μL of Qiagen TCL buffer with 1% B-mercaptoethanol and RNA was isolated using Qiagen Turbocapture tubes (Qiagen, Catalog #72251). cDNA was generated using SuperScript IV reverse transcriptase (Thermo, Catalog #18090050) in the presence of a template switching oligo (TSO) for 5′RACE. A first round PCR reaction was then performed using GoTaq polymerase (Promega, Catalog #M7422), a forward R1 primer, and a reverse equimolar mixture of primers specific for the constant regions of the rat heavy and kappa/lambda light chains. A second round of PCR, also using GoTaq polymerase, further amplified the PCR products and introduced Illumina MiSeq NGS adaptors and indices for high-throughput, multiplexed NGS sequencing. Mixed PCR2 pools of heavy, kappa, and lambda chain samples were bead purified at a ratio of 0.8× using Agencourt AMPure XP beads (Beckman Coulter, Catalog #A63881). Individual, indexed PCR products were then pooled, and gel purified using a Qiagen gel purification kit (Qiagen, Catalog #28704). The NGS libraries were sequenced with an Illumina MiSeq and antibody variable regions were determined using a bioinformatic analysis pipeline. Sequences that showed unique variable regions (framework and CDR regions) were recombinantly expressed for further testing.

### Affinity Characterization of Hybridoma Supernatants and Purified Protein

Six-week-old Lewis rats were co-immunized with a 1:1 mixture of human and mouse His-tagged Nectin-4 recombinant proteins. The rats were injected weekly, for a total of 7 injections, using a total of 25 μg of protein across three injection sites: subcutaneous, intraperitoneal, and footpad. Positive immunization titers were confirmed using ELISA assay with the human and mouse Nectin-4 recombinant proteins. Splenocytes and lymph nodes were harvested from the rats and cells were isolated using gentle homogenization with a glass mortal/pestle. Red blood cells were then lysed using ACK lysing buffer for 5 minutes. Harvested cells were counted and fused with the myeloma partner cell line, SP2/0-Ag14, using a Nepa Gene Bulldog electroporator, 2-pulse setting. The fused hybridoma cells were then plated in 96-well tissue culture plates using limited dilution. After 2 weeks of culture, the hybridoma supernatants were screened for binding to human or mouse Nectin-4 by ELISA and by SPR, to human Nectin-1 recombinant protein by ELISA, and to human Nectin-4-expressing BaF3 cells by flow cytometry.

### Screening and Selection of Anti-Nectin-4 Hybridomas

Hybridoma supernatants that showed strong binding to human and mouse Nectin-4 proteins and to human Nectin-4-expressing BaF3cells, and no binding to human Nectin-1 protein were selected for subcloning. Antibodies from the selected hybridomas were produced either recombinantly from small scale hybridoma cultures or as rat human chimeric antibodies using sequences derived from barcoded Next Generation Sequencing. The purified antibodies were then titrated and tested for binding to human or mouse Nectin-4 by ELISA and to T47D endogenous cells using flow cytometry. Additionally, the purified antibodies were characterized for affinity by SPR and epitope binning using the Octet.

### Hybridoma Supernatant or Purified Antibody Binding by ELISA

Microtiter 96-well assay plates were coated with either 25 μL/well with recombinant Nectin-4 protein (1 μg/mL in PBS buffer) and incubated overnight at 4° C. The next day, plates were washed 3 times with PBST (PBS+0.05% Tween® 20) and blocked with 25 μL/well of blocking buffer PBSF for 30 minutes at room temperature. Either hybridoma supernatant or titrated purified antibody was then transferred at 25 μL/well to the 96-well plates and incubated for 60 minutes at ambient temperature. The plates were then washed 3 times with PBST. Then 25 μL/well of anti-rat or anti-human IgG HRP conjugate (diluted in blocking buffer) was added to the plates and incubated for 60 minutes at ambient temperature. Plates were washed 5 times with PBST and developed by adding TMB reagent to the plates for 2 to 3 minutes. The reactions were stopped with 0.16 M sulfuric acid and the absorbance read at 450 nm and 650 nm using a spectrophotometer.

### Hybridoma Supernatant or Purified Antibody Cell Binding Assay by Flow Cytometry

Recombinant BaF3 cells expressing human Nectin-4 or T47D endogenous cells were cultured for flow staining. The harvested cells were stained with either 50 μL of titrated purified antibodies or hybridoma supernatant for 30 minutes and then washed once with PBSF flow buffer. Cells were stained with a fluorescently labeled secondary antibody (specific to the Fc domain of the hybridoma supernatant or purified recombinant antibody) for 30 minutes, spun down, and washed twice with PBSF. Cells were resuspended in 50 μL PBSF and then analyzed using an Intellicyt instrument (Essen Bioscience Inc (Sartorius), Michigan, USA).

### Example 3: Recombinant Antibody Production

The rat hybridoma antibodies clones SV011.3H9.1G1, SV011.15H3.1A1, SV011.52E1.1B2, SV011.43E9.1F1, SV011.54H11.1A4, and SV011.55D10.1C1, and mouse hybridoma clones, 14E4. E12, 14F6. B1, and 16H8. B1 were reformatted into Fabs and bispecific T cell engagers. The mouse Vh and VI sequences of the hybridoma antibodies were placed into a standard Fab format with human light (kappa or lambda) and human heavy constant (CH1) domains to form a chimeric Fab. The chimeric Fab sequences were further reformatted into bispecific T cell engagers by appending an anti-CD3 scFv to the N-terminal light or N-terminal heavy chain of the chimer Fabs. DNA encoding the chimeric Fabs and T cell engagers were cloned into the pcDNA3.4 vector used to transiently express the proteins in ExpiCHO cells. Fabs and T cell engagers were then screened for binding to Nectin-4 and when relevant CD3 antigen binding. TCEs were screened for their ability to redirect T cell mediated target cell lysis in tumor cell co-culture assays. Fabs and T cell engagers were selected based on potency and activity then humanized. Humanized Fabs and TCEs were produced as described for the chimeric Fabs and TCEs. During humanization any potential detrimental PTMs within or near the CDRs of the binding domains were mutated out.

### Example 4: Humanization of Antibodies

Humanization of 3 mouse sequences (14E4. H3, 14F6. B1, 16H8. B) and one Rat sequence (SV011.3H1.1G1) Nectin-4 binding regions were performed using the BioPhi Sapiens software package. Structural models were built of the input sequence using the AbodyBuilder2 software package, and suggested highest probability mutations within 5 Angstroms of were iteratively compared to mutations of observed but lower probability using the total score feature of the Rosetta modelling software. The two lowest total score set of mutations were compared to the highest log likelihood from the Sapiens software, as well as the Best Single Mutations output from the Biovia Discovery Antibody Humanization workflow. The humanized light chain variable sequences and light chain CDR sequences are shown in Table 11. The humanized heavy chain variable sequences heavy chain CDR sequences are shown in Table 12. The full-length light sequences and heavy sequences of the humanized antibodies are shown in Tables 13 and 14, respectively. The nucleotide sequences that encode the full-length light sequences and heavy sequences of the humanized antibodies are shown in Tables 15 and 16, respectively.

TABLE 11
- Light Chain Variable Sequences and Light Chain CDR Sequences of
Humanized Antibodies

| Sequence | | | | |
|---|---|---|---|---|
| Name | Light chain variable sequence | CDRL1 | CDRL2 | CDRL3 |
| DAB012120 | EIQMTQSHKTFSVSTGQRVTITCKA | KASQDVSIAVA | WASTRQT | QQYSSYPFT |
| | SQDVSIAVAWYQQKPGKAPKLLIY | (SEQ ID NO: 28) | (SEQ ID | (SEQ ID NO: |
| | WASTRQTGVPSRFSGSGSGTDFTL | | NO: 29) | 30) |
| | TISCVQSEDFAVYYCQQYSSYPFTF | | | |
| | GQGTKLEIK (SEQ ID NO: 24) | | | |
| DAB012121 | DIQMTQSPSFLSASVGDRVTITCKA | KASQDVSIAVA | WASTRQT | QQYSSYPFT |
| | SQDVSIAVAWYQQKPGKAPKLLIY | (SEQ ID NO: 28) | (SEQ ID | (SEQ ID NO: |
| | WASTRQTGVPSRFSGSGSGTDFTL | | NO: 29) | 30) |
| | TISSLQPEDFATYFCQQYSSYPFTF | | | |
| | GQGTKLEIK (SEQ ID NO: 25) | | | |
| DAB012122 | DIQMTQSPSFLSASVGERVTITCKA | KASQDVSIAVA | WASTRQT | QQYSSYPFT |
| | SQDVSIAVAWYQQKPGKAPKLLIY | (SEQ ID NO: 28) | (SEQ ID | (SEQ ID NO: |
| | WASTRQTGVPSRFSGSGSGTDFTL | | NO: 29) | 30) |
| | TISSLQPEDIATYFCQQYSSYPFTFG | | | |
| | QGTKLEIK (SEQ ID NO: 26) | | | |
| DAB012123 | DIQMTQSPSFLSASVGDRVTITCKA | KASQDVSIAVA | WASTRQT | QQYSSYPFT |
| | SQDVSIAVAWYQQKPGKAPKLLIY | (SEQ ID NO: 28) | (SEQ ID | (SEQ ID NO: |
| | WASTRQTGVPSRFSGSGSGTDFTL | | NO: 29) | 30) |
| | TISSLQPEDSATYFCQQYSSYPFTF | | | |
| | GQGTKVDIK (SEQ ID NO: 27) | | | |
| DAB012124 | EIQMTQSQSTLSVSLGDRVTITCKA | KASQNVVTAVA | SASHRFT | QQYSSYPYT |
| | SQNVVTAVAWYQQKPGQAPKLLI | (SEQ ID NO: 35) | (SEQ ID | (SEQ ID NO: |
| | YSASHRFTGVPSRFSGSGSGTDFTL | | NO: 36) | 37) |
| | IISCMQSEDFAVYYCQQYSSYPYTF | | | |
| | GQGTKLEIK (SEQ ID NO: 31) | | | |
| DAB012125 | DIQMTQSPSFLSASVGDRVTITCKA | KASQNVVTAVA | SASHRFT | QQYSSYPYT |
| | SQNVVTAVAWYQQKPGKAPKLLI | (SEQ ID NO: 35) | (SEQ ID | (SEQ ID NO: |
| | YSASHRFTGVPSRFSGSGSGTDFTL | | NO: 36) | 37) |
| | TISNLQSEDLATYFCQQYSSYPYTF | | | |
| | GQGTKLEIK (SEQ ID NO: 32) | | | |
| DAB012126 | DIQMTQSPSFLSASVGDRVTITCKA | KASQNVVTAVA | SASHRFT | QQYSSYPYT |
| | SQNVVTAVAWYQQKPGKAPKLLI | (SEQ ID NO: 35) | (SEQ ID | (SEQ ID NO: |
| | YSASHRFTGVPSRFSGSGSGTDFTL | | NO: 36) | 37) |
| | TISNLQPEDSATYFCQQYSSYPYTF | | | |
| | GQGTKLEIK (SEQ ID NO: 33) | | | |
| DAB012127 | DIQMTQSPSFLSASVGDRVTITCKA | KASQNVVTAVA | SASHRFT | QQYSSYPYT |
| | SQNVVTAVAWYQQKPGKAPKLLI | (SEQ ID NO: 35) | (SEQ ID | (SEQ ID NO: |
| | YSASHRFTGVPSRFSGSGSGTDFTL | | NO: 36) | 37) |
| | TISNLQPDDAATYFCQQYSSYPYTF | | | |
| | GQGTKLEIK (SEQ ID NO: 34) | | | |
| DAB012128 | AIQMTQSHKSFSVSTGQRVTITCK | KASQDVSTTVA | WASTRQT | QQYSSYPFT |
| | ASQDVSTTVAWYQQKPGQAPKLL | (SEQ ID NO: 42) | (SEQ ID | (SEQ ID NO: |
| | IYWASTRQTGVPSRFTGSGSGTDF | | NO: 29) | 30) |
| | TLTISCVQSEDFAVYYCQQYSSYPF | | | |
| | TFGQGTKLEIK (SEQ ID NO: 38) | | | |
| DAB012129 | DIQMTQSPSFLSASVGDRVTITCKA | KASQDVSTTVA | WASTRQT | QQYSSYPFT |
| | SQDVSTTVAWYQQKPGKAPKLLI | (SEQ ID NO: 42) | (SEQ ID | (SEQ ID NO: |
| | YWASTRQTGVPSRFSGSGSGTDFT | | NO: 29) | 30) |
| | LTISNLQPEDFATYFCQQYSSYPFT | | | |
| | FGQGTKLEIK (SEQ ID NO: 39) | | | |
| DAB012130 | DIQMTQSPSFLSASVGERVTITCKA | KASQDVSTTVA | WASTRQT | QQYSSYPFT |
| | SQDVSTTVAWYQQKPGKAPKLLI | (SEQ ID NO: 42) | (SEQ ID | (SEQ ID NO: |
| | YWASTRQTGVPSRFSGSGSGTDFT | | NO: 29) | 30) |
| | LTINSLQPEDVATYFCQQYSSYPFT | | | |
| | FGQGTKLEIK (SEQ ID NO: 40) | | | |
| DAB012131 | DIQMTQSPSFLSASVGERVTITCKA | KASQDVSTTVA | WASTRQT | QQYSSYPFT |
| | SQDVSTTVAWYQQKPGKAPKLLI | (SEQ ID NO: 42) | (SEQ ID | (SEQ ID NO: |
| | YWASTRQTGVPSRFSGSGSGTDFT | | NO: 29) | 30) |
| | LTISNLQPEDVATYFCQQYSSYPFT | | | |
| | FGQGTKLEIK (SEQ ID NO: 41) | | | |
| DAB012132 | DIQMTQSPSSVSLTVGQRVTITCLA | LATEDIFSYLA | GANRLKD | LOGAKFPLT |
| | TEDIFSYLAWYQQKPGKAPRLLIY | (SEQ ID NO: 20) | (SEQ ID | (SEQ ID NO: |
| | GANRLKDGVPSRFSGSGSGTQYTL | | NO: 21) | 22) |
| | RISSMQPEDFGVYYCLQGAKFPLT | | | |
| | FGPGTKLEIK (SEQ ID NO: 43) | | | |
| DAB012133 | DIQMTQSPSSLSASIGDTVTITCLAT | LATEDIFSYLA | GANRLKD | LQGAKFPLT |
| | EDIFSYLAWYQQRPGKAPKLLIYG | (SEQ ID NO: 20) | (SEQ ID | (SEQ ID NO: |
| | ANRLKDGVPSRFSGSGSGTDYTLTI | | NO: 21) | 22) |
| | SGLQPEDFVTYYCLQGAKFPLTFG | | | |
| | QGTKLDMK (SEQ ID NO: 44) | | | |
| DAB012134 | DIQMTQSPSSLSASVGDTVTITCLA | LATEDIFSYLA | GANRLKD | LQGAKFPLT |
| | TEDIFSYLAWYQQKPGKAPKLLIY | (SEQ ID NO: 20) | (SEQ ID | (SEQ ID NO: |
| | GANRLKDGVPSRFSGSGSGTDYTL | | NO: 21) | 22) |
| | TISGLQPEDFGTYYCLQGAKFPLTF | | | |
| | GQGTKLEIK (SEQ ID NO: 45) | | | |
| DAB012135 | DIQMTQSPSSLSASVGDTVTITCLA | LATEDIFSYLA | GANRLKD | LQGAKFPLT |
| | TEDIFSYLAWYQQKPGKAPKLLIY | (SEQ ID NO: 20) | (SEQ ID | (SEQ ID NO: |
| | GANRLKDGVPSRFSGGGSGTEYSL | | NO: 21) | 22) |
| | TISGLQPEDFGTYYCLQGAKFPLTF | | | |
| | GQGTKLDMK (SEQ ID NO: 46) | | | |
| DAB012135 | DIQMTQSPSSLSASVGDTVTITCLA | LATEDIFSYLA | GANRLKD | LQGAKFPLT |
| (modified) | TEDIFSYLAWYQQKPGKAPKLLIY | (SEQ ID NO: 20) | (SEQ ID | (SEQ ID NO: |
| | GANRLKDTVPSRFSGGGSGTEYSL | | NO: 21) | 22) |
| | TISGLQPEDFGTYYCLQGAKFPLTF | | | |
| | GQGTKLDMKR (SEQ ID NO: 47) | | | |

TABLE 12
Heavy Chain Variable Sequences and Heavy Chain CDR Sequences of
Humanized Antibodies

| Sequence | | | | |
|---|---|---|---|---|
| Name | Heavy chain variable sequence | CDRH1 | CDRH2 | CDRH3 |
| DAB012120 | QVQLLESGGELVQPGGSLRLSCAA | DYYMF | YISNGGGNTYY | PEARYYGN |
| | SGFTFSDYYMFWIRQTPQKRLEWV | (SEQ ID NO: | PDTVKG (SEQ | YPFPY (SEQ |
| | AYISNGGGNTYYPDTVKGRFTISR | 52) | ID NO: 53) | ID NO: 54) |
| | DNAKNQLYLQMRSLTPEDTAIYYC | | | |
| | ASPEARYYGNYPFPYWGQGTLVT | | | |
| | VSS (SEQ ID NO: 48) | | | |
| DAB012121 | QVQLVESGGGLVKPGGSLRLSCAA | DYYMF | YISNGGGNTYY | PEARYYGN |
| | SGFTFSDYYMFWIRQAPGKGLEW | (SEQ ID NO: | PDTVKG (SEQ | YPFPY |
| | VAYISNGGGNTYYPDTVKGRFTIS | 52) | ID NO: 53) | (SEQ ID NO: |
| | RDNAKNTLYLQMNSLRAEDTAVY | | | 54) |
| | YCASPEARYYGNYPFPYWGQGTL | | | |
| | VTVSS (SEQ ID NO: 49) | | | |
| DAB012122 | QVQLVESGGGVVKPGGSLRLSCA | DYYMF | YISNGGGNTYY | PEARYYGN |
| | ASGFTFSDYYMFWIRQAPGKGLE | (SEQ ID NO: | PDTVKG (SEQ | YPFPY |
| | WVAYISNGGGNTYYPDTVKGRFTI | 52) | ID NO: 53) | (SEQ ID NO: |
| | SRDNAKNTLYLQMNSLRAEDTAV | | | 54) |
| | YYCASPEARYYGNYPFPYWGQGT | | | |
| | LVTVSS (SEQ ID NO: 50) | | | |
| DAB012123 | QVQLVESGGGVVQPGGSLRLSCA | DYYMF | YISNGGGNTYY | PEARYYGN |
| | ASGFTFSDYYMFWIRQAPGKGLE | (SEQ ID NO: | PDTVKG (SEQ | YPFPY |
| | WVAYISNGGGNTYYPDTVKGRFTI | 52) | ID NO: 53) | (SEQ ID NO: |
| | SRDNAKNTLYLQMNSLRAEDTAV | | | 54) |
| | YYCASPEARYYGNYPFPYWGQGT | | | |
| | LVTVSS (SEQ ID NO: 51) | | | |
| DAB012124 | QVQLLQSGPGLVKPSATLSLTCTV | TYGVH (SEQ | VIWSGGSTDYN | TSHWYFDV |
| | SGFSLTTYGVHWIRQPPGKGLEW | ID NO: 59) | AAFIS (SEQ ID | (SEQ ID NO: |
| | MGVIWSGGSTDYNAAFISRLTISK | | NO: 60) | 61) |
| | DNSKNQVFLQLYSLRAEDTAIYYC | | | |
| | ARTSHWYFDVWGRGTLVTVSS | | | |
| | (SEQ ID NO: 55) | | | |
| DAB012125 | QVQLQESGPGLVKPSQTLSLTCTV | TYGVH (SEQ | VIWSGGSTDYN | TSHWYFDV |
| | SGFSLTTYGVHWVRQSPGKGLEW | ID NO: 59) | AAFIS (SEQ ID | (SEQ ID NO: |
| | LGVIWSGGSTDYNAAFISRLSISKD | | NO: 60) | 61) |
| | NSKSQVFLEMTSLTAADTAIYYCA | | | |
| | RTSHWYFDVWGRGTLVTVSS | | | |
| | (SEQ ID NO: 56) | | | |
| DAB012126 | QVQLQESGPGLVKPSQTLSLTCTV | TYGVH (SEQ | VIWSGGSTDYN | TSHWYFDV |
| | SGFSLTTYGVHWVRQAPEKGLEW | ID NO: 59) | AAFIS (SEQ ID | (SEQ ID NO: |
| | LGVIWSGGSTDYNAAFISRLSISKD | | NO: 60) | 61) |
| | NSKSQVFLEMTSLTADDTAIYYCA | | | |
| | RTSHWYFDVWGRGTLVTVSS | | | |
| | (SEQ ID NO: 57) | | | |
| DAB012127 | QVQLQESGPGLVKPSQTLSLTCTV | TYGVH (SEQ | VIWSGGSTDYN | TSHWYFDV |
| | SGFSLTTYGVHWVRQTPEKGLEW | ID NO: 59) | AAFIS (SEQ ID | (SEQ ID NO: |
| | LGVIWSGGSTDYNAAFISRLSISKD | | NO: 60) | 61) |
| | NSKSQVFLEMTSLTADDTAIYYCA | | | |
| | RTSHWYFDVWGRGTLVTVSS | | | |
| | (SEQ ID NO: 58) | | | |
| DAB012128 | QVQLLESGGGLVQPGGSLRLSCAA | DYYMY (SEQ | YISNGGGNTYY | PEARYYGN |
| | SGFTFSDYYMYWIRQTPQKRLEW | ID NO: 66) | SDTVKG (SEQ | FPFPY (SEQ |
| | VAYISNGGGNTYYSDTVKGRFTIS | | ID NO: 67) | ID NO: 68) |
| | RDNAKNQLYLQMRSLRPEDTAIY | | | |
| | YCASPEARYYGNFPFPYWGQGTL | | | |
| | VTVSS (SEQ ID NO: 62) | | | |
| DAB012129 | QVQLVESGGGLVKPGGSLRLSCAA | DYYMY (SEQ | YISNGGGNTYY | PEARYYGN |
| | SGFTFSDYYMYWIRQAPGKGLEW | ID NO: 66) | SDTVKG (SEQ | FPFPY (SEQ |
| | VAYISNGGGNTYYSDTVKGRFTIS | | ID NO: 67) | ID NO: 68) |
| | RDNAKNTLYLQMNSLRAEDTAVY | | | |
| | YCASPEARYYGNFPFPYWGQGTL | | | |
| | VTVSS (SEQ ID NO: 63) | | | |
| DAB012130 | QVQLVESGGGLVKPGGSLRLSCAA | DYYMY (SEQ | YISNGGGNTYY | PEARYYGN |
| | SGFTFSDYYMYWIRQAPGKGLEW | ID NO: 66) | SDTVKG (SEQ | FPFPY (SEQ |
| | VAYISNGGGNTYYSDTVKGRFTIS | | ID NO: 67) | ID NO: 68) |
| | RDNAKNTLYLQMNSLRAEDTAVY | | | |
| | YCASPEARYYGNFPFPYWGQGTL | | | |
| | VTVSS (SEQ ID NO: 64) | | | |
| DAB012131 | QVQLVESGGGLVKPGGSLRLSCAA | DYYMY (SEQ | YISNGGGNTYY | PEARYYGN |
| | SGFTFSDYYMYWIRQAPGKGLEW | ID NO: 66) | SDTVKG (SEQ | FPFPY (SEQ |
| | VAYISNGGGNTYYSDTVKGRFTIS | | ID NO: 67) | ID NO: 68) |
| | RDNAKNTLYLQMNSLRAEDTAVY | | | |
| | YCASPEARYYGNFPFPYWGQGTL | | | |
| | VTVSS (SEQ ID NO: 65) | | | |
| DAB012132 | QVQLVESGGGVVQPGRSLRLSCA | NYYMA (SEQ | SISTGGGNIYYR | QTAYYVM |
| | ASGFSFSNYYMAWVRQAPGKGLE | ID NO: 15) | DSVKG (SEQ ID | DA (SEQ ID |
| | WVASISTGGGNIYYRDSVKGRFTIS | | NO: 16) | NO: 17) |
| | RDNSKSTLYLQMRSLRSEDTAIYY | | | |
| | CARQTAYYVMDAWGQGTMVTVS | | | |
| | S (SEQ ID NO: 69) | | | |
| DAB012133 | EVQLVESGGGLVQPGGSLRLSCAA | NYYMA (SEQ | SISTGGGNIYYR | QTAYYVM |
| | SGFSFSNYYMAWVRQAPGKGLEW | ID NO: 15) | DSVKG (SEQ ID | DA (SEQ ID |
| | VASISTGGGNIYYRDSVKGRFTISR | | NO: 16) | NO: 17) |
| | DNAKNSLYLQMNSLRAEDTAVYY | | | |
| | CARQTAYYVMDAWGQGTTVTVS | | | |
| | S (SEQ ID NO: 70) | | | |
| DAB012134 | EVQLVESGGGLVQPGGSLRLSCAA | NYYMA (SEQ | SISTGGGNIYYR | QTAYYVM |
| | SGFSFSNYYMAWVRQAPGKGLEW | ID NO: 15) | DSVKG (SEQ ID | DA (SEQ ID |
| | VASISTGGGNIYYRDSVKGRFTISR | | NO: 16) | NO: 17) |
| | DNAKNSLYLQMNSLRAEDTAVYY | | | |
| | CARQTAYYVMDAWGQGTTVTVS | | | |
| | S (SEQ ID NO: 71) | | | |
| DAB012135 | EVQLVESGGGLVQPGGSLRLSCAA | NYYMA (SEQ | SISTGGGNIYYR | QTAYYVM |
| | SGFSFSNYYMAWVRQAPGKGLEW | ID NO: 15) | DSVKG (SEQ ID | DA (SEQ ID |
| | VASISTGGGNIYYRDSVKGRFTISR | | NO: 16) | NO: 17) |
| | DNAKNSLYLQMNSLRAEDTAVYY | | | |
| | CARQTAYYVMDAWGQGTTVTVS | | | |
| | S (SEQ ID NO: 72) | | | |

TABLE 13
Full Length Light Chain Sequences of Humanized Antibodies

| Sequence | |
|---|---|
| Name | Light chain sequence |
| DAB012120 | EIQMTQSHKTFSVSTGQRVTITCKASQDVSIAVA WYQQKPGKAPKLLIYWASTRQTGVP |
| | SRFSGSGSGTDFTLTISCVQSEDFAVYYCQQYSSYPFTFGQGTKLEIKRTVAAPSVFIFPPS |
| | DEQLKSGTASVVCLLNNFYPREAKVQWKVDNALQSGNSQESVTEQDSKDSTYSLSSTL |
| | TLSKADYEKHKVYACEVTHQGLSSPVTKSFNRGEC (SEQ ID NO: 127) |
| DAB012121 | DIQMTQSPSFLSASVGDRVTITCKASQDVSIAVAWYQQKPGKAPKLLIYWASTRQTGVP |
| | SRFSGSGSGTDFTLTISSLQPEDFATYFCQQYSSYPFTFGQGTKLEIKRTVAAPSVFIFPPS |
| | DEQLKSGTASVVCLLNNFYPREAKVQWKVDNALQSGNSQESVTEQDSKDSTYSLSSTL |
| | TLSKADYEKHKVYACEVTHQGLSSPVTKSFNRGEC (SEQ ID NO: 128) |
| DAB012122 | DIQMTQSPSFLSASVGERVTITCKASQDVSIAVAWYQQKPGKAPKLLIYWASTRQTGVP |
| | SRFSGSGSGTDFTLTISSLQPEDIATYFCQQYSSYPFTFGQGTKLEIKRTVAAPSVFIFPPSD |
| | EQLKSGTASVVCLLNNFYPREAKVQWKVDNALQSGNSQESVTEQDSKDSTYSLSSTLTL |
| | SKADYEKHKVYACEVTHQGLSSPVTKSFNRGEC (SEQ ID NO: 129) |
| DAB012123 | DIQMTQSPSFLSASVGDRVTITCKASQDVSIAVAWYQQKPGKAPKLLIYWASTRQTGVP |
| | SRFSGSGSGTDFTLTISSLQPEDSATYFCQQYSSYPFTFGQGTKVDIKRTVAAPSVFIFPPS |
| | DEQLKSGTASVVCLLNNFYPREAKVQWKVDNALQSGNSQESVTEQDSKDSTYSLSSTL |
| | TLSKADYEKHKVYACEVTHQGLSSPVTKSFNRGEC (SEQ ID NO: 130) |
| DAB012124 | EIQMTQSQSTLSVSLGDRVTITCKASQNVVTAVAWYQQKPGQAPKLLIYSASHRFTGVP |
| | SRFSGSGSGTDFTLIISCMQSEDFAVYYCQQYSSYPYTFGQGTKLEIKRTVAAPSVFIFPPS |
| | DEQLKSGTASVVCLLNNFYPREAKVQWKVDNALQSGNSQESVTEQDSKDSTYSLSSTL |
| | TLSKADYEKHKVYACEVTHQGLSSPVTKSFNRGEC (SEQ ID NO: 131) |
| DAB012125 | DIQMTQSPSFLSASVGDRVTITCKASQNVVTAVAWYQQKPGKAPKLLIYSASHRFTGVP |
| | SRFSGSGSGTDFTLTISNLQSEDLATYFCQQYSSYPYTFGQGTKLEIKRTVAAPSVFIFPPS |
| | DEQLKSGTASVVCLLNNFYPREAKVQWKVDNALQSGNSQESVTEQDSKDSTYSLSSTL |
| | TLSKADYEKHKVYACEVTHQGLSSPVTKSFNRGEC (SEQ ID NO: 132) |
| DAB012126 | DIQMTQSPSFLSASVGDRVTITCKASQNVVTAVAWYQQKPGKAPKLLIYSASHRFTGVP |
| | SRFSGSGSGTDFTLTISNLQPEDSATYFCQQYSSYPYTFGQGTKLEIKRTVAAPSVFIFPPS |
| | DEQLKSGTASVVCLLNNFYPREAKVQWKVDNALQSGNSQESVTEQDSKDSTYSLSSTL |
| | TLSKADYEKHKVYACEVTHQGLSSPVTKSFNRGEC (SEQ ID NO: 133) |
| DAB012127 | DIQMTQSPSFLSASVGDRVTITCKASQNVVTAVAWYQQKPGKAPKLLIYSASHRFTGVP |
| | SRFSGSGSGTDFTLTISNLQPDDAATYFCQQYSSYPYTFGQGTKLEIKRTVAAPSVFIFPPS |
| | DEQLKSGTASVVCLLNNFYPREAKVQWKVDNALQSGNSQESVTEQDSKDSTYSLSSTL |
| | TLSKADYEKHKVYACEVTHQGLSSPVTKSFNRGEC (SEQ ID NO: 134) |
| DAB012128 | AIQMTQSHKSFSVSTGQRVTITCKASQDVSTTVAWYQQKPGQAPKLLIYWASTRQTGVP |
| | SRFTGSGSGTDFTLTISCVQSEDFAVYYCQQYSSYPFTFGQGTKLEIKRTVAAPSVFIFPPS |
| | DEQLKSGTASVVCLLNNFYPREAKVQWKVDNALQSGNSQESVTEQDSKDSTYSLSSTL |
| | TLSKADYEKHKVYACEVTHQGLSSPVTKSFNRGEC (SEQ ID NO: 135) |
| DAB012129 | DIQMTQSPSFLSASVGDRVTITCKASQDVSTTVAWYQQKPGKAPKLLIYWASTRQTGVP |
| | SRFSGSGSGTDFTLTISNLQPEDFATYFCQQYSSYPFTFGQGTKLEIKRTVAAPSVFIFPPS |
| | DEQLKSGTASVVCLLNNFYPREAKVQWKVDNALQSGNSQESVTEQDSKDSTYSLSSTL |
| | TLSKADYEKHKVYACEVTHQGLSSPVTKSFNRGEC (SEQ ID NO: 136) |
| DAB012130 | DIQMTQSPSFLSASVGERVTITCKASQDVSTTVAWYQQKPGKAPKLLIYWASTRQTGVP |
| | SRFSGSGSGTDFTLTINSLQPEDVATYFCQQYSSYPFTFGQGTKLEIKRTVAAPSVFIFPPS |
| | DEQLKSGTASVVCLLNNFYPREAKVQWKVDNALQSGNSQESVTEQDSKDSTYSLSSTL |
| | TLSKADYEKHKVYACEVTHQGLSSPVTKSFNRGEC (SEQ ID NO: 137) |
| DAB012131 | DIQMTQSPSFLSASVGERVTITCKASQDVSTTVAWYQQKPGKAPKLLIYWASTRQTGVP |
| | SRFSGSGSGTDFTLTISNLQPEDVATYFCQQYSSYPFTFGQGTKLEIKRTVAAPSVFIFPPS |
| | DEQLKSGTASVVCLLNNFYPREAKVQWKVDNALQSGNSQESVTEQDSKDSTYSLSSTL |
| | TLSKADYEKHKVYACEVTHQGLSSPVTKSFNRGEC (SEQ ID NO: 138) |
| DAB012132 | DIQMTQSPSSVSLTVGQRVTITCLATEDIFSYLAWYQQKPGKAPRLLIYGANRLKDGVPS |
| | RFSGSGSGTQYTLRISSMQPEDFGVYYCLQGAKFPLTFGPGTKLEIKRTVAAPSVFIFPPS |
| | DEQLKSGTASVVCLLNNFYPREAKVQWKVDNALQSGNSQESVTEQDSKDSTYSLSSTL |
| | TLSKADYEKHKVYACEVTHQGLSSPVTKSFNRGEC (SEQ ID NO: 139) |
| DAB012133 | DIQMTQSPSSLSASIGDTVTITCLATEDIFSYLAWYQQRPGKAPKLLIYGANRLKDGVPSR |
| | FSGSGSGTDYTLTISGLQPEDFVTYYCLQGAKFPLTFGQGTKLDMKRTVAAPSVFIFPPS |
| | DEQLKSGTASVVCLLNNFYPREAKVQWKVDNALQSGNSQESVTEQDSKDSTYSLSSTL |
| | TLSKADYEKHKVYACEVTHQGLSSPVTKSFNRGEC (SEQ ID NO: 140) |
| DAB012134 | DIQMTQSPSSLSASVGDTVTITCLATEDIFSYLAWYQQKPGKAPKLLIYGANRLKDGVPS |
| | RFSGSGSGTDYTLTISGLQPEDFGTYYCLQGAKFPLTFGQGTKLEIKRTVAAPSVFIFPPS |
| | DEQLKSGTASVVCLLNNFYPREAKVQWKVDNALQSGNSQESVTEQDSKDSTYSLSSTL |
| | TLSKADYEKHKVYACEVTHQGLSSPVTKSFNRGEC (SEQ ID NO: 141) |
| DAB012135 | DIQMTQSPSSLSASVGDTVTITCLATEDIFSYLAWYQQKPGKAPKLLIYGANRLKDGVPS |
| | RFSGGGSGTEYSLTISGLQPEDFGTYYCLQGAKFPLTFGQGTKLDMKRTVAAPSVFIFPP |
| | SDEQLKSGTASVVCLLNNFYPREAKVQWKVDNALQSGNSQESVTEQDSKDSTYSLSST |
| | LTLSKADYEKHKVYACEVTHQGLSSPVTKSFNRGEC (SEQ ID NO: 142) |
| DAB012135 | DIQMTQSPSSLSASVGDTVTITCLATEDIFSYLAWYQQKPGKAPKLLIYGANRLKDTVPS |
| (modified) | RFSGGGSGTEYSLTISGLQPEDFGTYYCLQGAKFPLTFGQGTKLDMKRTVAAPSVFIFPP |
| | SDEQLKSGTASVVCLLNNFYPREAKVQWKVDNALQSGNSQESVTEQDSKDSTYSLSST |
| | LTLSKADYEKHKVYACEVTHQGLSSPVTKSFNRGEC (SEQ ID NO: 143) |

TABLE 14
Full Length Heavy Chain Sequences of Humanized Antibodies

| Sequence | |
|---|---|
| Name | Heavy chain sequence |
| DAB012120 | QVQLLESGGELVQPGGSLRLSCAASGFTFSDYYMFWIRQTPQKRLEWVAYISNGGGNT |
| | YYPDTVKGRFTISRDNAKNQLYLQMRSLTPEDTAIYYCASPEARYYGNYPFPYWGQGT |
| | LVTVSSASTKGPSVFPLAPSSKSTSGGTAALGCLVKDYFPEPVTVSWNSGALTSGVHTFP |
| | AVLQSSGLYSLSSVVTVPSSSLGTQTYICNVNHKPSNTKVDKKVEPKSCDKTHTCPPCPA |
| | PELLGGPSVFLFPPKPKDTLMISRTPEVTCVVVDVSHEDPEVKFNWYVDGVEVHNAKTK |
| | PREEQYNSTYRVVSVLTVLHQDWLNGKEYKCKVSNKALPAPIEKTISKAKGQPREPQV |
| | YTLPPSRDELTKNQVSLTCLVKGFYPSDIAVEWESNGQPENNYKTTPPVLDSDGSFFLYS |
| | KLTVDKSRWQQGNVFSCSVMHEALHNHYTQKSLSLSPGK (SEQ ID NO: 144) |
| DAB012121 | QVQLVESGGGLVKPGGSLRLSCAASGFTFSDYYMFWIRQAPGKGLEWVAYISNGGGNT |
| | YYPDTVKGRFTISRDNAKNTLYLQMNSLRAEDTAVYYCASPEARYYGNYPFPYWGQG |
| | TLVTVSSASTKGPSVFPLAPSSKSTSGGTAALGCLVKDYFPEPVTVSWNSGALTSGVHTF |
| | PAVLQSSGLYSLSSVVTVPSSSLGTQTYICNVNHKPSNTKVDKKVEPKSCDKTHTCPPCP |
| | APELLGGPSVFLFPPKPKDTLMISRTPEVTCVVVDVSHEDPEVKFNWYVDGVEVHNAKT |
| | KPREEQYNSTYRVVSVLTVLHQDWLNGKEYKCKVSNKALPAPIEKTISKAKGQPREPQ |
| | VYTLPPSRDELTKNQVSLTCLVKGFYPSDIAVEWESNGQPENNYKTTPPVLDSDGSFFLY |
| | SKLTVDKSRWQQGNVFSCSVMHEALHNHYTQKSLSLSPGK (SEQ ID NO: 145) |
| DAB012122 | QVQLVESGGGVVKPGGSLRLSCAASGFTFSDYYMFWIRQAPGKGLEWVAYISNGGGNT |
| | YYPDTVKGRFTISRDNAKNTLYLQMNSLRAEDTAVYYCASPEARYYGNYPFPYWGQG |
| | TLVTVSSASTKGPSVFPLAPSSKSTSGGTAALGCLVKDYFPEPVTVSWNSGALTSGVHTF |
| | PAVLQSSGLYSLSSVVTVPSSSLGTQTYICNVNHKPSNTKVDKKVEPKSCDKTHTCPPCP |
| | APELLGGPSVFLFPPKPKDTLMISRTPEVTCVVVDVSHEDPEVKFNWYVDGVEVHNAKT |
| | KPREEQYNSTYRVVSVLTVLHQDWLNGKEYKCKVSNKALPAPIEKTISKAKGQPREPQ |
| | VYTLPPSRDELTKNQVSLTCLVKGFYPSDIAVEWESNGQPENNYKTTPPVLDSDGSFFLY |
| | SKLTVDKSRWQQGNVFSCSVMHEALHNHYTQKSLSLSPGK (SEQ ID NO: 146) |
| DAB012123 | QVQLVESGGGVVQPGGSLRLSCAASGFTFSDYYMFWIRQAPGKGLEWVAYISNGGGNT |
| | YYPDTVKGRFTISRDNAKNTLYLQMNSLRAEDTAVYYCASPEARYYGNYPFPYWGQG |
| | TLVTVSSASTKGPSVFPLAPSSKSTSGGTAALGCLVKDYFPEPVTVSWNSGALTSGVHTF |
| | PAVLQSSGLYSLSSVVTVPSSSLGTQTYICNVNHKPSNTKVDKKVEPKSCDKTHTCPPCP |
| | APELLGGPSVFLFPPKPKDTLMISRTPEVTCVVVDVSHEDPEVKFNWYVDGVEVHNAKT |
| | KPREEQYNSTYRVVSVLTVLHQDWLNGKEYKCKVSNKALPAPIEKTISKAKGQPREPQ |
| | VYTLPPSRDELTKNQVSLTCLVKGFYPSDIAVEWESNGQPENNYKTTPPVLDSDGSFFLY |
| | SKLTVDKSRWQQGNVFSCSVMHEALHNHYTQKSLSLSPGK (SEQ ID NO: 147) |
| DAB012124 | QVQLLQSGPGLVKPSATLSLTCTVSGFSLTTYGVHWIRQPPGKGLEWMGVIWSGGSTD |
| | YNAAFISRLTISKDNSKNQVFLQLYSLRAEDTAIYYCARTSHWYFDVWGRGTLVTVSSA |
| | STKGPSVFPLAPSSKSTSGGTAALGCLVKDYFPEPVTVSWNSGALTSGVHTFPAVLQSSG |
| | LYSLSSVVTVPSSSLGTQTYICNVNHKPSNTKVDKKVEPKSCDKTHTCPPCPAPELLGGP |
| | SVFLFPPKPKDTLMISRTPEVTCVVVDVSHEDPEVKFNWYVDGVEVHNAKTKPREEQY |
| | NSTYRVVSVLTVLHQDWLNGKEYKCKVSNKALPAPIEKTISKAKGQPREPQVYTLPPSR |
| | DELTKNQVSLTCLVKGFYPSDIAVEWESNGQPENNYKTTPPVLDSDGSFFLYSKLTVDK |
| | SRWQQGNVFSCSVMHEALHNHYTQKSLSLSPGK (SEQ ID NO: 148) |
| DAB012125 | QVQLQESGPGLVKPSQTLSLTCTVSGFSLTTYGVHWVRQSPGKGLEWLGVIWSGGSTD |
| | YNAAFISRLSISKDNSKSQVFLEMTSLTAADTAIYYCARTSHWYFDVWGRGTLVTVSSA |
| | STKGPSVFPLAPSSKSTSGGTAALGCLVKDYFPEPVTVSWNSGALTSGVHTFPAVLQSSG |
| | LYSLSSVVTVPSSSLGTQTYICNVNHKPSNTKVDKKVEPKSCDKTHTCPPCPAPELLGGP |
| | SVFLFPPKPKDTLMISRTPEVTCVVVDVSHEDPEVKFNWYVDGVEVHNAKTKPREEQY |
| | NSTYRVVSVLTVLHQDWLNGKEYKCKVSNKALPAPIEKTISKAKGQPREPQVYTLPPSR |
| | DELTKNQVSLTCLVKGFYPSDIAVEWESNGQPENNYKTTPPVLDSDGSFFLYSKLTVDK |
| | SRWQQGNVFSCSVMHEALHNHYTQKSLSLSPGK (SEQ ID NO: 149) |
| DAB012126 | QVQLQESGPGLVKPSQTLSLTCTVSGFSLTTYGVHWVRQAPEKGLEWLGVIWSGGSTD |
| | YNAAFISRLSISKDNSKSQVFLEMTSLTADDTAIYYCARTSHWYFDVWGRGTLVTVSSA |
| | STKGPSVFPLAPSSKSTSGGTAALGCLVKDYFPEPVTVSWNSGALTSGVHTFPAVLQSSG |
| | LYSLSSVVTVPSSSLGTQTYICNVNHKPSNTKVDKKVEPKSCDKTHTCPPCPAPELLGGP |
| | SVFLFPPKPKDTLMISRTPEVTCVVVDVSHEDPEVKFNWYVDGVEVHNAKTKPREEQY |
| | NSTYRVVSVLTVLHQDWLNGKEYKCKVSNKALPAPIEKTISKAKGQPREPQVYTLPPSR |
| | DELTKNQVSLTCLVKGFYPSDIAVEWESNGQPENNYKTTPPVLDSDGSFFLYSKLTVDK |
| | SRWQQGNVFSCSVMHEALHNHYTQKSLSLSPGK (SEQ ID NO: 150) |
| DAB012127 | QVQLQESGPGLVKPSQTLSLTCTVSGFSLTTYGVHWVRQTPEKGLEWLGVIWSGGSTD |
| | YNAAFISRLSISKDNSKSQVFLEMTSLTADDTAIYYCARTSHWYFDVWGRGTLVTVSSA |
| | STKGPSVFPLAPSSKSTSGGTAALGCLVKDYFPEPVTVSWNSGALTSGVHTFPAVLQSSG |
| | LYSLSSVVTVPSSSLGTQTYICNVNHKPSNTKVDKKVEPKSCDKTHTCPPCPAPELLGGP |
| | SVFLFPPKPKDTLMISRTPEVTCVVVDVSHEDPEVKFNWYVDGVEVHNAKTKPREEQY |
| | NSTYRVVSVLTVLHQDWLNGKEYKCKVSNKALPAPIEKTISKAKGQPREPQVYTLPPSR |
| | DELTKNQVSLTCLVKGFYPSDIAVEWESNGQPENNYKTTPPVLDSDGSFFLYSKLTVDK |
| | SRWQQGNVFSCSVMHEALHNHYTQKSLSLSPGK (SEQ ID NO: 151) |
| DAB012128 | QVQLLESGGGLVQPGGSLRLSCAASGFTFSDYYMYWIRQTPQKRLEWVAYISNGGGNT |
| | YYSDTVKGRFTISRDNAKNQLYLQMRSLRPEDTAIYYCASPEARYYGNFPFPYWGQGTL |
| | VTVSSASTKGPSVFPLAPSSKSTSGGTAALGCLVKDYFPEPVTVSWNSGALTSGVHTFPA |
| | VLQSSGLYSLSSVVTVPSSSLGTQTYICNVNHKPSNTKVDKKVEPKSCDKTHTCPPCPAP |
| | ELLGGPSVFLFPPKPKDTLMISRTPEVTCVVVDVSHEDPEVKFNWYVDGVEVHNAKTKP |
| | REEQYNSTYRVVSVLTVLHQDWLNGKEYKCKVSNKALPAPIEKTISKAKGQPREPQVY |
| | TLPPSRDELTKNQVSLTCLVKGFYPSDIAVEWESNGQPENNYKTTPPVLDSDGSFFLYSK |
| | LTVDKSRWQQGNVFSCSVMHEALHNHYTQKSLSLSPGK (SEQ ID NO: 152) |
| DAB012129 | QVQLVESGGGLVKPGGSLRLSCAASGFTFSDYYMYWIRQAPGKGLEWVAYISNGGGNT |
| | YYSDTVKGRFTISRDNAKNTLYLQMNSLRAEDTAVYYCASPEARYYGNFPFPYWGQGT |
| | LVTVSSASTKGPSVFPLAPSSKSTSGGTAALGCLVKDYFPEPVTVSWNSGALTSGVHTFP |
| | AVLQSSGLYSLSSVVTVPSSSLGTQTYICNVNHKPSNTKVDKKVEPKSCDKTHTCPPCPA |
| | PELLGGPSVFLFPPKPKDTLMISRTPEVTCVVVDVSHEDPEVKFNWYVDGVEVHNAKTK |
| | PREEQYNSTYRVVSVLTVLHQDWLNGKEYKCKVSNKALPAPIEKTISKAKGQPREPQV |
| | YTLPPSRDELTKNQVSLTCLVKGFYPSDIAVEWESNGQPENNYKTTPPVLDSDGSFFLYS |
| | KLTVDKSRWQQGNVFSCSVMHEALHNHYTQKSLSLSPGK (SEQ ID NO: 153) |
| DAB012130 | QVQLVESGGGLVKPGGSLRLSCAASGFTFSDYYMYWIRQAPGKGLEWVAYISNGGGNT |
| | YYSDTVKGRFTISRDNAKNTLYLQMNSLRAEDTAVYYCASPEARYYGNFPFPYWGQGT |
| | LVTVSSASTKGPSVFPLAPSSKSTSGGTAALGCLVKDYFPEPVTVSWNSGALTSGVHTFP |
| | AVLQSSGLYSLSSVVTVPSSSLGTQTYICNVNHKPSNTKVDKKVEPKSCDKTHTCPPCPA |
| | PELLGGPSVFLFPPKPKDTLMISRTPEVTCVVVDVSHEDPEVKFNWYVDGVEVHNAKTK |
| | PREEQYNSTYRVVSVLTVLHQDWLNGKEYKCKVSNKALPAPIEKTISKAKGQPREPQV |
| | YTLPPSRDELTKNQVSLTCLVKGFYPSDIAVEWESNGQPENNYKTTPPVLDSDGSFFLYS |
| | KLTVDKSRWQQGNVFSCSVMHEALHNHYTQKSLSLSPGK (SEQ ID NO: 154) |
| DAB012131 | QVQLVESGGGLVKPGGSLRLSCAASGFTFSDYYMYWIRQAPGKGLEWVAYISNGGGNT |
| | YYSDTVKGRFTISRDNAKNTLYLQMNSLRAEDTAVYYCASPEARYYGNFPFPYWGQGT |
| | LVTVSSASTKGPSVFPLAPSSKSTSGGTAALGCLVKDYFPEPVTVSWNSGALTSGVHTFP |
| | AVLQSSGLYSLSSVVTVPSSSLGTQTYICNVNHKPSNTKVDKKVEPKSCDKTHTCPPCPA |
| | PELLGGPSVFLFPPKPKDTLMISRTPEVTCVVVDVSHEDPEVKFNWYVDGVEVHNAKTK |
| | PREEQYNSTYRVVSVLTVLHQDWLNGKEYKCKVSNKALPAPIEKTISKAKGQPREPQV |
| | YTLPPSRDELTKNQVSLTCLVKGFYPSDIAVEWESNGQPENNYKTTPPVLDSDGSFFLYS |
| | KLTVDKSRWQQGNVFSCSVMHEALHNHYTQKSLSLSPGK (SEQ ID NO: 155) |
| DAB012132 | QVQLVESGGGVVQPGRSLRLSCAASGFSFSNYYMAWVRQAPGKGLEWVASISTGGGNI |
| | YYRDSVKGRFTISRDNSKSTLYLQMRSLRSEDTAIYYCARQTAYYVMDAWGQGTMVT |
| | VSSASTKGPSVFPLAPSSKSTSGGTAALGCLVKDYFPEPVTVSWNSGALTSGVHTFPAVL |
| | QSSGLYSLSSVVTVPSSSLGTQTYICNVNHKPSNTKVDKKVEPKSCDKTHTCPPCPAPEL |
| | LGGPSVFLFPPKPKDTLMISRTPEVTCVVVDVSHEDPEVKFNWYVDGVEVHNAKTKPRE |
| | EQYNSTYRVVSVLTVLHQDWLNGKEYKCKVSNKALPAPIEKTISKAKGQPREPQVYTLP |
| | PSRDELTKNQVSLTCLVKGFYPSDIAVEWESNGQPENNYKTTPPVLDSDGSFFLYSKLTV |
| | DKSRWQQGNVFSCSVMHEALHNHYTQKSLSLSPGK (SEQ ID NO: 156) |
| DAB012133 | EVQLVESGGGLVQPGGSLRLSCAASGFSFSNYYMAWVRQAPGKGLEWVASISTGGGNI |
| | YYRDSVKGRFTISRDNAKNSLYLQMNSLRAEDTAVYYCARQTAYYVMDAWGQGTTV |
| | TVSSASTKGPSVFPLAPSSKSTSGGTAALGCLVKDYFPEPVTVSWNSGALTSGVHTFPAV |
| | LQSSGLYSLSSVVTVPSSSLGTQTYICNVNHKPSNTKVDKKVEPKSCDKTHTCPPCPAPE |
| | LLGGPSVFLFPPKPKDTLMISRTPEVTCVVVDVSHEDPEVKFNWYVDGVEVHNAKTKPR |
| | EEQYNSTYRVVSVLTVLHQDWLNGKEYKCKVSNKALPAPIEKTISKAKGQPREPQVYT |
| | LPPSRDELTKNQVSLTCLVKGFYPSDIAVEWESNGQPENNYKTTPPVLDSDGSFFLYSKL |
| | TVDKSRWQQGNVFSCSVMHEALHNHYTQKSLSLSPGK (SEQ ID NO: 157) |
| DAB012134 | EVQLVESGGGLVQPGGSLRLSCAASGFSFSNYYMAWVRQAPGKGLEWVASISTGGGNI |
| | YYRDSVKGRFTISRDNAKNSLYLQMNSLRAEDTAVYYCARQTAYYVMDAWGQGTTV |
| | TVSSASTKGPSVFPLAPSSKSTSGGTAALGCLVKDYFPEPVTVSWNSGALTSGVHTFPAV |
| | LQSSGLYSLSSVVTVPSSSLGTQTYICNVNHKPSNTKVDKKVEPKSCDKTHTCPPCPAPE |
| | LLGGPSVFLFPPKPKDTLMISRTPEVTCVVVDVSHEDPEVKFNWYVDGVEVHNAKTKPR |
| | EEQYNSTYRVVSVLTVLHQDWLNGKEYKCKVSNKALPAPIEKTISKAKGQPREPQVYT |
| | LPPSRDELTKNQVSLTCLVKGFYPSDIAVEWESNGQPENNYKTTPPVLDSDGSFFLYSKL |
| | TVDKSRWQQGNVFSCSVMHEALHNHYTQKSLSLSPGK (SEQ ID NO: 158) |
| DAB012135 | EVQLVESGGGLVQPGGSLRLSCAASGFSFSNYYMAWVRQAPGKGLEWVASISTGGGNI |
| | YYRDSVKGRFTISRDNAKNSLYLQMNSLRAEDTAVYYCARQTAYYVMDAWGQGTTV |
| | TVSSASTKGPSVFPLAPSSKSTSGGTAALGCLVKDYFPEPVTVSWNSGALTSGVHTFPAV |
| | LQSSGLYSLSSVVTVPSSSLGTQTYICNVNHKPSNTKVDKKVEPKSCDKTHTCPPCPAPE |
| | LLGGPSVFLFPPKPKDTLMISRTPEVTCVVVDVSHEDPEVKFNWYVDGVEVHNAKTKPR |
| | EEQYNSTYRVVSVLTVLHQDWLNGKEYKCKVSNKALPAPIEKTISKAKGQPREPQVYT |
| | LPPSRDELTKNQVSLTCLVKGFYPSDIAVEWESNGQPENNYKTTPPVLDSDGSFFLYSKL |
| | TVDKSRWQQGNVFSCSVMHEALHNHYTQKSLSLSPGK (SEQ ID NO: 159) |

TABLE 15
Full Length Light Chain Nucleotide Sequences of Humanized Antibodies

| Sequence | |
|---|---|
| Name | Light chain sequence |
| DAB012120 | GAGATCCAGATGACCCAGAGCCACAAGACCTTCAGCGTGAGCACCGGCCAGAGGGT |
| | GACCATCACCTGCAAGGCCAGCCAGGACGTGAGCATCGCCGTGGCCTGGTACCAGC |
| | AGAAGCCCGGCAAGGCCCCCAAGCTGCTGATCTACTGGGCCAGCACCAGGCAGACC |
| | GGCGTGCCCAGCAGGTTCAGCGGCAGCGGCAGCGGCACCGACTTCACCCTGACCAT |
| | CAGCTGCGTGCAGAGCGAGGACTTCGCCGTGTACTACTGCCAGCAGTACAGCAGCT |
| | ACCCCTTCACCTTCGGCCAGGGCACCAAGCTGGAGATCAAGCGAACGGTGGCTGCC |
| | CCCTCCGTGTTCATCTTCCCCCCCAGCGATGAGCAGCTGAAGAGCGGCACAGCCAGC |
| | GTGGTGTGCCTGCTGAACAACTTCTACCCCAGGGAGGCCAAGGTGCAGTGGAAGGT |
| | GGACAACGCCCTGCAGAGCGGCAACAGCCAGGAGTCCGTGACCGAGCAGGACAGC |
| | AAGGACAGCACCTACAGCCTGAGCAGCACACTGACCCTGAGCAAGGCCGACTACGA |
| | GAAGCACAAGGTGTACGCCTGCGAGGTGACCCATCAGGGCCTGAGCAGCCCCGTGA |
| | CCAAGAGCTTCAACAGGGGCGAGTGC (SEQ ID NO: 160) |
| DAB012121 | GACATCCAGATGACCCAGAGCCCCAGCTTCCTGAGCGCCAGCGTGGGCGACAGGGT |
| | GACCATCACCTGCAAGGCCAGCCAGGACGTGAGCATCGCCGTGGCCTGGTACCAGC |
| | AGAAGCCCGGCAAGGCCCCCAAGCTGCTGATCTACTGGGCCAGCACCAGGCAGACC |
| | GGCGTGCCCAGCAGGTTCAGCGGCAGCGGCAGCGGCACCGACTTCACCCTGACCAT |
| | CAGCAGCCTGCAGCCCGAGGACTTCGCCACCTACTTCTGCCAGCAGTACAGCAGCTA |
| | CCCCTTCACCTTCGGCCAGGGCACCAAGCTGGAGATCAAGCGAACGGTGGCTGCCC |
| | CCTCCGTGTTCATCTTCCCCCCCAGCGATGAGCAGCTGAAGAGCGGCACAGCCAGCG |
| | TGGTGTGCCTGCTGAACAACTTCTACCCCAGGGAGGCCAAGGTGCAGTGGAAGGTG |
| | GACAACGCCCTGCAGAGCGGCAACAGCCAGGAGTCCGTGACCGAGCAGGACAGCA |
| | AGGACAGCACCTACAGCCTGAGCAGCACACTGACCCTGAGCAAGGCCGACTACGAG |
| | AAGCACAAGGTGTACGCCTGCGAGGTGACCCATCAGGGCCTGAGCAGCCCCGTGAC |
| | CAAGAGCTTCAACAGGGGCGAGTGC (SEQ ID NO: 161) |
| DAB012122 | GACATCCAGATGACCCAGAGCCCCAGCTTCCTGAGCGCCAGCGTGGGCGAGAGGGT |
| | GACCATCACCTGCAAGGCCAGCCAGGACGTGAGCATCGCCGTGGCCTGGTACCAGC |
| | AGAAGCCCGGCAAGGCCCCCAAGCTGCTGATCTACTGGGCCAGCACCAGGCAGACC |
| | GGCGTGCCCAGCAGGTTCAGCGGCAGCGGCAGCGGCACCGACTTCACCCTGACCAT |
| | CAGCAGCCTGCAGCCCGAGGACATCGCCACCTACTTCTGCCAGCAGTACAGCAGCT |
| | ACCCCTTCACCTTCGGCCAGGGCACCAAGCTGGAGATCAAGCGAACGGTGGCTGCC |
| | CCCTCCGTGTTCATCTTCCCCCCCAGCGATGAGCAGCTGAAGAGCGGCACAGCCAGC |
| | GTGGTGTGCCTGCTGAACAACTTCTACCCCAGGGAGGCCAAGGTGCAGTGGAAGGT |
| | GGACAACGCCCTGCAGAGCGGCAACAGCCAGGAGTCCGTGACCGAGCAGGACAGC |
| | AAGGACAGCACCTACAGCCTGAGCAGCACACTGACCCTGAGCAAGGCCGACTACGA |
| | GAAGCACAAGGTGTACGCCTGCGAGGTGACCCATCAGGGCCTGAGCAGCCCCGTGA |
| | CCAAGAGCTTCAACAGGGGCGAGTGC (SEQ ID NO: 162) |
| DAB012123 | GACATCCAGATGACCCAGAGCCCCAGCTTCCTGAGCGCCAGCGTGGGCGACAGGGT |
| | GACCATCACCTGCAAGGCCAGCCAGGACGTGAGCATCGCCGTGGCCTGGTACCAGC |
| | AGAAGCCCGGCAAGGCCCCCAAGCTGCTGATCTACTGGGCCAGCACCAGGCAGACC |
| | GGCGTGCCCAGCAGGTTCAGCGGCAGCGGCAGCGGCACCGACTTCACCCTGACCAT |
| | CAGCAGCCTGCAGCCCGAGGACAGCGCCACCTACTTCTGCCAGCAGTACAGCAGCT |
| | ACCCCTTCACCTTCGGCCAGGGCACCAAGGTGGACATCAAGCGAACGGTGGCTGCC |
| | CCCTCCGTGTTCATCTTCCCCCCCAGCGATGAGCAGCTGAAGAGCGGCACAGCCAGC |
| | GTGGTGTGCCTGCTGAACAACTTCTACCCCAGGGAGGCCAAGGTGCAGTGGAAGGT |
| | GGACAACGCCCTGCAGAGCGGCAACAGCCAGGAGTCCGTGACCGAGCAGGACAGC |
| | AAGGACAGCACCTACAGCCTGAGCAGCACACTGACCCTGAGCAAGGCCGACTACGA |
| | GAAGCACAAGGTGTACGCCTGCGAGGTGACCCATCAGGGCCTGAGCAGCCCCGTGA |
| | CCAAGAGCTTCAACAGGGGCGAGTGC (SEQ ID NO: 163) |
| DAB012124 | GAGATCCAGATGACCCAGAGCCAGAGCACCCTGAGCGTGAGCCTGGGCGACAGGGT |
| | GACCATCACCTGCAAGGCCAGCCAGAACGTGGTGACCGCCGTGGCCTGGTACCAGC |
| | AGAAGCCCGGCCAGGCCCCCAAGCTGCTGATCTACAGCGCCAGCCACAGGTTCACC |
| | GGCGTGCCCAGCAGGTTCAGCGGCAGCGGCAGCGGCACCGACTTCACCCTGATCAT |
| | CAGCTGCATGCAGAGCGAGGACTTCGCCGTGTACTACTGCCAGCAGTACAGCAGCT |
| | ACCCCTACACCTTCGGCCAGGGCACCAAGCTGGAGATCAAGCGAACGGTGGCTGCC |
| | CCCTCCGTGTTCATCTTCCCCCCCAGCGATGAGCAGCTGAAGAGCGGCACAGCCAGC |
| | GTGGTGTGCCTGCTGAACAACTTCTACCCCAGGGAGGCCAAGGTGCAGTGGAAGGT |
| | GGACAACGCCCTGCAGAGCGGCAACAGCCAGGAGTCCGTGACCGAGCAGGACAGC |
| | AAGGACAGCACCTACAGCCTGAGCAGCACACTGACCCTGAGCAAGGCCGACTACGA |
| | GAAGCACAAGGTGTACGCCTGCGAGGTGACCCATCAGGGCCTGAGCAGCCCCGTGA |
| | CCAAGAGCTTCAACAGGGGCGAGTGC (SEQ ID NO: 164) |
| DAB012125 | GACATCCAGATGACCCAGAGCCCCAGCTTCCTGAGCGCCAGCGTGGGCGACAGGGT |
| | GACCATCACCTGCAAGGCCAGCCAGAACGTGGTGACCGCCGTGGCCTGGTACCAGC |
| | AGAAGCCCGGCAAGGCCCCCAAGCTGCTGATCTACAGCGCCAGCCACAGGTTCACC |
| | GGCGTGCCCAGCAGGTTCAGCGGCAGCGGCAGCGGCACCGACTTCACCCTGACCAT |
| | CAGCAACCTGCAGAGCGAGGACCTGGCCACCTACTTCTGCCAGCAGTACAGCAGCT |
| | ACCCCTACACCTTCGGCCAGGGCACCAAGCTGGAGATCAAGCGAACGGTGGCTGCC |
| | CCCTCCGTGTTCATCTTCCCCCCCAGCGATGAGCAGCTGAAGAGCGGCACAGCCAGC |
| | GTGGTGTGCCTGCTGAACAACTTCTACCCCAGGGAGGCCAAGGTGCAGTGGAAGGT |
| | GGACAACGCCCTGCAGAGCGGCAACAGCCAGGAGTCCGTGACCGAGCAGGACAGC |
| | AAGGACAGCACCTACAGCCTGAGCAGCACACTGACCCTGAGCAAGGCCGACTACGA |
| | GAAGCACAAGGTGTACGCCTGCGAGGTGACCCATCAGGGCCTGAGCAGCCCCGTGA |
| | CCAAGAGCTTCAACAGGGGCGAGTGC (SEQ ID NO: 165) |
| DAB012126 | GACATCCAGATGACCCAGAGCCCCAGCTTCCTGAGCGCCAGCGTGGGCGACAGGGT |
| | GACCATCACCTGCAAGGCCAGCCAGAACGTGGTGACCGCCGTGGCCTGGTACCAGC |
| | AGAAGCCCGGCAAGGCCCCCAAGCTGCTGATCTACAGCGCCAGCCACAGGTTCACC |
| | GGCGTGCCCAGCAGGTTCAGCGGCAGCGGCAGCGGCACCGACTTCACCCTGACCAT |
| | CAGCAACCTGCAGCCCGAGGACAGCGCCACCTACTTCTGCCAGCAGTACAGCAGCT |
| | ACCCCTACACCTTCGGCCAGGGCACCAAGCTGGAGATCAAGCGAACGGTGGCTGCC |
| | CCCTCCGTGTTCATCTTCCCCCCCAGCGATGAGCAGCTGAAGAGCGGCACAGCCAGC |
| | GTGGTGTGCCTGCTGAACAACTTCTACCCCAGGGAGGCCAAGGTGCAGTGGAAGGT |
| | GGACAACGCCCTGCAGAGCGGCAACAGCCAGGAGTCCGTGACCGAGCAGGACAGC |
| | AAGGACAGCACCTACAGCCTGAGCAGCACACTGACCCTGAGCAAGGCCGACTACGA |
| | GAAGCACAAGGTGTACGCCTGCGAGGTGACCCATCAGGGCCTGAGCAGCCCCGTGA |
| | CCAAGAGCTTCAACAGGGGCGAGTGC (SEQ ID NO: 166) |
| DAB012127 | GACATCCAGATGACCCAGAGCCCCAGCTTCCTGAGCGCCAGCGTGGGCGACAGGGT |
| | GACCATCACCTGCAAGGCCAGCCAGAACGTGGTGACCGCCGTGGCCTGGTACCAGC |
| | AGAAGCCCGGCAAGGCCCCCAAGCTGCTGATCTACAGCGCCAGCCACAGGTTCACC |
| | GGCGTGCCCAGCAGGTTCAGCGGCAGCGGCAGCGGCACCGACTTCACCCTGACCAT |
| | CAGCAACCTGCAGCCCGACGACGCCGCCACCTACTTCTGCCAGCAGTACAGCAGCT |
| | ACCCCTACACCTTCGGCCAGGGCACCAAGCTGGAGATCAAGCGAACGGTGGCTGCC |
| | CCCTCCGTGTTCATCTTCCCCCCCAGCGATGAGCAGCTGAAGAGCGGCACAGCCAGC |
| | GTGGTGTGCCTGCTGAACAACTTCTACCCCAGGGAGGCCAAGGTGCAGTGGAAGGT |
| | GGACAACGCCCTGCAGAGCGGCAACAGCCAGGAGTCCGTGACCGAGCAGGACAGC |
| | AAGGACAGCACCTACAGCCTGAGCAGCACACTGACCCTGAGCAAGGCCGACTACGA |
| | GAAGCACAAGGTGTACGCCTGCGAGGTGACCCATCAGGGCCTGAGCAGCCCCGTGA |
| | CCAAGAGCTTCAACAGGGGCGAGTGC (SEQ ID NO: 167) |
| DAB012128 | GCCATCCAGATGACCCAGAGCCACAAGAGCTTCAGCGTGAGCACCGGCCAGAGGGT |
| | GACCATCACCTGCAAGGCCAGCCAGGACGTGAGCACCACCGTGGCCTGGTACCAGC |
| | AGAAGCCCGGCCAGGCCCCCAAGCTGCTGATCTACTGGGCCAGCACCAGGCAGACC |
| | GGCGTGCCCAGCAGGTTCACCGGCAGCGGCAGCGGCACCGACTTCACCCTGACCAT |
| | CAGCTGCGTGCAGAGCGAGGACTTCGCCGTGTACTACTGCCAGCAGTACAGCAGCT |
| | ACCCCTTCACCTTCGGCCAGGGCACCAAGCTGGAGATCAAGCGAACGGTGGCTGCC |
| | CCCTCCGTGTTCATCTTCCCCCCCAGCGATGAGCAGCTGAAGAGCGGCACAGCCAGC |
| | GTGGTGTGCCTGCTGAACAACTTCTACCCCAGGGAGGCCAAGGTGCAGTGGAAGGT |
| | GGACAACGCCCTGCAGAGCGGCAACAGCCAGGAGTCCGTGACCGAGCAGGACAGC |
| | AAGGACAGCACCTACAGCCTGAGCAGCACACTGACCCTGAGCAAGGCCGACTACGA |
| | GAAGCACAAGGTGTACGCCTGCGAGGTGACCCATCAGGGCCTGAGCAGCCCCGTGA |
| | CCAAGAGCTTCAACAGGGGCGAGTGC (SEQ ID NO: 168) |
| DAB012129 | GACATCCAGATGACCCAGAGCCCCAGCTTCCTGAGCGCCAGCGTGGGCGACAGGGT |
| | GACCATCACCTGCAAGGCCAGCCAGGACGTGAGCACCACCGTGGCCTGGTACCAGC |
| | AGAAGCCCGGCAAGGCCCCCAAGCTGCTGATCTACTGGGCCAGCACCAGGCAGACC |
| | GGCGTGCCCAGCAGGTTCAGCGGCAGCGGCAGCGGCACCGACTTCACCCTGACCAT |
| | CAGCAACCTGCAGCCCGAGGACTTCGCCACCTACTTCTGCCAGCAGTACAGCAGCTA |
| | CCCCTTCACCTTCGGCCAGGGCACCAAGCTGGAGATCAAGCGAACGGTGGCTGCCC |
| | CCTCCGTGTTCATCTTCCCCCCCAGCGATGAGCAGCTGAAGAGCGGCACAGCCAGCG |
| | TGGTGTGCCTGCTGAACAACTTCTACCCCAGGGAGGCCAAGGTGCAGTGGAAGGTG |
| | GACAACGCCCTGCAGAGCGGCAACAGCCAGGAGTCCGTGACCGAGCAGGACAGCA |
| | AGGACAGCACCTACAGCCTGAGCAGCACACTGACCCTGAGCAAGGCCGACTACGAG |
| | AAGCACAAGGTGTACGCCTGCGAGGTGACCCATCAGGGCCTGAGCAGCCCCGTGAC |
| | CAAGAGCTTCAACAGGGGCGAGTGC (SEQ ID NO: 169) |
| DAB012130 | GACATCCAGATGACCCAGAGCCCCAGCTTCCTGAGCGCCAGCGTGGGCGAGAGGGT |
| | GACCATCACCTGCAAGGCCAGCCAGGACGTGAGCACCACCGTGGCCTGGTACCAGC |
| | AGAAGCCCGGCAAGGCCCCCAAGCTGCTGATCTACTGGGCCAGCACCAGGCAGACC |
| | GGCGTGCCCAGCAGGTTCAGCGGCAGCGGCAGCGGCACCGACTTCACCCTGACCAT |
| | CAACAGCCTGCAGCCCGAGGACGTGGCCACCTACTTCTGCCAGCAGTACAGCAGCT |
| | ACCCCTTCACCTTCGGCCAGGGCACCAAGCTGGAGATCAAGCGAACGGTGGCTGCC |
| | CCCTCCGTGTTCATCTTCCCCCCCAGCGATGAGCAGCTGAAGAGCGGCACAGCCAGC |
| | GTGGTGTGCCTGCTGAACAACTTCTACCCCAGGGAGGCCAAGGTGCAGTGGAAGGT |
| | GGACAACGCCCTGCAGAGCGGCAACAGCCAGGAGTCCGTGACCGAGCAGGACAGC |
| | AAGGACAGCACCTACAGCCTGAGCAGCACACTGACCCTGAGCAAGGCCGACTACGA |
| | GAAGCACAAGGTGTACGCCTGCGAGGTGACCCATCAGGGCCTGAGCAGCCCCGTGA |
| | CCAAGAGCTTCAACAGGGGCGAGTGC (SEQ ID NO: 170) |
| DAB012131 | GACATCCAGATGACCCAGAGCCCCAGCTTCCTGAGCGCCAGCGTGGGCGAGAGGGT |
| | GACCATCACCTGCAAGGCCAGCCAGGACGTGAGCACCACCGTGGCCTGGTACCAGC |
| | AGAAGCCCGGCAAGGCCCCCAAGCTGCTGATCTACTGGGCCAGCACCAGGCAGACC |
| | GGCGTGCCCAGCAGGTTCAGCGGCAGCGGCAGCGGCACCGACTTCACCCTGACCAT |
| | CAGCAACCTGCAGCCCGAGGACGTGGCCACCTACTTCTGCCAGCAGTACAGCAGCT |
| | ACCCCTTCACCTTCGGCCAGGGCACCAAGCTGGAGATCAAGCGAACGGTGGCTGCC |
| | CCCTCCGTGTTCATCTTCCCCCCCAGCGATGAGCAGCTGAAGAGCGGCACAGCCAGC |
| | GTGGTGTGCCTGCTGAACAACTTCTACCCCAGGGAGGCCAAGGTGCAGTGGAAGGT |
| | GGACAACGCCCTGCAGAGCGGCAACAGCCAGGAGTCCGTGACCGAGCAGGACAGC |
| | AAGGACAGCACCTACAGCCTGAGCAGCACACTGACCCTGAGCAAGGCCGACTACGA |
| | GAAGCACAAGGTGTACGCCTGCGAGGTGACCCATCAGGGCCTGAGCAGCCCCGTGA |
| | CCAAGAGCTTCAACAGGGGCGAGTGC (SEQ ID NO: 171) |
| DAB012132 | GACATCCAGATGACCCAGAGCCCCAGCAGCGTGAGCCTGACCGTGGGCCAGAGGGT |
| | GACCATCACCTGCCTGGCCACCGAGGACATCTTCAGCTACCTGGCCTGGTACCAGCA |
| | GAAGCCCGGCAAGGCCCCCAGGCTGCTGATCTACGGCGCCAACAGGCTGAAGGACG |
| | GCGTGCCCAGCAGGTTCAGCGGCAGCGGCAGCGGCACCCAGTACACCCTGAGGATC |
| | AGCAGCATGCAGCCCGAGGACTTCGGCGTGTACTACTGCCTGCAGGGCGCCAAGTT |
| | CCCCCTGACCTTCGGCCCCGGCACCAAGCTGGAGATCAAGCGAACGGTGGCTGCCC |
| | CCTCCGTGTTCATCTTCCCCCCCAGCGATGAGCAGCTGAAGAGCGGCACAGCCAGCG |
| | TGGTGTGCCTGCTGAACAACTTCTACCCCAGGGAGGCCAAGGTGCAGTGGAAGGTG |
| | GACAACGCCCTGCAGAGCGGCAACAGCCAGGAGTCCGTGACCGAGCAGGACAGCA |
| | AGGACAGCACCTACAGCCTGAGCAGCACACTGACCCTGAGCAAGGCCGACTACGAG |
| | AAGCACAAGGTGTACGCCTGCGAGGTGACCCATCAGGGCCTGAGCAGCCCCGTGAC |
| | CAAGAGCTTCAACAGGGGCGAGTGC (SEQ ID NO: 172) |
| DAB012133 | GACATCCAGATGACCCAGAGCCCCAGCAGCCTGAGCGCCAGCATCGGCGACACCGT |
| | GACCATCACCTGCCTGGCCACCGAGGACATCTTCAGCTACCTGGCCTGGTACCAGCA |
| | GAGGCCCGGCAAGGCCCCCAAGCTGCTGATCTACGGCGCCAACAGGCTGAAGGACG |
| | GCGTGCCCAGCAGGTTCAGCGGCAGCGGCAGCGGCACCGACTACACCCTGACCATC |
| | AGCGGCCTGCAGCCCGAGGACTTCGTGACCTACTACTGCCTGCAGGGCGCCAAGTTC |
| | CCCCTGACCTTCGGCCAGGGCACCAAGCTGGACATGAAGCGAACGGTGGCTGCCCC |
| | CTCCGTGTTCATCTTCCCCCCCAGCGATGAGCAGCTGAAGAGCGGCACAGCCAGCGT |
| | GGTGTGCCTGCTGAACAACTTCTACCCCAGGGAGGCCAAGGTGCAGTGGAAGGTGG |
| | ACAACGCCCTGCAGAGCGGCAACAGCCAGGAGTCCGTGACCGAGCAGGACAGCAA |
| | GGACAGCACCTACAGCCTGAGCAGCACACTGACCCTGAGCAAGGCCGACTACGAGA |
| | AGCACAAGGTGTACGCCTGCGAGGTGACCCATCAGGGCCTGAGCAGCCCCGTGACC |
| | AAGAGCTTCAACAGGGGCGAGTGC (SEQ ID NO: 173) |
| DAB012134 | GACATCCAGATGACCCAGAGCCCCAGCAGCCTGAGCGCCAGCGTGGGCGACACCGT |
| | GACCATCACCTGCCTGGCCACCGAGGACATCTTCAGCTACCTGGCCTGGTACCAGCA |
| | GAAGCCCGGCAAGGCCCCCAAGCTGCTGATCTACGGCGCCAACAGGCTGAAGGACG |
| | GCGTGCCCAGCAGGTTCAGCGGCAGCGGCAGCGGCACCGACTACACCCTGACCATC |
| | AGCGGCCTGCAGCCCGAGGACTTCGGCACCTACTACTGCCTGCAGGGCGCCAAGTT |
| | CCCCCTGACCTTCGGCCAGGGCACCAAGCTGGAGATCAAGCGAACGGTGGCTGCCC |
| | CCTCCGTGTTCATCTTCCCCCCCAGCGATGAGCAGCTGAAGAGCGGCACAGCCAGCG |
| | TGGTGTGCCTGCTGAACAACTTCTACCCCAGGGAGGCCAAGGTGCAGTGGAAGGTG |
| | GACAACGCCCTGCAGAGCGGCAACAGCCAGGAGTCCGTGACCGAGCAGGACAGCA |
| | AGGACAGCACCTACAGCCTGAGCAGCACACTGACCCTGAGCAAGGCCGACTACGAG |
| | AAGCACAAGGTGTACGCCTGCGAGGTGACCCATCAGGGCCTGAGCAGCCCCGTGAC |
| | CAAGAGCTTCAACAGGGGCGAGTGC (SEQ ID NO: 174) |
| DAB012135 | GACATCCAGATGACCCAGAGCCCCAGCAGCCTGAGCGCCAGCGTGGGCGACACCGT |
| | GACCATCACCTGCCTGGCCACCGAGGACATCTTCAGCTACCTGGCCTGGTACCAGCA |
| | GAAGCCCGGCAAGGCCCCCAAGCTGCTGATCTACGGCGCCAACAGGCTGAAGGACG |
| | GCGTGCCCAGCAGGTTCAGCGGCGGCGGCAGCGGCACCGAGTACAGCCTGACCATC |
| | AGCGGCCTGCAGCCCGAGGACTTCGGCACCTACTACTGCCTGCAGGGCGCCAAGTT |
| | CCCCCTGACCTTCGGCCAGGGCACCAAGCTGGACATGAAGCGAACGGTGGCTGCCC |
| | CCTCCGTGTTCATCTTCCCCCCCAGCGATGAGCAGCTGAAGAGCGGCACAGCCAGCG |
| | TGGTGTGCCTGCTGAACAACTTCTACCCCAGGGAGGCCAAGGTGCAGTGGAAGGTG |
| | GACAACGCCCTGCAGAGCGGCAACAGCCAGGAGTCCGTGACCGAGCAGGACAGCA |
| | AGGACAGCACCTACAGCCTGAGCAGCACACTGACCCTGAGCAAGGCCGACTACGAG |
| | AAGCACAAGGTGTACGCCTGCGAGGTGACCCATCAGGGCCTGAGCAGCCCCGTGAC |
| | CAAGAGCTTCAACAGGGGCGAGTGC (SEQ ID NO: 175) |
| DAB012135 | GACATCCAGATGACCCAGAGCCCCAGCAGCCTGAGCGCCAGCGTGGGCGACACCGT |
| (modified) | GACCATCACCTGCCTGGCCACCGAGGACATCTTCAGCTACCTGGCCTGGTACCAGCA |
| | GAAGCCCGGCAAGGCCCCCAAGCTGCTGATCTACGGCGCCAACAGGCTGAAGGACA |
| | CCGTGCCCAGCAGGTTCAGCGGCGGCGGCAGCGGCACCGAGTACAGCCTGACCATC |
| | AGCGGCCTGCAGCCCGAGGACTTCGGCACCTACTACTGCCTGCAGGGCGCCAAGTT |
| | CCCCCTGACCTTCGGCCAGGGCACCAAGCTGGACATGAAGCGAACGGTGGCTGCCC |
| | CCTCCGTGTTCATCTTCCCCCCCAGCGATGAGCAGCTGAAGAGCGGCACAGCCAGCG |
| | TGGTGTGCCTGCTGAACAACTTCTACCCCAGGGAGGCCAAGGTGCAGTGGAAGGTG |
| | GACAACGCCCTGCAGAGCGGCAACAGCCAGGAGTCCGTGACCGAGCAGGACAGCA |
| | AGGACAGCACCTACAGCCTGAGCAGCACACTGACCCTGAGCAAGGCCGACTACGAG |
| | AAGCACAAGGTGTACGCCTGCGAGGTGACCCATCAGGGCCTGAGCAGCCCCGTGAC |
| | CAAGAGCTTCAACAGGGGCGAGTGC (SEQ ID NO: 176) |

TABLE 16
Full Length Heavy Chain Nucleotide Sequences of Humanized Antibodies

| Sequence | |
|---|---|
| Name | Heavy chain sequence |
| DAB012120 | CAGGTGCAGCTGCTGGAGAGCGGCGGCGAGCTGGTGCAGCCCGGCGGCAGCCTGAG |
| | GCTGAGCTGCGCCGCCAGCGGCTTCACCTTCAGCGACTACTACATGTTCTGGATCAG |
| | GCAGACCCCCCAGAAGAGGCTGGAGTGGGTGGCCTACATCAGCAACGGCGGCGGCA |
| | ACACCTACTACCCCGACACCGTGAAGGGCAGGTTCACCATCAGCAGGGACAACGCC |
| | AAGAACCAGCTGTACCTGCAGATGAGGAGCCTGACCCCCGAGGACACCGCCATCTA |
| | CTACTGCGCCAGCCCCGAGGCCAGGTACTACGGCAACTACCCCTTCCCCTACTGGGG |
| | CCAGGGCACCCTGGTGACCGTGAGCAGCGCTAGCACCAAGGGCCCATCGGTCTTCC |
| | CCCTGGCACCCTCCTCCAAGAGCACCTCTGGGGGCACAGCGGCCCTGGGCTGCCTGG |
| | TCAAGGACTACTTCCCCGAACCGGTGACGGTGTCGTGGAACTCAGGCGCCCTGACC |
| | AGCGGCGTGCACACCTTCCCGGCTGTCCTACAGTCCTCAGGACTCTACTCCCTCAGC |
| | AGCGTGGTGACCGTGCCCTCCAGCAGCTTGGGCACCCAGACCTACATCTGCAACGTG |
| | AATCACAAGCCCAGCAACACCAAGGTGGACAAGAAAGTTGAGCCCAAATCTTGTGA |
| | CAAAACTCACACATGCCCACCGTGCCCAGCACCTGAACTCCTGGGGGGACCGTCAG |
| | TCTTCCTCTTCCCCCCAAAACCCAAGGACACCCTCATGATCTCCCGGACCCCTGAGG |
| | TCACATGCGTGGTGGTGGACGTGAGCCACGAAGACCCTGAGGTCAAGTTCAACTGG |
| | TACGTGGACGGCGTGGAGGTGCATAATGCCAAGACAAAGCCGCGGGAGGAGCAGT |
| | ACAACAGCACGTACCGGGTGGTCAGCGTCCTCACCGTCCTGCACCAGGACTGGCTG |
| | AATGGCAAGGAGTACAAGTGCAAGGTCTCCAACAAAGCCCTCCCAGCCCCCATCGA |
| | GAAAACCATCTCCAAAGCCAAAGGGCAGCCCCGAGAACCACAGGTGTACACCCTGC |
| | CCCCATCCCGGGATGAGCTGACCAAGAACCAGGTCAGCCTGACCTGCCTGGTCAAA |
| | GGCTTCTATCCCAGCGACATCGCCGTGGAGTGGGAGAGCAATGGGCAGCCGGAGAA |
| | CAACTACAAGACCACGCCTCCCGTGCTGGACTCCGACGGCTCCTTCTTCCTCTACAG |
| | CAAGCTCACCGTGGACAAGAGCAGGTGGCAGCAGGGGAACGTCTTCTCATGCTCCG |
| | TGATGCATGAGGCTCTGCACAACCACTACACGCAGAAGAGCCTCTCCCTGTCTCCGG |
| | GTAAA (SEQ ID NO: 177) |
| DAB012121 | CAGGTGCAGCTGGTGGAGAGCGGCGGCGGCCTGGTGAAGCCCGGCGGCAGCCTGAG |
| | GCTGAGCTGCGCCGCCAGCGGCTTCACCTTCAGCGACTACTACATGTTCTGGATCAG |
| | GCAGGCCCCCGGCAAGGGCCTGGAGTGGGTGGCCTACATCAGCAACGGCGGCGGCA |
| | ACACCTACTACCCCGACACCGTGAAGGGCAGGTTCACCATCAGCAGGGACAACGCC |
| | AAGAACACCCTGTACCTGCAGATGAACAGCCTGAGGGCCGAGGACACCGCCGTGTA |
| | CTACTGCGCCAGCCCCGAGGCCAGGTACTACGGCAACTACCCCTTCCCCTACTGGGG |
| | CCAGGGCACCCTGGTGACCGTGAGCAGCGCTAGCACCAAGGGCCCATCGGTCTTCC |
| | CCCTGGCACCCTCCTCCAAGAGCACCTCTGGGGGCACAGCGGCCCTGGGCTGCCTGG |
| | TCAAGGACTACTTCCCCGAACCGGTGACGGTGTCGTGGAACTCAGGCGCCCTGACC |
| | AGCGGCGTGCACACCTTCCCGGCTGTCCTACAGTCCTCAGGACTCTACTCCCTCAGC |
| | AGCGTGGTGACCGTGCCCTCCAGCAGCTTGGGCACCCAGACCTACATCTGCAACGTG |
| | AATCACAAGCCCAGCAACACCAAGGTGGACAAGAAAGTTGAGCCCAAATCTTGTGA |
| | CAAAACTCACACATGCCCACCGTGCCCAGCACCTGAACTCCTGGGGGGACCGTCAG |
| | TCTTCCTCTTCCCCCCAAAACCCAAGGACACCCTCATGATCTCCCGGACCCCTGAGG |
| | TCACATGCGTGGTGGTGGACGTGAGCCACGAAGACCCTGAGGTCAAGTTCAACTGG |
| | TACGTGGACGGCGTGGAGGTGCATAATGCCAAGACAAAGCCGCGGGAGGAGCAGT |
| | ACAACAGCACGTACCGGGTGGTCAGCGTCCTCACCGTCCTGCACCAGGACTGGCTG |
| | AATGGCAAGGAGTACAAGTGCAAGGTCTCCAACAAAGCCCTCCCAGCCCCCATCGA |
| | GAAAACCATCTCCAAAGCCAAAGGGCAGCCCCGAGAACCACAGGTGTACACCCTGC |
| | CCCCATCCCGGGATGAGCTGACCAAGAACCAGGTCAGCCTGACCTGCCTGGTCAAA |
| | GGCTTCTATCCCAGCGACATCGCCGTGGAGTGGGAGAGCAATGGGCAGCCGGAGAA |
| | CAACTACAAGACCACGCCTCCCGTGCTGGACTCCGACGGCTCCTTCTTCCTCTACAG |
| | CAAGCTCACCGTGGACAAGAGCAGGTGGCAGCAGGGGAACGTCTTCTCATGCTCCG |
| | TGATGCATGAGGCTCTGCACAACCACTACACGCAGAAGAGCCTCTCCCTGTCTCCGG |
| | GTAAA (SEQ ID NO: 178) |
| DAB012122 | CAGGTGCAGCTGGTGGAGAGCGGCGGCGGCGTGGTGAAGCCCGGCGGCAGCCTGA |
| | GGCTGAGCTGCGCCGCCAGCGGCTTCACCTTCAGCGACTACTACATGTTCTGGATCA |
| | GGCAGGCCCCCGGCAAGGGCCTGGAGTGGGTGGCCTACATCAGCAACGGCGGCGGC |
| | AACACCTACTACCCCGACACCGTGAAGGGCAGGTTCACCATCAGCAGGGACAACGC |
| | CAAGAACACCCTGTACCTGCAGATGAACAGCCTGAGGGCCGAGGACACCGCCGTGT |
| | ACTACTGCGCCAGCCCCGAGGCCAGGTACTACGGCAACTACCCCTTCCCCTACTGGG |
| | GCCAGGGCACCCTGGTGACCGTGAGCAGCGCTAGCACCAAGGGCCCATCGGTCTTC |
| | CCCCTGGCACCCTCCTCCAAGAGCACCTCTGGGGGCACAGCGGCCCTGGGCTGCCTG |
| | GTCAAGGACTACTTCCCCGAACCGGTGACGGTGTCGTGGAACTCAGGCGCCCTGAC |
| | CAGCGGCGTGCACACCTTCCCGGCTGTCCTACAGTCCTCAGGACTCTACTCCCTCAG |
| | CAGCGTGGTGACCGTGCCCTCCAGCAGCTTGGGCACCCAGACCTACATCTGCAACGT |
| | GAATCACAAGCCCAGCAACACCAAGGTGGACAAGAAAGTTGAGCCCAAATCTTGTG |
| | ACAAAACTCACACATGCCCACCGTGCCCAGCACCTGAACTCCTGGGGGGACCGTCA |
| | GTCTTCCTCTTCCCCCCAAAACCCAAGGACACCCTCATGATCTCCCGGACCCCTGAG |
| | GTCACATGCGTGGTGGTGGACGTGAGCCACGAAGACCCTGAGGTCAAGTTCAACTG |
| | GTACGTGGACGGCGTGGAGGTGCATAATGCCAAGACAAAGCCGCGGGAGGAGCAG |
| | TACAACAGCACGTACCGGGTGGTCAGCGTCCTCACCGTCCTGCACCAGGACTGGCTG |
| | AATGGCAAGGAGTACAAGTGCAAGGTCTCCAACAAAGCCCTCCCAGCCCCCATCGA |
| | GAAAACCATCTCCAAAGCCAAAGGGCAGCCCCGAGAACCACAGGTGTACACCCTGC |
| | CCCCATCCCGGGATGAGCTGACCAAGAACCAGGTCAGCCTGACCTGCCTGGTCAAA |
| | GGCTTCTATCCCAGCGACATCGCCGTGGAGTGGGAGAGCAATGGGCAGCCGGAGAA |
| | CAACTACAAGACCACGCCTCCCGTGCTGGACTCCGACGGCTCCTTCTTCCTCTACAG |
| | CAAGCTCACCGTGGACAAGAGCAGGTGGCAGCAGGGGAACGTCTTCTCATGCTCCG |
| | TGATGCATGAGGCTCTGCACAACCACTACACGCAGAAGAGCCTCTCCCTGTCTCCGG |
| | GTAAA (SEQ ID NO: 179) |
| DAB012123 | CAGGTGCAGCTGGTGGAGAGCGGCGGCGGCGTGGTGCAGCCCGGCGGCAGCCTGAG |
| | GCTGAGCTGCGCCGCCAGCGGCTTCACCTTCAGCGACTACTACATGTTCTGGATCAG |
| | GCAGGCCCCCGGCAAGGGCCTGGAGTGGGTGGCCTACATCAGCAACGGCGGCGGCA |
| | ACACCTACTACCCCGACACCGTGAAGGGCAGGTTCACCATCAGCAGGGACAACGCC |
| | AAGAACACCCTGTACCTGCAGATGAACAGCCTGAGGGCCGAGGACACCGCCGTGTA |
| | CTACTGCGCCAGCCCCGAGGCCAGGTACTACGGCAACTACCCCTTCCCCTACTGGGG |
| | CCAGGGCACCCTGGTGACCGTGAGCAGCGCTAGCACCAAGGGCCCATCGGTCTTCC |
| | CCCTGGCACCCTCCTCCAAGAGCACCTCTGGGGGCACAGCGGCCCTGGGCTGCCTGG |
| | TCAAGGACTACTTCCCCGAACCGGTGACGGTGTCGTGGAACTCAGGCGCCCTGACC |
| | AGCGGCGTGCACACCTTCCCGGCTGTCCTACAGTCCTCAGGACTCTACTCCCTCAGC |
| | AGCGTGGTGACCGTGCCCTCCAGCAGCTTGGGCACCCAGACCTACATCTGCAACGTG |
| | AATCACAAGCCCAGCAACACCAAGGTGGACAAGAAAGTTGAGCCCAAATCTTGTGA |
| | CAAAACTCACACATGCCCACCGTGCCCAGCACCTGAACTCCTGGGGGGACCGTCAG |
| | TCTTCCTCTTCCCCCCAAAACCCAAGGACACCCTCATGATCTCCCGGACCCCTGAGG |
| | TCACATGCGTGGTGGTGGACGTGAGCCACGAAGACCCTGAGGTCAAGTTCAACTGG |
| | TACGTGGACGGCGTGGAGGTGCATAATGCCAAGACAAAGCCGCGGGAGGAGCAGT |
| | ACAACAGCACGTACCGGGTGGTCAGCGTCCTCACCGTCCTGCACCAGGACTGGCTG |
| | AATGGCAAGGAGTACAAGTGCAAGGTCTCCAACAAAGCCCTCCCAGCCCCCATCGA |
| | GAAAACCATCTCCAAAGCCAAAGGGCAGCCCCGAGAACCACAGGTGTACACCCTGC |
| | CCCCATCCCGGGATGAGCTGACCAAGAACCAGGTCAGCCTGACCTGCCTGGTCAAA |
| | GGCTTCTATCCCAGCGACATCGCCGTGGAGTGGGAGAGCAATGGGCAGCCGGAGAA |
| | CAACTACAAGACCACGCCTCCCGTGCTGGACTCCGACGGCTCCTTCTTCCTCTACAG |
| | CAAGCTCACCGTGGACAAGAGCAGGTGGCAGCAGGGGAACGTCTTCTCATGCTCCG |
| | TGATGCATGAGGCTCTGCACAACCACTACACGCAGAAGAGCCTCTCCCTGTCTCCGG |
| | GTAAA (SEQ ID NO: 180) |
| DAB012124 | CAGGTGCAGCTGCTGCAGAGCGGCCCCGGCCTGGTGAAGCCCAGCGCCACCCTGAG |
| | CCTGACCTGCACCGTGAGCGGCTTCAGCCTGACCACCTACGGCGTGCACTGGATCAG |
| | GCAGCCCCCCGGCAAGGGCCTGGAGTGGATGGGCGTGATCTGGAGCGGCGGCAGCA |
| | CCGACTACAACGCCGCCTTCATCAGCAGGCTGACCATCAGCAAGGACAACAGCAAG |
| | AACCAGGTGTTCCTGCAGCTGTACAGCCTGAGGGCCGAGGACACCGCCATCTACTA |
| | CTGCGCCAGGACCAGCCACTGGTACTTCGACGTGTGGGGCAGGGGCACCCTGGTGA |
| | CCGTGAGCAGCGCTAGCACCAAGGGCCCATCGGTCTTCCCCCTGGCACCCTCCTCCA |
| | AGAGCACCTCTGGGGGCACAGCGGCCCTGGGCTGCCTGGTCAAGGACTACTTCCCC |
| | GAACCGGTGACGGTGTCGTGGAACTCAGGCGCCCTGACCAGCGGCGTGCACACCTT |
| | CCCGGCTGTCCTACAGTCCTCAGGACTCTACTCCCTCAGCAGCGTGGTGACCGTGCC |
| | CTCCAGCAGCTTGGGCACCCAGACCTACATCTGCAACGTGAATCACAAGCCCAGCA |
| | ACACCAAGGTGGACAAGAAAGTTGAGCCCAAATCTTGTGACAAAACTCACACATGC |
| | CCACCGTGCCCAGCACCTGAACTCCTGGGGGGACCGTCAGTCTTCCTCTTCCCCCCA |
| | AAACCCAAGGACACCCTCATGATCTCCCGGACCCCTGAGGTCACATGCGTGGTGGT |
| | GGACGTGAGCCACGAAGACCCTGAGGTCAAGTTCAACTGGTACGTGGACGGCGTGG |
| | AGGTGCATAATGCCAAGACAAAGCCGCGGGAGGAGCAGTACAACAGCACGTACCG |
| | GGTGGTCAGCGTCCTCACCGTCCTGCACCAGGACTGGCTGAATGGCAAGGAGTACA |
| | AGTGCAAGGTCTCCAACAAAGCCCTCCCAGCCCCCATCGAGAAAACCATCTCCAAA |
| | GCCAAAGGGCAGCCCCGAGAACCACAGGTGTACACCCTGCCCCCATCCCGGGATGA |
| | GCTGACCAAGAACCAGGTCAGCCTGACCTGCCTGGTCAAAGGCTTCTATCCCAGCG |
| | ACATCGCCGTGGAGTGGGAGAGCAATGGGCAGCCGGAGAACAACTACAAGACCAC |
| | GCCTCCCGTGCTGGACTCCGACGGCTCCTTCTTCCTCTACAGCAAGCTCACCGTGGA |
| | CAAGAGCAGGTGGCAGCAGGGGAACGTCTTCTCATGCTCCGTGATGCATGAGGCTC |
| | TGCACAACCACTACACGCAGAAGAGCCTCTCCCTGTCTCCGGGTAAA (SEQ ID NO: |
| | 181) |
| DAB012125 | CAGGTGCAGCTGCAGGAGAGCGGCCCCGGCCTGGTGAAGCCCAGCCAGACCCTGAG |
| | CCTGACCTGCACCGTGAGCGGCTTCAGCCTGACCACCTACGGCGTGCACTGGGTGAG |
| | GCAGAGCCCCGGCAAGGGCCTGGAGTGGCTGGGCGTGATCTGGAGCGGCGGCAGCA |
| | CCGACTACAACGCCGCCTTCATCAGCAGGCTGAGCATCAGCAAGGACAACAGCAAG |
| | AGCCAGGTGTTCCTGGAGATGACCAGCCTGACCGCCGCCGACACCGCCATCTACTAC |
| | TGCGCCAGGACCAGCCACTGGTACTTCGACGTGTGGGGCAGGGGCACCCTGGTGAC |
| | CGTGAGCAGCGCTAGCACCAAGGGCCCATCGGTCTTCCCCCTGGCACCCTCCTCCAA |
| | GAGCACCTCTGGGGGCACAGCGGCCCTGGGCTGCCTGGTCAAGGACTACTTCCCCG |
| | AACCGGTGACGGTGTCGTGGAACTCAGGCGCCCTGACCAGCGGCGTGCACACCTTC |
| | CCGGCTGTCCTACAGTCCTCAGGACTCTACTCCCTCAGCAGCGTGGTGACCGTGCCC |
| | TCCAGCAGCTTGGGCACCCAGACCTACATCTGCAACGTGAATCACAAGCCCAGCAA |
| | CACCAAGGTGGACAAGAAAGTTGAGCCCAAATCTTGTGACAAAACTCACACATGCC |
| | CACCGTGCCCAGCACCTGAACTCCTGGGGGGACCGTCAGTCTTCCTCTTCCCCCCAA |
| | AACCCAAGGACACCCTCATGATCTCCCGGACCCCTGAGGTCACATGCGTGGTGGTG |
| | GACGTGAGCCACGAAGACCCTGAGGTCAAGTTCAACTGGTACGTGGACGGCGTGGA |
| | GGTGCATAATGCCAAGACAAAGCCGCGGGAGGAGCAGTACAACAGCACGTACCGG |
| | GTGGTCAGCGTCCTCACCGTCCTGCACCAGGACTGGCTGAATGGCAAGGAGTACAA |
| | GTGCAAGGTCTCCAACAAAGCCCTCCCAGCCCCCATCGAGAAAACCATCTCCAAAG |
| | CCAAAGGGCAGCCCCGAGAACCACAGGTGTACACCCTGCCCCCATCCCGGGATGAG |
| | CTGACCAAGAACCAGGTCAGCCTGACCTGCCTGGTCAAAGGCTTCTATCCCAGCGAC |
| | ATCGCCGTGGAGTGGGAGAGCAATGGGCAGCCGGAGAACAACTACAAGACCACGC |
| | CTCCCGTGCTGGACTCCGACGGCTCCTTCTTCCTCTACAGCAAGCTCACCGTGGACA |
| | AGAGCAGGTGGCAGCAGGGGAACGTCTTCTCATGCTCCGTGATGCATGAGGCTCTG |
| | CACAACCACTACACGCAGAAGAGCCTCTCCCTGTCTCCGGGTAAA (SEQ ID NO: 182) |
| DAB012126 | CAGGTGCAGCTGCAGGAGAGCGGCCCCGGCCTGGTGAAGCCCAGCCAGACCCTGAG |
| | CCTGACCTGCACCGTGAGCGGCTTCAGCCTGACCACCTACGGCGTGCACTGGGTGAG |
| | GCAGGCCCCCGAGAAGGGCCTGGAGTGGCTGGGCGTGATCTGGAGCGGCGGCAGCA |
| | CCGACTACAACGCCGCCTTCATCAGCAGGCTGAGCATCAGCAAGGACAACAGCAAG |
| | AGCCAGGTGTTCCTGGAGATGACCAGCCTGACCGCCGACGACACCGCCATCTACTA |
| | CTGCGCCAGGACCAGCCACTGGTACTTCGACGTGTGGGGCAGGGGCACCCTGGTGA |
| | CCGTGAGCAGCGCTAGCACCAAGGGCCCATCGGTCTTCCCCCTGGCACCCTCCTCCA |
| | AGAGCACCTCTGGGGGCACAGCGGCCCTGGGCTGCCTGGTCAAGGACTACTTCCCC |
| | GAACCGGTGACGGTGTCGTGGAACTCAGGCGCCCTGACCAGCGGCGTGCACACCTT |
| | CCCGGCTGTCCTACAGTCCTCAGGACTCTACTCCCTCAGCAGCGTGGTGACCGTGCC |
| | CTCCAGCAGCTTGGGCACCCAGACCTACATCTGCAACGTGAATCACAAGCCCAGCA |
| | ACACCAAGGTGGACAAGAAAGTTGAGCCCAAATCTTGTGACAAAACTCACACATGC |
| | CCACCGTGCCCAGCACCTGAACTCCTGGGGGGACCGTCAGTCTTCCTCTTCCCCCCA |
| | AAACCCAAGGACACCCTCATGATCTCCCGGACCCCTGAGGTCACATGCGTGGTGGT |
| | GGACGTGAGCCACGAAGACCCTGAGGTCAAGTTCAACTGGTACGTGGACGGCGTGG |
| | AGGTGCATAATGCCAAGACAAAGCCGCGGGAGGAGCAGTACAACAGCACGTACCG |
| | GGTGGTCAGCGTCCTCACCGTCCTGCACCAGGACTGGCTGAATGGCAAGGAGTACA |
| | AGTGCAAGGTCTCCAACAAAGCCCTCCCAGCCCCCATCGAGAAAACCATCTCCAAA |
| | GCCAAAGGGCAGCCCCGAGAACCACAGGTGTACACCCTGCCCCCATCCCGGGATGA |
| | GCTGACCAAGAACCAGGTCAGCCTGACCTGCCTGGTCAAAGGCTTCTATCCCAGCG |
| | ACATCGCCGTGGAGTGGGAGAGCAATGGGCAGCCGGAGAACAACTACAAGACCAC |
| | GCCTCCCGTGCTGGACTCCGACGGCTCCTTCTTCCTCTACAGCAAGCTCACCGTGGA |
| | CAAGAGCAGGTGGCAGCAGGGGAACGTCTTCTCATGCTCCGTGATGCATGAGGCTC |
| | TGCACAACCACTACACGCAGAAGAGCCTCTCCCTGTCTCCGGGTAAA (SEQ ID NO: |
| | 183) |
| DAB012127 | CAGGTGCAGCTGCAGGAGAGCGGCCCCGGCCTGGTGAAGCCCAGCCAGACCCTGAG |
| | CCTGACCTGCACCGTGAGCGGCTTCAGCCTGACCACCTACGGCGTGCACTGGGTGAG |
| | GCAGACCCCCGAGAAGGGCCTGGAGTGGCTGGGCGTGATCTGGAGCGGCGGCAGCA |
| | CCGACTACAACGCCGCCTTCATCAGCAGGCTGAGCATCAGCAAGGACAACAGCAAG |
| | AGCCAGGTGTTCCTGGAGATGACCAGCCTGACCGCCGACGACACCGCCATCTACTA |
| | CTGCGCCAGGACCAGCCACTGGTACTTCGACGTGTGGGGCAGGGGCACCCTGGTGA |
| | CCGTGAGCAGCGCTAGCACCAAGGGCCCATCGGTCTTCCCCCTGGCACCCTCCTCCA |
| | AGAGCACCTCTGGGGGCACAGCGGCCCTGGGCTGCCTGGTCAAGGACTACTTCCCC |
| | GAACCGGTGACGGTGTCGTGGAACTCAGGCGCCCTGACCAGCGGCGTGCACACCTT |
| | CCCGGCTGTCCTACAGTCCTCAGGACTCTACTCCCTCAGCAGCGTGGTGACCGTGCC |
| | CTCCAGCAGCTTGGGCACCCAGACCTACATCTGCAACGTGAATCACAAGCCCAGCA |
| | ACACCAAGGTGGACAAGAAAGTTGAGCCCAAATCTTGTGACAAAACTCACACATGC |
| | CCACCGTGCCCAGCACCTGAACTCCTGGGGGGACCGTCAGTCTTCCTCTTCCCCCCA |
| | AAACCCAAGGACACCCTCATGATCTCCCGGACCCCTGAGGTCACATGCGTGGTGGT |
| | GGACGTGAGCCACGAAGACCCTGAGGTCAAGTTCAACTGGTACGTGGACGGCGTGG |
| | AGGTGCATAATGCCAAGACAAAGCCGCGGGAGGAGCAGTACAACAGCACGTACCG |
| | GGTGGTCAGCGTCCTCACCGTCCTGCACCAGGACTGGCTGAATGGCAAGGAGTACA |
| | AGTGCAAGGTCTCCAACAAAGCCCTCCCAGCCCCCATCGAGAAAACCATCTCCAAA |
| | GCCAAAGGGCAGCCCCGAGAACCACAGGTGTACACCCTGCCCCCATCCCGGGATGA |
| | GCTGACCAAGAACCAGGTCAGCCTGACCTGCCTGGTCAAAGGCTTCTATCCCAGCG |
| | ACATCGCCGTGGAGTGGGAGAGCAATGGGCAGCCGGAGAACAACTACAAGACCAC |
| | GCCTCCCGTGCTGGACTCCGACGGCTCCTTCTTCCTCTACAGCAAGCTCACCGTGGA |
| | CAAGAGCAGGTGGCAGCAGGGGAACGTCTTCTCATGCTCCGTGATGCATGAGGCTC |
| | TGCACAACCACTACACGCAGAAGAGCCTCTCCCTGTCTCCGGGTAAA (SEQ ID NO: |
| | 184) |
| DAB012128 | CAGGTGCAGCTGCTGGAGAGCGGCGGCGGCCTGGTGCAGCCCGGCGGCAGCCTGAG |
| | GCTGAGCTGCGCCGCCAGCGGCTTCACCTTCAGCGACTACTACATGTACTGGATCAG |
| | GCAGACCCCCCAGAAGAGGCTGGAGTGGGTGGCCTACATCAGCAACGGCGGCGGCA |
| | ACACCTACTACAGCGACACCGTGAAGGGCAGGTTCACCATCAGCAGGGACAACGCC |
| | AAGAACCAGCTGTACCTGCAGATGAGGAGCCTGAGGCCCGAGGACACCGCCATCTA |
| | CTACTGCGCCAGCCCCGAGGCCAGGTACTACGGCAACTTCCCCTTCCCCTACTGGGG |
| | CCAGGGCACCCTGGTGACCGTGAGCAGCGCTAGCACCAAGGGCCCATCGGTCTTCC |
| | CCCTGGCACCCTCCTCCAAGAGCACCTCTGGGGGCACAGCGGCCCTGGGCTGCCTGG |
| | TCAAGGACTACTTCCCCGAACCGGTGACGGTGTCGTGGAACTCAGGCGCCCTGACC |
| | AGCGGCGTGCACACCTTCCCGGCTGTCCTACAGTCCTCAGGACTCTACTCCCTCAGC |
| | AGCGTGGTGACCGTGCCCTCCAGCAGCTTGGGCACCCAGACCTACATCTGCAACGTG |
| | AATCACAAGCCCAGCAACACCAAGGTGGACAAGAAAGTTGAGCCCAAATCTTGTGA |
| | CAAAACTCACACATGCCCACCGTGCCCAGCACCTGAACTCCTGGGGGGACCGTCAG |
| | TCTTCCTCTTCCCCCCAAAACCCAAGGACACCCTCATGATCTCCCGGACCCCTGAGG |
| | TCACATGCGTGGTGGTGGACGTGAGCCACGAAGACCCTGAGGTCAAGTTCAACTGG |
| | TACGTGGACGGCGTGGAGGTGCATAATGCCAAGACAAAGCCGCGGGAGGAGCAGT |
| | ACAACAGCACGTACCGGGTGGTCAGCGTCCTCACCGTCCTGCACCAGGACTGGCTG |
| | AATGGCAAGGAGTACAAGTGCAAGGTCTCCAACAAAGCCCTCCCAGCCCCCATCGA |
| | GAAAACCATCTCCAAAGCCAAAGGGCAGCCCCGAGAACCACAGGTGTACACCCTGC |
| | CCCCATCCCGGGATGAGCTGACCAAGAACCAGGTCAGCCTGACCTGCCTGGTCAAA |
| | GGCTTCTATCCCAGCGACATCGCCGTGGAGTGGGAGAGCAATGGGCAGCCGGAGAA |
| | CAACTACAAGACCACGCCTCCCGTGCTGGACTCCGACGGCTCCTTCTTCCTCTACAG |
| | CAAGCTCACCGTGGACAAGAGCAGGTGGCAGCAGGGGAACGTCTTCTCATGCTCCG |
| | TGATGCATGAGGCTCTGCACAACCACTACACGCAGAAGAGCCTCTCCCTGTCTCCGG |
| | GTAAA (SEQ ID NO: 185) |
| DAB012129 | CAGGTGCAGCTGGTGGAGAGCGGCGGCGGCCTGGTGAAGCCCGGCGGCAGCCTGAG |
| | GCTGAGCTGCGCCGCCAGCGGCTTCACCTTCAGCGACTACTACATGTACTGGATCAG |
| | GCAGGCCCCCGGCAAGGGCCTGGAGTGGGTGGCCTACATCAGCAACGGCGGCGGCA |
| | ACACCTACTACAGCGACACCGTGAAGGGCAGGTTCACCATCAGCAGGGACAACGCC |
| | AAGAACACCCTGTACCTGCAGATGAACAGCCTGAGGGCCGAGGACACCGCCGTGTA |
| | CTACTGCGCCAGCCCCGAGGCCAGGTACTACGGCAACTTCCCCTTCCCCTACTGGGG |
| | CCAGGGCACCCTGGTGACCGTGAGCAGCGCTAGCACCAAGGGCCCATCGGTCTTCC |
| | CCCTGGCACCCTCCTCCAAGAGCACCTCTGGGGGCACAGCGGCCCTGGGCTGCCTGG |
| | TCAAGGACTACTTCCCCGAACCGGTGACGGTGTCGTGGAACTCAGGCGCCCTGACC |
| | AGCGGCGTGCACACCTTCCCGGCTGTCCTACAGTCCTCAGGACTCTACTCCCTCAGC |
| | AGCGTGGTGACCGTGCCCTCCAGCAGCTTGGGCACCCAGACCTACATCTGCAACGTG |
| | AATCACAAGCCCAGCAACACCAAGGTGGACAAGAAAGTTGAGCCCAAATCTTGTGA |
| | CAAAACTCACACATGCCCACCGTGCCCAGCACCTGAACTCCTGGGGGGACCGTCAG |
| | TCTTCCTCTTCCCCCCAAAACCCAAGGACACCCTCATGATCTCCCGGACCCCTGAGG |
| | TCACATGCGTGGTGGTGGACGTGAGCCACGAAGACCCTGAGGTCAAGTTCAACTGG |
| | TACGTGGACGGCGTGGAGGTGCATAATGCCAAGACAAAGCCGCGGGAGGAGCAGT |
| | ACAACAGCACGTACCGGGTGGTCAGCGTCCTCACCGTCCTGCACCAGGACTGGCTG |
| | AATGGCAAGGAGTACAAGTGCAAGGTCTCCAACAAAGCCCTCCCAGCCCCCATCGA |
| | GAAAACCATCTCCAAAGCCAAAGGGCAGCCCCGAGAACCACAGGTGTACACCCTGC |
| | CCCCATCCCGGGATGAGCTGACCAAGAACCAGGTCAGCCTGACCTGCCTGGTCAAA |
| | GGCTTCTATCCCAGCGACATCGCCGTGGAGTGGGAGAGCAATGGGCAGCCGGAGAA |
| | CAACTACAAGACCACGCCTCCCGTGCTGGACTCCGACGGCTCCTTCTTCCTCTACAG |
| | CAAGCTCACCGTGGACAAGAGCAGGTGGCAGCAGGGGAACGTCTTCTCATGCTCCG |
| | TGATGCATGAGGCTCTGCACAACCACTACACGCAGAAGAGCCTCTCCCTGTCTCCGG |
| | GTAAA (SEQ ID NO: 186) |
| DAB012130 | CAGGTGCAGCTGGTGGAGAGCGGCGGCGGCCTGGTGAAGCCCGGCGGCAGCCTGAG |
| | GCTGAGCTGCGCCGCCAGCGGCTTCACCTTCAGCGACTACTACATGTACTGGATCAG |
| | GCAGGCCCCCGGCAAGGGCCTGGAGTGGGTGGCCTACATCAGCAACGGCGGCGGCA |
| | ACACCTACTACAGCGACACCGTGAAGGGCAGGTTCACCATCAGCAGGGACAACGCC |
| | AAGAACACCCTGTACCTGCAGATGAACAGCCTGAGGGCCGAGGACACCGCCGTGTA |
| | CTACTGCGCCAGCCCCGAGGCCAGGTACTACGGCAACTTCCCCTTCCCCTACTGGGG |
| | CCAGGGCACCCTGGTGACCGTGAGCAGCGCTAGCACCAAGGGCCCATCGGTCTTCC |
| | CCCTGGCACCCTCCTCCAAGAGCACCTCTGGGGGCACAGCGGCCCTGGGCTGCCTGG |
| | TCAAGGACTACTTCCCCGAACCGGTGACGGTGTCGTGGAACTCAGGCGCCCTGACC |
| | AGCGGCGTGCACACCTTCCCGGCTGTCCTACAGTCCTCAGGACTCTACTCCCTCAGC |
| | AGCGTGGTGACCGTGCCCTCCAGCAGCTTGGGCACCCAGACCTACATCTGCAACGTG |
| | AATCACAAGCCCAGCAACACCAAGGTGGACAAGAAAGTTGAGCCCAAATCTTGTGA |
| | CAAAACTCACACATGCCCACCGTGCCCAGCACCTGAACTCCTGGGGGGACCGTCAG |
| | TCTTCCTCTTCCCCCCAAAACCCAAGGACACCCTCATGATCTCCCGGACCCCTGAGG |
| | TCACATGCGTGGTGGTGGACGTGAGCCACGAAGACCCTGAGGTCAAGTTCAACTGG |
| | TACGTGGACGGCGTGGAGGTGCATAATGCCAAGACAAAGCCGCGGGAGGAGCAGT |
| | ACAACAGCACGTACCGGGTGGTCAGCGTCCTCACCGTCCTGCACCAGGACTGGCTG |
| | AATGGCAAGGAGTACAAGTGCAAGGTCTCCAACAAAGCCCTCCCAGCCCCCATCGA |
| | GAAAACCATCTCCAAAGCCAAAGGGCAGCCCCGAGAACCACAGGTGTACACCCTGC |
| | CCCCATCCCGGGATGAGCTGACCAAGAACCAGGTCAGCCTGACCTGCCTGGTCAAA |
| | GGCTTCTATCCCAGCGACATCGCCGTGGAGTGGGAGAGCAATGGGCAGCCGGAGAA |
| | CAACTACAAGACCACGCCTCCCGTGCTGGACTCCGACGGCTCCTTCTTCCTCTACAG |
| | CAAGCTCACCGTGGACAAGAGCAGGTGGCAGCAGGGGAACGTCTTCTCATGCTCCG |
| | TGATGCATGAGGCTCTGCACAACCACTACACGCAGAAGAGCCTCTCCCTGTCTCCGG |
| | GTAAA (SEQ ID NO: 187) |
| DAB012131 | CAGGTGCAGCTGGTGGAGAGCGGCGGCGGCCTGGTGAAGCCCGGCGGCAGCCTGAG |
| | GCTGAGCTGCGCCGCCAGCGGCTTCACCTTCAGCGACTACTACATGTACTGGATCAG |
| | GCAGGCCCCCGGCAAGGGCCTGGAGTGGGTGGCCTACATCAGCAACGGCGGCGGCA |
| | ACACCTACTACAGCGACACCGTGAAGGGCAGGTTCACCATCAGCAGGGACAACGCC |
| | AAGAACACCCTGTACCTGCAGATGAACAGCCTGAGGGCCGAGGACACCGCCGTGTA |
| | CTACTGCGCCAGCCCCGAGGCCAGGTACTACGGCAACTTCCCCTTCCCCTACTGGGG |
| | CCAGGGCACCCTGGTGACCGTGAGCAGCGCTAGCACCAAGGGCCCATCGGTCTTCC |
| | CCCTGGCACCCTCCTCCAAGAGCACCTCTGGGGGCACAGCGGCCCTGGGCTGCCTGG |
| | TCAAGGACTACTTCCCCGAACCGGTGACGGTGTCGTGGAACTCAGGCGCCCTGACC |
| | AGCGGCGTGCACACCTTCCCGGCTGTCCTACAGTCCTCAGGACTCTACTCCCTCAGC |
| | AGCGTGGTGACCGTGCCCTCCAGCAGCTTGGGCACCCAGACCTACATCTGCAACGTG |
| | AATCACAAGCCCAGCAACACCAAGGTGGACAAGAAAGTTGAGCCCAAATCTTGTGA |
| | CAAAACTCACACATGCCCACCGTGCCCAGCACCTGAACTCCTGGGGGGACCGTCAG |
| | TCTTCCTCTTCCCCCCAAAACCCAAGGACACCCTCATGATCTCCCGGACCCCTGAGG |
| | TCACATGCGTGGTGGTGGACGTGAGCCACGAAGACCCTGAGGTCAAGTTCAACTGG |
| | TACGTGGACGGCGTGGAGGTGCATAATGCCAAGACAAAGCCGCGGGAGGAGCAGT |
| | ACAACAGCACGTACCGGGTGGTCAGCGTCCTCACCGTCCTGCACCAGGACTGGCTG |
| | AATGGCAAGGAGTACAAGTGCAAGGTCTCCAACAAAGCCCTCCCAGCCCCCATCGA |
| | GAAAACCATCTCCAAAGCCAAAGGGCAGCCCCGAGAACCACAGGTGTACACCCTGC |
| | CCCCATCCCGGGATGAGCTGACCAAGAACCAGGTCAGCCTGACCTGCCTGGTCAAA |
| | GGCTTCTATCCCAGCGACATCGCCGTGGAGTGGGAGAGCAATGGGCAGCCGGAGAA |
| | CAACTACAAGACCACGCCTCCCGTGCTGGACTCCGACGGCTCCTTCTTCCTCTACAG |
| | CAAGCTCACCGTGGACAAGAGCAGGTGGCAGCAGGGGAACGTCTTCTCATGCTCCG |
| | TGATGCATGAGGCTCTGCACAACCACTACACGCAGAAGAGCCTCTCCCTGTCTCCGG |
| | GTAAA (SEQ ID NO: 188) |
| DAB012132 | CAGGTGCAGCTGGTGGAGAGCGGCGGCGGCGTGGTGCAGCCCGGCAGGAGCCTGA |
| | GGCTGAGCTGCGCCGCCAGCGGCTTCAGCTTCAGCAACTACTACATGGCCTGGGTGA |
| | GGCAGGCCCCCGGCAAGGGCCTGGAGTGGGTGGCCAGCATCAGCACCGGCGGCGGC |
| | AACATCTACTACAGGGACAGCGTGAAGGGCAGGTTCACCATCAGCAGGGACAACAG |
| | CAAGAGCACCCTGTACCTGCAGATGAGGAGCCTGAGGAGCGAGGACACCGCCATCT |
| | ACTACTGCGCCAGGCAGACCGCCTACTACGTGATGGACGCCTGGGGCCAGGGCACC |
| | ATGGTGACCGTGAGCAGCGCTAGCACCAAGGGCCCATCGGTCTTCCCCCTGGCACCC |
| | TCCTCCAAGAGCACCTCTGGGGGCACAGCGGCCCTGGGCTGCCTGGTCAAGGACTA |
| | CTTCCCCGAACCGGTGACGGTGTCGTGGAACTCAGGCGCCCTGACCAGCGGCGTGC |
| | ACACCTTCCCGGCTGTCCTACAGTCCTCAGGACTCTACTCCCTCAGCAGCGTGGTGA |
| | CCGTGCCCTCCAGCAGCTTGGGCACCCAGACCTACATCTGCAACGTGAATCACAAGC |
| | CCAGCAACACCAAGGTGGACAAGAAAGTTGAGCCCAAATCTTGTGACAAAACTCAC |
| | ACATGCCCACCGTGCCCAGCACCTGAACTCCTGGGGGGACCGTCAGTCTTCCTCTTC |
| | CCCCCAAAACCCAAGGACACCCTCATGATCTCCCGGACCCCTGAGGTCACATGCGTG |
| | GTGGTGGACGTGAGCCACGAAGACCCTGAGGTCAAGTTCAACTGGTACGTGGACGG |
| | CGTGGAGGTGCATAATGCCAAGACAAAGCCGCGGGAGGAGCAGTACAACAGCACG |
| | TACCGGGTGGTCAGCGTCCTCACCGTCCTGCACCAGGACTGGCTGAATGGCAAGGA |
| | GTACAAGTGCAAGGTCTCCAACAAAGCCCTCCCAGCCCCCATCGAGAAAACCATCT |
| | CCAAAGCCAAAGGGCAGCCCCGAGAACCACAGGTGTACACCCTGCCCCCATCCCGG |
| | GATGAGCTGACCAAGAACCAGGTCAGCCTGACCTGCCTGGTCAAAGGCTTCTATCCC |
| | AGCGACATCGCCGTGGAGTGGGAGAGCAATGGGCAGCCGGAGAACAACTACAAGA |
| | CCACGCCTCCCGTGCTGGACTCCGACGGCTCCTTCTTCCTCTACAGCAAGCTCACCG |
| | TGGACAAGAGCAGGTGGCAGCAGGGGAACGTCTTCTCATGCTCCGTGATGCATGAG |
| | GCTCTGCACAACCACTACACGCAGAAGAGCCTCTCCCTGTCTCCGGGTAAA (SEQ ID |
| | NO: 189) |
| DAB012133 | GAGGTGCAGCTGGTGGAGAGCGGCGGCGGCCTGGTGCAGCCCGGCGGCAGCCTGAG |
| | GCTGAGCTGCGCCGCCAGCGGCTTCAGCTTCAGCAACTACTACATGGCCTGGGTGAG |
| | GCAGGCCCCCGGCAAGGGCCTGGAGTGGGTGGCCAGCATCAGCACCGGCGGCGGCA |
| | ACATCTACTACAGGGACAGCGTGAAGGGCAGGTTCACCATCAGCAGGGACAACGCC |
| | AAGAACAGCCTGTACCTGCAGATGAACAGCCTGAGGGCCGAGGACACCGCCGTGTA |
| | CTACTGCGCCAGGCAGACCGCCTACTACGTGATGGACGCCTGGGGCCAGGGCACCA |
| | CCGTGACCGTGAGCAGCGCTAGCACCAAGGGCCCATCGGTCTTCCCCCTGGCACCCT |
| | CCTCCAAGAGCACCTCTGGGGGCACAGCGGCCCTGGGCTGCCTGGTCAAGGACTAC |
| | TTCCCCGAACCGGTGACGGTGTCGTGGAACTCAGGCGCCCTGACCAGCGGCGTGCA |
| | CACCTTCCCGGCTGTCCTACAGTCCTCAGGACTCTACTCCCTCAGCAGCGTGGTGAC |
| | CGTGCCCTCCAGCAGCTTGGGCACCCAGACCTACATCTGCAACGTGAATCACAAGCC |
| | CAGCAACACCAAGGTGGACAAGAAAGTTGAGCCCAAATCTTGTGACAAAACTCACA |
| | CATGCCCACCGTGCCCAGCACCTGAACTCCTGGGGGGACCGTCAGTCTTCCTCTTCC |
| | CCCCAAAACCCAAGGACACCCTCATGATCTCCCGGACCCCTGAGGTCACATGCGTG |
| | GTGGTGGACGTGAGCCACGAAGACCCTGAGGTCAAGTTCAACTGGTACGTGGACGG |
| | CGTGGAGGTGCATAATGCCAAGACAAAGCCGCGGGAGGAGCAGTACAACAGCACG |
| | TACCGGGTGGTCAGCGTCCTCACCGTCCTGCACCAGGACTGGCTGAATGGCAAGGA |
| | GTACAAGTGCAAGGTCTCCAACAAAGCCCTCCCAGCCCCCATCGAGAAAACCATCT |
| | CCAAAGCCAAAGGGCAGCCCCGAGAACCACAGGTGTACACCCTGCCCCCATCCCGG |
| | GATGAGCTGACCAAGAACCAGGTCAGCCTGACCTGCCTGGTCAAAGGCTTCTATCCC |
| | AGCGACATCGCCGTGGAGTGGGAGAGCAATGGGCAGCCGGAGAACAACTACAAGA |
| | CCACGCCTCCCGTGCTGGACTCCGACGGCTCCTTCTTCCTCTACAGCAAGCTCACCG |
| | TGGACAAGAGCAGGTGGCAGCAGGGGAACGTCTTCTCATGCTCCGTGATGCATGAG |
| | GCTCTGCACAACCACTACACGCAGAAGAGCCTCTCCCTGTCTCCGGGTAAA (SEQ ID |
| | NO: 190) |
| DAB012134 | GAGGTGCAGCTGGTGGAGAGCGGCGGCGGCCTGGTGCAGCCCGGCGGCAGCCTGAG |
| | GCTGAGCTGCGCCGCCAGCGGCTTCAGCTTCAGCAACTACTACATGGCCTGGGTGAG |
| | GCAGGCCCCCGGCAAGGGCCTGGAGTGGGTGGCCAGCATCAGCACCGGCGGCGGCA |
| | ACATCTACTACAGGGACAGCGTGAAGGGCAGGTTCACCATCAGCAGGGACAACGCC |
| | AAGAACAGCCTGTACCTGCAGATGAACAGCCTGAGGGCCGAGGACACCGCCGTGTA |
| | CTACTGCGCCAGGCAGACCGCCTACTACGTGATGGACGCCTGGGGCCAGGGCACCA |
| | CCGTGACCGTGAGCAGCGCTAGCACCAAGGGCCCATCGGTCTTCCCCCTGGCACCCT |
| | CCTCCAAGAGCACCTCTGGGGGCACAGCGGCCCTGGGCTGCCTGGTCAAGGACTAC |
| | TTCCCCGAACCGGTGACGGTGTCGTGGAACTCAGGCGCCCTGACCAGCGGCGTGCA |
| | CACCTTCCCGGCTGTCCTACAGTCCTCAGGACTCTACTCCCTCAGCAGCGTGGTGAC |
| | CGTGCCCTCCAGCAGCTTGGGCACCCAGACCTACATCTGCAACGTGAATCACAAGCC |
| | CAGCAACACCAAGGTGGACAAGAAAGTTGAGCCCAAATCTTGTGACAAAACTCACA |
| | CATGCCCACCGTGCCCAGCACCTGAACTCCTGGGGGGACCGTCAGTCTTCCTCTTCC |
| | CCCCAAAACCCAAGGACACCCTCATGATCTCCCGGACCCCTGAGGTCACATGCGTG |
| | GTGGTGGACGTGAGCCACGAAGACCCTGAGGTCAAGTTCAACTGGTACGTGGACGG |
| | CGTGGAGGTGCATAATGCCAAGACAAAGCCGCGGGAGGAGCAGTACAACAGCACG |
| | TACCGGGTGGTCAGCGTCCTCACCGTCCTGCACCAGGACTGGCTGAATGGCAAGGA |
| | GTACAAGTGCAAGGTCTCCAACAAAGCCCTCCCAGCCCCCATCGAGAAAACCATCT |
| | CCAAAGCCAAAGGGCAGCCCCGAGAACCACAGGTGTACACCCTGCCCCCATCCCGG |
| | GATGAGCTGACCAAGAACCAGGTCAGCCTGACCTGCCTGGTCAAAGGCTTCTATCCC |
| | AGCGACATCGCCGTGGAGTGGGAGAGCAATGGGCAGCCGGAGAACAACTACAAGA |
| | CCACGCCTCCCGTGCTGGACTCCGACGGCTCCTTCTTCCTCTACAGCAAGCTCACCG |
| | TGGACAAGAGCAGGTGGCAGCAGGGGAACGTCTTCTCATGCTCCGTGATGCATGAG |
| | GCTCTGCACAACCACTACACGCAGAAGAGCCTCTCCCTGTCTCCGGGTAAA (SEQ ID |
| | NO: 191) |
| DAB012135 | GAGGTGCAGCTGGTGGAGAGCGGCGGCGGCCTGGTGCAGCCCGGCGGCAGCCTGAG |
| | GCTGAGCTGCGCCGCCAGCGGCTTCAGCTTCAGCAACTACTACATGGCCTGGGTGAG |
| | GCAGGCCCCCGGCAAGGGCCTGGAGTGGGTGGCCAGCATCAGCACCGGCGGCGGCA |
| | ACATCTACTACAGGGACAGCGTGAAGGGCAGGTTCACCATCAGCAGGGACAACGCC |
| | AAGAACAGCCTGTACCTGCAGATGAACAGCCTGAGGGCCGAGGACACCGCCGTGTA |
| | CTACTGCGCCAGGCAGACCGCCTACTACGTGATGGACGCCTGGGGCCAGGGCACCA |
| | CCGTGACCGTGAGCAGCGCTAGCACCAAGGGCCCATCGGTCTTCCCCCTGGCACCCT |
| | CCTCCAAGAGCACCTCTGGGGGCACAGCGGCCCTGGGCTGCCTGGTCAAGGACTAC |
| | TTCCCCGAACCGGTGACGGTGTCGTGGAACTCAGGCGCCCTGACCAGCGGCGTGCA |
| | CACCTTCCCGGCTGTCCTACAGTCCTCAGGACTCTACTCCCTCAGCAGCGTGGTGAC |
| | CGTGCCCTCCAGCAGCTTGGGCACCCAGACCTACATCTGCAACGTGAATCACAAGCC |
| | CAGCAACACCAAGGTGGACAAGAAAGTTGAGCCCAAATCTTGTGACAAAACTCACA |
| | CATGCCCACCGTGCCCAGCACCTGAACTCCTGGGGGGACCGTCAGTCTTCCTCTTCC |
| | CCCCAAAACCCAAGGACACCCTCATGATCTCCCGGACCCCTGAGGTCACATGCGTG |
| | GTGGTGGACGTGAGCCACGAAGACCCTGAGGTCAAGTTCAACTGGTACGTGGACGG |
| | CGTGGAGGTGCATAATGCCAAGACAAAGCCGCGGGAGGAGCAGTACAACAGCACG |
| | TACCGGGTGGTCAGCGTCCTCACCGTCCTGCACCAGGACTGGCTGAATGGCAAGGA |
| | GTACAAGTGCAAGGTCTCCAACAAAGCCCTCCCAGCCCCCATCGAGAAAACCATCT |
| | CCAAAGCCAAAGGGCAGCCCCGAGAACCACAGGTGTACACCCTGCCCCCATCCCGG |
| | GATGAGCTGACCAAGAACCAGGTCAGCCTGACCTGCCTGGTCAAAGGCTTCTATCCC |
| | AGCGACATCGCCGTGGAGTGGGAGAGCAATGGGCAGCCGGAGAACAACTACAAGA |
| | CCACGCCTCCCGTGCTGGACTCCGACGGCTCCTTCTTCCTCTACAGCAAGCTCACCG |
| | TGGACAAGAGCAGGTGGCAGCAGGGGAACGTCTTCTCATGCTCCGTGATGCATGAG |
| | GCTCTGCACAACCACTACACGCAGAAGAGCCTCTCCCTGTCTCCGGGTAAA (SEQ ID |
| | NO: 192) |

### Example 5: Testing Binding of Nectin-4 Humanized Antibodies

A surface plasmon resonance (SPR) assay was used to determine the binding affinity of Nectin-4 antibodies to Nectin-4 protein. Antibodies were captured using an anti-human Fc capture antibody (Cytiva, #BR100839) immobilized onto a Biacore CM5 standard surface sensor chip following manufacturer instructions. Recombinant human His-tagged Nectin-4 protein was titrated over captured antibody as a 3-fold serial dilutions starting from 400 nM. Association was monitored for 180 sec and dissociation was monitored for 300 sec. Binding assays were performed at 25° C. using HBS-EP+running buffer containing 10 mM HEPES, 150 mM NaCl, 3 mM EDTA, 0.05% Tween® 20. The surface of the CM5 chip was regenerated with 3M MgCl2 for 30 sec at 30 mL/min. The assay was run on a Biacore® T200 instrument (Cytiva, Marlborough, Massachusetts, USA). The data were “double referenced” by subtracting the response from the reference control flow cell and that from a buffer injection and were fit to a 1:1 binding model using Cytiva's Biacore® T200 Evaluation Software. The association rate constant (ka, M−1s−1) and dissociation rate constant (kd, s−1) were determined and used to calculate the equilibrium dissociation rate (KD, M)=kd/ka. The reported affinity (KD) was calculated from the average of 2 replicate interactions. Data is shown in Table 17.

TABLE 17
Binding Data for Nectin-4 Humanized Antibodies

| Analyte | Human Nectin-4 | | | | | |
|---|---|---|---|---|---|---|
| Sequence | ka | | | Cyno Nectin-4 | | |
| name/Lot # | (1/M-s) | kd (1/s) | KD (M) | ka (1/M-s) | kd (1/s) | KD (M) |
| DAB012120_001 | 2.00E+05 | 5.00E−04 | 2.50E−09 | 1.60E+05 | 5.30E−04 | 3.30E−09 |
| DAB012121_001 | 2.10E+05 | 5.70E−04 | 2.70E−09 | 1.70E+05 | 6.10E−04 | 3.70E−09 |
| DAB012122_001 | 1.70E+05 | 5.90E−04 | 3.50E−09 | 1.30E+05 | 6.00E−04 | 4.50E−09 |
| DAB012123_001 | 1.50E+05 | 5.40E−04 | 3.70E−09 | 1.10E+05 | 5.70E−04 | 5.10E−09 |
| DAB012124_001 | 8.40E+04 | 3.30E−04 | 3.90E−09 | 6.50E+04 | 3.20E−04 | 5.00E−09 |
| DAB012125_001 | 8.70E+04 | 3.10E−04 | 3.50E−09 | 6.50E+04 | 3.50E−04 | 5.40E−09 |
| DAB012126_001 | 9.60E+04 | 3.00E−04 | 3.10E−09 | 7.10E+04 | 3.20E−04 | 4.50E−09 |
| DAB012127_001 | 9.20E+04 | 3.20E−04 | 3.50E−09 | 7.10E+04 | 3.50E−04 | 5.00E−09 |
| DAB012128_001 | 1.50E+05 | 9.90E−04 | 6.50E−09 | 1.20E+05 | 9.80E−04 | 8.50E−09 |
| DAB012129_001 | 2.50E+05 | 1.60E−03 | 6.50E−09 | 1.90E+05 | 1.60E−03 | 8.60E−09 |
| DAB012130_001 | 2.90E+05 | 2.50E−03 | 8.70E−09 | 2.20E+05 | 2.40E−03 | 1.10E−08 |
| DAB012131_001 | 2.80E+05 | 2.60E−03 | 9.20E−09 | 2.20E+05 | 2.50E−03 | 1.10E−08 |
| DAB012132_001 | 2.00E+05 | 1.10E−03 | 5.30E−09 | 1.30E+05 | 3.20E−03 | 2.40E−08 |
| DAB012133_001 | 3.80E+05 | 1.00E−03 | 2.70E−09 | 2.70E+05 | 3.10E−03 | 1.10E−08 |
| DAB012134_001 | 3.90E+05 | 9.90E−04 | 2.50E−09 | 2.90E+05 | 2.80E−03 | 9.80E−09 |
| DAB012135_001 | 3.80E+05 | 7.70E−04 | 2.00E−09 | 2.80E+05 | 2.20E−03 | 7.80E−09 |

### Example 6: Synthesis of Nectin-4 Mask Sequences

Phage Biopanning: Biopanning with ml3 phagemid p8 or p3 displayed peptide libraries was either performed with immobilized antibody or antibody fragments of interest coated on 96-well ELISA plates or with biotin-conjugated antibody or antibody fragments of interest immobilized on streptavidin coated paramagnetic beads. Following binding to target and washing steps, specifically bound phage were recovered by elution using acidic buffer. Enrichment of specific binding clones was accomplished by 3-4 rounds of successive biopanning and amplification. After 3 or 4 rounds of biopanning phage pools were infected into TGI cells and plated out on LB-ampicillin/agar plates for clonal isolation and subsequent characterization. Phage Hit Identification: After plating TG1 infected with phage pool eluates,

individual colonies were grown in 96 well plates for several hours and infected with helper phage to produce peptide displayed phagemid following an overnight growth. The next day the plates were centrifuged to separate the soluble phagemid from the E. coli cells. The phagemid containing supernatants were then combined with PBS Tween 20 (0.05%)+BSA (1%) pH neutral blocking buffer and incubated in previously target antibody or antibody fragment coated and blocked wells. After a short incubation, plates were washed and bound phage were detected by anti-ml3 HRP conjugated antibodies using standard TMB-based chromogenic ELISA procedures. Daughter plates or individual wells were subjected to standard DNA sequencing for peptide identification. Phagemid peptide clones were next tested to determine whether they bound within the antigen binding space of the antibody, by target-based competition assay. Target antibody or antibody fragments thereof were immobilized and blocked in a 96-well ELISA plate format. Human Nectin-4 antigen was added to the wells to block the antigen binding site of the plated coated antibody or antibody fragment. After a brief incubation period phagemid supernatants were added to the wells. Following another short incubation period the plates were washed and specifically bound phage were detected by anti-ml3 HRP conjugated antibodies using standard TMB-based chromogenic ELISA procedures. Phagemid clones were determined to bind within the antigenic binding pocket of the target antibody if a decrease in the phage binding signal was observed compared to a well lacking nectin-4 antigen blockade.

Peptide Synthesis: Peptides of interest identified through phage biopanning were chemically synthesized by standard solid phase peptide synthesis techniques know in the art.

Peptide Binding Assays for Mask Sequences (ELISA and Octet): Equilibrium binding experiments (ELISA)-Peptides were evaluated for their ability to bind to target anti-Nectin-4 antibody or antibody fragments thereof in a standard enzyme linked immunosorbent assay (ELISA) format. Specifically, peptides were evaluated for their ability to bind antibody or antibody fragments whose cognate antigen is human Nectin-4. Briefly, biotinylated peptides or biotinylated Nectin-4 antigen were captured on neutravidin coated plates. Target antibody or antibody fragments were diluted in buffer and added to the peptide or antigen captured plates. Bound antibody was detected using a standard horse radish peroxidase conjugate secondary antibody. The concentration of antibody or antibody fragment thereof required to achieve 50% maximal signal (EC50) was calculated using Graphpad Prism software.

Kinetic binding experiments (Octet)-Kinetic binding of peptides to target antibody or antibody fragments thereof were evaluated using biolayer interferometry (BLI). Briefly, biotinylated peptides or biotinylated Nectin-4 antigen were loaded onto a streptavidin coated Octet® SAX biosensor, quenched in biocytin, and baselined in buffer. Target antibody or and antibody fragment thereof was titrated in solution and associated onto the peptide loaded sensor. After a short association period, sensors were transferred into buffer and the dissociation of bound antibody or antibody fragment thereof was measured. Association and dissociation signals were recorded in real time and analyzed using a 1:1 binding model within the instrument software. Analysis using a 1:1 binding model enabled the calculation of the on and off rate constants as well as affinity, KD.

Peptide competitive binding assays—The ability of peptides to bind and inhibit target antibody or antibody fragments thereof was determined in standard competitive ELISA binding experiments. Peptides were evaluated for their ability to inhibit anti-Nectin-4 antibodies or fragments thereof from binding to the Nectin-4 antigen in a standard ELISA format. Briefly, biotinylated nectin-4 antigen was captured on neutravidin coated plates. Anti-Nectin4 antibody or antibody fragments fixed at 2 nanomolar (nM) concentration was pre-incubated with 0-100 micromolar (μM) titrated peptides. After a short pre-incubation period, the mixture of titrated peptide with fixed Nectin-4 antibody or antibody fragment were added to the Nectin-4 antigen coated plates. After a short incubation on the plates, bound antibody or antibody fragment was detected with a standard horse radish peroxidase conjugated secondary antibody. The concentration of peptide required to reduce the maximum signal by 50% (IC50) was calculated in Graphpad Prism software.

Alanine Scanning of Peptides in Mask Sequences: Sequence activity relationships of select peptides were established using standard alanine scan techniques known in the art. Each residue of select peptides was mutated to Alanine and resulting mutant peptides were characterized for their ability to bind the antibody or antibody fragment of interest as well as their ability to inhibit antibody or antibody fragment of interest from binding Nectin-4 protein antigen. Critical residues within the peptide were then identified as those that lost significant binding affinity to the antibody or antibody fragment of interest when mutated to alanine. Peptide residues that maintained binding to target antibody or antibody fragment were identified as a non-critical residue. This information was used to synthesize directed evolution peptide libraries to strengthen peptide affinity for the target antibody or antibody fragment of interest.

Peptide Optimization of Mask Sequences Via Directed Evolution of Phage Library: Directed evolution peptide libraries were synthesized with select mutagenesis of non-critical peptide residues. Critical peptide residues were minimally mutated in the directed evolution libraries. The subsequent peptide libraries were displayed on m13 phagemid via p3 and subject to biopanning as described above.

Select Nectin-4 Mask sequences that were synthesized using this process are shown in in Table 18.

TABLE 18
Nectin-4 Mask Sequences

| Mask Sequence | Peptide Sequence |
|---|---|
| Nectin-4 Mask sequence #1 (JXP1957) | GGWSCAPDEDTWLCPAGG (SEQ ID NO: 18) |
| Nectin-4 Mask sequence #2 (JXP1952) | GGWFCAPDENTWLCPNAGG (SEQ ID NO: 23) |
| Nectin-4 Mask sequence #3 (JXP1956) | GGWTCADDEDTWLCPIGG (SEQ ID NO: 125) |
| Nectin-4 Mask sequence #4 (JXP1963) | GGWACAPDEDTWLCPIGG (SEQ ID NO: 126) |

### Example 7: Cell Culture of the Nectin-4 TRACTr Molecule

HD-BIOP3 (a rAAV-modified GS double knockout CHO host cell line licensed from Horizon Discovery) was used as the host cell line for transfection. These cells were maintained in CD CHO medium (Cat #10743029 Thermo Fisher) supplemented with 1× HT (Cat #10743029 Thermo Fisher) and 8 mM L-glutamine (Cat #25030081 Thermo Fisher). Cells were passaged every 2-3 days at a seeding density of 0.3×10\hat{ }6 cells/ml in shake flasks then placed on a shaker set to 140 rpm with a 25 mm orbit. Host cells were maintained in an incubator set to 36.5C, 5% CO2 and 80% humidity.

The heavy chain (SEQ ID NO: 194) (see Table 19)) and light chain (SEQ ID NO: 193 (see Table 19)) genes were separately cloned into different cargo vectors (spB007) which was purchased from Transposagen Biopharmaceuticals. The spB007 vector contained ITR sites to facilitate PiggyBac integration into the host cell genome. Both the LC and HC were controlled by a CMV promoter and the glutamine synthetase gene was controlled by a SV40 promoter. The entire expression cassette (SV40-GS-CMV-HC or LC) was cloned between the ITR sites in the spB007 vector. Helper plasmid coding for the PiggyBac transposase was purchased from Transposagen Biopharmaceuticals (Cat #spb-DNA-25).

Two days prior to transfection, the host cells were seeded at 0.7×10\hat{ }6 cells/ml in the host cell media described above. On the day of transfections cells were counted and then centrifuged at 1000 rpm for 10 minutes. They were then resuspended in the host cell media containing DMSO with a cell concentration of 6×10\hat{ }6 cells/ml. The cells were then placed back on a shaker set to 140 rpm with a 25 mm orbit for 3 hours. The incubator settings were 36.5C with 5% CO₂ and 80% humidity. Cultures were then co-transfected with the expression vectors SPB007-HC, spB007-LC and the helper plasmid spB-DNA using PEI at (2.5:1, PEI: DNA ratio). Cells were placed back in the incubator for 2 days to recover. After 48-hours of recovery, the cells were passaged into selection media: CDCHO, 1× HT and 12.5 μM MSX (Cat #GSS-1015-F, Sigma). Transfected cells were split to 0.3×10\hat{ }6 cells/ml every 3-4 days in the selection media until the viability reached above 90% which was in 2 weeks. At this point, a stable pool was established.

After recovery from selection, the stable pool was placed in fed-batch production. In this process, the basal medium used was Dynamis (Cat #A2661501 Thermo Fisher), 1× HT and 1 μM CuSO4—Sigma (C8027). The stable pool was seeded in the above production media at 0.5×10\hat{ }6 cells/ml and placed in a 36.5C, 5% CO2, 80% humidity incubator on a shaker at 140 rpm and 25 mm orbit. Three days post inoculation the stable pools were temp-shifted to 32° C. and maintained there for the duration of the culture. The production culture was fed on day 3, 7 and 10 with 10% Feed C (Cat #A2503104, Thermo Fisher.) Glucose was monitored and maintained at 6 g/L throughout the fed-batch production. After 12 days in fed-batch production, the cell supernatant was prepared for purification by clarification through a 0.2 μm filter.

TABLE 19
Cloning Sequences for Nectin-4 TRACTr Molecule

| | |
|---|---|
| Sequence encoding | GGAGGATGGTCCTGCGCCCCCGACGAGGACACCTGGCTGTGCCCCGCCG |
| light chain of | GCGGAGGAGGCTCTGGAGGACTGTCTGGCAGGTCCGACGCCGGCTCCCC |
| Nectin-4 TRACTr | TCTGGGCCTGGCTGGCAGCGGCGGCTCTGACATCCAGATGACACAGTCC |
| molecule | CCATCCAGCCTGTCCGCCTCCGTGGGCGACACCGTGACAATCACCTGTCT |
| | GGCCACCGAGGACATCTTCTCCTACCTGGCTTGGTATCAGCAGAAGCCAG |
| | GCAAGGCCCCCAAGCTGCTGATCTACGGCGCTAATAGGCTGAAGGACAC |
| | AGTGCCATCCCGGTTCAGCGGAGGAGGCTCCGGCACAGAGTATTCTCTG |
| | ACCATCTCCGGCCTGCAGCCTGAGGATTTTGGCACCTACTATTGCCTGCA |
| | GGGCGCCAAGTTCCCACTGACATTTGGCCAGGGCACCAAGCTGGACATG |
| | AAGAGAACAGTGGCCGCTCCCTCCGTGTTCATCTTTCCCCCTAGCGATGA |
| | GCAGCTGAAGAGCGGCACCGCTTCTGTGGTGTGCCTGCTGAACAATTTCT |
| | ACCCTCGCGAGGCCAAGGTGCAGTGGAAGGTGGACAACGCTCTGCAGTC |
| | TGGCAATTCCCAGGAGAGCGTGACAGAGCAGGACTCTAAGGATTCCACC |
| | TATAGCCTGTCTTCCACACTGACCCTGTCTAAGGCCGATTACGAGAAGCA |
| | CAAGGTGTATGCTTGCGAGGTGACACATCAGGGCCTGTCCTCCCCCGTGA |
| | CCAAGTCCTTTAACAGAGGCGAGTGT (SEQ ID NO: 193) |
| Sequence encoding | GAGGTGCAGCTGGTGGAGTCCGGAGGAGGACTGGTGCAGCCTGGCGGCT |
| heavy chain of | CCCTGAGACTGAGCTGTGCCGCTTCTGGCTCCACATTCTACACCGCCGTG |
| Nectin-4 TRACTr | ATGGGATGGGTGAGGCAGGCTCCAGGCAAGGGCCTGGAGTGGGTGGCTG |
| molecule | CTATCAGGTGGACAGCCCTGACCACATCTTATGCTGACTCCGTGAAGGGC |
| | AGATTCACCATCTCCCGCGATGGCGCCAAGACCACACTGTACCTGCAGAT |
| | GAACAGCCTGAGACCTGAGGACACAGCCGTGTACTATTGCGCTGCTCGC |
| | GGCACCCTGGGACTGTTTACCACAGCTGACTCCTACGATTATTGGGGCCA |
| | GGGCACACTGGTGACCGTGTCCAGCGGCGGAGGAGGCAGCGGAGGAGG |
| | CTCTGGCGGCGTGTACTGCGGCCCAGAGTTCGACGAGTCCGTGGGCTGTA |
| | TGGGCGGCGGAGGCAGCGGAGGAGGACTGTCCGGCAGAAGCGATGCTG |
| | GCTCCCCACTGGGCCTGGCTGGCTCCGGAGGAGGCAGCGAAGTCCAGCT |
| | GGTGGAGAGCGGCGGCGGCCTGGTCCAGCCTGGCGGCTCTCTGAAGCTG |
| | TCCTGTGCCGCCTCCGGCTTCACCTTTAACAAGTATGCCATGAATTGGGT |
| | GCGCCAGGCTCCCGGCAAGGGCCTGGAGTGGGTAGCCAGGATCAGGTCC |
| | AAGTACAACAATTATGCCACCTACTACGCCGACTCCGTGAAGGATAGGT |
| | TCACAATCTCTCGGGACGATTCCAAGAACACCGCCTACCTGCAGATGAA |
| | CAATCTGAAGACAGAGGACACCGCCGTGTACTATTGCGTGAGGCACGGC |
| | AACTTTGGCAATTCTTACATCTCCTATTGGGCTTACTGGGGTCAGGGCAC |
| | ACTGGTCACCGTGTCTTCCGGAGGAGGAGGCTCCGGCGGCGGAGGCAGC |
| | GGCGGCGGCGGCTCTCAGACAGTGGTGACCCAGGAGCCAAGCCTGACCG |
| | TGTCTCCCGGCGGCACCGTGACACTGACCTGTGGCAGCTCTACAGGAGCT |
| | GTGACCAGCGGAAACTATCCAAATTGGGTGCAGCAGAAGCCTGGCCAGG |
| | CTCCTAGAGGCCTGATCGGAGGCACAAAGTTCCTGGCCCCAGGCACCCC |
| | AGCTCGCTTTAGCGGCTCTCTGCTGGGAGGCAAGGCCGCTCTGACCCTGA |
| | GCGGAGTGCAGCCAGAGGATGAGGCCGAGTACTATTGCGTGCTGTGGTA |
| | CTCTAACAGATGGGTGTTTGGCGGCGGCACAAAGCTGACCGTGCTGGGA |
| | GGAGGAGGCAGCGAAGTGCAGCTGGTCGAGTCTGGCGGCGGCTTAGTCC |
| | AACCTGGCGGCTCCCTGAGGCTGTCTTGCGCCGCTTCTGGCTTCTCCTTTA |
| | GCAACTACTATATGGCTTGGGTGCGGCAGGCTCCTGGCAAGGGCCTGGA |
| | GTGGGTCGCCTCTATCTCCACAGGCGGCGGCAATATCTACTATCGGGACT |
| | CTGTGAAGGGCAGGTTCACCATCTCCAGGGACAACGCTAAGAATAGCCT |
| | GTATCTGCAGATGAACTCCCTGAGGGCCGAAGATACTGCCGTGTACTACT |
| | GCGCCCGGCAGACCGCTTACTATGTGATGGATGCCTGGGGCCAGGGCAC |
| | CACAGTGACAGTGTCCAGCGCCTCCACCAAGGGCCCTAGCGTGTTCCCTC |
| | TGGCTCCATCTTCCAAGAGCACATCTGGAGGCACCGCCGCTCTGGGATGT |
| | CTGGTGAAGGACTACTTCCCCGAGCCTGTGACCGTGAGCTGGAACTCTGG |
| | CGCCCTGACATCTGGCGTGCACACCTTTCCCGCTGTGCTGCAGTCCTCCG |
| | GCCTGTATTCCCTGTCCAGCGTGGTGACAGTGCCTTCTTCCAGCCTGGGC |
| | ACACAGACCTACATCTGCAACGTGAATCATAAGCCTAGCAATACCAAGG |
| | TGGATAAGAAGGTGGAGCCAAAGTCTTGT (SEQ ID NO: 194) |

### Example 8: Purification of Nectin-4 TRACTr Molecule

The Nectin-4 TRACTr molecule was expressed in stable CHO-GS cells and purified. The Nectin-4 TRACTr molecule was purified from culture supernatant (HCCF) by affinity chromatography using a Protein A coupled resin (Ampshere A3, JSR Bioscience). Then, it was washed with ten column volume of 1× phosphate buffered saline, pH 7.4. The protein A was eluted with 50 mM sodium acetate buffer, pH 4.0 for 4 to 5 column volume until the absorbance is below 1. The elute was performed SEC to determine sample purity. Next, the eluted was prepare for the CEX polishing step by dialyzing into 50 mM MES buffer, pH 6.0 overnight at 4 degree. In the last polishing step, the protein was purified by cation exchanger chromatography (Capto SP Impres) to remove aggregates and other impurities and the purity was assessed by UPSEC and RP. The protein was then dialyzed into formulation buffer consisting of 10 mM Histidine, 8% Sucrose, pH 6.0, and the final purity of the protein was checked by SEC, CE-SDS, SDSPAGE, SECMALS, Mass spec, and RP chromatography methods.

### Example 9: T-cell Dependent Cellular Cytotoxicity (TDCC)

TDCC assays were used to measure in vitro potency of the Nectin-4 TRACTr molecule (SEQ ID NO: 1 and SEQ ID NO: 2) in co-cultures of effector cells (primary human pan T cells or peripheral blood mononuclear cells (PBMCs) or non-human primate (NHP) PBMCs) with target cells (luciferase- and Nectin-4-expressing human cancer cell lines, e.g., Sk-Br-3, Panc08.13, MDA-MB-468).

Controls: Controls that were used include a T-cell engager that includes the antibodies in the Nectin-4 TRACTr molecule (TCE), a non-targeting TCE isotype (RSV TCE), and a Nectin-4 TRACTr molecule in which the protease cleavage sites have been removed (Uncleavable Nectin-4 TRACTr).

The TDCC assays were performed with primary human T cells, primary human PBMC, or primary NHP PBMCs as effectors cells co-cultured with luciferase- and Nectin-4-expressing human cancer cell lines as target cells.

Briefly, effector cells were thawed with warm RPMI-10% FBS, washed twice, and resuspended in culture medium. Target cells were washed with culture medium and resuspended in culture medium. Effectors cells were cocultured with target cells at fixed a effector: target (E: T) cell ratio of 3:1 (human pan T) or 10:1 (human peripheral blood mononuclear cells (PBMC) or non-human primate (NHP) PBMC) in a final volume of 200 μL per well. The cell cocultures were then treated with a serial dilution of Nectin-4 TRACTr (starting at 170 nM, 5-fold dilution, 10-point) or with a serial dilution of TCE (starting at 6 nM, 5-fold dilution, 10-point) and incubated for 96 h at 37° C. Wells containing only effector cells and target cells were used as controls for target cell viability. In some experiments, cell cocultures were also treated with negative control molecules: RSV TCE (non-targeting TCE) and uncleavable Nectin-4 TRACTr molecule.

At the end of incubation period, luciferase reagent was added into each well to quantify viable target cells. The culture plates were incubated for 10 minutes under shaking conditions at room temperature. Luminescence was measured using a Spectromax microplate reader, and specific cytotoxicity of treated wells was calculated relative to the control wells.

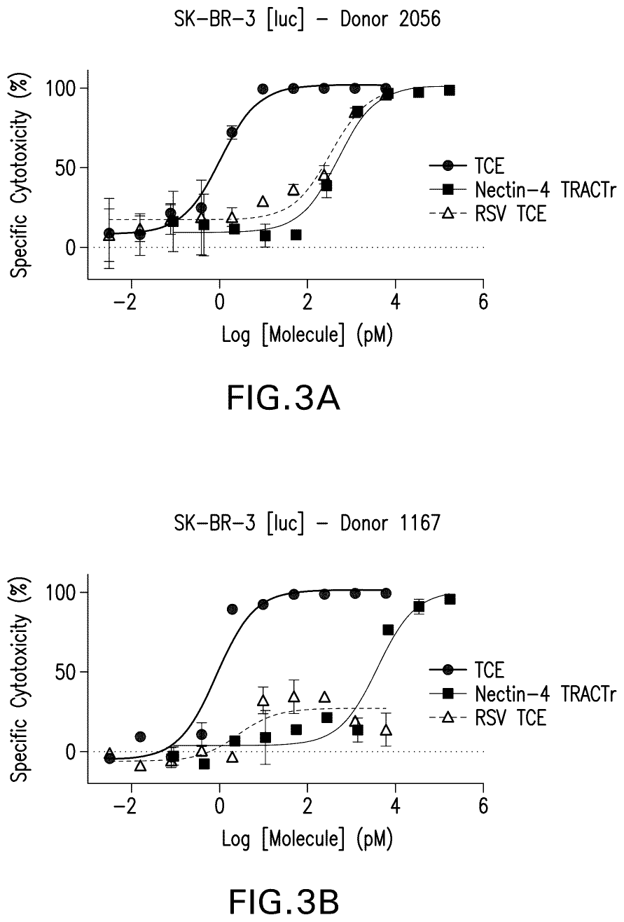

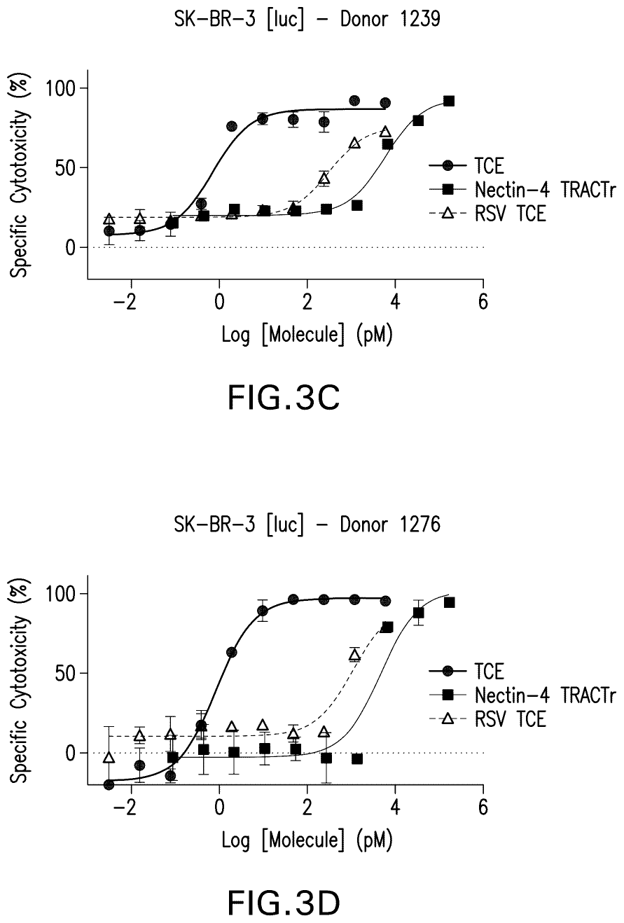

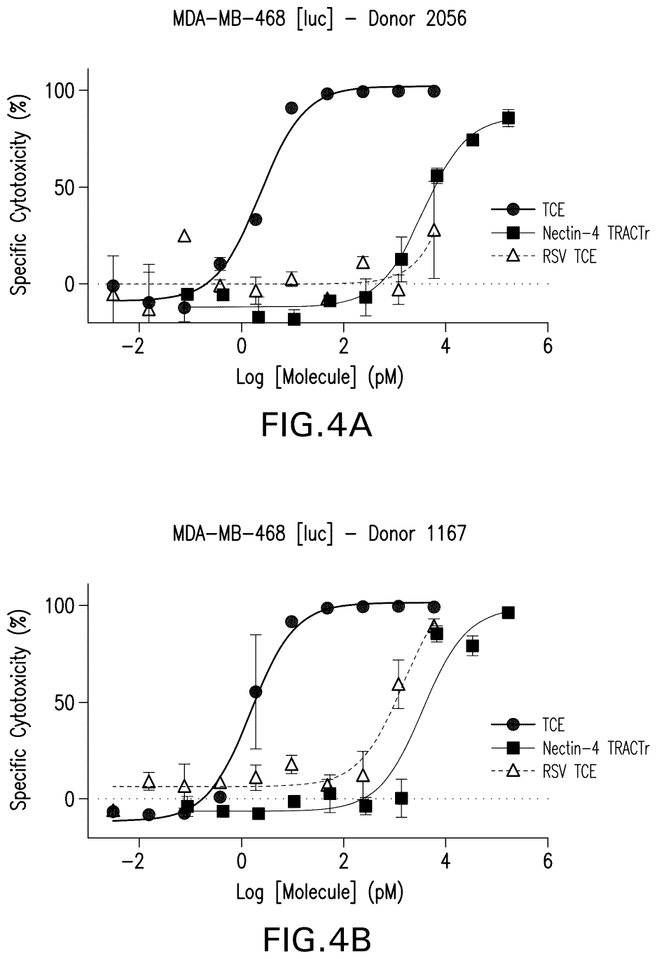

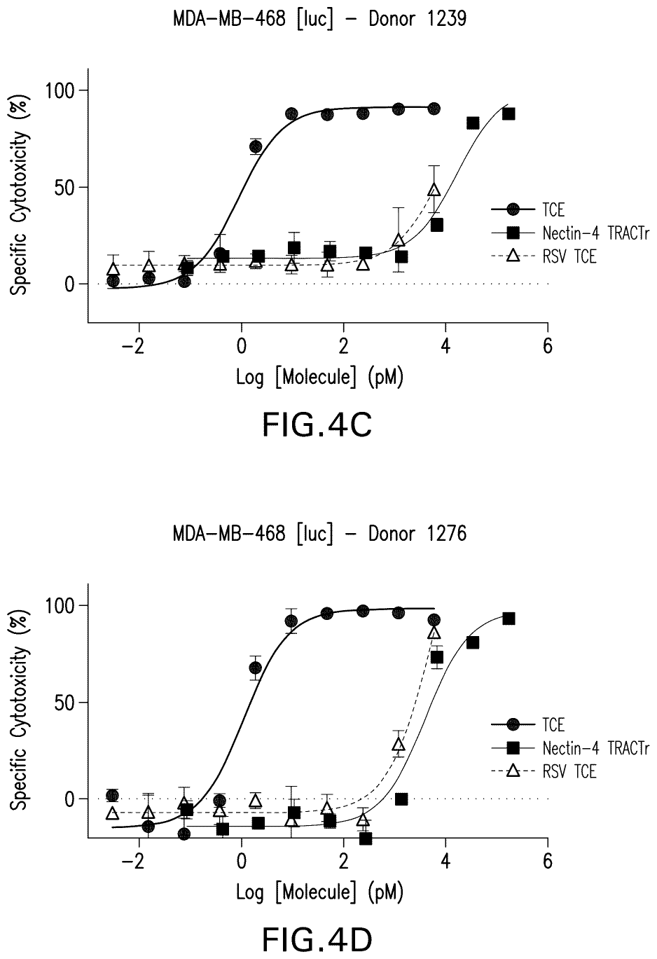

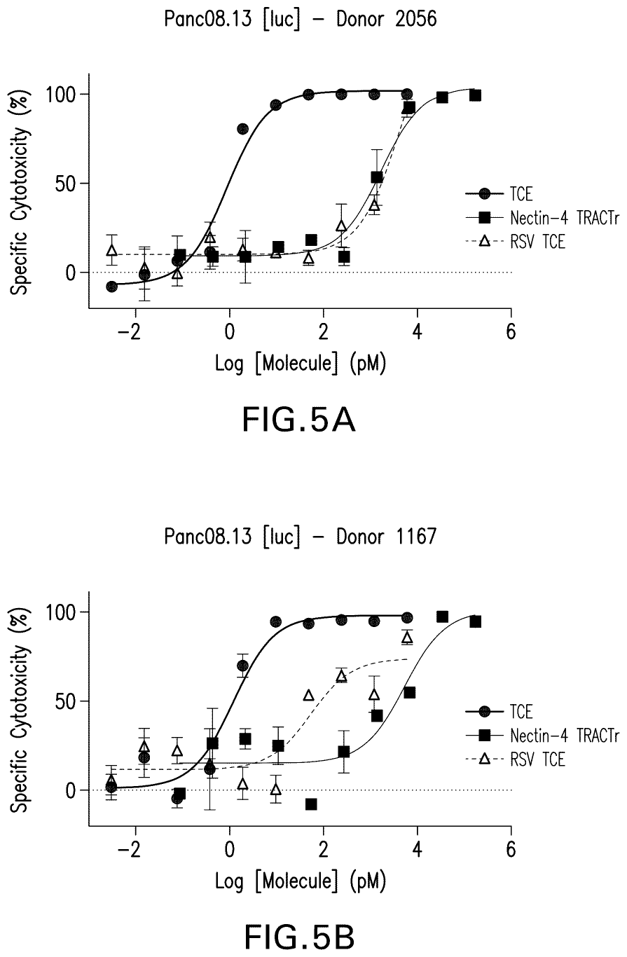

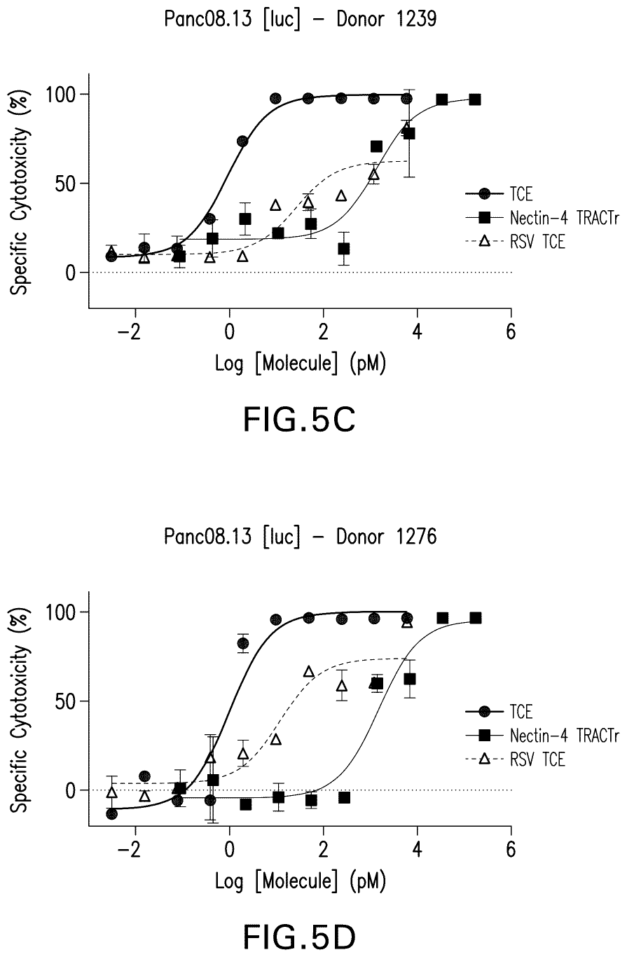

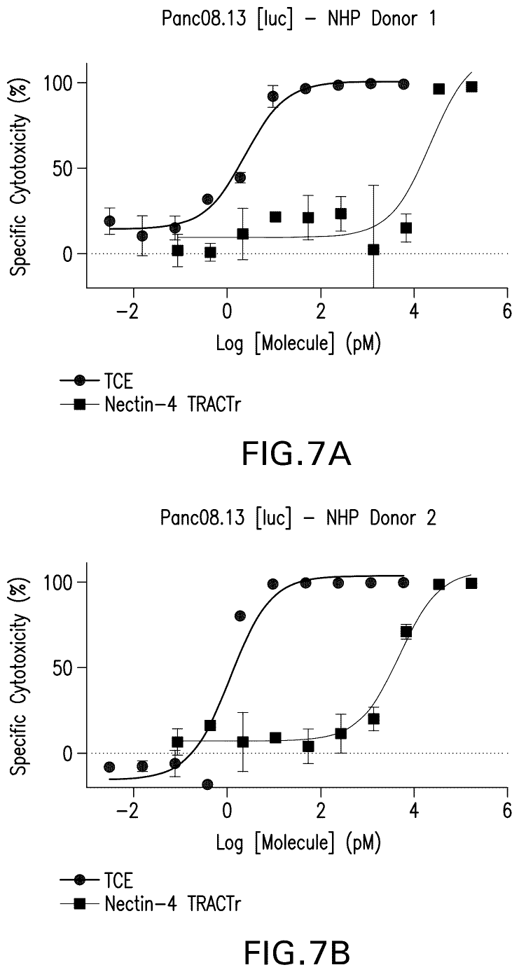

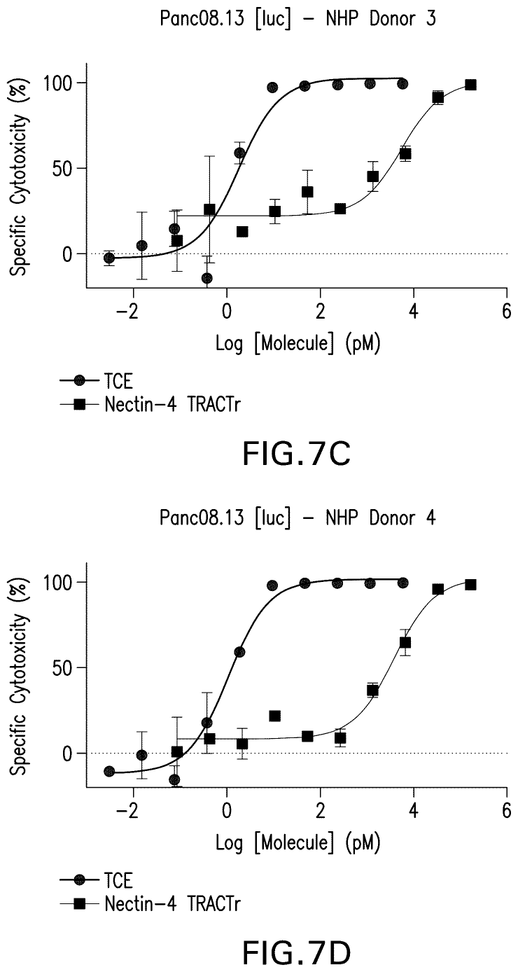

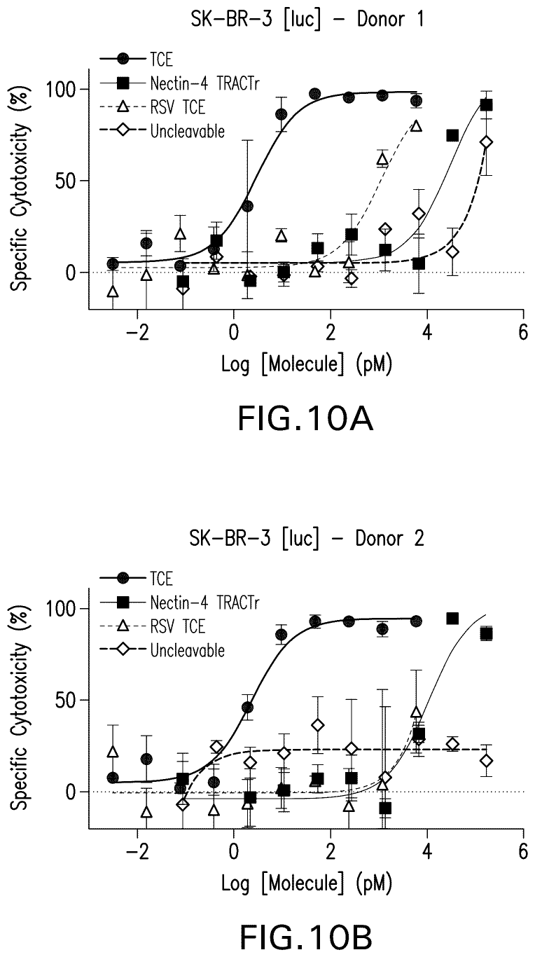

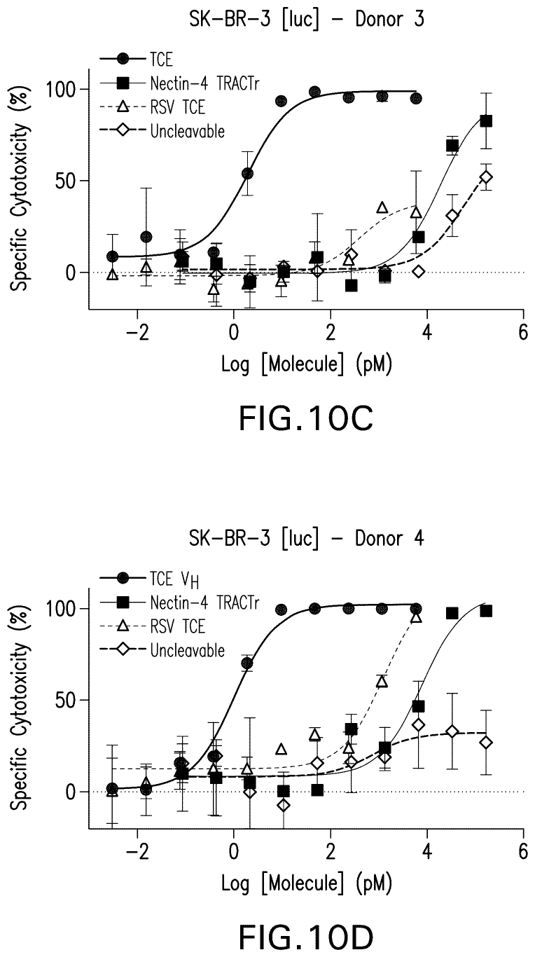

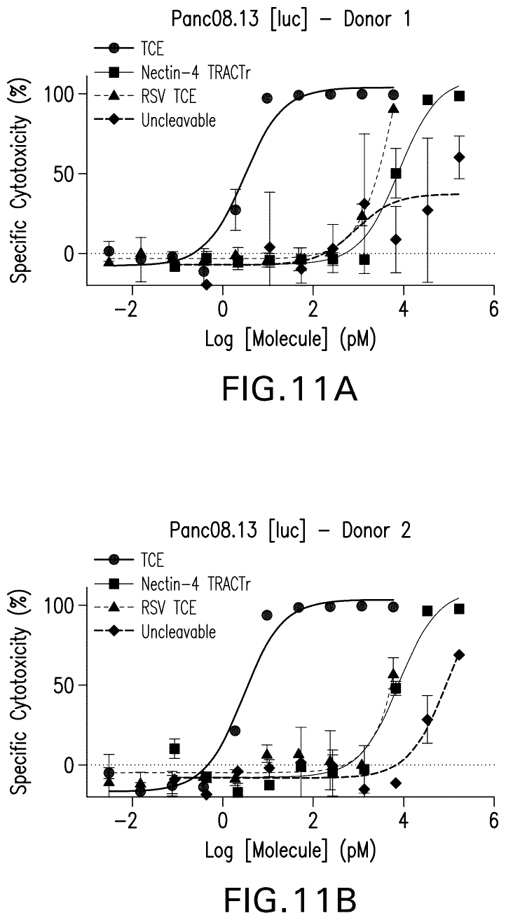

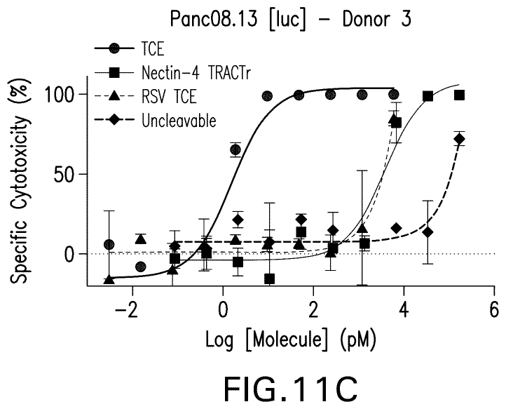

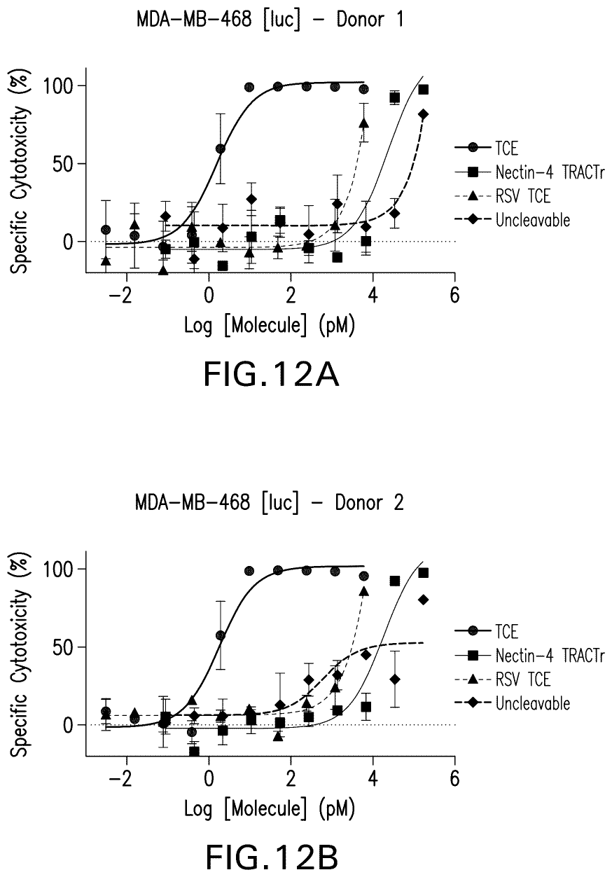

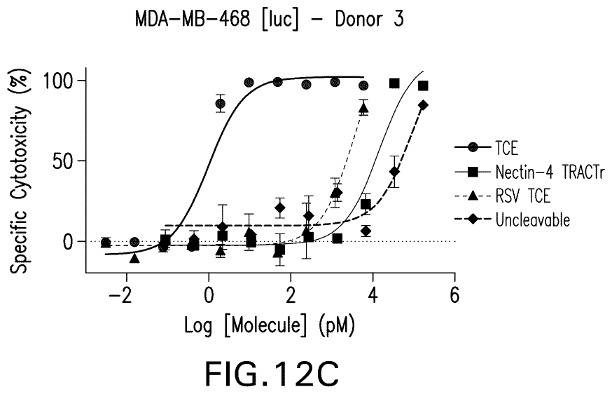

Cytotoxicity was calculated in vitro donor cells from humans and non-human primates. The calculated in vitro cytotoxicity for TDCC with human pan T cells are shown in FIGS. 3A-3D, 4A-4D, and 5A-5D. The calculated in vitro cytotoxicity with NHP PBMCs is shown in FIGS. 7A-7D. The calculated in vitro cytotoxicity with human PBMC is in FIGS. 10A-10D, 11A-11C, and 12A-12C. Nectin-4 TRACTr potency demonstrated as EC50 values are shown in Table 20.

TABLE 20
EC₅₀ Values From TDCC Assays With Four Human
Cancer Cell Lines and Human Effector Cells

| | EC₅₀ values (Mean, pM) | | | | | |
|---|---|---|---|---|---|---|
| | SK-BR-3 [Luc] | | MDA-MB-468 [Luc] | | Panc08.13 [Luc] | |
| | Nectin-4 | | Nectin-4 | | Nectin-4 | |
| Effector cells | TRACTr | TCE | TRACTr | TCE | TRACTr | TCE |
| Human T cells^{a} | 8.13 | 0.77 | 11.67 | 1.35 | 9.20 × 10³ | 1.06 |
| Human PBMCs^{b} | 17.64 × 10³ | 1.7 | 18.78 × 10³ | 1.4 | 23.88 × 10³ | 2.2 |
| NHP PBMCs^{c} | NA | NA | NA | NA | 9.20 × 10³ | 1.65 |

EC₅₀ = half- maximal efficient concentration; h = hour(s); Luc = luciferase; NA = not applicable; NC = not calculated; NHP = non-human primate; PBMC = peripheral blood mononuclear cell; TCE = T-cell engager; TDCC = T cell-dependent cellular cytotoxicity.
^{a}TDCC assay was performed with human T cells (effector cells) cocultured with human (target cells) at a E:T ratio of 3:1 with a serial dilution of Nectin-4 TRACTr starting at 170 nM, 5-fold dilution, 10 point) or in presence of a serial dilution of TCE (starting at 6 nM, 5-fold dilution, 10-point) and incubated for 96 h at 37° C. Data are shown as the mean from 5 healthy donors.
^{b}TDCC assay was performed with human PBMCs (effector cells) cocultured with human tumor cell lines (target cells) at a E:T ratio of 10:1 with a serial dilution of Nectin-4 TRACTr (starting at 170 nM, 5-fold dilution, 10 point) or with a serial dilution of TCE (starting at 6 nM, 5-fold dilution, 10-point) and incubated for 96 h at 37° C. Data are shown as the mean from 6 healthy donors for SK-BR-3 [Luc], 3 healthy donors for MDA-MB-468 [Luc] and 10 healthy donors for Panc08.13 [Luc] cell lines.
^{c}TDCC assay was performed with rhesus monkey PBMCs (effector cells) cocultured with human tumor cell lines (target cells) at a E:T ratio of 10:1 with a serial dilution of Nectin-4 TRACTr (starting at 170 nM, 5-fold dilution, 10 point) or with a serial dilution of TCE (starting at 6 nM, 5-fold dilution, 10-point) and incubated for 96 h at 37° C. Data are shown as the mean from 4 rhesus monkeys.

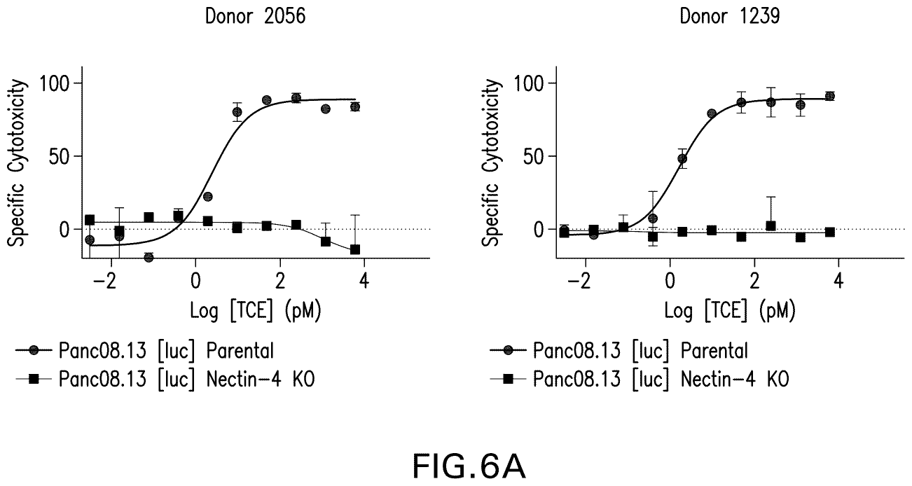

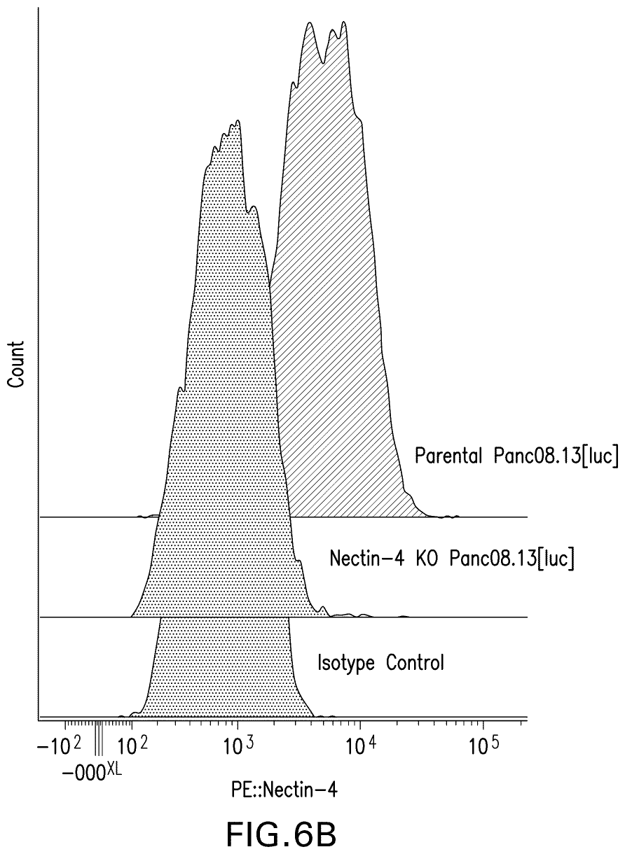

A TDCC assay was performed with human T cells and Panc08.13 [Luc] parental or Panc08.13 [Luc] Nectin-4 knock-out (KO) cell lines to evaluate the impact of Nectin-4 in TCE functional activity. Nectin-4 KO cells were not killed in TDCC, demonstrating the requirement for Nectin-4 expression. Data is shown in FIG. 6A-6B.

### Example 10: Cytokine Concentration Analyzed by Immunoassay

Immunoassay of supernatants collected from above-described TDCC assays were collected to demonstrate Nectin-4 TRACTr-induced IFNγ and TNFα secretion.

After 48 h of human pan T cell coculture with target cells and Nectin-4 TRACTr, TCE, RSV TCE, or Uncleavable Nectin-4 TRACTr, cell culture supernatants were collected and IFNγ and TNFα concentration were measured using an electro chemiluminescent (ECL)-based immunoassay platform following manufacturer's protocols.

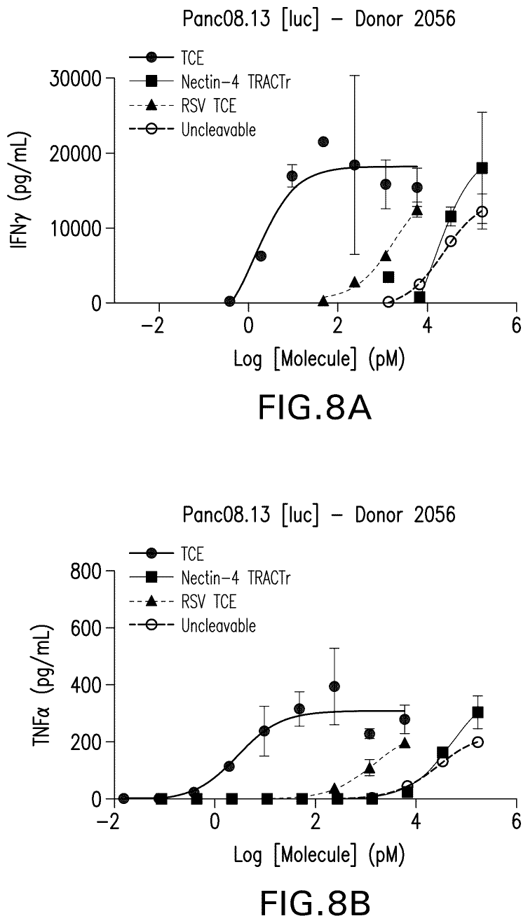

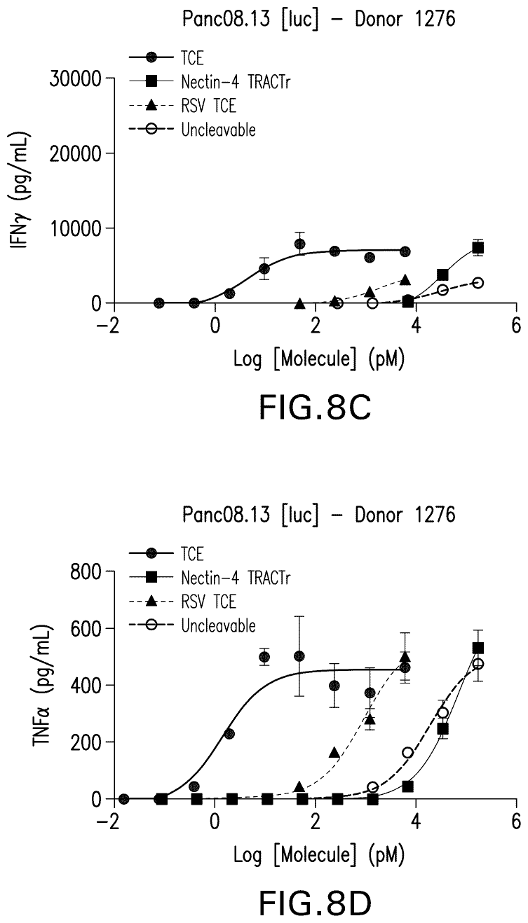

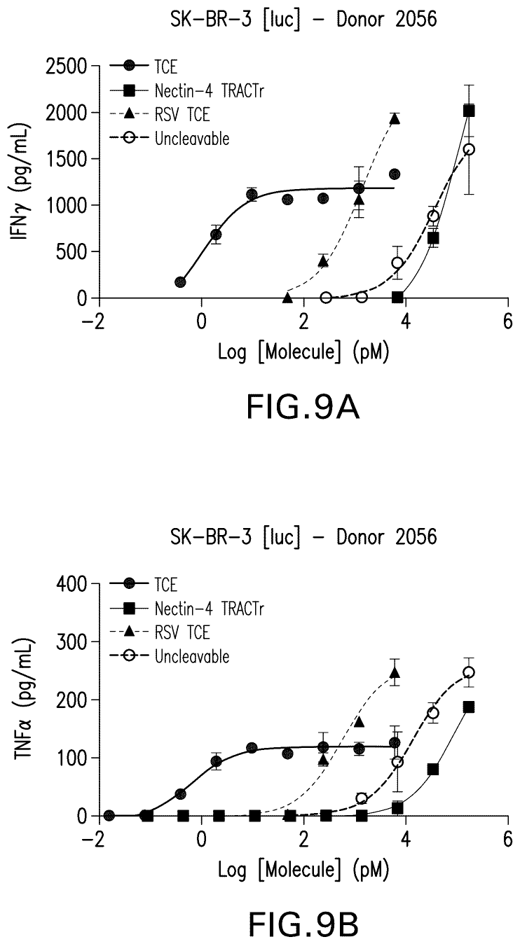

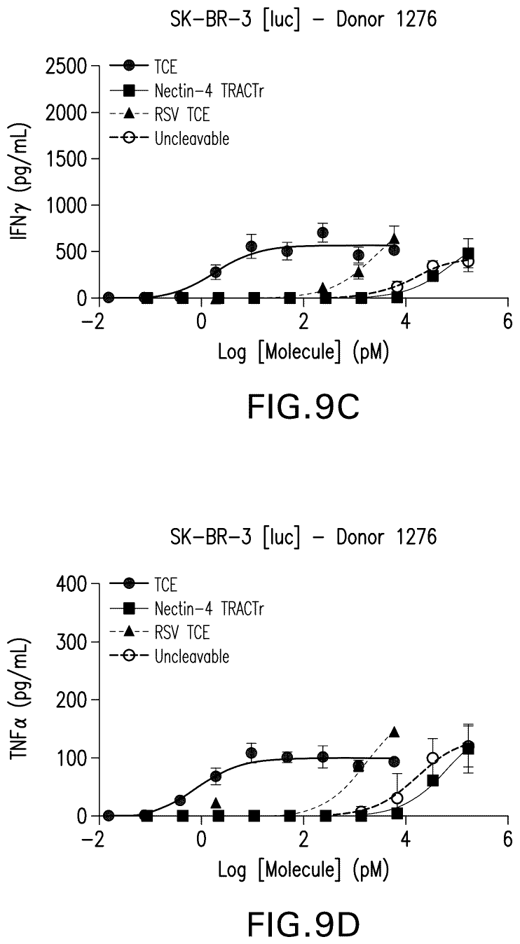

Briefly, multi-array plates pre-coated with anti-cytokine capture antibodies were washed 3 times with wash buffer (provided by manufacturer). Standards, control dilutions and supernatant samples (50 μL) were added to the plates and incubated for 2 hours under shaking conditions at room temperature. After washes with wash buffer, detection antibody solution was added to the wells and incubated for 2 hours at room temperature under shaking conditions. Wells were washed 3 times with wash buffer, then 150 μL of 2× Read Buffer T was added into each well, and ECL was measured on a SECTOR® S600 instrument (Meso Scale Discovery). IFNγ and TNFα concentrations in the samples were calculated using ECL values of samples and a standard curve. Nectin-4 TRACTr-induced IFNγ and TNFα secretion is shown in FIGS. 8A-8D and 9A-9D. EC50 values are shown in Table 21.

TABLE 21
Cytokine Concentration Analyzed From TDCC Assay
Supernatants Treated With Nectin-4 TRACTr

| | Nectin-4 TRACTr Treatment, | | TCE Treatment, | |
|---|---|---|---|---|
| | EC₅₀ (Mean, ng/mL) | | EC₅₀ (Mean, ng/mL) | |
| Cell Line | IFNγ | TNFα | IFNγ | TNFα |
| SK-BR-3 | 8875 | 7178 | 0.10 | 0.05 |
| [Luc] | | | | |
| Panc08.13 | 2328 | 6011 | 0.21 | 0.16 |
| [Luc] | | | | |

IFNγ = interferon gamma; Luc = luciferase; TCE = t-cell engager; TDCC = T-cell-dependent cellular cytotoxicity; TNFα = tumor necrosis factor alpha.
TDCC assay was performed with human T cells isolated from 2 healthy donors (effector cells) cocultured with human tumor cell lines (target cells) at a E:T ratio of 3:1 with a serial dilution of Nectin-4 TRACTr(starting at 170 nM, 5-fold dilution, 10 point) or with a serial dilution of TCE (starting at 6 nM, 5-fold dilution, 10 point) and incubated for 48 h at 37° C. At the end of incubation, cytokine level was measured by a Meso Scale Discovery immunoassay. Data are shown as the mean from 2 human healthy donors.

A mix of human T cells and human tumor cell lines at a E: T ratio of 3:1 was incubated for 48 h in presence of a serial dilution of Nectin-4 TRACTr, or TCE. At the end of the incubation, supernatants were collected and IFNγ and TNFα concentration level were measured using a Meso Scale Discovery immunoassay.

### Example 11: Xenograft Studies

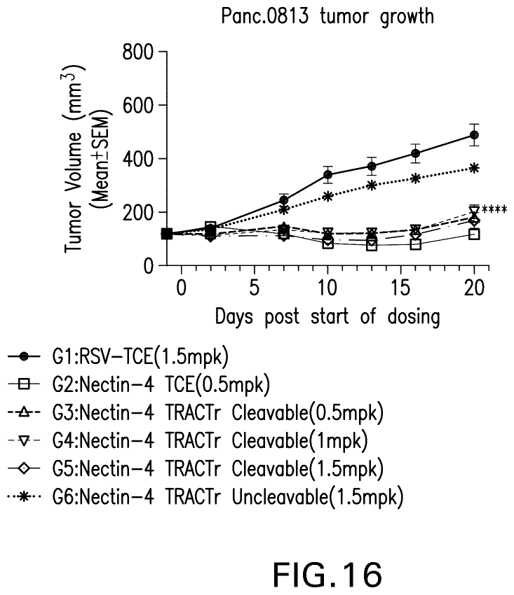

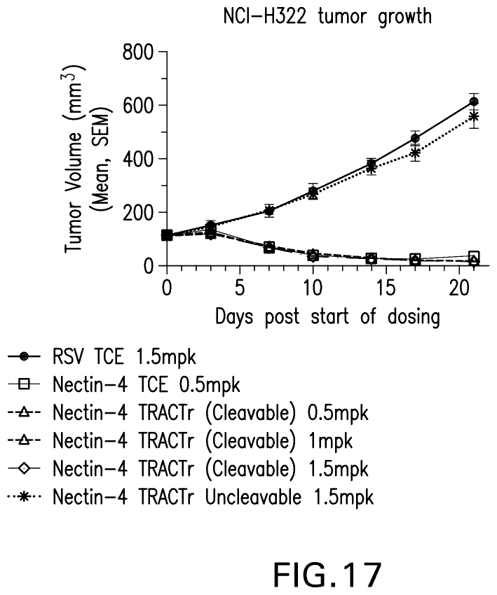

In vivo studies were conducted in the human PBMC-transferred Panc08.13 (FIG. 16) and human T-cell transferred NCI-H322 (FIG. 17) xenograft mouse tumor models. Nectin-4 TRACTr (SEQ ID NO: 1 and SEQ ID NO: 2) showed tumor growth inhibition.

Controls include: a non-targeting TCE isotype (RSV TCE), a Nectin-4 TRACTr molecule in which the protease cleavage sites have been removed (Nectin-4 TRACTr uncleavable), and a TCE engager that includes Nectin-4 antibody sequences (Nectin-4 TCE).

1×10⁶ Panc08.13 tumor cells in Matrigel™ diluted 1:2 in serum-free RPMI media were implanted via subcutaneous administration in the lower right flank of MHC I/II double-knockout NSG mice (Jackson Laboratories). Seven days after tumor inoculation, each mouse received IV (retro-orbital) an administration of 10×10⁶ human PBMCs. After 7 days, mice received via retro-orbital IV injection once daily a dose of Nectin-4 TRACTr (SEQ ID NO: 1 and SEQ ID NO: 2) (0.5, 1, or 1.5 mg/kg), or a dose of 0.5 mg/kg of positive control Nectin-4 TCE, or 1.5 mg/kg of negative control articles (RSV-TCE or Nectin-4 TRACTr uncleavable form), for a total of 10 doses for a period of 11 days. Tumor volumes were measured biweekly throughout the study. Nectin-4 TRACTr and positive control Nectin-4 TCE treatments induced significant antitumor activity with reduction of tumor volume compared to negative control RSV-TCE starting at Day 7. No antitumor activity was observed with negative control Nectin-4 TRACTr uncleavable treatment.

2.5×10⁶ NCI-H322 tumor cells in Matrigel™ diluted 1:1 in serum-free RPMI were implanted via subcutaneous administration in the lower right flank of MHC I/II double-knockout NSG mice (Jackson Laboratories). Fourteen days after tumor inoculation, mice received IV (retro-orbital) an administration of 10×10⁶ human T cells. After 7 days, mice received via retro-orbital IV injection once daily a dose of Nectin-4 TRACTr (SEQ ID NO: 1 and SEQ ID NO: 2) (0.5, 1, or 1.5 mg/kg), or a dose of 0.5 mg/kg of positive control Nectin-4 TCE, or 1.5 mg/kg of negative control articles (RSV-TCE or Nectin-4 TRACTr uncleavable form), for a total of 10 doses for a period of 11 days. Tumor volumes were measured biweekly throughout the study. Nectin-4 TRACTr and positive control Nectin-4 TCE treatments induced significant antitumor activity with reduction of tumor volume compared to negative control RSV-TCE starting at Day 7 (see FIGS. 16 and 17). No antitumor activity was observed with the negative control Nectin-4 TRACTr uncleavable treatment (see FIGS. 16 and 17).

The disclosed subject matter is not to be limited in scope by the specific embodiments and examples described herein. Indeed, various modifications of the disclosure in addition to those described will become apparent to those skilled in the art from the foregoing description and accompanying figures. Such modifications are intended to fall within the scope of the appended claims.

All references (e.g., publications or patents or patent applications) cited herein are incorporated herein by reference in their entirety and for all purposes to the same extent as if each individual reference (e.g., publication or patent or patent application) was specifically and individually indicated to be incorporated by reference in its entirety for all purposes. Other embodiments are within the following claims.

The following sequences are disclosed herein.

TABLE 22
Sequences
Sequences

| SEQ ID | | |
|---|---|---|
| NO: | Description | Sequence |
| 1 | Nectin-4 TRACTr | MGWSCIILFLVATATGVHSEVQLVESGGGLVQPGGSLRLSCAASGST |
| | heavy chain | FYTAVMGWVRQAPGKGLEWVAAIRWTALTTSYADSVKGRFTISRD |
| | | GAKTTLYLQMNSLRPEDTAVYYCAARGTLGLFTTADSYDYWGQGT |
| | | LVTVSSGGGGSGGGSGGVYCGPEFDESVGCMGGGGSGGGLSGRSD |
| | | AGSPLGLAGSGGGSEVOLVESGGGLVQPGGSLKLSCAASGFTFNKY |
| | | AMNWVRQAPGKGLEWVARIRSKYNNYATYYADSVKDRFTISRDDS |
| | | KNTAYLQMNNLKTEDTAVYYCVRHGNFGNSYISYWAYWGQGTLV |
| | | TVSSGGGGSGGGGSGGGGSQTVVTQEPSLTVSPGGTVTLTCGSSTGA |
| | | VTSGNYPNWVQQKPGQAPRGLIGGTKFLAPGTPARFSGSLLGGKAA |
| | | LTLSGVQPEDEAEYYCVLWYSNRWVFGGGTKLTVLGGGGSEVQLV |
| | | ESGGGLVQPGGSLRLSCAASGFSFSNYYMAWVRQAPGKGLEWVASI |
| | | STGGGNIYYRDSVKGRFTISRDNAKNSLYLQMNSLRAEDTAVYYCA |
| | | RQTAYYVMDAWGQGTTVTVSSASTKGPSVFPLAPSSKSTSGGTAAL |
| | | GCLVKDYFPEPVTVSWNSGALTSGVHTFPAVLQSSGLYSLSSVVTVP |
| | | SSSLGTQTYICNVNHKPSNTKVDKKVEPKSC |
| 2 | Nectin-4 TRACTr | MGWSCIILFLVATATGVHSGGWSCAPDEDTWLCPAGGGGSGGLSGR |
| | light chain | SDAGSPLGLAGSGGSDIQMTQSPSSLSASVGDTVTITCLATEDIFSYL |
| | | AWYQQKPGKAPKLLIYGANRLKDTVPSRFSGGGSGTEYSLTISGLQP |
| | | EDFGTYYCLQGAKFPLTFGQGTKLDMKRTVAAPSVFIFPPSDEQLKS |
| | | GTASVVCLLNNFYPREAKVQWKVDNALQSGNSQESVTEQDSKDST |
| | | YSLSSTLTLSKADYEKHKVYACEVTHQGLSSPVTKSFNRGEC |
| 3 | Leader Sequence | MGWSCIILFLVATATGVHS |
| | (Nectin-4 TRACTr) | |
| 4 | Anti-Albumin | TAVMG |
| | CDRH1 | |
| | (Nectin-4 TRACTr) | |
| 5 | Anti-Albumin | AAIRWTALTTSYADSVKG |
| | CDRH2 | |
| | (Nectin-4 TRACTr) | |
| 6 | Anti-Albumin | RGTLGLFTTADSYDY |
| | CDRH3 | |
| | (Nectin-4 TRACTr) | |
| 7 | CD3 Mask | GGVYCGPEFDESVGCMGG |
| | (Nectin-4 TRACTr) | |
| 8 | Protease cleavage | LSGRSDAGSPLGLAG |
| | site heavy chain) | |
| | (Nectin-4 TRACTr) | |
| 9 | CD3 CDRH1 | KYAM |
| | (Nectin-4 TRACTr) | |
| 10 | CD3 CDRH2 | RIRSKYNNYATYYADSVKD |
| | (Nectin-4 TRACTr) | |
| 11 | CD3 CDRH3 | HGNFGNSYISYWAY |
| | (Nectin-4 TRACTr) | |
| 12 | CD3 CDRL1 | GSSTGAVT |
| 13 | CD3 CDRL2 | GTKFLAP |
| | (Nectin-4 TRACTr) | |
| 14 | CD3 CDRL3 | VLWYSNRWV |
| | (Nectin-4 TRACTr) | |
| 15 | Nectin-4 CDRH1 | NYYMA |
| | (Nectin-4 TRACTr) | |
| 16 | Nectin-4 CDRH2 | SISTGGGNIYYRDSVKG |
| | (Nectin-4 TRACTr) | |
| 17 | Nectin-4 CDRH3 | QTAYYVMDA |
| | (Nectin-4 TRACTr) | |
| 18 | Nectin-4 Mask #1 | GGWSCAPDEDTWLCPAGG |
| | (Nectin-4 TRACTr) | |
| 19 | Protease cleavage | LSGRSDAGSPLGLAG |
| | site (light chain) | |
| | (Nectin-4 TRACTr) | |
| 20 | Nectin-4 CDRL1 | LATEDIFSYLA |
| | (Nectin-4 TRACTr) | |
| 21 | Nectin-4 CDRL2 | GANRLKD |
| | (Nectin-4 TRACTr) | |
| 22 | Nectin-4 CDRL3 | LOGAKFPLT |
| | (Nectin-4 TRACTr) | |
| 23 | Nectin-4 Mask #2 | GGWFCAPDENTWLCPNAGG |
| 24 | Nectin-4 light chain | EIQMTQSHKTFSVSTGQRVTITCKASQDVSIAVAWYQQKPGKAPKLL |
| | variable sequence | IYWASTRQTGVPSRFSGSGSGTDFTLTISCVQSEDFAVYYCQQYSSYP |
| | (1) | FTFGQGTKLEIK |
| | (sequence name: | |
| | DAB012120) | |
| | (humanized) | |
| 25 | Nectin-4 light chain | DIQMTQSPSFLSASVGDRVTITCKASQDVSIAVAWYQQKPGKAPKLL |
| | variable sequence | IYWASTRQTGVPSRFSGSGSGTDFTLTISSLQPEDFATYFCQQYSSYPF |
| | (2) | TFGQGTKLEIK |
| | (sequence name: | |
| | DAB012121) | |
| | (humanized) | |
| 26 | Nectin-4 light chain | DIQMTQSPSFLSASVGERVTITCKASQDVSIAVAWYQQKPGKAPKLL |
| | variable sequence | IYWASTRQTGVPSRFSGSGSGTDFTLTISSLQPEDIATYFCQQYSSYPF |
| | (3) | TFGQGTKLEIK |
| | (sequence name: | |
| | DAB012122) | |
| | (humanized) | |
| 27 | Nectin-4 light chain | DIQMTQSPSFLSASVGDRVTITCKASQDVSIAVAWYQQKPGKAPKLL |
| | variable sequence | IYWASTRQTGVPSRFSGSGSGTDFTLTISSLQPEDSATYFCQQYSSYPF |
| | (4) | TFGQGTKVDIK |
| | (sequence name: | |
| | DAB012123) | |
| | (humanized) | |
| 28 | Nectin-4 CDRL1 | KASQDVSIAVA |
| | (1) (rat) | |
| 29 | Nectin-4 CDRL2 | WASTRQT |
| | (1) (rat) | |
| 30 | Nectin-4 CDRL3 | QQYSSYPFT |
| | (1) (rat) | |
| 31 | Nectin-4 light chain | EIQMTQSQSTLSVSLGDRVTITCKASQNVVTAVAWYQQKPGQAPKL |
| | variable sequence | LIYSASHRFTGVPSRFSGSGSGTDFTLIISCMQSEDFAVYYCQQYSSYP |
| | (5) (sequence name: | YTFGQGTKLEIK |
| | DAB012124) | |
| | (humanized) | |
| 32 | Nectin-4 light chain | DIQMTQSPSFLSASVGDRVTITCKASQNVVTAVAWYQQKPGKAPKL |
| | variable sequence | LIYSASHRFTGVPSRFSGSGSGTDFTLTISNLQSEDLATYFCQQYSSYP |
| | (6) (sequence name: | YTFGQGTKLEIK |
| | DAB012125) | |
| | (humanized) | |
| 33 | Nectin-4 light chain | DIQMTQSPSFLSASVGDRVTITCKASQNVVTAVAWYQQKPGKAPKL |
| | variable sequence | LIYSASHRFTGVPSRFSGSGSGTDFTLTISNLQPEDSATYFCQQYSSYP |
| | (7) (sequence name: | YTFGQGTKLEIK |
| | DAB012126) | |
| | (humanized) | |
| 34 | Nectin-4 light chain | DIQMTQSPSFLSASVGDRVTITCKASQNVVTAVAWYQQKPGKAPKL |
| | variable sequence | LIYSASHRFTGVPSRFSGSGSGTDFTLTISNLQPDDAATYFCQQYSSYP |
| | (8) (sequence name: | YTFGQGTKLEIK |
| | DAB012127) | |
| | (humanized) | |
| 35 | Nectin-4 CDRL1 | KASQNVVTAVA |
| | (2) (rat) | |
| 36 | Nectin-4 CDRL2 | SASHRFT |
| | (2) (rat) | |
| 37 | Nectin-4 CDRL3 | QQYSSYPYT |
| | (2) (rat) | |
| 38 | Nectin-4 light chain | AIQMTQSHKSFSVSTGQRVTITCKASQDVSTTVAWYQQKPGQAPKL |
| | variable sequence | LIYWASTRQTGVPSRFTGSGSGTDFTLTISCVQSEDFAVYYCQQYSSY |
| | (9) (sequence name: | PFTFGQGTKLEIK |
| | DAB012128) | |
| | (humanized) | |
| 39 | Nectin-4 light chain | DIQMTQSPSFLSASVGDRVTITCKASQDVSTTVAWYQQKPGKAPKLL |
| | variable sequence | IYWASTRQTGVPSRFSGSGSGTDFTLTISNLQPEDFATYFCQQYSSYP |
| | (10) (sequence | FTFGQGTKLEIK |
| | name: DAB012129) | |
| | (humanized) | |
| 40 | Nectin-4 light chain | DIQMTQSPSFLSASVGERVTITCKASQDVSTTVAWYQQKPGKAPKLL |
| | variable sequence | IYWASTRQTGVPSRFSGSGSGTDFTLTINSLQPEDVATYFCQQYSSYP |
| | (11) (sequence | FTFGQGTKLEIK |
| | name: DAB012130) | |
| | (humanized) | |
| 41 | Nectin-4 light chain | DIQMTQSPSFLSASVGERVTITCKASQDVSTTVAWYQQKPGKAPKLL |
| | variable sequence | IYWASTRQTGVPSRFSGSGSGTDFTLTISNLQPEDVATYFCQQYSSYP |
| | (12) (sequence | FTFGQGTKLEIK |
| | name: DAB012131) | |
| | (humanized) | |
| 42 | Nectin-4 CDRL1 | KASQDVSTTVA |
| | (3) (rat) | |
| 43 | Nectin-4 light chain | DIQMTQSPSSVSLTVGQRVTITCLATEDIFSYLAWYQQKPGKAPRLLI |
| | variable sequence | YGANRLKDGVPSRFSGSGSGTQYTLRISSMQPEDFGVYYCLQGAKFP |
| | (13) (sequence | LTFGPGTKLEIK |
| | name: DAB012132) | |
| | (humanized) | |
| 44 | Nectin-4 light chain | DIQMTQSPSSLSASIGDTVTITCLATEDIFSYLAWYQQRPGKAPKLLIY |
| | variable sequence | GANRLKDGVPSRFSGSGSGTDYTLTISGLQPEDFVTYYCLQGAKFPL |
| | (14) (sequence | TFGQGTKLDMK |
| | name: DAB012133) | |
| | (humanized) | |
| 45 | Nectin-4 light chain | DIQMTQSPSSLSASVGDTVTITCLATEDIFSYLAWYQQKPGKAPKLLI |
| | variable sequence | YGANRLKDGVPSRFSGSGSGTDYTLTISGLQPEDFGTYYCLQGAKFP |
| | (15) (sequence | LTFGQGTKLEIK |
| | name: DAB012134) | |
| | (humanized) | |
| 46 | Nectin-4 light chain | DIQMTQSPSSLSASVGDTVTITCLATEDIFSYLAWYQQKPGKAPKLLI |
| | variable sequence | YGANRLKDGVPSRFSGGGSGTEYSLTISGLQPEDFGTYYCLQGAKFP |
| | (16) (sequence | LTFGQGTKLDMK |
| | name: DAB012135) | |
| | (humanized) | |
| 47 | Nectin-4 light chain | DIQMTQSPSSLSASVGDTVTITCLATEDIFSYLAWYQQKPGKAPKLLI |
| | variable sequence | YGANRLKDTVPSRFSGGGSGTEYSLTISGLQPEDFGTYYCLQGAKFP |
| | (17) (sequence | LTFGQGTKLDMKR |
| | name: | |
| | DAB0121315) | |
| | (modified) | |
| | (humanized) | |
| 48 | Nectin-4 heavy | QVQLLESGGELVQPGGSLRLSCAASGFTFSDYYMFWIRQTPQKRLE |
| | chain variable | WVAYISNGGGNTYYPDTVKGRFTISRDNAKNQLYLQMRSLTPEDTA |
| | sequence (1) | IYYCASPEARYYGNYPFPYWGQGTLVTVSS |
| | (sequence name: | |
| | DAB012120) | |
| | (humanized) | |
| 49 | Nectin-4 heavy | QVQLVESGGGLVKPGGSLRLSCAASGFTFSDYYMFWIRQAPGKGLE |
| | chain variable | WVAYISNGGGNTYYPDTVKGRFTISRDNAKNTLYLQMNSLRAEDTA |
| | sequence (2) | VYYCASPEARYYGNYPFPYWGQGTLVTVSS |
| | (sequence name: | |
| | DAB012121) | |
| | (humanized) | |
| 50 | Nectin-4 heavy | QVQLVESGGGVVKPGGSLRLSCAASGFTFSDYYMFWIRQAPGKGLE |
| | chain variable | WVAYISNGGGNTYYPDTVKGRFTISRDNAKNTLYLQMNSLRAEDTA |
| | sequence (3) | VYYCASPEARYYGNYPFPYWGQGTLVTVSS |
| | (sequence name: | |
| | DAB012122) | |
| | (humanized) | |
| 51 | Nectin-4 heavy | QVQLVESGGGVVQPGGSLRLSCAASGFTFSDYYMFWIRQAPGKGLE |
| | chain variable | WVAYISNGGGNTYYPDTVKGRFTISRDNAKNTLYLQMNSLRAEDTA |
| | sequence (4) | VYYCASPEARYYGNYPFPYWGQGTLVTVSS |
| | (sequence name: | |
| | DAB012123) | |
| | (humanized) | |
| 52 | Nectin-4 CDRH1 | DYYMF |
| | (1) (rat) | |
| 53 | Nectin-4 CDRH2 | YISNGGGNTYYPDTVKG |
| | (1) (rat) | |
| | Nectin-4 CDRH (2) | |
| | 14E4.H3 (mouse) | |
| 54 | Nectin-4 CDRH3 | PEARYYGNYPFPY |
| | (1) | |
| 55 | Nectin-4 heavy | QVQLLQSGPGLVKPSATLSLTCTVSGFSLTTYGVHWIRQPPGKGLEW |
| | chain variable | MGVIWSGGSTDYNAAFISRLTISKDNSKNQVFLQLYSLRAEDTAIYY |
| | sequence (5) | CARTSHWYFDVWGRGTLVTVSS |
| | (sequence name: | |
| | DAB012124) | |
| | (humanized) | |
| 56 | Nectin-4 heavy | QVQLQESGPGLVKPSQTLSLTCTVSGFSLTTYGVHWVRQSPGKGLE |
| | chain variable | WLGVIWSGGSTDYNAAFISRLSISKDNSKSQVFLEMTSLTAADTAIY |
| | sequence (6) | YCARTSHWYFDVWGRGTLVTVSS |
| | (sequence name: | |
| | DAB012125) | |
| | (humanized) | |
| 57 | Nectin-4 heavy | QVQLQESGPGLVKPSQTLSLTCTVSGFSLTTYGVHWVRQAPEKGLE |
| | chain variable | WLGVIWSGGSTDYNAAFISRLSISKDNSKSQVFLEMTSLTADDTAIY |
| | sequence (7) | YCARTSHWYFDVWGRGTLVTVSS |
| | (sequence name: | |
| | DAB012126) | |
| | (humanized) | |
| 58 | Nectin-4 heavy | QVQLQESGPGLVKPSQTLSLTCTVSGFSLTTYGVHWVRQTPEKGLE |
| | chain variable | WLGVIWSGGSTDYNAAFISRLSISKDNSKSQVFLEMTSLTADDTAIY |
| | sequence (8) | YCARTSHWYFDVWGRGTLVTVSS |
| | (sequence name: | |
| | DAB012127) | |
| | (humanized) | |
| 59 | Nectin-4 CDRH1 | TYGVH |
| | (2) | |
| 60 | Nectin-4 CDRH2 | VIWSGGSTDYNAAFIS |
| | (2) (rat) | |
| | Nectin-4 CDRH2 | |
| | 14F6.B1 (mouse) | |
| 61 | Nectin-4 CDRH3 | TSHWYFDV |
| | (2) (rat) | |
| 62 | Nectin-4 heavy | QVQLLESGGGLVQPGGSLRLSCAASGFTFSDYYMYWIRQTPQKRLE |
| | chain variable | WVAYISNGGGNTYYSDTVKGRFTISRDNAKNQLYLQMRSLRPEDTA |
| | sequence (9) | IYYCASPEARYYGNFPFPYWGQGTLVTVSS |
| | (sequence name: | |
| | DAB012128) | |
| | (humanized) | |
| 63 | Nectin-4 heavy | QVQLVESGGGLVKPGGSLRLSCAASGFTFSDYYMYWIRQAPGKGLE |
| | chain variable | WVAYISNGGGNTYYSDTVKGRFTISRDNAKNTLYLQMNSLRAEDTA |
| | sequence (10) | VYYCASPEARYYGNFPFPYWGQGTLVTVSS |
| | (sequence name: | |
| | DAB012129) | |
| | (humanized) | |
| 64 | Nectin-4 heavy | QVQLVESGGGLVKPGGSLRLSCAASGFTFSDYYMYWIRQAPGKGLE |
| | chain variable | WVAYISNGGGNTYYSDTVKGRFTISRDNAKNTLYLQMNSLRAEDTA |
| | sequence (11) | VYYCASPEARYYGNFPFPYWGQGTLVTVSS |
| | (sequence name: | |
| | DAB012130) | |
| | (humanized) | |
| 65 | Nectin-4 heavy | QVQLVESGGGLVKPGGSLRLSCAASGFTFSDYYMYWIRQAPGKGLE |
| | chain variable | WVAYISNGGGNTYYSDTVKGRFTISRDNAKNTLYLQMNSLRAEDTA |
| | sequence (12) | VYYCASPEARYYGNFPFPYWGQGTLVTVSS |
| | (sequence name: | |
| | DAB012131) | |
| | (humanized) | |
| 66 | Nectin-4 CDRH1 | DYYMY |
| | (3) | |
| 67 | Nectin-4 CDRH2 | YISNGGGNTYYSDTVKG |
| | (3) | |
| | Nectin-4 CDRH2 | |
| | 16H8.B1 (mouse) | |
| 68 | Nectin-4 CDRH3 | PEARYYGNFPFPY |
| | (3) | |
| 69 | Nectin-4 heavy | QVQLVESGGGVVQPGRSLRLSCAASGFSFSNYYMAWVRQAPGKGL |
| | chain variable | EWVASISTGGGNIYYRDSVKGRFTISRDNSKSTLYLQMRSLRSEDTAI |
| | sequence (13) | YYCARQTAYYVMDAWGQGTMVTVSS |
| | (sequence name: | |
| | DAB012132) | |
| | (humanized) | |
| 70 | Nectin-4 heavy | EVQLVESGGGLVQPGGSLRLSCAASGFSFSNYYMAWVRQAPGKGLE |
| | chain variable | WVASISTGGGNIYYRDSVKGRFTISRDNAKNSLYLQMNSLRAEDTA |
| | sequence (14) | VYYCARQTAYYVMDAWGQGTTVTVSS |
| | (sequence name: | |
| | DAB012133) | |
| | (humanized) | |
| 71 | Nectin-4 heavy | EVQLVESGGGLVQPGGSLRLSCAASGFSFSNYYMAWVRQAPGKGLE |
| | chain variable | WVASISTGGGNIYYRDSVKGRFTISRDNAKNSLYLQMNSLRAEDTA |
| | sequence (15) | VYYCARQTAYYVMDAWGQGTTVTVSS |
| | (sequence name: | |
| | DAB012134) | |
| | (humanized) | |
| 72 | Nectin-4 heavy | EVQLVESGGGLVQPGGSLRLSCAASGFSFSNYYMAWVRQAPGKGLE |
| | chain variable | WVASISTGGGNIYYRDSVKGRFTISRDNAKNSLYLQMNSLRAEDTA |
| | sequence (16) | VYYCARQTAYYVMDAWGQGTTVTVSS |
| | (sequence name: | |
| | DAB012135) | |
| | (humanized) | |
| 73 | Rat clone | DIQMTQSPASLSVYLGETVSIECLATEDIFSYLAWYQQKPGKSPQLLI |
| | 3H9.1G1 | YGANRLKDGVPSRFSGSGTGTQYSLRISGMQPEDEGDYYCLQGAKF |
| | Light chain variable | PLTFGSGTKLEIK |
| | sequence | |
| 74 | Rat clone | DIQMTQSPASLSASLGETVSIECLASEDIYSYLAWFQQKSGKSPQLLI |
| | 55D10.1C1 | YAANRLQDGVPSRFSGSGSGTQYSLRISGMQPEDEGDYFCLQGSQFP |
| | Light chain variable | WTFGGGTKLELK |
| | sequence | |
| 75 | Rat clone | DIQMTQSPASLSASLGETVSIECLASEDIHNKLAWYQQKPGKSPQLLI |
| | 15H3.1A1 | YYGSNLQDGVPSRFSGSGSGTQYYLKINSLESEDVATYFCLQDSKNP |
| | Light chain variable | WTFGGGTKLEMK |
| | sequence | |
| 76 | Rat clone | DIVLTQSPALDVSLGQRATISCSASQSVSISRYNLIHWYQQKPGQQPK |
| | 54H11.1A4 | LLIWRTSILTSGIPARFSGRGSGTDFTLTINPVQADDIATYYCQQSRES |
| | Light chain variable | PFTFGAGTRLELK |
| | sequence | |
| 77 | Rat clone | NIVLTQSPATLSVTPGESVSLSCRASQSISTGIHWYQQKSNESPRLLIK |
| | 52E1.1B2 | FASQSISGIPSRFSGSGSGTDFTLSINRVESEDFSIYYCQQRDSSLFTFG |
| | Light chain variable | AGTKLELK |
| | sequence | |
| 78 | Rat clone | TYELIQPPSTSVTLGNTVSLTCVGDDLPRRYAYWYQQKPDQSIVRVI |
| | 54E9.1F1 | YEDSKRPSGISDRFSGSSSGTTATLTIRDAQAEDEADYYCHSTYSDDK |
| | Light chain variable | VRVFGGGTKLTVL |
| | sequence | |
| 79 | Rat clone | EVQLVESGGGLVQPGRSMRLSCAASGFSFSNYYMAWVRQAPRKGL |
| | 3H9.1G1 | DWVASISTGGGNIYYRDSVKGRFTISRDNAKSTLYLQMDSLRSEDTA |
| | Heavy chain | TYYCARQTAYYVMDAWGQGASVTVSS |
| | variable sequence | |
| 80 | Rat clone | EVQLVESGGGLVQPGRSLKLSCAASGFTFSNYDMAWVRQVPTKGLE |
| | 55D10.1C1 | WVASISPRGGRIYYRDSVKGRFTVSRDNAKSCLYLQMESLRSDDTAT |
| | Heavy chain | YYCARIHHGYWYFDFWGPGTMVTVSS |
| | variable sequence | |
| 81 | Rat clone | EVKLLESGGGLVQPGGSMRLSCAASGFTFTDFYMIWIRQPAGKAPE |
| | 15H3.1A1 | WLGFIRNKANGYTTDYNPSVKGRFTISRDNTQNMLYLQMITLRAED |
| | Heavy chain | TATYYCARPLYYGYTPRYWGQGVMVTVSS |
| | variable sequence | |
| 82 | Rat clone | EVQLVESGGGLVQPGRPLKLSCAASGFSFSHYDMAWVRQAPTKGLE |
| | 54H11.1A4 | WVAAISPSGGSTYYRDSVKGRFTVSRDKAKNSLYLQMDSLRSEDTA |
| | Heavy chain | TYYCARQGPSYGYYFDYWGQGVMVTVSS |
| | variable sequence | |
| 83 | Rat clone | EVQLVESGGGLVQPGRSLKVSCAASGFTFSNYDMAWVRQAPTKGL |
| | 52E1.1B2 | EWVASINPGGISTYYRDSVKGRFTVSRDNEKSTLYLQMDSLRSEDTA |
| | Heavy chain | TYYCARRQPYFDYWGQGVMVTVSS |
| | variable sequence | |
| 84 | Rat clone | QVTLKESGTGILQPSQALSLTCSISGFSLNTTGICVSWIRQPLGQGLEW |
| | 54E9.1F1 | LADICWDDGKGYNPSLKNRLSISKDTSNNQAFLKITRVDTTDTATYY |
| | Heavy chain | CARNYGGNPFDYWGQGVMVTVSS |
| | variable sequence | |
| 85 | Rat clone | LASEDIYSYLA |
| | 55D10.1C1 | |
| | CDRLI sequence | |
| 86 | Rat clone | AANRLQD |
| | 55D10.1C1 | |
| | CDRL2 sequence | |
| 87 | Rat clone | LQGSQFPWT |
| | 55D10.1C1 | |
| | CDRL3 sequence | |
| 88 | Rat clone | LASEDIHNKLA |
| | 15H3.1A1 | |
| | CDRL1 sequence | |
| 89 | Rat clone | YGSNLQD |
| | 15H3.1A1 | |
| | CDRL2 sequence | |
| 90 | Rat clone | LQDSKNPWT |
| | 15H3.1A1 | |
| | CDRL3 sequence | |
| 91 | Rat clone | SASQSVSISRYNLIH |
| | 54H11.1A4 | |
| | CDRL1 sequence | |
| 92 | Rat clone | RTSILTS |
| | 54H11.1A4 | |
| | CDRL2 sequence | |
| 93 | Rat clone | QQSRESPFT |
| | 54H11.1A4 | |
| | CDRL3 sequence | |
| 94 | Rat clone | RASQSISTGIH |
| | 52E1.1B2 | |
| | CDRLI sequence | |
| 95 | Rat clone | FASQSIS |
| | 52E1.1B2 | |
| | CDRL2 sequence | |
| 96 | Rat clone | QQRDSSLFT |
| | 52E1.1B2 | |
| | CDRL3 sequence | |
| 97 | Rat clone | VGDDLPRRYAY |
| | 54E9.1F1 | |
| | CDRL1 sequence | |
| 98 | Rat clone | EDSKRPS |
| | 54E9.1F1 | |
| | CDRL2 sequence | |
| 99 | Rat clone | HSTYSDDKVRV |
| | 54E9.1F1 | |
| | CDRL3 sequence | |
| 100 | Rat clone | NYDMA |
| | 55D10.1C1 | |
| | CDRH1 sequence | |
| | Rat clone | |
| | 52E1.1B2 | |
| | CDRH1 sequence | |
| 101 | Rat clone | SISPRGGRIYYRDSVKG |
| | 55D10.1C1 | |
| | CDRH2 sequence | |
| 102 | Rat clone | IHHGYWYFDF |
| | 55D10.1C1 | |
| | CDRH3 sequence | |
| 103 | Rat clone | DFYMI |
| | 15H3.1A1 | |
| | CDRH1 sequence | |
| 104 | Rat clone | FIRNKANGYTTDYNPSVKG |
| | 15H3.1A1 | |
| | CDRH2 sequence | |
| 105 | Rat clone | PLYYGYTPRY |
| | 15H3.1A1 | |
| | CDRH3 sequence | |
| 106 | Rat clone | HYDMA |
| | 54H11.1A4 | |
| | CDRH1 sequence | |
| 107 | Rat clone | AISPSGGSTYYRDSVKG |
| | 54H11.1A4 | |
| | CDRH2 sequence | |
| 108 | Rat clone | QGPSYGYYFDY |
| | 54H11.1A4 | |
| | CDRH3 sequence | |
| 109 | Rat clone | SINPGGISTYYRDSVKG |
| | 52E1.1B2 | |
| | CDRH2 sequence | |
| 110 | Rat clone | RQPYFDY |
| | 52E1.1B2 | |
| | CDRH3 sequence | |
| 111 | Rat clone | VGDDLPRRYAY |
| | 54E9.1F1 | |
| | CDRH1 sequence | |
| 112 | Rat clone | EDSKRPS |
| | 54E9.1F1 | |
| | CDRH2 sequence | |
| 113 | Rat clone | HSTYSDDKVRV |
| | 54E9.1F1 | |
| | CDRH3 sequence | |
| 114 | Mouse clone | EVQLQESGGDLVQPGGSLKLSCAASGFTFSDYYMFWIRQTPQKRLE |
| | 14E4.H3 | WVAYISNGGGNTYYPDTVKGRFTISRDNAKNTLYLQMSRLKSEDTA |
| | Heavy chain | MYYCASPEARYYGNYPFPYWGQGTLVTVSA |
| | variable sequence | |
| 115 | Mouse clone | DIVMTQSHKFMSTSVGGRVTITCKASQDVSIAVAWYQQKPGQSPKL |
| | 14E4.H3 | LIYWASTRQTGVPDRFAGSGSGTDFTLTISNVQSEDLADYFCQQYSS |
| | Light chain variable | YPFTFGSGTKLEIK |
| | sequence | |
| 116 | Mouse clone | QVQLKQSGPGLVQPSQSLSITCTVSGFSLTTYGVHWVRQSPGKGLE |
| | 14F6.B1 Heavy | WLGVIWSGGSTDYNAAFISRLSISKDNSKSQVFFEMYSLQADDTAIY |
| | | YCARTSHWYFDVWGTGTTVTVSS |
| | chain variable | |
| | sequence | |
| 117 | Mouse clone | DIVMTQSQKFMSTTVGDRVSITCKASQNVVTAVAWYQQKPGQSPKL |
| | 14F6.B1 Light | LIYSASHRFTGVPDRFTGSGSGTDFTLIISNMQSEDLADYFCQQYSSY |
| | chain variable | PYTFGGGTKLEIK |
| | sequence | |
| 118 | Mouse clone | EVQLQESGGGLVQPGGSLKLSCAASGFTFSDYYMYWIRQTPQKRLE |
| | 16H8.B1 | WVAYISNGGGNTYYSDTVKGRFTISRDNAKNTLYLQMSRLKSEDTA |
| | Heavy chain | MYYCASPEARYYGNFPFPYWGQGTLVTVSA |
| | variable sequence | |
| 119 | Mouse clone | DIVMTQSHKFMSTSVGGRVTITCKASQDVSTTVAWYQQKPGQSPKL |
| | 16H8.B1 | LIYWASTRQTGVPDRFTGSGSGTDFTLTISNVQSEDLADYFCQQYSS |
| | Light chain variable | YPFTFGSGTKLEIK |
| | sequence | |
| 120 | CDRH1 | GFTFSDYY |
| | 14E4.H3 (mouse) | |
| | CDRH1 | |
| | Clone 16H8.B1 | |
| | (mouse) | |
| 121 | CDRH3 | ASPEARYYGNYPFPY |
| | Clone 14E4.H3 | |
| | (mouse) | |
| 122 | CDRH1 | GFSLTTYG |
| | Clone 14F6.B1 | |
| | (mouse) | |
| 123 | CDRH3 | ARTSHWYFDV |
| | Clone 14F6.B1 | |
| | (mouse) | |
| 124 | CDRH3 | ASPEARYYGNFPFPY |
| | Clone 16H8.B1 | |
| | (mouse) | |
| 125 | Nectin-4 Mask #3 | GGWTCADDEDTWLCPIGG |
| 126 | Nectin-4 Mask #4 | GGWACAPDEDTWLCPIGG |
| 127 | Full-length light | EIQMTQSHKTFSVSTGQRVTITCKASQDVSIAVAWYQQKPGKAPKLL |
| | chain sequence | IYWASTRQTGVPSRFSGSGSGTDFTLTISCVQSEDFAVYYCQQYSSYP |
| | Sequence Name: | FTFGQGTKLEIKRTVAAPSVFIFPPSDEQLKSGTASVVCLLNNFYPRE |
| | DAB012120 | AKVQWKVDNALQSGNSQESVTEQDSKDSTYSLSSTLTLSKADYEKH |
| | | KVYACEVTHQGLSSPVTKSFNRGEC |
| 128 | Full-length light | DIQMTQSPSFLSASVGDRVTITCKASQDVSIAVAWYQQKPGKAPKLL |
| | chain sequence | IYWASTRQTGVPSRFSGSGSGTDFTLTISSLQPEDFATYFCQQYSSYPF |
| | Sequence Name: | TFGQGTKLEIKRTVAAPSVFIFPPSDEQLKSGTASVVCLLNNFYPREA |
| | DAB012121 | KVQWKVDNALQSGNSQESVTEQDSKDSTYSLSSTLTLSKADYEKHK |
| | | VYACEVTHQGLSSPVTKSFNRGEC |
| 129 | Full-length light | DIQMTQSPSFLSASVGERVTITCKASQDVSIAVAWYQQKPGKAPKLL |
| | chain sequence | IYWASTRQTGVPSRFSGSGSGTDFTLTISSLQPEDIATYFCQQYSSYPF |
| | Sequence Name: | TFGQGTKLEIKRTVAAPSVFIFPPSDEQLKSGTASVVCLLNNFYPREA |
| | DAB012122 | KVQWKVDNALQSGNSQESVTEQDSKDSTYSLSSTLTLSKADYEKHK |
| | | VYACEVTHQGLSSPVTKSFNRGEC |
| 130 | Full-length light | DIQMTQSPSFLSASVGDRVTITCKASQDVSIAVAWYQQKPGKAPKLL |
| | chain sequence | IYWASTRQTGVPSRFSGSGSGTDFTLTISSLQPEDSATYFCQQYSSYPF |
| | Sequence Name: | TFGQGTKVDIKRTVAAPSVFIFPPSDEQLKSGTASVVCLLNNFYPREA |
| | DAB012123 | KVQWKVDNALQSGNSQESVTEQDSKDSTYSLSSTLTLSKADYEKHK |
| | | VYACEVTHQGLSSPVTKSFNRGEC |
| 131 | Full-length light | EIQMTQSQSTLSVSLGDRVTITCKASQNVVTAVAWYQQKPGQAPKL |
| | chain sequence | LIYSASHRFTGVPSRFSGSGSGTDFTLIISCMQSEDFAVYYCQQYSSYP |
| | Sequence Name: | YTFGQGTKLEIKRTVAAPSVFIFPPSDEQLKSGTASVVCLLNNFYPRE |
| | DAB012124 | AKVQWKVDNALQSGNSQESVTEQDSKDSTYSLSSTLTLSKADYEKH |
| | | KVYACEVTHQGLSSPVTKSFNRGEC |
| 132 | Full-length light | DIQMTQSPSFLSASVGDRVTITCKASQNVVTAVAWYQQKPGKAPKL |
| | chain sequence | LIYSASHRFTGVPSRFSGSGSGTDFTLTISNLQSEDLATYFCQQYSSYP |
| | Sequence Name: | YTFGQGTKLEIKRTVAAPSVFIFPPSDEQLKSGTASVVCLLNNFYPRE |
| | DAB012125 | AKVQWKVDNALQSGNSQESVTEQDSKDSTYSLSSTLTLSKADYEKH |
| | | KVYACEVTHQGLSSPVTKSFNRGEC |
| 133 | Full-length light | DIQMTQSPSFLSASVGDRVTITCKASQNVVTAVAWYQQKPGKAPKL |
| | chain sequence | LIYSASHRFTGVPSRFSGSGSGTDFTLTISNLQPEDSATYFCQQYSSYP |
| | Sequence Name: | YTFGQGTKLEIKRTVAAPSVFIFPPSDEQLKSGTASVVCLLNNFYPRE |
| | DAB012126 | AKVQWKVDNALQSGNSQESVTEQDSKDSTYSLSSTLTLSKADYEKH |
| | | KVYACEVTHQGLSSPVTKSFNRGEC |
| 134 | Full-length light | DIQMTQSPSFLSASVGDRVTITCKASQNVVTAVAWYQQKPGKAPKL |
| | chain sequence | LIYSASHRFTGVPSRFSGSGSGTDFTLTISNLQPDDAATYFCQQYSSYP |
| | Sequence Name: | YTFGQGTKLEIKRTVAAPSVFIFPPSDEQLKSGTASVVCLLNNFYPRE |
| | DAB012127 | AKVQWKVDNALQSGNSQESVTEQDSKDSTYSLSSTLTLSKADYEKH |
| | | KVYACEVTHQGLSSPVTKSFNRGEC |
| 135 | Full-length light | AIQMTQSHKSFSVSTGQRVTITCKASQDVSTTVAWYQQKPGQAPKL |
| | chain sequence | LIYWASTRQTGVPSRFTGSGSGTDFTLTISCVQSEDFAVYYCQQYSSY |
| | Sequence Name: | PFTFGQGTKLEIKRTVAAPSVFIFPPSDEQLKSGTASVVCLLNNFYPRE |
| | DAB012128 | AKVQWKVDNALQSGNSQESVTEQDSKDSTYSLSSTLTLSKADYEKH |
| | | KVYACEVTHQGLSSPVTKSFNRGEC |
| 136 | Full-length light | DIQMTQSPSFLSASVGDRVTITCKASQDVSTTVAWYQQKPGKAPKLL |
| | chain sequence | IYWASTRQTGVPSRFSGSGSGTDFTLTISNLQPEDFATYFCQQYSSYP |
| | Sequence Name: | FTFGQGTKLEIKRTVAAPSVFIFPPSDEQLKSGTASVVCLLNNFYPRE |
| | DAB012129 | AKVQWKVDNALQSGNSQESVTEQDSKDSTYSLSSTLTLSKADYEKH |
| | | KVYACEVTHQGLSSPVTKSFNRGEC |
| 137 | Full-length light | DIQMTQSPSFLSASVGERVTITCKASQDVSTTVAWYQQKPGKAPKLL |
| | chain sequence | IYWASTRQTGVPSRFSGSGSGTDFTLTINSLQPEDVATYFCQQYSSYP |
| | Sequence Name: | FTFGQGTKLEIKRTVAAPSVFIFPPSDEQLKSGTASVVCLLNNFYPRE |
| | DAB012130 | AKVQWKVDNALQSGNSQESVTEQDSKDSTYSLSSTLTLSKADYEKH |
| | | KVYACEVTHQGLSSPVTKSFNRGEC |
| 138 | Full-length light | DIQMTQSPSFLSASVGERVTITCKASQDVSTTVAWYQQKPGKAPKLL |
| | chain sequence | IYWASTRQTGVPSRFSGSGSGTDFTLTISNLQPEDVATYFCQQYSSYP |
| | Sequence Name: | FTFGQGTKLEIKRTVAAPSVFIFPPSDEQLKSGTASVVCLLNNFYPRE |
| | DAB012131 | AKVQWKVDNALQSGNSQESVTEQDSKDSTYSLSSTLTLSKADYEKH |
| | | KVYACEVTHQGLSSPVTKSFNRGEC |
| 139 | Full-length light | DIQMTQSPSSVSLTVGQRVTITCLATEDIFSYLAWYQQKPGKAPRLLI |
| | chain sequence | YGANRLKDGVPSRFSGSGSGTQYTLRISSMQPEDFGVYYCLQGAKFP |
| | Sequence Name: | LTFGPGTKLEIKRTVAAPSVFIFPPSDEQLKSGTASVVCLLNNFYPRE |
| | DAB012132 | AKVQWKVDNALQSGNSQESVTEQDSKDSTYSLSSTLTLSKADYEKH |
| | | KVYACEVTHQGLSSPVTKSFNRGEC |
| 140 | Full-length light | DIQMTQSPSSLSASIGDTVTITCLATEDIFSYLAWYQQRPGKAPKLLIY |
| | chain sequence | GANRLKDGVPSRFSGSGSGTDYTLTISGLQPEDFVTYYCLQGAKFPL |
| | Sequence Name: | TFGQGTKLDMKRTVAAPSVFIFPPSDEQLKSGTASVVCLLNNFYPRE |
| | DAB012133 | AKVQWKVDNALQSGNSQESVTEQDSKDSTYSLSSTLTLSKADYEKH |
| | | KVYACEVTHQGLSSPVTKSFNRGEC |
| 141 | Full-length light | DIQMTQSPSSLSASVGDTVTITCLATEDIFSYLAWYQQKPGKAPKLLI |
| | chain sequence | YGANRLKDGVPSRFSGSGSGTDYTLTISGLQPEDFGTYYCLQGAKFP |
| | Sequence Name: | LTFGQGTKLEIKRTVAAPSVFIFPPSDEQLKSGTASVVCLLNNFYPRE |
| | DAB012134 | AKVQWKVDNALQSGNSQESVTEQDSKDSTYSLSSTLTLSKADYEKH |
| | | KVYACEVTHQGLSSPVTKSFNRGEC |
| 142 | Full-length light | DIQMTQSPSSLSASVGDTVTITCLATEDIFSYLAWYQQKPGKAPKLLI |
| | chain sequence | YGANRLKDGVPSRFSGGGSGTEYSLTISGLQPEDFGTYYCLQGAKFP |
| | Sequence Name: | LTFGQGTKLDMKRTVAAPSVFIFPPSDEQLKSGTASVVCLLNNFYPR |
| | DAB012135 | EAKVQWKVDNALQSGNSQESVTEQDSKDSTYSLSSTLTLSKADYEK |
| | | HKVYACEVTHQGLSSPVTKSFNRGEC |
| 143 | Full-length light | DIQMTQSPSSLSASVGDTVTITCLATEDIFSYLAWYQQKPGKAPKLLI |
| | chain sequence | YGANRLKDTVPSRFSGGGSGTEYSLTISGLQPEDFGTYYCLQGAKFP |
| | Sequence Name: | LTFGQGTKLDMKRTVAAPSVFIFPPSDEQLKSGTASVVCLLNNFYPR |
| | DAB012135 | EAKVQWKVDNALQSGNSQESVTEQDSKDSTYSLSSTLTLSKADYEK |
| | (modified) | HKVYACEVTHQGLSSPVTKSFNRGEC |
| 144 | Full-length heavy | QVQLLESGGELVQPGGSLRLSCAASGFTFSDYYMFWIRQTPQKRLE |
| | chain sequence | WVAYISNGGGNTYYPDTVKGRFTISRDNAKNQLYLQMRSLTPEDTA |
| | Sequence Name: | IYYCASPEARYYGNYPFPYWGQGTLVTVSSASTKGPSVFPLAPSSKS |
| | DAB012120 | TSGGTAALGCLVKDYFPEPVTVSWNSGALTSGVHTFPAVLQSSGLYS |
| | | LSSVVTVPSSSLGTQTYICNVNHKPSNTKVDKKVEPKSCDKTHTCPP |
| | | CPAPELLGGPSVFLFPPKPKDTLMISRTPEVTCVVVDVSHEDPEVKFN |
| | | WYVDGVEVHNAKTKPREEQYNSTYRVVSVLTVLHQDWLNGKEYK |
| | | CKVSNKALPAPIEKTISKAKGQPREPQVYTLPPSRDELTKNQVSLTCL |
| | | VKGFYPSDIAVEWESNGQPENNYKTTPPVLDSDGSFFLYSKLTVDKS |
| | | RWQQGNVFSCSVMHEALHNHYTQKSLSLSPGK |
| 145 | Full-length heavy | QVQLVESGGGLVKPGGSLRLSCAASGFTFSDYYMFWIRQAPGKGLE |
| | chain sequence | WVAYISNGGGNTYYPDTVKGRFTISRDNAKNTLYLQMNSLRAEDTA |
| | Sequence Name: | VYYCASPEARYYGNYPFPYWGQGTLVTVSSASTKGPSVFPLAPSSKS |
| | DAB012120 | TSGGTAALGCLVKDYFPEPVTVSWNSGALTSGVHTFPAVLQSSGLYS |
| | | LSSVVTVPSSSLGTQTYICNVNHKPSNTKVDKKVEPKSCDKTHTCPP |
| | | CPAPELLGGPSVFLFPPKPKDTLMISRTPEVTCVVVDVSHEDPEVKFN |
| | | WYVDGVEVHNAKTKPREEQYNSTYRVVSVLTVLHQDWLNGKEYK |
| | | CKVSNKALPAPIEKTISKAKGQPREPQVYTLPPSRDELTKNQVSLTCL |
| | | VKGFYPSDIAVEWESNGQPENNYKTTPPVLDSDGSFFLYSKLTVDKS |
| | | RWQQGNVFSCSVMHEALHNHYTQKSLSLSPGK |
| 146 | Full-length heavy | QVQLVESGGGVVKPGGSLRLSCAASGFTFSDYYMFWIRQAPGKGLE |
| | chain sequence | WVAYISNGGGNTYYPDTVKGRFTISRDNAKNTLYLQMNSLRAEDTA |
| | Sequence Name: | VYYCASPEARYYGNYPFPYWGQGTLVTVSSASTKGPSVFPLAPSSKS |
| | DAB012122 | TSGGTAALGCLVKDYFPEPVTVSWNSGALTSGVHTFPAVLQSSGLYS |
| | | LSSVVTVPSSSLGTQTYICNVNHKPSNTKVDKKVEPKSCDKTHTCPP |
| | | CPAPELLGGPSVFLFPPKPKDTLMISRTPEVTCVVVDVSHEDPEVKFN |
| | | WYVDGVEVHNAKTKPREEQYNSTYRVVSVLTVLHQDWLNGKEYK |
| | | CKVSNKALPAPIEKTISKAKGQPREPQVYTLPPSRDELTKNQVSLTCL |
| | | VKGFYPSDIAVEWESNGQPENNYKTTPPVLDSDGSFFLYSKLTVDKS |
| | | RWQQGNVFSCSVMHEALHNHYTQKSLSLSPGK |
| 147 | Full-length heavy | QVQLVESGGGVVQPGGSLRLSCAASGFTFSDYYMFWIRQAPGKGLE |
| | chain sequence | WVAYISNGGGNTYYPDTVKGRFTISRDNAKNTLYLQMNSLRAEDTA |
| | Sequence Name: | VYYCASPEARYYGNYPFPYWGQGTLVTVSSASTKGPSVFPLAPSSKS |
| | DAB012123 | TSGGTAALGCLVKDYFPEPVTVSWNSGALTSGVHTFPAVLQSSGLYS |
| | | LSSVVTVPSSSLGTQTYICNVNHKPSNTKVDKKVEPKSCDKTHTCPP |
| | | CPAPELLGGPSVFLFPPKPKDTLMISRTPEVTCVVVDVSHEDPEVKFN |
| | | WYVDGVEVHNAKTKPREEQYNSTYRVVSVLTVLHQDWLNGKEYK |
| | | CKVSNKALPAPIEKTISKAKGQPREPQVYTLPPSRDELTKNQVSLTCL |
| | | VKGFYPSDIAVEWESNGQPENNYKTTPPVLDSDGSFFLYSKLTVDKS |
| | | RWQQGNVFSCSVMHEALHNHYTQKSLSLSPGK |
| 148 | Full-length heavy | QVQLLQSGPGLVKPSATLSLTCTVSGFSLTTYGVHWIRQPPGKGLEW |
| | chain sequence | MGVIWSGGSTDYNAAFISRLTISKDNSKNQVFLQLYSLRAEDTAIYY |
| | Sequence Name: | CARTSHWYFDVWGRGTLVTVSSASTKGPSVFPLAPSSKSTSGGTAAL |
| | DAB012124 | GCLVKDYFPEPVTVSWNSGALTSGVHTFPAVLQSSGLYSLSSVVTVP |
| | | SSSLGTQTYICNVNHKPSNTKVDKKVEPKSCDKTHTCPPCPAPELLG |
| | | GPSVFLFPPKPKDTLMISRTPEVTCVVVDVSHEDPEVKFNWYVDGVE |
| | | VHNAKTKPREEQYNSTYRVVSVLTVLHQDWLNGKEYKCKVSNKAL |
| | | PAPIEKTISKAKGQPREPQVYTLPPSRDELTKNQVSLTCLVKGFYPSDI |
| | | AVEWESNGQPENNYKTTPPVLDSDGSFFLYSKLTVDKSRWQQGNVF |
| | | SCSVMHEALHNHYTQKSLSLSPGK |
| 149 | Full-length heavy | QVQLQESGPGLVKPSQTLSLTCTVSGFSLTTYGVHWVRQSPGKGLE |
| | chain sequence | WLGVIWSGGSTDYNAAFISRLSISKDNSKSQVFLEMTSLTAADTAIY |
| | Sequence Name: | YCARTSHWYFDVWGRGTLVTVSSASTKGPSVFPLAPSSKSTSGGTA |
| | DAB012125 | ALGCLVKDYFPEPVTVSWNSGALTSGVHTFPAVLQSSGLYSLSSVVT |
| | | VPSSSLGTQTYICNVNHKPSNTKVDKKVEPKSCDKTHTCPPCPAPEL |
| | | LGGPSVFLFPPKPKDTLMISRTPEVTCVVVDVSHEDPEVKFNWYVDG |
| | | VEVHNAKTKPREEQYNSTYRVVSVLTVLHQDWLNGKEYKCKVSNK |
| | | ALPAPIEKTISKAKGQPREPQVYTLPPSRDELTKNQVSLTCLVKGFYP |
| | | SDIAVEWESNGQPENNYKTTPPVLDSDGSFFLYSKLTVDKSRWQQG |
| | | NVFSCSVMHEALHNHYTQKSLSLSPGK |
| 150 | Full-length heavy | QVQLQESGPGLVKPSQTLSLTCTVSGFSLTTYGVHWVRQAPEKGLE |
| | chain sequence | WLGVIWSGGSTDYNAAFISRLSISKDNSKSQVFLEMTSLTADDTAIY |
| | Sequence Name: | YCARTSHWYFDVWGRGTLVTVSSASTKGPSVFPLAPSSKSTSGGTA |
| | DAB012126 | ALGCLVKDYFPEPVTVSWNSGALTSGVHTFPAVLQSSGLYSLSSVVT |
| | | VPSSSLGTQTYICNVNHKPSNTKVDKKVEPKSCDKTHTCPPCPAPEL |
| | | LGGPSVFLFPPKPKDTLMISRTPEVTCVVVDVSHEDPEVKFNWYVDG |
| | | VEVHNAKTKPREEQYNSTYRVVSVLTVLHQDWLNGKEYKCKVSNK |
| | | ALPAPIEKTISKAKGQPREPQVYTLPPSRDELTKNQVSLTCLVKGFYP |
| | | SDIAVEWESNGQPENNYKTTPPVLDSDGSFFLYSKLTVDKSRWQQG |
| | | NVFSCSVMHEALHNHYTQKSLSLSPGK |
| 151 | Full-length heavy | QVQLQESGPGLVKPSQTLSLTCTVSGFSLTTYGVHWVRQTPEKGLE |
| | chain sequence | WLGVIWSGGSTDYNAAFISRLSISKDNSKSQVFLEMTSLTADDTAIY |
| | Sequence Name: | YCARTSHWYFDVWGRGTLVTVSSASTKGPSVFPLAPSSKSTSGGTA |
| | DAB012127 | ALGCLVKDYFPEPVTVSWNSGALTSGVHTFPAVLQSSGLYSLSSVVT |
| | | VPSSSLGTQTYICNVNHKPSNTKVDKKVEPKSCDKTHTCPPCPAPEL |
| | | LGGPSVFLFPPKPKDTLMISRTPEVTCVVVDVSHEDPEVKFNWYVDG |
| | | VEVHNAKTKPREEQYNSTYRVVSVLTVLHQDWLNGKEYKCKVSNK |
| | | ALPAPIEKTISKAKGQPREPQVYTLPPSRDELTKNQVSLTCLVKGFYP |
| | | SDIAVEWESNGQPENNYKTTPPVLDSDGSFFLYSKLTVDKSRWQQG |
| | | NVFSCSVMHEALHNHYTQKSLSLSPGK |
| 152 | Full-length heavy | QVQLLESGGGLVQPGGSLRLSCAASGFTFSDYYMYWIRQTPQKRLE |
| | chain sequence | WVAYISNGGGNTYYSDTVKGRFTISRDNAKNQLYLQMRSLRPEDTA |
| | Sequence Name: | IYYCASPEARYYGNFPFPYWGQGTLVTVSSASTKGPSVFPLAPSSKST |
| | DAB012128 | SGGTAALGCLVKDYFPEPVTVSWNSGALTSGVHTFPAVLQSSGLYSL |
| | | SSVVTVPSSSLGTQTYICNVNHKPSNTKVDKKVEPKSCDKTHTCPPC |
| | | PAPELLGGPSVFLFPPKPKDTLMISRTPEVTCVVVDVSHEDPEVKFN |
| | | WYVDGVEVHNAKTKPREEQYNSTYRVVSVLTVLHQDWLNGKEYK |
| | | CKVSNKALPAPIEKTISKAKGQPREPQVYTLPPSRDELTKNQVSLTCL |
| | | VKGFYPSDIAVEWESNGQPENNYKTTPPVLDSDGSFFLYSKLTVDKS |
| | | RWQQGNVFSCSVMHEALHNHYTQKSLSLSPGK |
| 153 | Full-length heavy | QVQLVESGGGLVKPGGSLRLSCAASGFTFSDYYMYWIRQAPGKGLE |
| | chain sequence | WVAYISNGGGNTYYSDTVKGRFTISRDNAKNTLYLQMNSLRAEDTA |
| | Sequence Name: | VYYCASPEARYYGNFPFPYWGQGTLVTVSSASTKGPSVFPLAPSSKS |
| | DAB012129 | TSGGTAALGCLVKDYFPEPVTVSWNSGALTSGVHTFPAVLQSSGLYS |
| | | LSSVVTVPSSSLGTQTYICNVNHKPSNTKVDKKVEPKSCDKTHTCPP |
| | | CPAPELLGGPSVFLFPPKPKDTLMISRTPEVTCVVVDVSHEDPEVKFN |
| | | WYVDGVEVHNAKTKPREEQYNSTYRVVSVLTVLHQDWLNGKEYK |
| | | CKVSNKALPAPIEKTISKAKGQPREPQVYTLPPSRDELTKNQVSLTCL |
| | | VKGFYPSDIAVEWESNGQPENNYKTTPPVLDSDGSFFLYSKLTVDKS |
| | | RWQQGNVFSCSVMHEALHNHYTQKSLSLSPGK |
| 154 | Full-length heavy | QVQLVESGGGLVKPGGSLRLSCAASGFTFSDYYMYWIRQAPGKGLE |
| | chain sequence | WVAYISNGGGNTYYSDTVKGRFTISRDNAKNTLYLQMNSLRAEDTA |
| | Sequence Name: | VYYCASPEARYYGNFPFPYWGQGTLVTVSSASTKGPSVFPLAPSSKS |
| | DAB012130 | TSGGTAALGCLVKDYFPEPVTVSWNSGALTSGVHTFPAVLQSSGLYS |
| | | LSSVVTVPSSSLGTQTYICNVNHKPSNTKVDKKVEPKSCDKTHTCPP |
| | | CPAPELLGGPSVFLFPPKPKDTLMISRTPEVTCVVVDVSHEDPEVKFN |
| | | WYVDGVEVHNAKTKPREEQYNSTYRVVSVLTVLHQDWLNGKEYK |
| | | CKVSNKALPAPIEKTISKAKGQPREPQVYTLPPSRDELTKNQVSLTCL |
| | | VKGFYPSDIAVEWESNGQPENNYKTTPPVLDSDGSFFLYSKLTVDKS |
| | | RWQQGNVFSCSVMHEALHNHYTQKSLSLSPGK |
| 155 | Full-length heavy | QVQLVESGGGLVKPGGSLRLSCAASGFTFSDYYMYWIRQAPGKGLE |
| | chain sequence | WVAYISNGGGNTYYSDTVKGRFTISRDNAKNTLYLQMNSLRAEDTA |
| | Sequence Name: | VYYCASPEARYYGNFPFPYWGQGTLVTVSSASTKGPSVFPLAPSSKS |
| | DAB012131 | TSGGTAALGCLVKDYFPEPVTVSWNSGALTSGVHTFPAVLQSSGLYS |
| | | LSSVVTVPSSSLGTQTYICNVNHKPSNTKVDKKVEPKSCDKTHTCPP |
| | | CPAPELLGGPSVFLFPPKPKDTLMISRTPEVTCVVVDVSHEDPEVKFN |
| | | WYVDGVEVHNAKTKPREEQYNSTYRVVSVLTVLHQDWLNGKEYK |
| | | CKVSNKALPAPIEKTISKAKGQPREPQVYTLPPSRDELTKNQVSLTCL |
| | | VKGFYPSDIAVEWESNGQPENNYKTTPPVLDSDGSFFLYSKLTVDKS |
| | | RWQQGNVFSCSVMHEALHNHYTQKSLSLSPGK |
| 156 | Full-length heavy | QVQLVESGGGVVQPGRSLRLSCAASGFSFSNYYMAWVRQAPGKGL |
| | chain sequence | EWVASISTGGGNIYYRDSVKGRFTISRDNSKSTLYLQMRSLRSEDTAI |
| | Sequence Name: | YYCARQTAYYVMDAWGQGTMVTVSSASTKGPSVFPLAPSSKSTSG |
| | DAB012132 | GTAALGCLVKDYFPEPVTVSWNSGALTSGVHTFPAVLQSSGLYSLSS |
| | | VVTVPSSSLGTQTYICNVNHKPSNTKVDKKVEPKSCDKTHTCPPCPA |
| | | PELLGGPSVFLFPPKPKDTLMISRTPEVTCVVVDVSHEDPEVKFNWY |
| | | VDGVEVHNAKTKPREEQYNSTYRVVSVLTVLHQDWLNGKEYKCK |
| | | VSNKALPAPIEKTISKAKGQPREPQVYTLPPSRDELTKNQVSLTCLVK |
| | | GFYPSDIAVEWESNGQPENNYKTTPPVLDSDGSFFLYSKLTVDKSRW |
| | | QQGNVFSCSVMHEALHNHYTQKSLSLSPGK |
| 157 | Full-length heavy | EVQLVESGGGLVQPGGSLRLSCAASGFSFSNYYMAWVRQAPGKGLE |
| | chain sequence | WVASISTGGGNIYYRDSVKGRFTISRDNAKNSLYLQMNSLRAEDTA |
| | Sequence Name: | VYYCARQTAYYVMDAWGQGTTVTVSSASTKGPSVFPLAPSSKSTSG |
| | DAB012133 | GTAALGCLVKDYFPEPVTVSWNSGALTSGVHTFPAVLQSSGLYSLSS |
| | | VVTVPSSSLGTQTYICNVNHKPSNTKVDKKVEPKSCDKTHTCPPCPA |
| | | PELLGGPSVFLFPPKPKDTLMISRTPEVTCVVVDVSHEDPEVKFNWY |
| | | VDGVEVHNAKTKPREEQYNSTYRVVSVLTVLHQDWLNGKEYKCK |
| | | VSNKALPAPIEKTISKAKGQPREPQVYTLPPSRDELTKNQVSLTCLVK |
| | | GFYPSDIAVEWESNGQPENNYKTTPPVLDSDGSFFLYSKLTVDKSRW |
| | | QQGNVFSCSVMHEALHNHYTQKSLSLSPGK |
| 158 | Full-length heavy | EVQLVESGGGLVQPGGSLRLSCAASGFSFSNYYMAWVRQAPGKGLE |
| | chain sequence | WVASISTGGGNIYYRDSVKGRFTISRDNAKNSLYLQMNSLRAEDTA |
| | Sequence Name: | VYYCARQTAYYVMDAWGQGTTVTVSSASTKGPSVFPLAPSSKSTSG |
| | DAB012134 | GTAALGCLVKDYFPEPVTVSWNSGALTSGVHTFPAVLQSSGLYSLSS |
| | | VVTVPSSSLGTQTYICNVNHKPSNTKVDKKVEPKSCDKTHTCPPCPA |
| | | PELLGGPSVFLFPPKPKDTLMISRTPEVTCVVVDVSHEDPEVKFNWY |
| | | VDGVEVHNAKTKPREEQYNSTYRVVSVLTVLHQDWLNGKEYKCK |
| | | VSNKALPAPIEKTISKAKGQPREPQVYTLPPSRDELTKNQVSLTCLVK |
| | | GFYPSDIAVEWESNGQPENNYKTTPPVLDSDGSFFLYSKLTVDKSRW |
| | | QQGNVFSCSVMHEALHNHYTQKSLSLSPGK |
| 159 | Full-length heavy | EVQLVESGGGLVQPGGSLRLSCAASGFSFSNYYMAWVRQAPGKGLE |
| | chain sequence | WVASISTGGGNIYYRDSVKGRFTISRDNAKNSLYLQMNSLRAEDTA |
| | Sequence Name: | VYYCARQTAYYVMDAWGQGTTVTVSSASTKGPSVFPLAPSSKSTSG |
| | DAB012135 | GTAALGCLVKDYFPEPVTVSWNSGALTSGVHTFPAVLQSSGLYSLSS |
| | | VVTVPSSSLGTQTYICNVNHKPSNTKVDKKVEPKSCDKTHTCPPCPA |
| | | PELLGGPSVFLFPPKPKDTLMISRTPEVTCVVVDVSHEDPEVKFNWY |
| | | VDGVEVHNAKTKPREEQYNSTYRVVSVLTVLHQDWLNGKEYKCK |
| | | VSNKALPAPIEKTISKAKGQPREPQVYTLPPSRDELTKNQVSLTCLVK |
| | | GFYPSDIAVEWESNGQPENNYKTTPPVLDSDGSFFLYSKLTVDKSRW |
| | | QQGNVFSCSVMHEALHNHYTQKSLSLSPGK |
| 160 | Full-length light | GAGATCCAGATGACCCAGAGCCACAAGACCTTCAGCGTGAGCAC |
| | chain nucleotide | CGGCCAGAGGGTGACCATCACCTGCAAGGCCAGCCAGGACGTGA |
| | sequence | GCATCGCCGTGGCCTGGTACCAGCAGAAGCCCGGCAAGGCCCCC |
| | Sequence Name: | AAGCTGCTGATCTACTGGGCCAGCACCAGGCAGACCGGCGTGCC |
| | DAB012120 | CAGCAGGTTCAGCGGCAGCGGCAGCGGCACCGACTTCACCCTGA |
| | | CCATCAGCTGCGTGCAGAGCGAGGACTTCGCCGTGTACTACTGCC |
| | | AGCAGTACAGCAGCTACCCCTTCACCTTCGGCCAGGGCACCAAGC |
| | | TGGAGATCAAGCGAACGGTGGCTGCCCCCTCCGTGTTCATCTTCC |
| | | CCCCCAGCGATGAGCAGCTGAAGAGCGGCACAGCCAGCGTGGTG |
| | | TGCCTGCTGAACAACTTCTACCCCAGGGAGGCCAAGGTGCAGTGG |
| | | AAGGTGGACAACGCCCTGCAGAGCGGCAACAGCCAGGAGTCCGT |
| | | GACCGAGCAGGACAGCAAGGACAGCACCTACAGCCTGAGCAGCA |
| | | CACTGACCCTGAGCAAGGCCGACTACGAGAAGCACAAGGTGTAC |
| | | GCCTGCGAGGTGACCCATCAGGGCCTGAGCAGCCCCGTGACCAA |
| | | GAGCTTCAACAGGGGCGAGTGC |
| 161 | Full-length light | GACATCCAGATGACCCAGAGCCCCAGCTTCCTGAGCGCCAGCGTG |
| | chain nucleotide | GGCGACAGGGTGACCATCACCTGCAAGGCCAGCCAGGACGTGAG |
| | sequence | CATCGCCGTGGCCTGGTACCAGCAGAAGCCCGGCAAGGCCCCCA |
| | Sequence Name: | AGCTGCTGATCTACTGGGCCAGCACCAGGCAGACCGGCGTGCCC |
| | DAB012121 | AGCAGGTTCAGCGGCAGCGGCAGCGGCACCGACTTCACCCTGAC |
| | | CATCAGCAGCCTGCAGCCCGAGGACTTCGCCACCTACTTCTGCCA |
| | | GCAGTACAGCAGCTACCCCTTCACCTTCGGCCAGGGCACCAAGCT |
| | | GGAGATCAAGCGAACGGTGGCTGCCCCCTCCGTGTTCATCTTCCC |
| | | CCCCAGCGATGAGCAGCTGAAGAGCGGCACAGCCAGCGTGGTGT |
| | | GCCTGCTGAACAACTTCTACCCCAGGGAGGCCAAGGTGCAGTGG |
| | | AAGGTGGACAACGCCCTGCAGAGCGGCAACAGCCAGGAGTCCGT |
| | | GACCGAGCAGGACAGCAAGGACAGCACCTACAGCCTGAGCAGCA |
| | | CACTGACCCTGAGCAAGGCCGACTACGAGAAGCACAAGGTGTAC |
| | | GCCTGCGAGGTGACCCATCAGGGCCTGAGCAGCCCCGTGACCAA |
| | | GAGCTTCAACAGGGGCGAGTGC |
| 162 | Full-length light | GACATCCAGATGACCCAGAGCCCCAGCTTCCTGAGCGCCAGCGTG |
| | chain nucleotide | GGCGAGAGGGTGACCATCACCTGCAAGGCCAGCCAGGACGTGAG |
| | sequence | CATCGCCGTGGCCTGGTACCAGCAGAAGCCCGGCAAGGCCCCCA |
| | Sequence Name: | AGCTGCTGATCTACTGGGCCAGCACCAGGCAGACCGGCGTGCCC |
| | DAB012122 | AGCAGGTTCAGCGGCAGCGGCAGCGGCACCGACTTCACCCTGAC |
| | | CATCAGCAGCCTGCAGCCCGAGGACATCGCCACCTACTTCTGCCA |
| | | GCAGTACAGCAGCTACCCCTTCACCTTCGGCCAGGGCACCAAGCT |
| | | GGAGATCAAGCGAACGGTGGCTGCCCCCTCCGTGTTCATCTTCCC |
| | | CCCCAGCGATGAGCAGCTGAAGAGCGGCACAGCCAGCGTGGTGT |
| | | GCCTGCTGAACAACTTCTACCCCAGGGAGGCCAAGGTGCAGTGG |
| | | AAGGTGGACAACGCCCTGCAGAGCGGCAACAGCCAGGAGTCCGT |
| | | GACCGAGCAGGACAGCAAGGACAGCACCTACAGCCTGAGCAGCA |
| | | CACTGACCCTGAGCAAGGCCGACTACGAGAAGCACAAGGTGTAC |
| | | GCCTGCGAGGTGACCCATCAGGGCCTGAGCAGCCCCGTGACCAA |
| | | GAGCTTCAACAGGGGCGAGTGC |
| 163 | Full-length light | GACATCCAGATGACCCAGAGCCCCAGCTTCCTGAGCGCCAGCGTG |
| | chain nucleotide | GGCGACAGGGTGACCATCACCTGCAAGGCCAGCCAGGACGTGAG |
| | sequence | CATCGCCGTGGCCTGGTACCAGCAGAAGCCCGGCAAGGCCCCCA |
| | Sequence Name: | AGCTGCTGATCTACTGGGCCAGCACCAGGCAGACCGGCGTGCCC |
| | DAB012123 | AGCAGGTTCAGCGGCAGCGGCAGCGGCACCGACTTCACCCTGAC |
| | | CATCAGCAGCCTGCAGCCCGAGGACAGCGCCACCTACTTCTGCCA |
| | | GCAGTACAGCAGCTACCCCTTCACCTTCGGCCAGGGCACCAAGGT |
| | | GGACATCAAGCGAACGGTGGCTGCCCCCTCCGTGTTCATCTTCCC |
| | | CCCCAGCGATGAGCAGCTGAAGAGCGGCACAGCCAGCGTGGTGT |
| | | GCCTGCTGAACAACTTCTACCCCAGGGAGGCCAAGGTGCAGTGG |
| | | AAGGTGGACAACGCCCTGCAGAGCGGCAACAGCCAGGAGTCCGT |
| | | GACCGAGCAGGACAGCAAGGACAGCACCTACAGCCTGAGCAGCA |
| | | CACTGACCCTGAGCAAGGCCGACTACGAGAAGCACAAGGTGTAC |
| | | GCCTGCGAGGTGACCCATCAGGGCCTGAGCAGCCCCGTGACCAA |
| | | GAGCTTCAACAGGGGCGAGTGC |
| 164 | Full-length light | GAGATCCAGATGACCCAGAGCCAGAGCACCCTGAGCGTGAGCCT |
| | chain nucleotide | GGGCGACAGGGTGACCATCACCTGCAAGGCCAGCCAGAACGTGG |
| | sequence | TGACCGCCGTGGCCTGGTACCAGCAGAAGCCCGGCCAGGCCCCC |
| | Sequence Name: | AAGCTGCTGATCTACAGCGCCAGCCACAGGTTCACCGGCGTGCCC |
| | DAB012124 | AGCAGGTTCAGCGGCAGCGGCAGCGGCACCGACTTCACCCTGAT |
| | | CATCAGCTGCATGCAGAGCGAGGACTTCGCCGTGTACTACTGCCA |
| | | GCAGTACAGCAGCTACCCCTACACCTTCGGCCAGGGCACCAAGCT |
| | | GGAGATCAAGCGAACGGTGGCTGCCCCCTCCGTGTTCATCTTCCC |
| | | CCCCAGCGATGAGCAGCTGAAGAGCGGCACAGCCAGCGTGGTGT |
| | | GCCTGCTGAACAACTTCTACCCCAGGGAGGCCAAGGTGCAGTGG |
| | | AAGGTGGACAACGCCCTGCAGAGCGGCAACAGCCAGGAGTCCGT |
| | | GACCGAGCAGGACAGCAAGGACAGCACCTACAGCCTGAGCAGCA |
| | | CACTGACCCTGAGCAAGGCCGACTACGAGAAGCACAAGGTGTAC |
| | | GCCTGCGAGGTGACCCATCAGGGCCTGAGCAGCCCCGTGACCAA |
| | | GAGCTTCAACAGGGGCGAGTGC |
| 165 | Full-length light | GACATCCAGATGACCCAGAGCCCCAGCTTCCTGAGCGCCAGCGTG |
| | chain nucleotide | GGCGACAGGGTGACCATCACCTGCAAGGCCAGCCAGAACGTGGT |
| | sequence | GACCGCCGTGGCCTGGTACCAGCAGAAGCCCGGCAAGGCCCCCA |
| | Sequence Name: | AGCTGCTGATCTACAGCGCCAGCCACAGGTTCACCGGCGTGCCCA |
| | DAB012125 | GCAGGTTCAGCGGCAGCGGCAGCGGCACCGACTTCACCCTGACC |
| | | ATCAGCAACCTGCAGAGCGAGGACCTGGCCACCTACTTCTGCCAG |
| | | CAGTACAGCAGCTACCCCTACACCTTCGGCCAGGGCACCAAGCTG |
| | | GAGATCAAGCGAACGGTGGCTGCCCCCTCCGTGTTCATCTTCCCC |
| | | CCCAGCGATGAGCAGCTGAAGAGCGGCACAGCCAGCGTGGTGTG |
| | | CCTGCTGAACAACTTCTACCCCAGGGAGGCCAAGGTGCAGTGGA |
| | | AGGTGGACAACGCCCTGCAGAGCGGCAACAGCCAGGAGTCCGTG |
| | | ACCGAGCAGGACAGCAAGGACAGCACCTACAGCCTGAGCAGCAC |
| | | ACTGACCCTGAGCAAGGCCGACTACGAGAAGCACAAGGTGTACG |
| | | CCTGCGAGGTGACCCATCAGGGCCTGAGCAGCCCCGTGACCAAG |
| | | AGCTTCAACAGGGGCGAGTGC |
| 166 | Full-length light | GACATCCAGATGACCCAGAGCCCCAGCTTCCTGAGCGCCAGCGTG |
| | chain nucleotide | GGCGACAGGGTGACCATCACCTGCAAGGCCAGCCAGAACGTGGT |
| | sequence | GACCGCCGTGGCCTGGTACCAGCAGAAGCCCGGCAAGGCCCCCA |
| | Sequence Name: | AGCTGCTGATCTACAGCGCCAGCCACAGGTTCACCGGCGTGCCCA |
| | DAB012126 | GCAGGTTCAGCGGCAGCGGCAGCGGCACCGACTTCACCCTGACC |
| | | ATCAGCAACCTGCAGCCCGAGGACAGCGCCACCTACTTCTGCCAG |
| | | CAGTACAGCAGCTACCCCTACACCTTCGGCCAGGGCACCAAGCTG |
| | | GAGATCAAGCGAACGGTGGCTGCCCCCTCCGTGTTCATCTTCCCC |
| | | CCCAGCGATGAGCAGCTGAAGAGCGGCACAGCCAGCGTGGTGTG |
| | | CCTGCTGAACAACTTCTACCCCAGGGAGGCCAAGGTGCAGTGGA |
| | | AGGTGGACAACGCCCTGCAGAGCGGCAACAGCCAGGAGTCCGTG |
| | | ACCGAGCAGGACAGCAAGGACAGCACCTACAGCCTGAGCAGCAC |
| | | ACTGACCCTGAGCAAGGCCGACTACGAGAAGCACAAGGTGTACG |
| | | CCTGCGAGGTGACCCATCAGGGCCTGAGCAGCCCCGTGACCAAG |
| | | AGCTTCAACAGGGGCGAGTGC |
| 167 | Full-length light | GACATCCAGATGACCCAGAGCCCCAGCTTCCTGAGCGCCAGCGTG |
| | chain nucleotide | GGCGACAGGGTGACCATCACCTGCAAGGCCAGCCAGAACGTGGT |
| | sequence | GACCGCCGTGGCCTGGTACCAGCAGAAGCCCGGCAAGGCCCCCA |
| | Sequence Name: | AGCTGCTGATCTACAGCGCCAGCCACAGGTTCACCGGCGTGCCCA |
| | DAB012127 | GCAGGTTCAGCGGCAGCGGCAGCGGCACCGACTTCACCCTGACC |
| | | ATCAGCAACCTGCAGCCCGACGACGCCGCCACCTACTTCTGCCAG |
| | | CAGTACAGCAGCTACCCCTACACCTTCGGCCAGGGCACCAAGCTG |
| | | GAGATCAAGCGAACGGTGGCTGCCCCCTCCGTGTTCATCTTCCCC |
| | | CCCAGCGATGAGCAGCTGAAGAGCGGCACAGCCAGCGTGGTGTG |
| | | CCTGCTGAACAACTTCTACCCCAGGGAGGCCAAGGTGCAGTGGA |
| | | AGGTGGACAACGCCCTGCAGAGCGGCAACAGCCAGGAGTCCGTG |
| | | ACCGAGCAGGACAGCAAGGACAGCACCTACAGCCTGAGCAGCAC |
| | | ACTGACCCTGAGCAAGGCCGACTACGAGAAGCACAAGGTGTACG |
| | | CCTGCGAGGTGACCCATCAGGGCCTGAGCAGCCCCGTGACCAAG |
| | | AGCTTCAACAGGGGCGAGTGC |
| 168 | Full-length light | GCCATCCAGATGACCCAGAGCCACAAGAGCTTCAGCGTGAGCAC |
| | chain nucleotide | CGGCCAGAGGGTGACCATCACCTGCAAGGCCAGCCAGGACGTGA |
| | sequence | GCACCACCGTGGCCTGGTACCAGCAGAAGCCCGGCCAGGCCCCC |
| | Sequence Name: | AAGCTGCTGATCTACTGGGCCAGCACCAGGCAGACCGGCGTGCC |
| | DAB012128 | CAGCAGGTTCACCGGCAGCGGCAGCGGCACCGACTTCACCCTGA |
| | | CCATCAGCTGCGTGCAGAGCGAGGACTTCGCCGTGTACTACTGCC |
| | | AGCAGTACAGCAGCTACCCCTTCACCTTCGGCCAGGGCACCAAGC |
| | | TGGAGATCAAGCGAACGGTGGCTGCCCCCTCCGTGTTCATCTTCC |
| | | CCCCCAGCGATGAGCAGCTGAAGAGCGGCACAGCCAGCGTGGTG |
| | | TGCCTGCTGAACAACTTCTACCCCAGGGAGGCCAAGGTGCAGTGG |
| | | AAGGTGGACAACGCCCTGCAGAGCGGCAACAGCCAGGAGTCCGT |
| | | GACCGAGCAGGACAGCAAGGACAGCACCTACAGCCTGAGCAGCA |
| | | CACTGACCCTGAGCAAGGCCGACTACGAGAAGCACAAGGTGTAC |
| | | GCCTGCGAGGTGACCCATCAGGGCCTGAGCAGCCCCGTGACCAA |
| | | GAGCTTCAACAGGGGCGAGTGC |
| 169 | Full-length light | GACATCCAGATGACCCAGAGCCCCAGCTTCCTGAGCGCCAGCGTG |
| | chain nucleotide | GGCGACAGGGTGACCATCACCTGCAAGGCCAGCCAGGACGTGAG |
| | sequence | CACCACCGTGGCCTGGTACCAGCAGAAGCCCGGCAAGGCCCCCA |
| | Sequence Name: | AGCTGCTGATCTACTGGGCCAGCACCAGGCAGACCGGCGTGCCC |
| | DAB012129 | AGCAGGTTCAGCGGCAGCGGCAGCGGCACCGACTTCACCCTGAC |
| | | CATCAGCAACCTGCAGCCCGAGGACTTCGCCACCTACTTCTGCCA |
| | | GCAGTACAGCAGCTACCCCTTCACCTTCGGCCAGGGCACCAAGCT |
| | | GGAGATCAAGCGAACGGTGGCTGCCCCCTCCGTGTTCATCTTCCC |
| | | CCCCAGCGATGAGCAGCTGAAGAGCGGCACAGCCAGCGTGGTGT |
| | | GCCTGCTGAACAACTTCTACCCCAGGGAGGCCAAGGTGCAGTGG |
| | | AAGGTGGACAACGCCCTGCAGAGCGGCAACAGCCAGGAGTCCGT |
| | | GACCGAGCAGGACAGCAAGGACAGCACCTACAGCCTGAGCAGCA |
| | | CACTGACCCTGAGCAAGGCCGACTACGAGAAGCACAAGGTGTAC |
| | | GCCTGCGAGGTGACCCATCAGGGCCTGAGCAGCCCCGTGACCAA |
| | | GAGCTTCAACAGGGGCGAGTGC |
| 170 | Full-length light | GACATCCAGATGACCCAGAGCCCCAGCTTCCTGAGCGCCAGCGTG |
| | chain nucleotide | GGCGAGAGGGTGACCATCACCTGCAAGGCCAGCCAGGACGTGAG |
| | sequence | CACCACCGTGGCCTGGTACCAGCAGAAGCCCGGCAAGGCCCCCA |
| | Sequence Name: | AGCTGCTGATCTACTGGGCCAGCACCAGGCAGACCGGCGTGCCC |
| | DAB012130 | AGCAGGTTCAGCGGCAGCGGCAGCGGCACCGACTTCACCCTGAC |
| | | CATCAACAGCCTGCAGCCCGAGGACGTGGCCACCTACTTCTGCCA |
| | | GCAGTACAGCAGCTACCCCTTCACCTTCGGCCAGGGCACCAAGCT |
| | | GGAGATCAAGCGAACGGTGGCTGCCCCCTCCGTGTTCATCTTCCC |
| | | CCCCAGCGATGAGCAGCTGAAGAGCGGCACAGCCAGCGTGGTGT |
| | | GCCTGCTGAACAACTTCTACCCCAGGGAGGCCAAGGTGCAGTGG |
| | | AAGGTGGACAACGCCCTGCAGAGCGGCAACAGCCAGGAGTCCGT |
| | | GACCGAGCAGGACAGCAAGGACAGCACCTACAGCCTGAGCAGCA |
| | | CACTGACCCTGAGCAAGGCCGACTACGAGAAGCACAAGGTGTAC |
| | | GCCTGCGAGGTGACCCATCAGGGCCTGAGCAGCCCCGTGACCAA |
| | | GAGCTTCAACAGGGGCGAGTGC |
| 171 | Full-length light | GACATCCAGATGACCCAGAGCCCCAGCTTCCTGAGCGCCAGCGTG |
| | chain nucleotide | GGCGAGAGGGTGACCATCACCTGCAAGGCCAGCCAGGACGTGAG |
| | sequence | CACCACCGTGGCCTGGTACCAGCAGAAGCCCGGCAAGGCCCCCA |
| | Sequence Name: | AGCTGCTGATCTACTGGGCCAGCACCAGGCAGACCGGCGTGCCC |
| | DAB012131 | AGCAGGTTCAGCGGCAGCGGCAGCGGCACCGACTTCACCCTGAC |
| | | CATCAGCAACCTGCAGCCCGAGGACGTGGCCACCTACTTCTGCCA |
| | | GCAGTACAGCAGCTACCCCTTCACCTTCGGCCAGGGCACCAAGCT |
| | | GGAGATCAAGCGAACGGTGGCTGCCCCCTCCGTGTTCATCTTCCC |
| | | CCCCAGCGATGAGCAGCTGAAGAGCGGCACAGCCAGCGTGGTGT |
| | | GCCTGCTGAACAACTTCTACCCCAGGGAGGCCAAGGTGCAGTGG |
| | | AAGGTGGACAACGCCCTGCAGAGCGGCAACAGCCAGGAGTCCGT |
| | | GACCGAGCAGGACAGCAAGGACAGCACCTACAGCCTGAGCAGCA |
| | | CACTGACCCTGAGCAAGGCCGACTACGAGAAGCACAAGGTGTAC |
| | | GCCTGCGAGGTGACCCATCAGGGCCTGAGCAGCCCCGTGACCAA |
| | | GAGCTTCAACAGGGGCGAGTGC |
| 172 | Full-length light | GACATCCAGATGACCCAGAGCCCCAGCAGCGTGAGCCTGACCGT |
| | chain nucleotide | GGGCCAGAGGGTGACCATCACCTGCCTGGCCACCGAGGACATCTT |
| | sequence | CAGCTACCTGGCCTGGTACCAGCAGAAGCCCGGCAAGGCCCCCA |
| | Sequence Name: | GGCTGCTGATCTACGGCGCCAACAGGCTGAAGGACGGCGTGCCC |
| | DAB012132 | AGCAGGTTCAGCGGCAGCGGCAGCGGCACCCAGTACACCCTGAG |
| | | GATCAGCAGCATGCAGCCCGAGGACTTCGGCGTGTACTACTGCCT |
| | | GCAGGGCGCCAAGTTCCCCCTGACCTTCGGCCCCGGCACCAAGCT |
| | | GGAGATCAAGCGAACGGTGGCTGCCCCCTCCGTGTTCATCTTCCC |
| | | CCCCAGCGATGAGCAGCTGAAGAGCGGCACAGCCAGCGTGGTGT |
| | | GCCTGCTGAACAACTTCTACCCCAGGGAGGCCAAGGTGCAGTGG |
| | | AAGGTGGACAACGCCCTGCAGAGCGGCAACAGCCAGGAGTCCGT |
| | | GACCGAGCAGGACAGCAAGGACAGCACCTACAGCCTGAGCAGCA |
| | | CACTGACCCTGAGCAAGGCCGACTACGAGAAGCACAAGGTGTAC |
| | | GCCTGCGAGGTGACCCATCAGGGCCTGAGCAGCCCCGTGACCAA |
| | | GAGCTTCAACAGGGGCGAGTGC |
| 173 | Full-length light | GACATCCAGATGACCCAGAGCCCCAGCAGCCTGAGCGCCAGCAT |
| | chain nucleotide | CGGCGACACCGTGACCATCACCTGCCTGGCCACCGAGGACATCTT |
| | sequence | CAGCTACCTGGCCTGGTACCAGCAGAGGCCCGGCAAGGCCCCCA |
| | Sequence Name: | AGCTGCTGATCTACGGCGCCAACAGGCTGAAGGACGGCGTGCCC |
| | DAB012133 | AGCAGGTTCAGCGGCAGCGGCAGCGGCACCGACTACACCCTGAC |
| | | CATCAGCGGCCTGCAGCCCGAGGACTTCGTGACCTACTACTGCCT |
| | | GCAGGGCGCCAAGTTCCCCCTGACCTTCGGCCAGGGCACCAAGCT |
| | | GGACATGAAGCGAACGGTGGCTGCCCCCTCCGTGTTCATCTTCCC |
| | | CCCCAGCGATGAGCAGCTGAAGAGCGGCACAGCCAGCGTGGTGT |
| | | GCCTGCTGAACAACTTCTACCCCAGGGAGGCCAAGGTGCAGTGG |
| | | AAGGTGGACAACGCCCTGCAGAGCGGCAACAGCCAGGAGTCCGT |
| | | GACCGAGCAGGACAGCAAGGACAGCACCTACAGCCTGAGCAGCA |
| | | CACTGACCCTGAGCAAGGCCGACTACGAGAAGCACAAGGTGTAC |
| | | GCCTGCGAGGTGACCCATCAGGGCCTGAGCAGCCCCGTGACCAA |
| | | GAGCTTCAACAGGGGCGAGTGC |
| 174 | Full-length light | GACATCCAGATGACCCAGAGCCCCAGCAGCCTGAGCGCCAGCGT |
| | chain nucleotide | GGGCGACACCGTGACCATCACCTGCCTGGCCACCGAGGACATCTT |
| | sequence | CAGCTACCTGGCCTGGTACCAGCAGAAGCCCGGCAAGGCCCCCA |
| | Sequence Name: | AGCTGCTGATCTACGGCGCCAACAGGCTGAAGGACGGCGTGCCC |
| | DAB012134 | AGCAGGTTCAGCGGCAGCGGCAGCGGCACCGACTACACCCTGAC |
| | | CATCAGCGGCCTGCAGCCCGAGGACTTCGGCACCTACTACTGCCT |
| | | GCAGGGCGCCAAGTTCCCCCTGACCTTCGGCCAGGGCACCAAGCT |
| | | GGAGATCAAGCGAACGGTGGCTGCCCCCTCCGTGTTCATCTTCCC |
| | | CCCCAGCGATGAGCAGCTGAAGAGCGGCACAGCCAGCGTGGTGT |
| | | GCCTGCTGAACAACTTCTACCCCAGGGAGGCCAAGGTGCAGTGG |
| | | AAGGTGGACAACGCCCTGCAGAGCGGCAACAGCCAGGAGTCCGT |
| | | GACCGAGCAGGACAGCAAGGACAGCACCTACAGCCTGAGCAGCA |
| | | CACTGACCCTGAGCAAGGCCGACTACGAGAAGCACAAGGTGTAC |
| | | GCCTGCGAGGTGACCCATCAGGGCCTGAGCAGCCCCGTGACCAA |
| | | GAGCTTCAACAGGGGCGAGTGC |
| 175 | Full-length light | GACATCCAGATGACCCAGAGCCCCAGCAGCCTGAGCGCCAGCGT |
| | chain nucleotide | GGGCGACACCGTGACCATCACCTGCCTGGCCACCGAGGACATCTT |
| | sequence | CAGCTACCTGGCCTGGTACCAGCAGAAGCCCGGCAAGGCCCCCA |
| | Sequence Name: | AGCTGCTGATCTACGGCGCCAACAGGCTGAAGGACGGCGTGCCC |
| | DAB012135 | AGCAGGTTCAGCGGCGGCGGCAGCGGCACCGAGTACAGCCTGAC |
| | | CATCAGCGGCCTGCAGCCCGAGGACTTCGGCACCTACTACTGCCT |
| | | GCAGGGCGCCAAGTTCCCCCTGACCTTCGGCCAGGGCACCAAGCT |
| | | GGACATGAAGCGAACGGTGGCTGCCCCCTCCGTGTTCATCTTCCC |
| | | CCCCAGCGATGAGCAGCTGAAGAGCGGCACAGCCAGCGTGGTGT |
| | | GCCTGCTGAACAACTTCTACCCCAGGGAGGCCAAGGTGCAGTGG |
| | | AAGGTGGACAACGCCCTGCAGAGCGGCAACAGCCAGGAGTCCGT |
| | | GACCGAGCAGGACAGCAAGGACAGCACCTACAGCCTGAGCAGCA |
| | | CACTGACCCTGAGCAAGGCCGACTACGAGAAGCACAAGGTGTAC |
| | | GCCTGCGAGGTGACCCATCAGGGCCTGAGCAGCCCCGTGACCAA |
| | | GAGCTTCAACAGGGGCGAGTGC |
| 176 | Full-length light | GACATCCAGATGACCCAGAGCCCCAGCAGCCTGAGCGCCAGCGT |
| | chain nucleotide | GGGCGACACCGTGACCATCACCTGCCTGGCCACCGAGGACATCTT |
| | sequence | CAGCTACCTGGCCTGGTACCAGCAGAAGCCCGGCAAGGCCCCCA |
| | Sequence Name: | AGCTGCTGATCTACGGCGCCAACAGGCTGAAGGACACCGTGCCC |
| | DAB012135 | AGCAGGTTCAGCGGCGGCGGCAGCGGCACCGAGTACAGCCTGAC |
| | (modified) | CATCAGCGGCCTGCAGCCCGAGGACTTCGGCACCTACTACTGCCT |
| | | GCAGGGCGCCAAGTTCCCCCTGACCTTCGGCCAGGGCACCAAGCT |
| | | GGACATGAAGCGAACGGTGGCTGCCCCCTCCGTGTTCATCTTCCC |
| | | CCCCAGCGATGAGCAGCTGAAGAGCGGCACAGCCAGCGTGGTGT |
| | | GCCTGCTGAACAACTTCTACCCCAGGGAGGCCAAGGTGCAGTGG |
| | | AAGGTGGACAACGCCCTGCAGAGCGGCAACAGCCAGGAGTCCGT |
| | | GACCGAGCAGGACAGCAAGGACAGCACCTACAGCCTGAGCAGCA |
| | | CACTGACCCTGAGCAAGGCCGACTACGAGAAGCACAAGGTGTAC |
| | | GCCTGCGAGGTGACCCATCAGGGCCTGAGCAGCCCCGTGACCAA |
| | | GAGCTTCAACAGGGGCGAGTGC |
| 177 | Full-length heavy | CAGGTGCAGCTGCTGGAGAGCGGCGGCGAGCTGGTGCAGCCCGG |
| | chain nucleotide | CGGCAGCCTGAGGCTGAGCTGCGCCGCCAGCGGCTTCACCTTCAG |
| | sequence | CGACTACTACATGTTCTGGATCAGGCAGACCCCCCAGAAGAGGCT |
| | Sequence Name: | GGAGTGGGTGGCCTACATCAGCAACGGCGGCGGCAACACCTACT |
| | DAB012120 | ACCCCGACACCGTGAAGGGCAGGTTCACCATCAGCAGGGACAAC |
| | | GCCAAGAACCAGCTGTACCTGCAGATGAGGAGCCTGACCCCCGA |
| | | GGACACCGCCATCTACTACTGCGCCAGCCCCGAGGCCAGGTACTA |
| | | CGGCAACTACCCCTTCCCCTACTGGGGCCAGGGCACCCTGGTGAC |
| | | CGTGAGCAGCGCTAGCACCAAGGGCCCATCGGTCTTCCCCCTGGC |
| | | ACCCTCCTCCAAGAGCACCTCTGGGGGCACAGCGGCCCTGGGCTG |
| | | CCTGGTCAAGGACTACTTCCCCGAACCGGTGACGGTGTCGTGGAA |
| | | CTCAGGCGCCCTGACCAGCGGCGTGCACACCTTCCCGGCTGTCCT |
| | | ACAGTCCTCAGGACTCTACTCCCTCAGCAGCGTGGTGACCGTGCC |
| | | CTCCAGCAGCTTGGGCACCCAGACCTACATCTGCAACGTGAATCA |
| | | CAAGCCCAGCAACACCAAGGTGGACAAGAAAGTTGAGCCCAAAT |
| | | CTTGTGACAAAACTCACACATGCCCACCGTGCCCAGCACCTGAAC |
| | | TCCTGGGGGGACCGTCAGTCTTCCTCTTCCCCCCAAAACCCAAGG |
| | | ACACCCTCATGATCTCCCGGACCCCTGAGGTCACATGCGTGGTGG |
| | | TGGACGTGAGCCACGAAGACCCTGAGGTCAAGTTCAACTGGTAC |
| | | GTGGACGGCGTGGAGGTGCATAATGCCAAGACAAAGCCGCGGGA |
| | | GGAGCAGTACAACAGCACGTACCGGGTGGTCAGCGTCCTCACCG |
| | | TCCTGCACCAGGACTGGCTGAATGGCAAGGAGTACAAGTGCAAG |
| | | GTCTCCAACAAAGCCCTCCCAGCCCCCATCGAGAAAACCATCTCC |
| | | AAAGCCAAAGGGCAGCCCCGAGAACCACAGGTGTACACCCTGCC |
| | | CCCATCCCGGGATGAGCTGACCAAGAACCAGGTCAGCCTGACCT |
| | | GCCTGGTCAAAGGCTTCTATCCCAGCGACATCGCCGTGGAGTGGG |
| | | AGAGCAATGGGCAGCCGGAGAACAACTACAAGACCACGCCTCCC |
| | | GTGCTGGACTCCGACGGCTCCTTCTTCCTCTACAGCAAGCTCACC |
| | | GTGGACAAGAGCAGGTGGCAGCAGGGGAACGTCTTCTCATGCTC |
| | | CGTGATGCATGAGGCTCTGCACAACCACTACACGCAGAAGAGCC |
| | | TCTCCCTGTCTCCGGGTAAA |
| 178 | Full-length heavy | CAGGTGCAGCTGGTGGAGAGCGGCGGCGGCCTGGTGAAGCCCGG |
| | chain nucleotide | CGGCAGCCTGAGGCTGAGCTGCGCCGCCAGCGGCTTCACCTTCAG |
| | sequence | CGACTACTACATGTTCTGGATCAGGCAGGCCCCCGGCAAGGGCCT |
| | Sequence Name: | GGAGTGGGTGGCCTACATCAGCAACGGCGGCGGCAACACCTACT |
| | DAB012121 | ACCCCGACACCGTGAAGGGCAGGTTCACCATCAGCAGGGACAAC |
| | | GCCAAGAACACCCTGTACCTGCAGATGAACAGCCTGAGGGCCGA |
| | | GGACACCGCCGTGTACTACTGCGCCAGCCCCGAGGCCAGGTACTA |
| | | CGGCAACTACCCCTTCCCCTACTGGGGCCAGGGCACCCTGGTGAC |
| | | CGTGAGCAGCGCTAGCACCAAGGGCCCATCGGTCTTCCCCCTGGC |
| | | ACCCTCCTCCAAGAGCACCTCTGGGGGCACAGCGGCCCTGGGCTG |
| | | CCTGGTCAAGGACTACTTCCCCGAACCGGTGACGGTGTCGTGGAA |
| | | CTCAGGCGCCCTGACCAGCGGCGTGCACACCTTCCCGGCTGTCCT |
| | | ACAGTCCTCAGGACTCTACTCCCTCAGCAGCGTGGTGACCGTGCC |
| | | CTCCAGCAGCTTGGGCACCCAGACCTACATCTGCAACGTGAATCA |
| | | CAAGCCCAGCAACACCAAGGTGGACAAGAAAGTTGAGCCCAAAT |
| | | CTTGTGACAAAACTCACACATGCCCACCGTGCCCAGCACCTGAAC |
| | | TCCTGGGGGGACCGTCAGTCTTCCTCTTCCCCCCAAAACCCAAGG |
| | | ACACCCTCATGATCTCCCGGACCCCTGAGGTCACATGCGTGGTGG |
| | | TGGACGTGAGCCACGAAGACCCTGAGGTCAAGTTCAACTGGTAC |
| | | GTGGACGGCGTGGAGGTGCATAATGCCAAGACAAAGCCGCGGGA |
| | | GGAGCAGTACAACAGCACGTACCGGGTGGTCAGCGTCCTCACCG |
| | | TCCTGCACCAGGACTGGCTGAATGGCAAGGAGTACAAGTGCAAG |
| | | GTCTCCAACAAAGCCCTCCCAGCCCCCATCGAGAAAACCATCTCC |
| | | AAAGCCAAAGGGCAGCCCCGAGAACCACAGGTGTACACCCTGCC |
| | | CCCATCCCGGGATGAGCTGACCAAGAACCAGGTCAGCCTGACCT |
| | | GCCTGGTCAAAGGCTTCTATCCCAGCGACATCGCCGTGGAGTGGG |
| | | AGAGCAATGGGCAGCCGGAGAACAACTACAAGACCACGCCTCCC |
| | | GTGCTGGACTCCGACGGCTCCTTCTTCCTCTACAGCAAGCTCACC |
| | | GTGGACAAGAGCAGGTGGCAGCAGGGGAACGTCTTCTCATGCTC |
| | | CGTGATGCATGAGGCTCTGCACAACCACTACACGCAGAAGAGCC |
| | | TCTCCCTGTCTCCGGGTAAA |
| 179 | Full-length heavy | CAGGTGCAGCTGGTGGAGAGCGGCGGCGGCGTGGTGAAGCCCGG |
| | chain nucleotide | CGGCAGCCTGAGGCTGAGCTGCGCCGCCAGCGGCTTCACCTTCAG |
| | sequence | CGACTACTACATGTTCTGGATCAGGCAGGCCCCCGGCAAGGGCCT |
| | Sequence Name: | GGAGTGGGTGGCCTACATCAGCAACGGCGGCGGCAACACCTACT |
| | DAB012122 | ACCCCGACACCGTGAAGGGCAGGTTCACCATCAGCAGGGACAAC |
| | | GCCAAGAACACCCTGTACCTGCAGATGAACAGCCTGAGGGCCGA |
| | | GGACACCGCCGTGTACTACTGCGCCAGCCCCGAGGCCAGGTACTA |
| | | CGGCAACTACCCCTTCCCCTACTGGGGCCAGGGCACCCTGGTGAC |
| | | CGTGAGCAGCGCTAGCACCAAGGGCCCATCGGTCTTCCCCCTGGC |
| | | ACCCTCCTCCAAGAGCACCTCTGGGGGCACAGCGGCCCTGGGCTG |
| | | CCTGGTCAAGGACTACTTCCCCGAACCGGTGACGGTGTCGTGGAA |
| | | CTCAGGCGCCCTGACCAGCGGCGTGCACACCTTCCCGGCTGTCCT |
| | | ACAGTCCTCAGGACTCTACTCCCTCAGCAGCGTGGTGACCGTGCC |
| | | CTCCAGCAGCTTGGGCACCCAGACCTACATCTGCAACGTGAATCA |
| | | CAAGCCCAGCAACACCAAGGTGGACAAGAAAGTTGAGCCCAAAT |
| | | CTTGTGACAAAACTCACACATGCCCACCGTGCCCAGCACCTGAAC |
| | | TCCTGGGGGGACCGTCAGTCTTCCTCTTCCCCCCAAAACCCAAGG |
| | | ACACCCTCATGATCTCCCGGACCCCTGAGGTCACATGCGTGGTGG |
| | | TGGACGTGAGCCACGAAGACCCTGAGGTCAAGTTCAACTGGTAC |
| | | GTGGACGGCGTGGAGGTGCATAATGCCAAGACAAAGCCGCGGGA |
| | | GGAGCAGTACAACAGCACGTACCGGGTGGTCAGCGTCCTCACCG |
| | | TCCTGCACCAGGACTGGCTGAATGGCAAGGAGTACAAGTGCAAG |
| | | GTCTCCAACAAAGCCCTCCCAGCCCCCATCGAGAAAACCATCTCC |
| | | AAAGCCAAAGGGCAGCCCCGAGAACCACAGGTGTACACCCTGCC |
| | | CCCATCCCGGGATGAGCTGACCAAGAACCAGGTCAGCCTGACCT |
| | | GCCTGGTCAAAGGCTTCTATCCCAGCGACATCGCCGTGGAGTGGG |
| | | AGAGCAATGGGCAGCCGGAGAACAACTACAAGACCACGCCTCCC |
| | | GTGCTGGACTCCGACGGCTCCTTCTTCCTCTACAGCAAGCTCACC |
| | | GTGGACAAGAGCAGGTGGCAGCAGGGGAACGTCTTCTCATGCTC |
| | | CGTGATGCATGAGGCTCTGCACAACCACTACACGCAGAAGAGCC |
| | | TCTCCCTGTCTCCGGGTAAA |
| 180 | Full-length heavy | CAGGTGCAGCTGGTGGAGAGCGGCGGCGGCGTGGTGCAGCCCGG |
| | chain nucleotide | CGGCAGCCTGAGGCTGAGCTGCGCCGCCAGCGGCTTCACCTTCAG |
| | sequence | CGACTACTACATGTTCTGGATCAGGCAGGCCCCCGGCAAGGGCCT |
| | Sequence Name: | GGAGTGGGTGGCCTACATCAGCAACGGCGGCGGCAACACCTACT |
| | DAB012123 | ACCCCGACACCGTGAAGGGCAGGTTCACCATCAGCAGGGACAAC |
| | | GCCAAGAACACCCTGTACCTGCAGATGAACAGCCTGAGGGCCGA |
| | | GGACACCGCCGTGTACTACTGCGCCAGCCCCGAGGCCAGGTACTA |
| | | CGGCAACTACCCCTTCCCCTACTGGGGCCAGGGCACCCTGGTGAC |
| | | CGTGAGCAGCGCTAGCACCAAGGGCCCATCGGTCTTCCCCCTGGC |
| | | ACCCTCCTCCAAGAGCACCTCTGGGGGCACAGCGGCCCTGGGCTG |
| | | CCTGGTCAAGGACTACTTCCCCGAACCGGTGACGGTGTCGTGGAA |
| | | CTCAGGCGCCCTGACCAGCGGCGTGCACACCTTCCCGGCTGTCCT |
| | | ACAGTCCTCAGGACTCTACTCCCTCAGCAGCGTGGTGACCGTGCC |
| | | CTCCAGCAGCTTGGGCACCCAGACCTACATCTGCAACGTGAATCA |
| | | CAAGCCCAGCAACACCAAGGTGGACAAGAAAGTTGAGCCCAAAT |
| | | CTTGTGACAAAACTCACACATGCCCACCGTGCCCAGCACCTGAAC |
| | | TCCTGGGGGGACCGTCAGTCTTCCTCTTCCCCCCAAAACCCAAGG |
| | | ACACCCTCATGATCTCCCGGACCCCTGAGGTCACATGCGTGGTGG |
| | | TGGACGTGAGCCACGAAGACCCTGAGGTCAAGTTCAACTGGTAC |
| | | GTGGACGGCGTGGAGGTGCATAATGCCAAGACAAAGCCGCGGGA |
| | | GGAGCAGTACAACAGCACGTACCGGGTGGTCAGCGTCCTCACCG |
| | | TCCTGCACCAGGACTGGCTGAATGGCAAGGAGTACAAGTGCAAG |
| | | GTCTCCAACAAAGCCCTCCCAGCCCCCATCGAGAAAACCATCTCC |
| | | AAAGCCAAAGGGCAGCCCCGAGAACCACAGGTGTACACCCTGCC |
| | | CCCATCCCGGGATGAGCTGACCAAGAACCAGGTCAGCCTGACCT |
| | | GCCTGGTCAAAGGCTTCTATCCCAGCGACATCGCCGTGGAGTGGG |
| | | AGAGCAATGGGCAGCCGGAGAACAACTACAAGACCACGCCTCCC |
| | | GTGCTGGACTCCGACGGCTCCTTCTTCCTCTACAGCAAGCTCACC |
| | | GTGGACAAGAGCAGGTGGCAGCAGGGGAACGTCTTCTCATGCTC |
| | | CGTGATGCATGAGGCTCTGCACAACCACTACACGCAGAAGAGCC |
| | | TCTCCCTGTCTCCGGGTAAA |
| 181 | Full-length heavy | CAGGTGCAGCTGCTGCAGAGCGGCCCCGGCCTGGTGAAGCCCAG |
| | chain nucleotide | CGCCACCCTGAGCCTGACCTGCACCGTGAGCGGCTTCAGCCTGAC |
| | sequence | CACCTACGGCGTGCACTGGATCAGGCAGCCCCCCGGCAAGGGCC |
| | Sequence Name: | TGGAGTGGATGGGCGTGATCTGGAGCGGCGGCAGCACCGACTAC |
| | DAB012124 | AACGCCGCCTTCATCAGCAGGCTGACCATCAGCAAGGACAACAG |
| | | CAAGAACCAGGTGTTCCTGCAGCTGTACAGCCTGAGGGCCGAGG |
| | | ACACCGCCATCTACTACTGCGCCAGGACCAGCCACTGGTACTTCG |
| | | ACGTGTGGGGCAGGGGCACCCTGGTGACCGTGAGCAGCGCTAGC |
| | | ACCAAGGGCCCATCGGTCTTCCCCCTGGCACCCTCCTCCAAGAGC |
| | | ACCTCTGGGGGCACAGCGGCCCTGGGCTGCCTGGTCAAGGACTAC |
| | | TTCCCCGAACCGGTGACGGTGTCGTGGAACTCAGGCGCCCTGACC |
| | | AGCGGCGTGCACACCTTCCCGGCTGTCCTACAGTCCTCAGGACTC |
| | | TACTCCCTCAGCAGCGTGGTGACCGTGCCCTCCAGCAGCTTGGGC |
| | | ACCCAGACCTACATCTGCAACGTGAATCACAAGCCCAGCAACAC |
| | | CAAGGTGGACAAGAAAGTTGAGCCCAAATCTTGTGACAAAACTC |
| | | ACACATGCCCACCGTGCCCAGCACCTGAACTCCTGGGGGGACCGT |
| | | CAGTCTTCCTCTTCCCCCCAAAACCCAAGGACACCCTCATGATCT |
| | | CCCGGACCCCTGAGGTCACATGCGTGGTGGTGGACGTGAGCCAC |
| | | GAAGACCCTGAGGTCAAGTTCAACTGGTACGTGGACGGCGTGGA |
| | | GGTGCATAATGCCAAGACAAAGCCGCGGGAGGAGCAGTACAACA |
| | | GCACGTACCGGGTGGTCAGCGTCCTCACCGTCCTGCACCAGGACT |
| | | GGCTGAATGGCAAGGAGTACAAGTGCAAGGTCTCCAACAAAGCC |
| | | CTCCCAGCCCCCATCGAGAAAACCATCTCCAAAGCCAAAGGGCA |
| | | GCCCCGAGAACCACAGGTGTACACCCTGCCCCCATCCCGGGATGA |
| | | GCTGACCAAGAACCAGGTCAGCCTGACCTGCCTGGTCAAAGGCTT |
| | | CTATCCCAGCGACATCGCCGTGGAGTGGGAGAGCAATGGGCAGC |
| | | CGGAGAACAACTACAAGACCACGCCTCCCGTGCTGGACTCCGAC |
| | | GGCTCCTTCTTCCTCTACAGCAAGCTCACCGTGGACAAGAGCAGG |
| | | TGGCAGCAGGGGAACGTCTTCTCATGCTCCGTGATGCATGAGGCT |
| | | CTGCACAACCACTACACGCAGAAGAGCCTCTCCCTGTCTCCGGGT |
| | | AAA |
| 182 | Full-length heavy | CAGGTGCAGCTGCAGGAGAGCGGCCCCGGCCTGGTGAAGCCCAG |
| | chain nucleotide | CCAGACCCTGAGCCTGACCTGCACCGTGAGCGGCTTCAGCCTGAC |
| | sequence | CACCTACGGCGTGCACTGGGTGAGGCAGAGCCCCGGCAAGGGCC |
| | Sequence Name: | TGGAGTGGCTGGGCGTGATCTGGAGCGGCGGCAGCACCGACTAC |
| | DAB012125 | AACGCCGCCTTCATCAGCAGGCTGAGCATCAGCAAGGACAACAG |
| | | CAAGAGCCAGGTGTTCCTGGAGATGACCAGCCTGACCGCCGCCG |
| | | ACACCGCCATCTACTACTGCGCCAGGACCAGCCACTGGTACTTCG |
| | | ACGTGTGGGGCAGGGGCACCCTGGTGACCGTGAGCAGCGCTAGC |
| | | ACCAAGGGCCCATCGGTCTTCCCCCTGGCACCCTCCTCCAAGAGC |
| | | ACCTCTGGGGGCACAGCGGCCCTGGGCTGCCTGGTCAAGGACTAC |
| | | TTCCCCGAACCGGTGACGGTGTCGTGGAACTCAGGCGCCCTGACC |
| | | AGCGGCGTGCACACCTTCCCGGCTGTCCTACAGTCCTCAGGACTC |
| | | TACTCCCTCAGCAGCGTGGTGACCGTGCCCTCCAGCAGCTTGGGC |
| | | ACCCAGACCTACATCTGCAACGTGAATCACAAGCCCAGCAACAC |
| | | CAAGGTGGACAAGAAAGTTGAGCCCAAATCTTGTGACAAAACTC |
| | | ACACATGCCCACCGTGCCCAGCACCTGAACTCCTGGGGGGACCGT |
| | | CAGTCTTCCTCTTCCCCCCAAAACCCAAGGACACCCTCATGATCT |
| | | CCCGGACCCCTGAGGTCACATGCGTGGTGGTGGACGTGAGCCAC |
| | | GAAGACCCTGAGGTCAAGTTCAACTGGTACGTGGACGGCGTGGA |
| | | GGTGCATAATGCCAAGACAAAGCCGCGGGAGGAGCAGTACAACA |
| | | GCACGTACCGGGTGGTCAGCGTCCTCACCGTCCTGCACCAGGACT |
| | | GGCTGAATGGCAAGGAGTACAAGTGCAAGGTCTCCAACAAAGCC |
| | | CTCCCAGCCCCCATCGAGAAAACCATCTCCAAAGCCAAAGGGCA |
| | | GCCCCGAGAACCACAGGTGTACACCCTGCCCCCATCCCGGGATGA |
| | | GCTGACCAAGAACCAGGTCAGCCTGACCTGCCTGGTCAAAGGCTT |
| | | CTATCCCAGCGACATCGCCGTGGAGTGGGAGAGCAATGGGCAGC |
| | | CGGAGAACAACTACAAGACCACGCCTCCCGTGCTGGACTCCGAC |
| | | GGCTCCTTCTTCCTCTACAGCAAGCTCACCGTGGACAAGAGCAGG |
| | | TGGCAGCAGGGGAACGTCTTCTCATGCTCCGTGATGCATGAGGCT |
| | | CTGCACAACCACTACACGCAGAAGAGCCTCTCCCTGTCTCCGGGT |
| | | AAA |
| 183 | Full-length heavy | CAGGTGCAGCTGCAGGAGAGCGGCCCCGGCCTGGTGAAGCCCAG |
| | chain nucleotide | CCAGACCCTGAGCCTGACCTGCACCGTGAGCGGCTTCAGCCTGAC |
| | sequence | CACCTACGGCGTGCACTGGGTGAGGCAGGCCCCCGAGAAGGGCC |
| | Sequence Name: | TGGAGTGGCTGGGCGTGATCTGGAGCGGCGGCAGCACCGACTAC |
| | DAB012126 | AACGCCGCCTTCATCAGCAGGCTGAGCATCAGCAAGGACAACAG |
| | | CAAGAGCCAGGTGTTCCTGGAGATGACCAGCCTGACCGCCGACG |
| | | ACACCGCCATCTACTACTGCGCCAGGACCAGCCACTGGTACTTCG |
| | | ACGTGTGGGGCAGGGGCACCCTGGTGACCGTGAGCAGCGCTAGC |
| | | ACCAAGGGCCCATCGGTCTTCCCCCTGGCACCCTCCTCCAAGAGC |
| | | ACCTCTGGGGGCACAGCGGCCCTGGGCTGCCTGGTCAAGGACTAC |
| | | TTCCCCGAACCGGTGACGGTGTCGTGGAACTCAGGCGCCCTGACC |
| | | AGCGGCGTGCACACCTTCCCGGCTGTCCTACAGTCCTCAGGACTC |
| | | TACTCCCTCAGCAGCGTGGTGACCGTGCCCTCCAGCAGCTTGGGC |
| | | ACCCAGACCTACATCTGCAACGTGAATCACAAGCCCAGCAACAC |
| | | CAAGGTGGACAAGAAAGTTGAGCCCAAATCTTGTGACAAAACTC |
| | | ACACATGCCCACCGTGCCCAGCACCTGAACTCCTGGGGGGACCGT |
| | | CAGTCTTCCTCTTCCCCCCAAAACCCAAGGACACCCTCATGATCT |
| | | CCCGGACCCCTGAGGTCACATGCGTGGTGGTGGACGTGAGCCAC |
| | | GAAGACCCTGAGGTCAAGTTCAACTGGTACGTGGACGGCGTGGA |
| | | GGTGCATAATGCCAAGACAAAGCCGCGGGAGGAGCAGTACAACA |
| | | GCACGTACCGGGTGGTCAGCGTCCTCACCGTCCTGCACCAGGACT |
| | | GGCTGAATGGCAAGGAGTACAAGTGCAAGGTCTCCAACAAAGCC |
| | | CTCCCAGCCCCCATCGAGAAAACCATCTCCAAAGCCAAAGGGCA |
| | | GCCCCGAGAACCACAGGTGTACACCCTGCCCCCATCCCGGGATGA |
| | | GCTGACCAAGAACCAGGTCAGCCTGACCTGCCTGGTCAAAGGCTT |
| | | CTATCCCAGCGACATCGCCGTGGAGTGGGAGAGCAATGGGCAGC |
| | | CGGAGAACAACTACAAGACCACGCCTCCCGTGCTGGACTCCGAC |
| | | GGCTCCTTCTTCCTCTACAGCAAGCTCACCGTGGACAAGAGCAGG |
| | | TGGCAGCAGGGGAACGTCTTCTCATGCTCCGTGATGCATGAGGCT |
| | | CTGCACAACCACTACACGCAGAAGAGCCTCTCCCTGTCTCCGGGT |
| | | AAA |
| 184 | Full-length heavy | CAGGTGCAGCTGCAGGAGAGCGGCCCCGGCCTGGTGAAGCCCAG |
| | chain nucleotide | CCAGACCCTGAGCCTGACCTGCACCGTGAGCGGCTTCAGCCTGAC |
| | sequence | CACCTACGGCGTGCACTGGGTGAGGCAGACCCCCGAGAAGGGCC |
| | Sequence Name: | TGGAGTGGCTGGGCGTGATCTGGAGCGGCGGCAGCACCGACTAC |
| | DAB012127 | AACGCCGCCTTCATCAGCAGGCTGAGCATCAGCAAGGACAACAG |
| | | CAAGAGCCAGGTGTTCCTGGAGATGACCAGCCTGACCGCCGACG |
| | | ACACCGCCATCTACTACTGCGCCAGGACCAGCCACTGGTACTTCG |
| | | ACGTGTGGGGCAGGGGCACCCTGGTGACCGTGAGCAGCGCTAGC |
| | | ACCAAGGGCCCATCGGTCTTCCCCCTGGCACCCTCCTCCAAGAGC |
| | | ACCTCTGGGGGCACAGCGGCCCTGGGCTGCCTGGTCAAGGACTAC |
| | | TTCCCCGAACCGGTGACGGTGTCGTGGAACTCAGGCGCCCTGACC |
| | | AGCGGCGTGCACACCTTCCCGGCTGTCCTACAGTCCTCAGGACTC |
| | | TACTCCCTCAGCAGCGTGGTGACCGTGCCCTCCAGCAGCTTGGGC |
| | | ACCCAGACCTACATCTGCAACGTGAATCACAAGCCCAGCAACAC |
| | | CAAGGTGGACAAGAAAGTTGAGCCCAAATCTTGTGACAAAACTC |
| | | ACACATGCCCACCGTGCCCAGCACCTGAACTCCTGGGGGGACCGT |
| | | CAGTCTTCCTCTTCCCCCCAAAACCCAAGGACACCCTCATGATCT |
| | | CCCGGACCCCTGAGGTCACATGCGTGGTGGTGGACGTGAGCCAC |
| | | GAAGACCCTGAGGTCAAGTTCAACTGGTACGTGGACGGCGTGGA |
| | | GGTGCATAATGCCAAGACAAAGCCGCGGGAGGAGCAGTACAACA |
| | | GCACGTACCGGGTGGTCAGCGTCCTCACCGTCCTGCACCAGGACT |
| | | GGCTGAATGGCAAGGAGTACAAGTGCAAGGTCTCCAACAAAGCC |
| | | CTCCCAGCCCCCATCGAGAAAACCATCTCCAAAGCCAAAGGGCA |
| | | GCCCCGAGAACCACAGGTGTACACCCTGCCCCCATCCCGGGATGA |
| | | GCTGACCAAGAACCAGGTCAGCCTGACCTGCCTGGTCAAAGGCTT |
| | | CTATCCCAGCGACATCGCCGTGGAGTGGGAGAGCAATGGGCAGC |
| | | CGGAGAACAACTACAAGACCACGCCTCCCGTGCTGGACTCCGAC |
| | | GGCTCCTTCTTCCTCTACAGCAAGCTCACCGTGGACAAGAGCAGG |
| | | TGGCAGCAGGGGAACGTCTTCTCATGCTCCGTGATGCATGAGGCT |
| | | CTGCACAACCACTACACGCAGAAGAGCCTCTCCCTGTCTCCGGGT |
| | | AAA |
| 185 | Full-length heavy | CAGGTGCAGCTGCTGGAGAGCGGCGGCGGCCTGGTGCAGCCCGG |
| | chain nucleotide | CGGCAGCCTGAGGCTGAGCTGCGCCGCCAGCGGCTTCACCTTCAG |
| | sequence | CGACTACTACATGTACTGGATCAGGCAGACCCCCCAGAAGAGGC |
| | Sequence Name: | TGGAGTGGGTGGCCTACATCAGCAACGGCGGCGGCAACACCTAC |
| | DAB012128 | TACAGCGACACCGTGAAGGGCAGGTTCACCATCAGCAGGGACAA |
| | | CGCCAAGAACCAGCTGTACCTGCAGATGAGGAGCCTGAGGCCCG |
| | | AGGACACCGCCATCTACTACTGCGCCAGCCCCGAGGCCAGGTACT |
| | | ACGGCAACTTCCCCTTCCCCTACTGGGGCCAGGGCACCCTGGTGA |
| | | CCGTGAGCAGCGCTAGCACCAAGGGCCCATCGGTCTTCCCCCTGG |
| | | CACCCTCCTCCAAGAGCACCTCTGGGGGCACAGCGGCCCTGGGCT |
| | | GCCTGGTCAAGGACTACTTCCCCGAACCGGTGACGGTGTCGTGGA |
| | | ACTCAGGCGCCCTGACCAGCGGCGTGCACACCTTCCCGGCTGTCC |
| | | TACAGTCCTCAGGACTCTACTCCCTCAGCAGCGTGGTGACCGTGC |
| | | CCTCCAGCAGCTTGGGCACCCAGACCTACATCTGCAACGTGAATC |
| | | ACAAGCCCAGCAACACCAAGGTGGACAAGAAAGTTGAGCCCAAA |
| | | TCTTGTGACAAAACTCACACATGCCCACCGTGCCCAGCACCTGAA |
| | | CTCCTGGGGGGACCGTCAGTCTTCCTCTTCCCCCCAAAACCCAAG |
| | | GACACCCTCATGATCTCCCGGACCCCTGAGGTCACATGCGTGGTG |
| | | GTGGACGTGAGCCACGAAGACCCTGAGGTCAAGTTCAACTGGTA |
| | | CGTGGACGGCGTGGAGGTGCATAATGCCAAGACAAAGCCGCGGG |
| | | AGGAGCAGTACAACAGCACGTACCGGGTGGTCAGCGTCCTCACC |
| | | GTCCTGCACCAGGACTGGCTGAATGGCAAGGAGTACAAGTGCAA |
| | | GGTCTCCAACAAAGCCCTCCCAGCCCCCATCGAGAAAACCATCTC |
| | | CAAAGCCAAAGGGCAGCCCCGAGAACCACAGGTGTACACCCTGC |
| | | CCCCATCCCGGGATGAGCTGACCAAGAACCAGGTCAGCCTGACCT |
| | | GCCTGGTCAAAGGCTTCTATCCCAGCGACATCGCCGTGGAGTGGG |
| | | AGAGCAATGGGCAGCCGGAGAACAACTACAAGACCACGCCTCCC |
| | | GTGCTGGACTCCGACGGCTCCTTCTTCCTCTACAGCAAGCTCACC |
| | | GTGGACAAGAGCAGGTGGCAGCAGGGGAACGTCTTCTCATGCTC |
| | | CGTGATGCATGAGGCTCTGCACAACCACTACACGCAGAAGAGCC |
| | | TCTCCCTGTCTCCGGGTAAA |
| 186 | Full-length heavy | CAGGTGCAGCTGGTGGAGAGCGGCGGCGGCCTGGTGAAGCCCGG |
| | chain nucleotide | CGGCAGCCTGAGGCTGAGCTGCGCCGCCAGCGGCTTCACCTTCAG |
| | sequence | CGACTACTACATGTACTGGATCAGGCAGGCCCCCGGCAAGGGCCT |
| | Sequence Name: | GGAGTGGGTGGCCTACATCAGCAACGGCGGCGGCAACACCTACT |
| | DAB012129 | ACAGCGACACCGTGAAGGGCAGGTTCACCATCAGCAGGGACAAC |
| | | GCCAAGAACACCCTGTACCTGCAGATGAACAGCCTGAGGGCCGA |
| | | GGACACCGCCGTGTACTACTGCGCCAGCCCCGAGGCCAGGTACTA |
| | | CGGCAACTTCCCCTTCCCCTACTGGGGCCAGGGCACCCTGGTGAC |
| | | CGTGAGCAGCGCTAGCACCAAGGGCCCATCGGTCTTCCCCCTGGC |
| | | ACCCTCCTCCAAGAGCACCTCTGGGGGCACAGCGGCCCTGGGCTG |
| | | CCTGGTCAAGGACTACTTCCCCGAACCGGTGACGGTGTCGTGGAA |
| | | CTCAGGCGCCCTGACCAGCGGCGTGCACACCTTCCCGGCTGTCCT |
| | | ACAGTCCTCAGGACTCTACTCCCTCAGCAGCGTGGTGACCGTGCC |
| | | CTCCAGCAGCTTGGGCACCCAGACCTACATCTGCAACGTGAATCA |
| | | CAAGCCCAGCAACACCAAGGTGGACAAGAAAGTTGAGCCCAAAT |
| | | CTTGTGACAAAACTCACACATGCCCACCGTGCCCAGCACCTGAAC |
| | | TCCTGGGGGGACCGTCAGTCTTCCTCTTCCCCCCAAAACCCAAGG |
| | | ACACCCTCATGATCTCCCGGACCCCTGAGGTCACATGCGTGGTGG |
| | | TGGACGTGAGCCACGAAGACCCTGAGGTCAAGTTCAACTGGTAC |
| | | GTGGACGGCGTGGAGGTGCATAATGCCAAGACAAAGCCGCGGGA |
| | | GGAGCAGTACAACAGCACGTACCGGGTGGTCAGCGTCCTCACCG |
| | | TCCTGCACCAGGACTGGCTGAATGGCAAGGAGTACAAGTGCAAG |
| | | GTCTCCAACAAAGCCCTCCCAGCCCCCATCGAGAAAACCATCTCC |
| | | AAAGCCAAAGGGCAGCCCCGAGAACCACAGGTGTACACCCTGCC |
| | | CCCATCCCGGGATGAGCTGACCAAGAACCAGGTCAGCCTGACCT |
| | | GCCTGGTCAAAGGCTTCTATCCCAGCGACATCGCCGTGGAGTGGG |
| | | AGAGCAATGGGCAGCCGGAGAACAACTACAAGACCACGCCTCCC |
| | | GTGCTGGACTCCGACGGCTCCTTCTTCCTCTACAGCAAGCTCACC |
| | | GTGGACAAGAGCAGGTGGCAGCAGGGGAACGTCTTCTCATGCTC |
| | | CGTGATGCATGAGGCTCTGCACAACCACTACACGCAGAAGAGCC |
| | | TCTCCCTGTCTCCGGGTAAA |
| 187 | Full-length heavy | CAGGTGCAGCTGGTGGAGAGCGGCGGCGGCCTGGTGAAGCCCGG |
| | chain nucleotide | CGGCAGCCTGAGGCTGAGCTGCGCCGCCAGCGGCTTCACCTTCAG |
| | sequence | CGACTACTACATGTACTGGATCAGGCAGGCCCCCGGCAAGGGCCT |
| | Sequence Name: | GGAGTGGGTGGCCTACATCAGCAACGGCGGCGGCAACACCTACT |
| | DAB012130 | ACAGCGACACCGTGAAGGGCAGGTTCACCATCAGCAGGGACAAC |
| | | GCCAAGAACACCCTGTACCTGCAGATGAACAGCCTGAGGGCCGA |
| | | GGACACCGCCGTGTACTACTGCGCCAGCCCCGAGGCCAGGTACTA |
| | | CGGCAACTTCCCCTTCCCCTACTGGGGCCAGGGCACCCTGGTGAC |
| | | CGTGAGCAGCGCTAGCACCAAGGGCCCATCGGTCTTCCCCCTGGC |
| | | ACCCTCCTCCAAGAGCACCTCTGGGGGCACAGCGGCCCTGGGCTG |
| | | CCTGGTCAAGGACTACTTCCCCGAACCGGTGACGGTGTCGTGGAA |
| | | CTCAGGCGCCCTGACCAGCGGCGTGCACACCTTCCCGGCTGTCCT |
| | | ACAGTCCTCAGGACTCTACTCCCTCAGCAGCGTGGTGACCGTGCC |
| | | CTCCAGCAGCTTGGGCACCCAGACCTACATCTGCAACGTGAATCA |
| | | CAAGCCCAGCAACACCAAGGTGGACAAGAAAGTTGAGCCCAAAT |
| | | CTTGTGACAAAACTCACACATGCCCACCGTGCCCAGCACCTGAAC |
| | | TCCTGGGGGGACCGTCAGTCTTCCTCTTCCCCCCAAAACCCAAGG |
| | | ACACCCTCATGATCTCCCGGACCCCTGAGGTCACATGCGTGGTGG |
| | | TGGACGTGAGCCACGAAGACCCTGAGGTCAAGTTCAACTGGTAC |
| | | GTGGACGGCGTGGAGGTGCATAATGCCAAGACAAAGCCGCGGGA |
| | | GGAGCAGTACAACAGCACGTACCGGGTGGTCAGCGTCCTCACCG |
| | | TCCTGCACCAGGACTGGCTGAATGGCAAGGAGTACAAGTGCAAG |
| | | GTCTCCAACAAAGCCCTCCCAGCCCCCATCGAGAAAACCATCTCC |
| | | AAAGCCAAAGGGCAGCCCCGAGAACCACAGGTGTACACCCTGCC |
| | | CCCATCCCGGGATGAGCTGACCAAGAACCAGGTCAGCCTGACCT |
| | | GCCTGGTCAAAGGCTTCTATCCCAGCGACATCGCCGTGGAGTGGG |
| | | AGAGCAATGGGCAGCCGGAGAACAACTACAAGACCACGCCTCCC |
| | | GTGCTGGACTCCGACGGCTCCTTCTTCCTCTACAGCAAGCTCACC |
| | | GTGGACAAGAGCAGGTGGCAGCAGGGGAACGTCTTCTCATGCTC |
| | | CGTGATGCATGAGGCTCTGCACAACCACTACACGCAGAAGAGCC |
| | | TCTCCCTGTCTCCGGGTAAA |
| 188 | Full-length heavy | CAGGTGCAGCTGGTGGAGAGCGGCGGCGGCCTGGTGAAGCCCGG |
| | chain nucleotide | CGGCAGCCTGAGGCTGAGCTGCGCCGCCAGCGGCTTCACCTTCAG |
| | sequence | CGACTACTACATGTACTGGATCAGGCAGGCCCCCGGCAAGGGCCT |
| | Sequence Name: | GGAGTGGGTGGCCTACATCAGCAACGGCGGCGGCAACACCTACT |
| | DAB012131 | ACAGCGACACCGTGAAGGGCAGGTTCACCATCAGCAGGGACAAC |
| | | GCCAAGAACACCCTGTACCTGCAGATGAACAGCCTGAGGGCCGA |
| | | GGACACCGCCGTGTACTACTGCGCCAGCCCCGAGGCCAGGTACTA |
| | | CGGCAACTTCCCCTTCCCCTACTGGGGCCAGGGCACCCTGGTGAC |
| | | CGTGAGCAGCGCTAGCACCAAGGGCCCATCGGTCTTCCCCCTGGC |
| | | ACCCTCCTCCAAGAGCACCTCTGGGGGCACAGCGGCCCTGGGCTG |
| | | CCTGGTCAAGGACTACTTCCCCGAACCGGTGACGGTGTCGTGGAA |
| | | CTCAGGCGCCCTGACCAGCGGCGTGCACACCTTCCCGGCTGTCCT |
| | | ACAGTCCTCAGGACTCTACTCCCTCAGCAGCGTGGTGACCGTGCC |
| | | CTCCAGCAGCTTGGGCACCCAGACCTACATCTGCAACGTGAATCA |
| | | CAAGCCCAGCAACACCAAGGTGGACAAGAAAGTTGAGCCCAAAT |
| | | CTTGTGACAAAACTCACACATGCCCACCGTGCCCAGCACCTGAAC |
| | | TCCTGGGGGGACCGTCAGTCTTCCTCTTCCCCCCAAAACCCAAGG |
| | | ACACCCTCATGATCTCCCGGACCCCTGAGGTCACATGCGTGGTGG |
| | | TGGACGTGAGCCACGAAGACCCTGAGGTCAAGTTCAACTGGTAC |
| | | GTGGACGGCGTGGAGGTGCATAATGCCAAGACAAAGCCGCGGGA |
| | | GGAGCAGTACAACAGCACGTACCGGGTGGTCAGCGTCCTCACCG |
| | | TCCTGCACCAGGACTGGCTGAATGGCAAGGAGTACAAGTGCAAG |
| | | GTCTCCAACAAAGCCCTCCCAGCCCCCATCGAGAAAACCATCTCC |
| | | AAAGCCAAAGGGCAGCCCCGAGAACCACAGGTGTACACCCTGCC |
| | | CCCATCCCGGGATGAGCTGACCAAGAACCAGGTCAGCCTGACCT |
| | | GCCTGGTCAAAGGCTTCTATCCCAGCGACATCGCCGTGGAGTGGG |
| | | AGAGCAATGGGCAGCCGGAGAACAACTACAAGACCACGCCTCCC |
| | | GTGCTGGACTCCGACGGCTCCTTCTTCCTCTACAGCAAGCTCACC |
| | | GTGGACAAGAGCAGGTGGCAGCAGGGGAACGTCTTCTCATGCTC |
| | | CGTGATGCATGAGGCTCTGCACAACCACTACACGCAGAAGAGCC |
| | | TCTCCCTGTCTCCGGGTAAA |
| 189 | Full-length heavy | CAGGTGCAGCTGGTGGAGAGCGGCGGCGGCGTGGTGCAGCCCGG |
| | chain nucleotide | CAGGAGCCTGAGGCTGAGCTGCGCCGCCAGCGGCTTCAGCTTCAG |
| | sequence | CAACTACTACATGGCCTGGGTGAGGCAGGCCCCCGGCAAGGGCC |
| | Sequence Name: | TGGAGTGGGTGGCCAGCATCAGCACCGGCGGCGGCAACATCTAC |
| | DAB012132 | TACAGGGACAGCGTGAAGGGCAGGTTCACCATCAGCAGGGACAA |
| | | CAGCAAGAGCACCCTGTACCTGCAGATGAGGAGCCTGAGGAGCG |
| | | AGGACACCGCCATCTACTACTGCGCCAGGCAGACCGCCTACTACG |
| | | TGATGGACGCCTGGGGCCAGGGCACCATGGTGACCGTGAGCAGC |
| | | GCTAGCACCAAGGGCCCATCGGTCTTCCCCCTGGCACCCTCCTCC |
| | | AAGAGCACCTCTGGGGGCACAGCGGCCCTGGGCTGCCTGGTCAA |
| | | GGACTACTTCCCCGAACCGGTGACGGTGTCGTGGAACTCAGGCGC |
| | | CCTGACCAGCGGCGTGCACACCTTCCCGGCTGTCCTACAGTCCTC |
| | | AGGACTCTACTCCCTCAGCAGCGTGGTGACCGTGCCCTCCAGCAG |
| | | CTTGGGCACCCAGACCTACATCTGCAACGTGAATCACAAGCCCAG |
| | | CAACACCAAGGTGGACAAGAAAGTTGAGCCCAAATCTTGTGACA |
| | | AAACTCACACATGCCCACCGTGCCCAGCACCTGAACTCCTGGGGG |
| | | GACCGTCAGTCTTCCTCTTCCCCCCAAAACCCAAGGACACCCTCA |
| | | TGATCTCCCGGACCCCTGAGGTCACATGCGTGGTGGTGGACGTGA |
| | | GCCACGAAGACCCTGAGGTCAAGTTCAACTGGTACGTGGACGGC |
| | | GTGGAGGTGCATAATGCCAAGACAAAGCCGCGGGAGGAGCAGTA |
| | | CAACAGCACGTACCGGGTGGTCAGCGTCCTCACCGTCCTGCACCA |
| | | GGACTGGCTGAATGGCAAGGAGTACAAGTGCAAGGTCTCCAACA |
| | | AAGCCCTCCCAGCCCCCATCGAGAAAACCATCTCCAAAGCCAAA |
| | | GGGCAGCCCCGAGAACCACAGGTGTACACCCTGCCCCCATCCCG |
| | | GGATGAGCTGACCAAGAACCAGGTCAGCCTGACCTGCCTGGTCA |
| | | AAGGCTTCTATCCCAGCGACATCGCCGTGGAGTGGGAGAGCAAT |
| | | GGGCAGCCGGAGAACAACTACAAGACCACGCCTCCCGTGCTGGA |
| | | CTCCGACGGCTCCTTCTTCCTCTACAGCAAGCTCACCGTGGACAA |
| | | GAGCAGGTGGCAGCAGGGGAACGTCTTCTCATGCTCCGTGATGCA |
| | | TGAGGCTCTGCACAACCACTACACGCAGAAGAGCCTCTCCCTGTC |
| | | TCCGGGTAAA |
| 190 | Full-length heavy | GAGGTGCAGCTGGTGGAGAGCGGCGGCGGCCTGGTGCAGCCCGG |
| | chain nucleotide | CGGCAGCCTGAGGCTGAGCTGCGCCGCCAGCGGCTTCAGCTTCAG |
| | sequence | CAACTACTACATGGCCTGGGTGAGGCAGGCCCCCGGCAAGGGCC |
| | Sequence Name: | TGGAGTGGGTGGCCAGCATCAGCACCGGCGGCGGCAACATCTAC |
| | DAB012133 | TACAGGGACAGCGTGAAGGGCAGGTTCACCATCAGCAGGGACAA |
| | | CGCCAAGAACAGCCTGTACCTGCAGATGAACAGCCTGAGGGCCG |
| | | AGGACACCGCCGTGTACTACTGCGCCAGGCAGACCGCCTACTACG |
| | | TGATGGACGCCTGGGGCCAGGGCACCACCGTGACCGTGAGCAGC |
| | | GCTAGCACCAAGGGCCCATCGGTCTTCCCCCTGGCACCCTCCTCC |
| | | AAGAGCACCTCTGGGGGCACAGCGGCCCTGGGCTGCCTGGTCAA |
| | | GGACTACTTCCCCGAACCGGTGACGGTGTCGTGGAACTCAGGCGC |
| | | CCTGACCAGCGGCGTGCACACCTTCCCGGCTGTCCTACAGTCCTC |
| | | AGGACTCTACTCCCTCAGCAGCGTGGTGACCGTGCCCTCCAGCAG |
| | | CTTGGGCACCCAGACCTACATCTGCAACGTGAATCACAAGCCCAG |
| | | CAACACCAAGGTGGACAAGAAAGTTGAGCCCAAATCTTGTGACA |
| | | AAACTCACACATGCCCACCGTGCCCAGCACCTGAACTCCTGGGGG |
| | | GACCGTCAGTCTTCCTCTTCCCCCCAAAACCCAAGGACACCCTCA |
| | | TGATCTCCCGGACCCCTGAGGTCACATGCGTGGTGGTGGACGTGA |
| | | GCCACGAAGACCCTGAGGTCAAGTTCAACTGGTACGTGGACGGC |
| | | GTGGAGGTGCATAATGCCAAGACAAAGCCGCGGGAGGAGCAGTA |
| | | CAACAGCACGTACCGGGTGGTCAGCGTCCTCACCGTCCTGCACCA |
| | | GGACTGGCTGAATGGCAAGGAGTACAAGTGCAAGGTCTCCAACA |
| | | AAGCCCTCCCAGCCCCCATCGAGAAAACCATCTCCAAAGCCAAA |
| | | GGGCAGCCCCGAGAACCACAGGTGTACACCCTGCCCCCATCCCG |
| | | GGATGAGCTGACCAAGAACCAGGTCAGCCTGACCTGCCTGGTCA |
| | | AAGGCTTCTATCCCAGCGACATCGCCGTGGAGTGGGAGAGCAAT |
| | | GGGCAGCCGGAGAACAACTACAAGACCACGCCTCCCGTGCTGGA |
| | | CTCCGACGGCTCCTTCTTCCTCTACAGCAAGCTCACCGTGGACAA |
| | | GAGCAGGTGGCAGCAGGGGAACGTCTTCTCATGCTCCGTGATGCA |
| | | TGAGGCTCTGCACAACCACTACACGCAGAAGAGCCTCTCCCTGTC |
| | | TCCGGGTAAA |
| 191 | Full-length heavy | GAGGTGCAGCTGGTGGAGAGCGGCGGCGGCCTGGTGCAGCCCGG |
| | chain nucleotide | CGGCAGCCTGAGGCTGAGCTGCGCCGCCAGCGGCTTCAGCTTCAG |
| | sequence | CAACTACTACATGGCCTGGGTGAGGCAGGCCCCCGGCAAGGGCC |
| | Sequence Name: | TGGAGTGGGTGGCCAGCATCAGCACCGGCGGCGGCAACATCTAC |
| | DAB012134 | TACAGGGACAGCGTGAAGGGCAGGTTCACCATCAGCAGGGACAA |
| | | CGCCAAGAACAGCCTGTACCTGCAGATGAACAGCCTGAGGGCCG |
| | | AGGACACCGCCGTGTACTACTGCGCCAGGCAGACCGCCTACTACG |
| | | TGATGGACGCCTGGGGCCAGGGCACCACCGTGACCGTGAGCAGC |
| | | GCTAGCACCAAGGGCCCATCGGTCTTCCCCCTGGCACCCTCCTCC |
| | | AAGAGCACCTCTGGGGGCACAGCGGCCCTGGGCTGCCTGGTCAA |
| | | GGACTACTTCCCCGAACCGGTGACGGTGTCGTGGAACTCAGGCGC |
| | | CCTGACCAGCGGCGTGCACACCTTCCCGGCTGTCCTACAGTCCTC |
| | | AGGACTCTACTCCCTCAGCAGCGTGGTGACCGTGCCCTCCAGCAG |
| | | CTTGGGCACCCAGACCTACATCTGCAACGTGAATCACAAGCCCAG |
| | | CAACACCAAGGTGGACAAGAAAGTTGAGCCCAAATCTTGTGACA |
| | | AAACTCACACATGCCCACCGTGCCCAGCACCTGAACTCCTGGGGG |
| | | GACCGTCAGTCTTCCTCTTCCCCCCAAAACCCAAGGACACCCTCA |
| | | TGATCTCCCGGACCCCTGAGGTCACATGCGTGGTGGTGGACGTGA |
| | | GCCACGAAGACCCTGAGGTCAAGTTCAACTGGTACGTGGACGGC |
| | | GTGGAGGTGCATAATGCCAAGACAAAGCCGCGGGAGGAGCAGTA |
| | | CAACAGCACGTACCGGGTGGTCAGCGTCCTCACCGTCCTGCACCA |
| | | GGACTGGCTGAATGGCAAGGAGTACAAGTGCAAGGTCTCCAACA |
| | | AAGCCCTCCCAGCCCCCATCGAGAAAACCATCTCCAAAGCCAAA |
| | | GGGCAGCCCCGAGAACCACAGGTGTACACCCTGCCCCCATCCCG |
| | | GGATGAGCTGACCAAGAACCAGGTCAGCCTGACCTGCCTGGTCA |
| | | AAGGCTTCTATCCCAGCGACATCGCCGTGGAGTGGGAGAGCAAT |
| | | GGGCAGCCGGAGAACAACTACAAGACCACGCCTCCCGTGCTGGA |
| | | CTCCGACGGCTCCTTCTTCCTCTACAGCAAGCTCACCGTGGACAA |
| | | GAGCAGGTGGCAGCAGGGGAACGTCTTCTCATGCTCCGTGATGCA |
| | | TGAGGCTCTGCACAACCACTACACGCAGAAGAGCCTCTCCCTGTC |
| | | TCCGGGTAAA |
| 192 | Full-length heavy | GAGGTGCAGCTGGTGGAGAGCGGCGGCGGCCTGGTGCAGCCCGG |
| | chain nucleotide | CGGCAGCCTGAGGCTGAGCTGCGCCGCCAGCGGCTTCAGCTTCAG |
| | sequence | CAACTACTACATGGCCTGGGTGAGGCAGGCCCCCGGCAAGGGCC |
| | Sequence Name: | TGGAGTGGGTGGCCAGCATCAGCACCGGCGGCGGCAACATCTAC |
| | DAB012135 | TACAGGGACAGCGTGAAGGGCAGGTTCACCATCAGCAGGGACAA |
| | | CGCCAAGAACAGCCTGTACCTGCAGATGAACAGCCTGAGGGCCG |
| | | AGGACACCGCCGTGTACTACTGCGCCAGGCAGACCGCCTACTACG |
| | | TGATGGACGCCTGGGGCCAGGGCACCACCGTGACCGTGAGCAGC |
| | | GCTAGCACCAAGGGCCCATCGGTCTTCCCCCTGGCACCCTCCTCC |
| | | AAGAGCACCTCTGGGGGCACAGCGGCCCTGGGCTGCCTGGTCAA |
| | | GGACTACTTCCCCGAACCGGTGACGGTGTCGTGGAACTCAGGCGC |
| | | CCTGACCAGCGGCGTGCACACCTTCCCGGCTGTCCTACAGTCCTC |
| | | AGGACTCTACTCCCTCAGCAGCGTGGTGACCGTGCCCTCCAGCAG |
| | | CTTGGGCACCCAGACCTACATCTGCAACGTGAATCACAAGCCCAG |
| | | CAACACCAAGGTGGACAAGAAAGTTGAGCCCAAATCTTGTGACA |
| | | AAACTCACACATGCCCACCGTGCCCAGCACCTGAACTCCTGGGGG |
| | | GACCGTCAGTCTTCCTCTTCCCCCCAAAACCCAAGGACACCCTCA |
| | | TGATCTCCCGGACCCCTGAGGTCACATGCGTGGTGGTGGACGTGA |
| | | GCCACGAAGACCCTGAGGTCAAGTTCAACTGGTACGTGGACGGC |
| | | GTGGAGGTGCATAATGCCAAGACAAAGCCGCGGGAGGAGCAGTA |
| | | CAACAGCACGTACCGGGTGGTCAGCGTCCTCACCGTCCTGCACCA |
| | | GGACTGGCTGAATGGCAAGGAGTACAAGTGCAAGGTCTCCAACA |
| | | AAGCCCTCCCAGCCCCCATCGAGAAAACCATCTCCAAAGCCAAA |
| | | GGGCAGCCCCGAGAACCACAGGTGTACACCCTGCCCCCATCCCG |
| | | GGATGAGCTGACCAAGAACCAGGTCAGCCTGACCTGCCTGGTCA |
| | | AAGGCTTCTATCCCAGCGACATCGCCGTGGAGTGGGAGAGCAAT |
| | | GGGCAGCCGGAGAACAACTACAAGACCACGCCTCCCGTGCTGGA |
| | | CTCCGACGGCTCCTTCTTCCTCTACAGCAAGCTCACCGTGGACAA |
| | | GAGCAGGTGGCAGCAGGGGAACGTCTTCTCATGCTCCGTGATGCA |
| | | TGAGGCTCTGCACAACCACTACACGCAGAAGAGCCTCTCCCTGTC |
| | | TCCGGGTAAA |
| 193 | Sequence encoding | GGAGGATGGTCCTGCGCCCCCGACGAGGACACCTGGCTGTGCCCC |
| | the light chain | GCCGGCGGAGGAGGCTCTGGAGGACTGTCTGGCAGGTCCGACGC |
| | of the | CGGCTCCCCTCTGGGCCTGGCTGGCAGCGGCGGCTCTGACATCCA |
| | Nectin-4 TRACTr | GATGACACAGTCCCCATCCAGCCTGTCCGCCTCCGTGGGCGACAC |
| | molecule | CGTGACAATCACCTGTCTGGCCACCGAGGACATCTTCTCCTACCT |
| | | GGCTTGGTATCAGCAGAAGCCAGGCAAGGCCCCCAAGCTGCTGA |
| | | TCTACGGCGCTAATAGGCTGAAGGACACAGTGCCATCCCGGTTCA |
| | | GCGGAGGAGGCTCCGGCACAGAGTATTCTCTGACCATCTCCGGCC |
| | | TGCAGCCTGAGGATTTTGGCACCTACTATTGCCTGCAGGGCGCCA |
| | | AGTTCCCACTGACATTTGGCCAGGGCACCAAGCTGGACATGAAG |
| | | AGAACAGTGGCCGCTCCCTCCGTGTTCATCTTTCCCCCTAGCGAT |
| | | GAGCAGCTGAAGAGCGGCACCGCTTCTGTGGTGTGCCTGCTGAAC |
| | | AATTTCTACCCTCGCGAGGCCAAGGTGCAGTGGAAGGTGGACAA |
| | | CGCTCTGCAGTCTGGCAATTCCCAGGAGAGCGTGACAGAGCAGG |
| | | ACTCTAAGGATTCCACCTATAGCCTGTCTTCCACACTGACCCTGTC |
| | | TAAGGCCGATTACGAGAAGCACAAGGTGTATGCTTGCGAGGTGA |
| | | CACATCAGGGCCTGTCCTCCCCCGTGACCAAGTCCTTTAACAGAG |
| | | GCGAGTGT |
| 194 | Sequence encoding | GAGGTGCAGCTGGTGGAGTCCGGAGGAGGACTGGTGCAGCCTGG |
| | the heavy chain of | CGGCTCCCTGAGACTGAGCTGTGCCGCTTCTGGCTCCACATTCTA |
| | the Nectin-4 | CACCGCCGTGATGGGATGGGTGAGGCAGGCTCCAGGCAAGGGCC |
| | TRACTr molecule | TGGAGTGGGTGGCTGCTATCAGGTGGACAGCCCTGACCACATCTT |
| | | ATGCTGACTCCGTGAAGGGCAGATTCACCATCTCCCGCGATGGCG |
| | | CCAAGACCACACTGTACCTGCAGATGAACAGCCTGAGACCTGAG |
| | | GACACAGCCGTGTACTATTGCGCTGCTCGCGGCACCCTGGGACTG |
| | | TTTACCACAGCTGACTCCTACGATTATTGGGGCCAGGGCACACTG |
| | | GTGACCGTGTCCAGCGGCGGAGGAGGCAGCGGAGGAGGCTCTGG |
| | | CGGCGTGTACTGCGGCCCAGAGTTCGACGAGTCCGTGGGCTGTAT |
| | | GGGCGGCGGAGGCAGCGGAGGAGGACTGTCCGGCAGAAGCGAT |
| | | GCTGGCTCCCCACTGGGCCTGGCTGGCTCCGGAGGAGGCAGCGA |
| | | AGTCCAGCTGGTGGAGAGCGGCGGCGGCCTGGTCCAGCCTGGCG |
| | | GCTCTCTGAAGCTGTCCTGTGCCGCCTCCGGCTTCACCTTTAACAA |
| | | GTATGCCATGAATTGGGTGCGCCAGGCTCCCGGCAAGGGCCTGG |
| | | AGTGGGTAGCCAGGATCAGGTCCAAGTACAACAATTATGCCACCT |
| | | ACTACGCCGACTCCGTGAAGGATAGGTTCACAATCTCTCGGGACG |
| | | ATTCCAAGAACACCGCCTACCTGCAGATGAACAATCTGAAGACA |
| | | GAGGACACCGCCGTGTACTATTGCGTGAGGCACGGCAACTTTGGC |
| | | AATTCTTACATCTCCTATTGGGCTTACTGGGGTCAGGGCACACTG |
| | | GTCACCGTGTCTTCCGGAGGAGGAGGCTCCGGCGGCGGAGGCAG |
| | | CGGCGGCGGCGGCTCTCAGACAGTGGTGACCCAGGAGCCAAGCC |
| | | TGACCGTGTCTCCCGGCGGCACCGTGACACTGACCTGTGGCAGCT |
| | | CTACAGGAGCTGTGACCAGCGGAAACTATCCAAATTGGGTGCAG |
| | | CAGAAGCCTGGCCAGGCTCCTAGAGGCCTGATCGGAGGCACAAA |
| | | GTTCCTGGCCCCAGGCACCCCAGCTCGCTTTAGCGGCTCTCTGCT |
| | | GGGAGGCAAGGCCGCTCTGACCCTGAGCGGAGTGCAGCCAGAGG |
| | | ATGAGGCCGAGTACTATTGCGTGCTGTGGTACTCTAACAGATGGG |
| | | TGTTTGGCGGCGGCACAAAGCTGACCGTGCTGGGAGGAGGAGGC |
| | | AGCGAAGTGCAGCTGGTCGAGTCTGGCGGCGGCTTAGTCCAACCT |
| | | GGCGGCTCCCTGAGGCTGTCTTGCGCCGCTTCTGGCTTCTCCTTTA |
| | | GCAACTACTATATGGCTTGGGTGCGGCAGGCTCCTGGCAAGGGCC |
| | | TGGAGTGGGTCGCCTCTATCTCCACAGGCGGCGGCAATATCTACT |
| | | ATCGGGACTCTGTGAAGGGCAGGTTCACCATCTCCAGGGACAAC |
| | | GCTAAGAATAGCCTGTATCTGCAGATGAACTCCCTGAGGGCCGAA |
| | | GATACTGCCGTGTACTACTGCGCCCGGCAGACCGCTTACTATGTG |
| | | ATGGATGCCTGGGGCCAGGGCACCACAGTGACAGTGTCCAGCGC |
| | | CTCCACCAAGGGCCCTAGCGTGTTCCCTCTGGCTCCATCTTCCAA |
| | | GAGCACATCTGGAGGCACCGCCGCTCTGGGATGTCTGGTGAAGG |
| | | ACTACTTCCCCGAGCCTGTGACCGTGAGCTGGAACTCTGGCGCCC |
| | | TGACATCTGGCGTGCACACCTTTCCCGCTGTGCTGCAGTCCTCCG |
| | | GCCTGTATTCCCTGTCCAGCGTGGTGACAGTGCCTTCTTCCAGCCT |
| | | GGGCACACAGACCTACATCTGCAACGTGAATCATAAGCCTAGCA |
| | | ATACCAAGGTGGATAAGAAGGTGGAGCCAAAGTCTTGT |

What is claimed is:

1. A polypeptide complex, comprising:
(1) a first polypeptide chain comprising:
a. an anti-albumin (ALB) heavy chain variable region comprising a CDRH1 comprising the amino acid sequence of SEQ ID NO: 4; a CDRH2 comprising the amino acid sequence of SEQ ID NO: 5; and a CDRH3 comprising the amino acid sequence of SEQ ID NO: 6;
b. a first mask sequence comprising the amino acid sequence of SEQ ID NO: 7;
c. a first protease cleavage site comprising the amino acid sequence of SEQ ID NO: 8;
d. an anti-CD3 heavy chain variable region comprising a CDRH1 comprising the amino acid sequence of SEQ ID NO: 9; a CDRH2 comprising the amino acid sequence of SEQ ID NO: 10; and a CDRH3 comprising the amino acid sequence of SEQ ID NO: 11;
e. an anti-CD3 light chain variable region comprising a CDRL1 comprising the amino acid sequence of SEQ ID NO: 12; a CDRL2 comprising the amino acid sequence of SEQ ID NO: 13; and a CDRL3 comprising the amino acid sequence of SEQ ID NO: 14; and
f. an anti-Nectin-4 heavy chain variable region comprising a CDRH1 comprising the amino acid sequence of SEQ ID NO: 15; a CDRH2 comprising the amino acid sequence of SEQ ID NO: 16; a CDRH3 comprising the amino acid sequence of SEQ ID NO: 17,
and
(ii) a second polypeptide chain comprising:
a. a second mask sequence comprising the amino acid sequence of SEQ ID NO: 18;
b. a second protease cleavage site comprising the amino acid sequence of SEQ ID NO: 19; and
c. an anti-Nectin-4 light chain variable region comprising a CDRL1 comprising the amino acid sequence of SEQ ID NO: 20; a CDRL2 comprising the amino acid sequence of SEQ ID NO: 21; and a CDRL3 comprising the amino acid sequence of SEQ ID NO: 22.

2. The polypeptide complex of claim 1, wherein the first protease cleavage site is upstream of the anti-CD3 heavy chain variable region and upstream of the anti-CD3 light chain variable region, and wherein the first protease cleavage site is downstream of the first mask sequence.

3. The polypeptide complex of claim 1, wherein the second protease cleavage site is upstream of the anti-Nectin-4 light chain variable region and wherein the second protease cleavage site is downstream of the second mask sequence.

4. The polypeptide complex of claim 1, wherein the first polypeptide chain further comprises a leader sequence comprising the amino acid sequence of claim 3.

5. The polypeptide complex of claim 1, wherein the second polypeptide chain further comprises a leader sequence comprising the amino acid sequence of claim 3.

6. The polypeptide complex of claim 1, wherein the first mask sequence prevents binding of the anti-CD3 heavy chain variable region to CD3 and wherein the first mask sequence prevents binding of the anti-CD3 light chain variable region variable region to CD3.

7. The polypeptide complex of claim 1, wherein the second mask sequence prevents binding of the anti-Nectin-4 light chain variable region to Nectin-4.

8. The polypeptide complex of claim 1, wherein the anti-Nectin-4 heavy chain variable region comprises the any one of the amino acid sequences of SEQ ID NOs: 69, 70, 71, and 72.

9. The polypeptide complex of claim 8, wherein the anti-Nectin-4 heavy chain variable region comprises the amino acid sequence of SEQ ID NO: 72.

10. The polypeptide complex of claim 1, wherein the anti-Nectin-4 light chain variable region comprises any one of the amino acid sequences of SEQ ID NO: 43, 44, 45, 46, and 47.

11. The polypeptide complex of claim 10, wherein the anti-Nectin-4 light chain variable region comprises the amino acid sequence of SEQ ID NO: 47.

12. A polypeptide complex, comprising:
(i) a first polypeptide chain comprising an amino acid sequence that is at least 95% identical to SEQ ID NO: 1; and
(ii) a second polypeptide chain comprising an amino acid sequence that is at least 95% identical to SEQ ID NO: 2;
wherein the polypeptide complex binds to Nectin-4 and CD3.

13. The polypeptide complex of claim 12, wherein the first polypeptide chain comprises the amino acid sequence of SEQ ID NO: 1.

14. The polypeptide complex of claim 12, wherein the second polypeptide chain comprises the amino acid sequence of SEQ ID NO: 2.

15. The polypeptide complex of claim 12, wherein the first polypeptide chain comprises the amino acid sequence of SEQ ID NO: 1 and the second polypeptide chain comprises the amino acid sequence of SEQ ID NO: 2.

16. A polypeptide complex, comprising:
(i) a first polypeptide chain comprising:
a. an anti-albumin (ALB) heavy chain variable region comprising a CDRH1 comprising the amino acid sequence of SEQ ID NO: 4; a CDRH2 comprising the amino acid sequence of SEQ ID NO: 5; and a CDRH3 comprising the amino acid sequence of SEQ ID NO: 6;
b. a first mask sequence comprising the amino acid sequence of SEQ ID NO: 7;
c. a first protease cleavage site comprising the amino acid sequence of SEQ ID NO: 8;
d. an anti-CD3 heavy chain variable region comprising a CDRH1 comprising the amino acid sequence of SEQ ID NO: 9; a CDRH2 comprising the amino acid sequence of SEQ ID NO: 10; and a CDRH3 comprising the amino acid sequence of SEQ ID NO: 11;
e. an anti-CD3 light chain variable region comprising a CDRL1 comprising the amino acid sequence of SEQ ID NO: 12; a CDRL2 comprising the amino acid sequence of SEQ ID NO: 13; and a CDRL3 comprising the amino acid sequence of SEQ ID NO: 14; and
f. a Nectin-4 heavy chain variable region comprising:
1. aCDRH1 comprising the amino acid sequence of SEQ ID NO: 52; a CDRH2 comprising the amino acid sequence of SEQ ID NO: 53; and a CDRH3 comprising the amino acid sequence of SEQ ID NO: 54;
2. a CDRH1 comprising the amino acid sequence of SEQ ID NO: 59; a CDRH2 comprising the amino acid sequence of SEQ ID NO: 60; and a CDRH3 comprising the amino acid sequence of SEQ ID NO: 61; or
3. a CDRH1 comprising the amino acid sequence of SEQ ID NO: 66; a CDRH2 comprising the amino acid sequence of SEQ ID NO: 67; and a CDRH3 comprising the amino acid sequence of SEQ ID NO: 68,
and
(ii) a second polypeptide chain comprising:
a. a second mask sequence comprising the amino acid sequence of any one of SEQ ID NOs: 18, 23, 125, and 126;
b. a second protease cleavage site comprising the amino acid sequence of SEQ ID NO: 19; and
c. an anti-Nectin-4 light chain variable region comprising:
1. a CDRL1 comprising the amino acid sequence of SEQ ID NO: 28; a CDRL2 comprising the amino acid sequence of SEQ ID NO: 29; and a CDRL3 comprising the amino acid sequence of SEQ ID NO: 30;
2. a CDRL1 comprising the amino acid sequence of SEQ ID NO: 35; a CDRL2 comprising the amino acid sequence of SEQ ID NO: 36; and a CDRL3 comprising the amino acid sequence of SEQ ID NO: 37; or
3. a CDRL1 comprising the amino acid sequence of SEQ ID NO: 42; a CDRL2 comprising the amino acid sequence of SEQ ID NO: 29; and a CDRL3 comprising the amino acid sequence of SEQ ID NO: 30.

17. The polypeptide complex of claim 16, wherein the anti-Nectin-4 heavy chain variable region comprises a CDRH1 comprising the amino acid sequence of SEQ ID NO: 52; a CDRH2 comprising the amino acid sequence of SEQ ID NO: 53; and a CDRH3 comprising the amino acid sequence of SEQ ID NO: 54, and
wherein the anti-Nectin-4 light chain variable region comprises a CDRL1 comprising the amino acid sequence of SEQ ID NO: 28; a CDRL2 comprising the amino acid sequence of SEQ ID NO: 29; and a CDRL3 comprising the amino acid sequence of SEQ ID NO: 30.

18. The polypeptide complex of claim 16, wherein
the anti-Nectin-4 heavy chain variable region comprises an amino acid sequence that is at least 95% identical to any one of SEQ ID NOs: 48, 49, 50, and 51, and
the anti-Nectin-4 light chain variable region comprises a sequence that is at least 95% identical to any one of SEQ ID NOs: 24, 25, 26, and 27, wherein the polypeptide complex binds to Nectin-4.

19. The polypeptide complex of claim 16, wherein
the anti-Nectin-4 heavy chain variable region comprises a CDRH1 comprising the amino acid sequence of SEQ ID NO: 59; a CDRH2 comprising the amino acid sequence of SEQ ID NO: 60;
and a CDRH3 comprising the amino acid sequence of SEQ ID NO: 61, and the anti-Nectin-4 light chain variable region comprises a CDRL1 comprising the amino acid sequence of SEQ ID NO: 35; a CDRL2 comprising the amino acid sequence of SEQ ID NO: 36;
and a CDRL3 comprising the amino acid sequence of SEQ ID NO: 37.

20. The polypeptide complex of claim 16, wherein
the anti-Nectin-4 heavy chain variable region comprises an amino acid sequence that is at least 95% identical to the amino acid sequence of any one of SEQ ID NOs: 55, 56, 58, and 59, and the anti-Nectin-4 light chain variable region comprises an amino acid sequence that is at least 95% identical to the amino acid sequence of any one of SEQ ID NOs: 31, 32, 33, and 34, wherein the polypeptide complex is capable of binding to Nectin-4.

21. The polypeptide complex of claim 16, wherein
the anti-Nectin-4 heavy chain variable region comprises a CDRH1 comprising the amino acid sequence of SEQ ID NO: 66; a CDRH2 comprising the amino acid sequence of SEQ ID NO: 67;
and a CDRH3 comprising the amino acid sequence of SEQ ID NO: 68, and the anti-Nectin-4 light chain variable region comprises a CDRL1 comprising the amino acid sequence of SEQ ID NO: 42; a CDRL2 comprising the amino acid sequence of SEQ ID NO: 29;
and a CDRL3 comprising the amino acid sequence of SEQ ID NO: 30.

22. The polypeptide complex of claim 16, wherein
the anti-Nectin-4 heavy chain variable region comprises an amino acid sequence that is at least 95% identical to the amino acid sequence of any one of SEQ ID NOs: 62, 63, 64, and 65, and the anti-Nectin-4 light chain variable region comprises an amino acid sequence that is at least 95% identical to the amino acid sequence of any one of SEQ ID NOs: 38, 39, 40, and 41, wherein the polypeptide complex is capable of binding to Nectin-4.

23. The polypeptide complex of claim 16, wherein the second mask sequence comprises SEQ ID NO: 18.

24. The polypeptide complex of claim 16, wherein the second mask sequence comprises SEQ ID NO: 23.

25. The polypeptide complex of claim 16, wherein the second mask sequence comprises SEQ ID NO: 125.

26. The polypeptide complex of claim 16, wherein the second mask sequence comprises SEQ ID NO: 126.

27. The polypeptide complex of claim 16, wherein the first mask sequence prevents binding of the CD3 heavy chain to CD3 and wherein the first mask sequence prevents binding of the CD3 light chain variable region variable region to CD3.

28. The polypeptide complex of claim 16, wherein the second mask sequence prevents binding of the Nectin-4 light chain variable region to Nectin-4.

29. The polypeptide complex of claim 16, wherein the first polypeptide chain further comprises a leader sequence comprising the amino acid sequence of claim 3.

30. The polypeptide complex of claim 16, wherein the second polypeptide chain further comprises a leader sequence comprising the amino acid sequence of claim 3.

31. A composition comprising the polypeptide complex of claim 15 and a pharmaceutically acceptable carrier.

32. A method of treating cancer in a subject in need thereof comprising administering to the subject an effective amount of a polypeptide complex of claim 15.

33. The method of claim 32, wherein the cancer comprises breast cancer, bladder cancer, lung cancer, urothelial cancer, colorectal cancer, cervical cancer, pancreatic cancer, head and neck squamous cell carcinoma (HNSCC), or ovarian cancer.

34. The method of claim 32, wherein the cancer comprises bladder cancer, HNSCC, cervical cancer, breast cancer, or pancreatic cancer.

35. A polynucleotide encoding the first polypeptide chain and/or the second polypeptide chain of the polypeptide complex of claim 15.

36. An expression vector comprising the polynucleotide of claim 35.

37. A host cell comprising the polynucleotide of claim 35.

38. A method of producing a polypeptide complex that binds to Nectin-4 and CD3 comprising:
(a) culturing a host cell in culture medium, wherein the host cell comprises one or more vectors comprising a polynucleotide encoding the first polypeptide chain and a polynucleotide encoding the second polypeptide chain of the polypeptide complex of claim 15, under conditions that allow expression of the polypeptide chains, and
(b) optionally recovering the polypeptide complex from the host cell or culture medium.

39. The polypeptide complex of claim 15 for use in a method of treating cancer.

40. The polypeptide complex or the composition for use of claim 39, wherein the cancer comprises bladder cancer, HNSCC, cervical cancer, breast cancer, or pancreatic cancer.
